(19) United States
(12) Patent Application Publication
(10) Pub. No.: US 2012/0010241 A1
(43) Pub. Date: Jan. 12, 2012

(54) HSP90 INHIBITING INDAZOLE DERIVATIVES, COMPOSITIONS CONTAINING SAME AND USE THEREOF

(75) Inventors: Luc Bertin, Paris (FR); Jean-Christophe Carry, Paris (FR); Patrick Mailliet, Paris (FR); Herve Minoux, Paris (FR); Fabienne Pilorge, Paris (FR); Jean-Marie Ruxer, Paris (FR)

(73) Assignee: SANOFI, Paris (FR)

(21) Appl. No.: 13/257,516

(22) PCT Filed: Mar. 18, 2010
(86) PCT No.: PCT/FR10/50483

§ 371 Date: Sep. 19, 2011

(30) Foreign Application Priority Data
Mar. 19, 2009 (FR) 0901274
Jan. 19, 2010 (FR) 1050341

(51) Int. Cl. A61K 31/4709; A61P 35/00; A61K 31/4184; C07D 401/10; C07D 403/10

(52) U.S. Cl. 514/314; 546/173; 548/305.1; 514/394

(57) ABSTRACT

The invention relates to novel products having formula (I), wherein: R4 represents H, CH3, CH2CH3, CF3, F, Cl, Br, I; Het represents a heterocycle optionally substituted by one or more R1 or R′1 radicals selected from H, halogen, CF3, nitro, cyano, alkyl, hydroxy, mercapto, amino, alkylamino, dialkylamino, alkoxy, phenylalkoxy, alkylthio, carboxy that is free or sterified with an alkyl radical, carboxamide, CO—NH(alkyl), CON(alkyl)2, NH—CO-alkyl, sulfonamide, NH—SO2-alkyl, S(O)2-NHalkyl, S(O2)-N(alkyl)2, all of the alkyl, alkoxy and alkylthio radicals being optionally substituted; R being selected from the group comprising (A′), (B), (C), (D) and (F), wherein W1, W2, W3 represent independently CH or N, X represents O, S, NR2, C(O), S(O) or S(O)2; V represents H, Hal, —O—R2 or —NH—R2 with R2 representing H, alkyl, cycloalkyl or heterocycloalkyl, optionally substituted; said products being in all isomer forms, as well as the salts and intended for use as drugs.

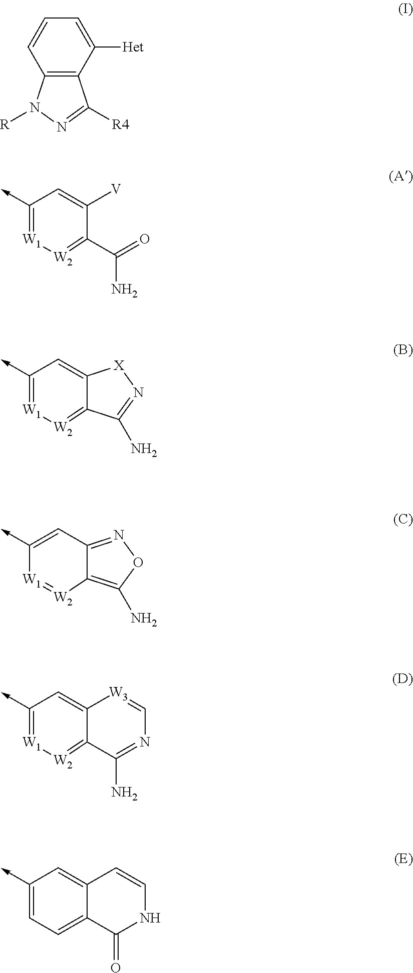

The present invention relates to novel chemical compounds which are heterocyclic derivatives of indazole, to the compositions which contain them, and to the use thereof as medicaments.

More particularly, according to a first aspect, the invention relates to novel heterocyclic derivatives of indazole displaying anticancer activity, and in particular Hsp90 chaperone protein-inhibiting activity, and more particularly via inhibition of the ATPase-type catalytic activity of the Hsp90 chaperone protein.

### Chaperone Proteins:

The molecular chaperones of the “Heat Shock Protein” (HSP) family, which are classified according to their molecular weight (Hsp27, Hsp70, Hsp90, etc.) are key elements in the equilibrium between the synthesis and the degradation of cellular proteins responsible for correct protein folding. They play a vital role in response to cellular stress. HSPs, and in particular Hsp90, are also involved in the regulation of various very important functions of the cell, via their association with various client proteins involved in cell proliferation or apoptosis (Jolly C. and Morimoto R. I., J. N. Cancer Inst. (2000), 92, 1564-72; Smith D. F. et al., Pharmacological Rev. (1998), 50, 493-513; Smith D. F., Molecular Chaperones in the Cell, 165-178, Oxford University Press 2001).

### Hsp90 Chaperone and Hsp90 Inhibitors in Cancer Treatment:

The Hsp90 chaperone, which represents 1 to 2% of the protein content of the cell, has recently been demonstrated as a particularly promising target in anticancer therapy (cf. for review: Moloney A. and Workman P., Expert Opin. Biol. Ther. (2002), 2(1), 3-24; Chiosis et al, Drug Discovery Today (2004), 9, 881-888). This interest relates in particular to the cytoplasmic interactions of Hsp90 with the main client proteins of Hsp90, which proteins are involved in the six mechanisms of tumour progression, as defined by Hanahan D. and Weinberg R. A. (Cell (2002), 100, 57-70), namely;

an ability to proliferate in the absence of growth factors; EGFR-R/HER2, Src, Akt, Raf, MEK, Bcr-Abl, Flt-3, etc.,

an ability to evade apoptosis: mutated form of p53, Akt, survivin, etc.,

an insensitivity to signals to halt proliferation: Cdk4, Plk, Wee1, etc.,

an ability to activate angiogenesis: VEGF-R, FAK, HIF-1, Akt, etc.,

an ability to proliferate without replicative limit; hTert, etc.,

an ability to invade new tissues and to metastasize: c-Met.

Among the other client proteins of Hsp90, steroid hormone receptors, such as the oestrogen receptor or the androgen receptor, are also of considerable interest in the context of anticancer therapies.

It has recently been shown that the alpha form of Hsp90 also has an extracellular role via its interaction with the MMP-2 metalloprotease, which is itself involved in tumour invasion (Eustace B. K. et al, Nature Cell Biology (2004), 6, 507-514). Hsp90 is made up of two N- and C-terminal domains separated by a highly charged region. The dynamic interaction between these two domains, coordinated by the binding of nucleotides and of co-chaperones, determines the conformation of the chaperone and its state of activation. The association of the client proteins depends mainly on the nature of the co-chaperones Hsp70/Hsp40. Hop60, etc., and on the nature of the ADP or ATP nucleotide bound to the N-terminal domain of Hsp90. Thus, the hydrolysis of ATP to ADP and the ADP/ATP exchange factor control all of the chaperone “machinery”, and it has been shown that it is sufficient to prevent the hydrolysis of ATP to ADP—ATPase activity of Hsp90—in order to release client proteins in the cytoplasm, which client proteins will then be degraded by the proteasome (Neckers Land Neckers K. Expert Opin, Emerging Drugs (2002), 7, 277-288; Neckers L, Current Medicinal Chemistry, (2003), 10, 733-739; Piper P. W., Current Opin. Invest. New Drugs (2001), 2, 1606-1610).

Role of Hsp90 and of Inhibitors Thereof in Pathologies Other than Cancer:

Various human pathologies are the consequence of incorrect folding of key proteins, resulting in particular in neurodegenerative diseases following the aggregation of certain proteins, such as in Alzheimer's disease and Huntington's disease or prion-related diseases (Tytell M. and Hooper P. L., Emerging Ther. Targets (2001), 5, 267-287). In these pathologies, approaches aimed at inhibiting Hsp90 for the purpose of activating the stress pathways (Hsp70, for example) could be beneficial (Nature Reviews Neuroscience 6: 11, 2005). Some examples are mentioned below:
i) Huntington's disease: This neurodegenerative disease is due to an extension of CAG triplets in exon 1 of the gene encoding the huntingtin protein. It has been shown that geldanamycin inhibits the aggregation of this protein due to the overexpression of the Hsp70 and Hsp40 chaperones (Human Molecular Genetics 10: 1307, 2001).
ii) Parkinson's disease: This disease is due to the progressive loss of dopaminergic neurons and is characterized by aggregation of the alpha-synuclein protein. It has been shown that geldanamycin is capable of protecting drosophila against the toxicity of alpha-synuclein on dopaminergic neurons.
iii) Focal cerebral ischaemia: It has been shown, in a rat animal model, that geldanamycin protects the brain against cerebral ischaemia, due to the effect of stimulation of the transcription of genes encoding the heat shock proteins by an Hsp90 inhibitor.
iv) Alzheimer's disease and multiple sclerosis: These diseases are due in part to the expression of pro-inflammatory cytokines and of the inducible form of NOS (Nitric Oxide Synthase) in the brain, and this harmful expression is suppressed by the response to stress. In particular, the Hsp90 inhibitors are capable of garnering this response to stress, and it has been shown, in vitro, that geldanamycin and 17-AAG exhibit anti-inflammatory activity in brain gliale cells (J. Neuroscience Res. 67: 461, 2002).
v) Amyotrophic lateral sclerosis: This neurodegenerative disease is due to the progressive loss of motor neurons. It has been shown that arimoclomol, an inducer of heat-shock proteins, delays the progression of the disease in an animal model (Nature Medicine 10: 402, 2004). Given that an Hsp90 inhibitor is also an inducer of heat-shock proteins (Mol. Cell. Biol. 19: 8033, 1999; Mol. Cell. Biol. 18: 4949, 1998), it is probable that a beneficial effect could also be obtained in this pathology for inhibitors of this type.

Furthermore, an inhibitor of the Hsp90 protein could potentially be of use in various diseases, other than cancer mentioned above, such as parasitic, viral or fungal diseases or neurodegenerative diseases, by virtue of a direct action on Hsp90 and specific client proteins. Some examples are given below:
vi) Malaria: the Hsp90 protein of Plasmodium falciparum exhibits 59% identity and 69% similarity with the human Hsp90 protein, and it has been shown that geldanamycin inhibits the growth of the parasite in vitro (Malaria Journal 2: 30, 2003; J. Biol. Chem. 278: 18336, 2003; J. Biol, Chem. 279: 46692, 2004).
vii) Brugia filariasis and Bancroft's filariasis: these lymphatic filarial parasites possess an Hsp90 protein which can potentially be inhibited with inhibitors of the human protein. In fact, it has been shown, for another similar parasite, Brugia pahangi, that the latter is sensitive to inhibition with geldanamycin. The B. pahangi and human sequences are 80% identical and 87% similar (Int. J. for Parasitology 35: 627, 2005).
viii) Toxoplasmosis: Toxoplasma gondii, the parasite responsible for toxoplasmosis, has an Hsp90 chaperone protein for which induction has been shown during tachyzoite-bradyzoite conversion, corresponding to passage from chronic infection to active toxoplasmosis. Furthermore, geldanamycin blocks this tachyzoite-bradyzoite conversion in vitro (J. Mol. Biol. 350: 723, 2005).
ix) Treatment-resistant mycoses: It is possible that the Hsp90 protein potentiates the evolution of drug resistance by allowing new mutations to develop. Consequently, an Hsp90 inhibitor, alone or in combination with another antifungal treatment, could prove to be of use in the treatment of certain resistant strains (Science 309: 2185, 2005). Furthermore, the anti-Hsp90 antibody developed by Neu Tec Pharma demonstrates an activity against C. albicans, which is sensitive and resistant to fluconazole, C. krusei, C. tropicalis, C. glabrata, C. lusitaniae and C. parapsilosis in vivo (Current Molecular Medicine 5: 403, 2005).
x) Hepatitis B: Hsp90 is one of the host proteins which interacts with the reverse transcriptase of the hepatitis B virus during the replication cycle of the virus. It has been shown that geldanamycin inhibits replication of the viral DNA and encapsulation of the viral RNA (Proc. Natl. Acad. Sci. USA 93: 1060, 1996).
xi) Hepatitis C; The human Hsp90 protein participates in the step consisting of cleavage between the NS2 and NS3 proteins by the viral protease. Geldanamycin and radicicol are capable of inhibiting this NS2/3 cleavage in vitro (Proc. Natl. Acad. Sci. USA 98: 13931, 2001).
xii) The Herpes virus: Geldanamycin has demonstrated inhibitory activities on HSV-1 virus replication in vitro, with a good therapeutic index (Antimicrobial Agents and Chemotherapy 48: 867, 2004). The authors have also found geldanamycin activity on the other viruses HSV-2, VSV, Cox B3, HIV-1 and the SARS coronavirus (data not shown).

xiii) Dengue (or tropical flu); It has been shown that the human Hsp90 protein participates in the virus entry step, by forming a complex also containing Hsp70 which serves as a receptor for the virus; an anti-Hsp90 antibody decreases the infectious capacity of the virus in vitro (J. of Virology 79: 4557, 2005)
xiv) Spinal and bulbar muscular atrophy (SBMA): A hereditary neurodegenerative disease characterized by an extension of CAG triplets in the androgen receptor gene. It has been shown that 17-AAG, a geldanamycin derivative, exhibits activity in vivo on transgenic animals used as experimental models for this disease (Nature Medicine 11: 1088, 2005).

### Hsp90 Inhibitors:

The first known Hsp90 inhibitors are compounds of the ansamycin family, in particular geldanamycin (1) and herbimycin A. X-ray studies have shown that geldanamycin binds to the ATP site of the N-terminal domain of Hsp90, where it inhibits the ATPase activity of the chaperone (Prodromou C. et al, Cell (1997), 90, 65-75).

Currently, the NIH and Kosan BioSciences are carrying out the clinical development of 17-AAG (2), which is an Hsp90 inhibitor derived from geldanamycin (1), which blocks the ATPase activity of Hsp90 by binding to the N-terminal ATP recognition site. The results of phase I clinical trials for 17-AAG (1) have now led to phase II trials being started, but have also directed research towards derivatives which are more soluble, such as analogue 3 (17-DMAG from Kosan BioSciences), which carries a dimethyl amino chain in place of the methoxy residue, and towards optimized formulations of 17AAG (CNF1010 from Conforma Therapeutics):

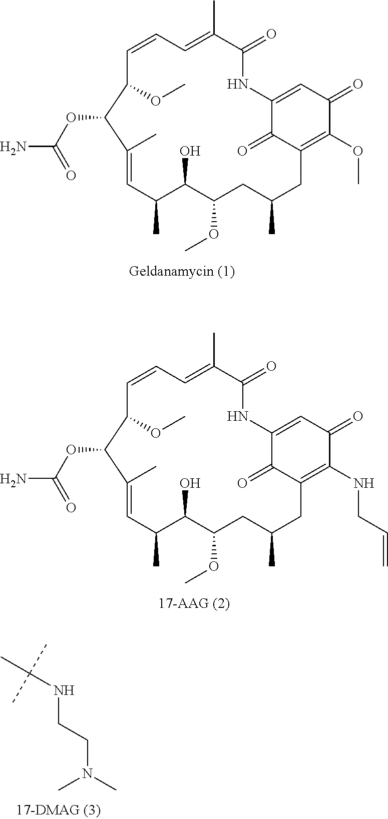

The reduced analogue of 17AAG (WO 2005/063714/US 2006/019941) has also since relatively recently been undergoing phase I clinical studies by the company Infinity Pharmaceuticals. Novel geldanamycin derivatives or ansamycin derivatives have recently been described (WO2006/016773/U.S. Pat. No. 6,855,705/US 2005/026894/WO2006/050477/US2006/205705/WO2007/001049/WO2007/064926/WO2007/074347/WO2007/098229/WO2007/128827/WO2007/128829).

Radicicol (4) is also an Hsp90 inhibitor of natural origin (Roe S. M. et al, J. Med. Chem. (1999), 42, 260-66). However, although the latter is by far the best in vitro inhibitor of Hsp90, its metabolic instability with respect to sulphur-containing nucleophiles makes it difficult to use in vivo. Oxime derivatives that are much more stable, such as KF 55823 (5) or KF 25706, have been developed by the company Kyowa Hakko Kogyo (Soga et al, Cancer Research (1999), 59, 2931-2938).

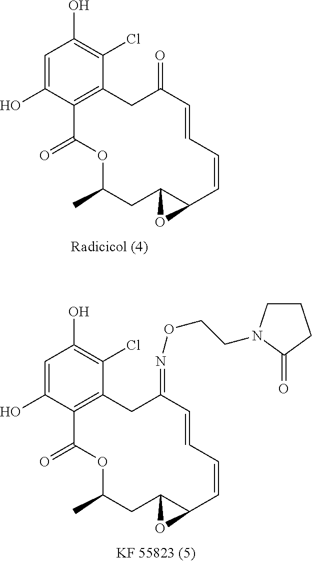

Structures of natural origin related to radicicol have also recently been described, such as zearalenone (6) by the company Conforma Therapeutics (WO 2003/041643) or compounds (7-9).

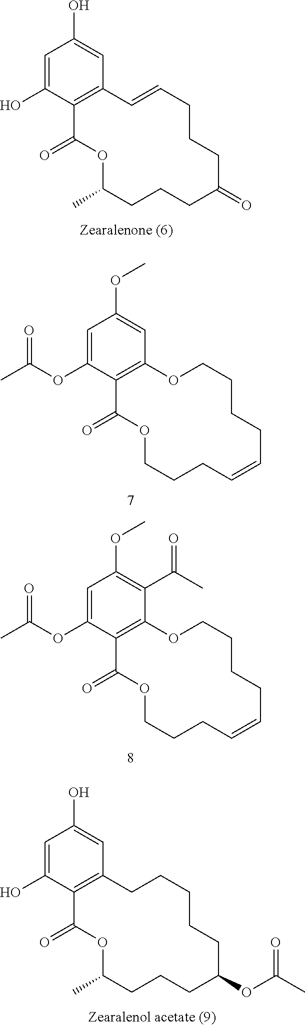

Patent application US 2006/089495 describes mixed compounds comprising a quinone ring, such as the ansamycin derivatives, and a resorcinol ring, such as the radicicol analogues, as Hsp90 inhibitors.

An Hsp90 inhibitor of natural origin, novobiocin (10), binds to a different ATP site located in the C-terminal domain of the protein (Itoh H. et al, Biochem J. (1999), 343, 697-703). Simplified analogues of novobiocin have recently been identified as more powerful inhibitors of Hsp90 than novobiocin itself (J. Amer. Chem. Soc, (2005), 127(37), 12778-12779).

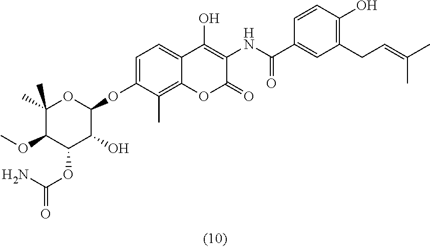

Patent applications WO2006/050501 and US2007/270452 claim novobiocin analogues as Hsp90 inhibitors.

Patent application WO2007/117466 claims derivatives of celastrol and of gedunine as Hsp90 inhibitors,

A depsipeptide, called pipalamycin or 101101, has also been described as a non-competitive inhibitor of the ATP site of Hsp90 (J. Pharmacol. Exp. Ther. (2004), 310, 1288-1295).

Sherperdine, a KHSSGCAFL nonapeptide, mimics a part of the K79-K90 sequence (KHSSGCAFLSVK) of survivin and blocks the interaction of proteins of the IAP family with Hsp90 in vitro (WO 2006/014744).

Small peptides, comprising a sequence of otoferlin-type (YSLPGYMVKKLLGA), have recently been described as Hsp90 inhibitors (WO 2005/072766).

Purines, such as the compounds PU3 (11) (Chiosis et al, Chem. Biol. (2001), 8, 289-299) and PU24FCl (12) (Chiosis et al, Curr. Canc. Drug Targets (2003), 3, 371-376; WO 2002/036075) have also been described as Hsp90 inhibitors:

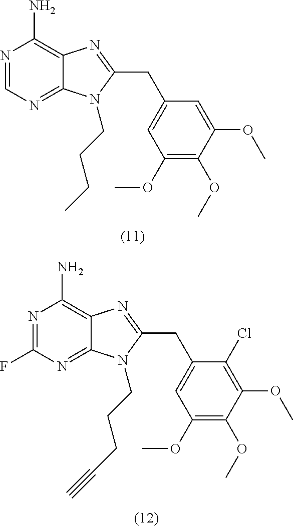

A purine derivative, CNF2024 (13), has recently been introduced clinically by the company Conforma Therapeutics, in collaboration with the Sloan Kettering Memorial Institute for Cancer Research (WO 2006/084030).

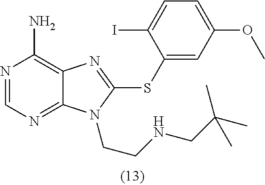

Patent application FR 2880540 (Aventis) claims another family of Hsp90-inhibiting purines.

Patent application WO 2004/072080 (Cellular Genomics) claims a family of 8-heteroaryl-6-phenylimidazo[1,2-a]pyrazines as modulators of Hsp90 activity.

Patent application WO 2004/028434 (Conforma Therapeutics) claims aminopurines, aminopyrrolopyrimidines, aminopyrazolopyrimidines and aminotriazolopyrimidines as Hsp90 inhibitors.

Patent application WO 2004/050087 (RibotargetNernalis) claims a family of pyrazoles that can be used for treating pathologies related to the inhibition of heat-shock proteins such as the Hsp90 chaperone.

Patent application WO 2004/056782 (Vernalis) claims a novel family of pyrazoles that can be used for treating pathologies related to the inhibition of heat-shock proteins such as the Hsp90 chaperone.

Patent application WO 2004/072051 (Vernalis) claims arylisoxazole derivatives that can be used for treating pathologies related to the inhibition of heat-shock proteins such as the Hsp90 chaperone.

Patent application WO 2004/096212 (Vernalis) claims a third family of pyrazoles that can be used for treating pathologies related to the inhibition of heat-shock proteins such as the Hsp90 chaperone.

Patent application WO 2005/000300 (Vernalis) claims, more generally, 5-membered heterocycles, substituted with aryl radicals, that can be used for treating pathologies related to the inhibition of heat-shock proteins such as the Hsp90 chaperone.

Patent application JP 2005/225787 (Nippon Kayaku) claims another family of pyrazoles as Hsp90 inhibitors.

Patent application WO2005/000778 (Kyowa Hakko Kogyo) claims a family of benzophenone derivatives as Hsp90 inhibitors, that can be used for the treatment of tumours.

Patent application WO2005/063222 (Kyowa Hakko Kogyo) claims a family of resorcinol derivatives as Hsp90 inhibitors.

Patent application WO2005/051808 (Kyowa Hakko Kogyo) claims a family of resorcinylbenzoic acid derivatives as Hsp90 inhibitors. Patent applications WO2005/021552, WO2005/034950, WO2006/008503, WO2006/079789 and WO2006/090094 (Vernalis) claim families of pyrimidothiophenes or of pyridothiophenes, that can be used for treating pathologies related to the inhibition of heat-shock proteins such as the Hsp90 chaperone.

Application WO2006/018082 (Merck) claims another family of pyrazoles as Hsp90 inhibitors.

Application WO2006/010595 (Novartis) claims a family of indazoles as Hsp90 inhibitors.

Application WO2006/010594 (Novartis) claims a family of dihydrobenzimidazolones as Hsp90 inhibitors.

Patent application WO2006/055760 (Synta Pharma) claims a family of diaryltriazoles as Hsp90 inhibitors.

Patent application WO2006/087077 (Merck) claims a family of (s-triazol-3-yl)phenols as Hsp90 inhibitors.

Patent application FR2882361 (Aventis) claims a family of 3-aryl-1,2-benzisoxazoles as Hsp90 inhibitors.

Patent application WO2006/091963 (Serenex) claims families of tetrahydroindolones and of tetrahydroindazolones as Hsp90 inhibitors. Patent application DE10200509440 (Merck) claims a family of thienopyridines as Hsp90 inhibitors.

Patent application WO2006/095783 (Nippon Kayaku) claims a family of triazoles as Hsp90 inhibitor&

Patent application WO2006/101052 (Nippon Kayaku) claims a family of acetylene derivatives as Hsp90 inhibitors.

Patent application WO2006/105372 (Conforma Therapeutics) claims a family of alkynyl pyrrolo[2,3-d]pyrimidines as Hsp90 inhibitors. Patent application FR2884252 (Aventis) claims a family of isoindoles as Hsp90 inhibitors.

Patent application WO2006/109075 (Astex Therapeutics) claims a family of benzamides as Hsp90 inhibitors,

Patent application WO2006/109085 (Astex Therapeutics) claims a family of hydroxybenzamides as Hsp90 inhibitors.

Patent application WO2006/113498 (Chiron) claims a family of 2-aminoquinazolin-5-ones as Hsp90 inhibitors.

Patent application JP200606755 (Nippon Kayaku) claims a family of pyrazoles as Hsp90 inhibitors.

Patent application WO2006/117669 (Pfizer) claims a family of hydroxyarylcarboxamides as Hsp90 inhibitors.

Patent applications WO2006/122631 and DE102006008890 (Merck GmbH) claim a family of amino-2-phenyl-4-quinazolines as Hsp90 inhibitors.

Patent application WO2006/123061 (Aventis) claims a family of azabenzimidazolylfluorene or benzimidazolylfluorene derivatives as Hsp90 inhibitors.

Patent application WO2006/123065 (Astex Therapeutics) claims a family of azinamines (amino-2-pyrimidines or triazines) as Hsp90 inhibitors.

Patent application WO2006/125531 (Merck GmbH) claims a family of thieno[2,3b]pyridines as Hsp90 inhibitors.

Patent applications WO2006/125813 and WO2006/125815 (Altana Pharma) claim a family of tetrahydropyridothiophenes as Hsp90 inhibitors.

Patent application WO2007/017069 (Merck GmbH) claims a family of adenine derivatives as Hsp90 inhibitors.

Patent applications WO2007/021877 and WO2007/01966 (Synta Pharma) claim, respectively, families of arylpyrazoles and of arylimidazoles as Hsp90 inhibitors.

Patent application WO2007/022042 (Novartis) claims a family of pyrimidylaminobenzamides as Hsp90 inhibitors.

Patent application WO2007/034185 (Vernalis) claims a family of heteroarylpurines as Hsp90 inhibitors.

Patent application WO2007/041362 (Novartis) claims a family of 2-amino-7,8-dihydro-6H-pyrido[4,3-d]pyrimidin-5-ones as Hsp90 inhibitors.

Patent application WO2007/104944 (Vernalis) claims a family of pyrrolo[2,3b]pyridines as Hsp90 inhibitors.

Patent application US2007/105862 claims a family of azole derivatives as Hsp90 inhibitors.

Patent application WO2007/129062 (Astex Therapeutics) claims a family of diazoles (aryl pyrazoles) as Hsp90 inhibitors.

Patent application US2007/129334 (Conforma Therapeutics) claims a family of arylthiopurines as Hsp90 inhibitors, which are active orally. Patent application WO2007/155809 (Synta Pharma) claims families of phenyltriazoles as Hsp90 inhibitors.

Patent application WO2007/092496 (Conforma Therapeutics) claims a family of 7,9-dihydropurin-8-ones as Hsp90 inhibitors.

Patent application WO2007/207984 (Serenex) claims a family of cyclohexylaminobenzene derivatives as Hsp90 inhibitors.

Patent applications DE10206023336 and DE10206023337 (Merck GmbH) claim, respectively, families of 1,5-diphenylpyrazoles and of 1,5-diphenyltriazoles as Hsp90 inhibitors.

Patent application WO2007/134298 (Myriad Genetics) claims a family of purinamines as Hsp90 inhibitors.

Patent application WO2007/138994 (Chugai) claims families of 2-aminopyrimidines or of 2-aminotriazines as Hsp90 inhibitors.

Patent applications WO2007/139951, WO2007/139952, WO2007/139960, WO2007/139967, WO2007/139968, WO2007/139955 and WO2007/140002 (Synta Pharma) claim families of triazoles as Hsp90 inhibitors and agents for treating non-Hodgkin's lymphomas.

Patent application WO 2008/003396 (Merck GmbH) claims a family of indazoles for the treatment of diseases induced by Hsp90.

Patent application WO 2008/021213 claims a family of macrocyclic compounds, of resorcinyl lactone oxime type, as inhibitors of kinases and of Hsp90.

Patent application WO 2008/020045 (Nycomed) claims a family of tetrahydrobenzothiophenes as antiproliferative and proapoptotic agents which inhibit Hsp90.

Patent application WO 2008/020024 (Nycomed) claims a family of tetrahydropyridothiophenes as anticancer agents which inhibit Hsp90.

Patent application WO 2008/024961 (Serenex) claims families of dihydropyrazines, of tetrahydropyridines, of chromanones and of dihydronaphthalenones as Hsp90 inhibitors.

Patent application WO 2008/024974 (Serenex) claims families of pyridines and of pyrazines as Hsp90 inhibitors.

Patent application VVO 2008/024981 (Serenex) claims a family of purinylindazoles as Hsp90 inhibitors.

Patent application VVO 2008/024977 (Serenex) claims families of isoquinolines, of quinazolines and of phthalazines as Hsp90 inhibitors.

Patent application WO 2008/024978 (Serenex) claims families of benzenes, of pyridines and of pyridazines as Hsp90 inhibitors.

Patent application WO 2008/024980 (Serenex) claims families of pyrroles, of thiophenes, of furans, of imidazoles, of oxazoles and of thiazoles as Hsp90 inhibitors.

Patent application WO 2008/035629 (Daiichi Sankyo) claims pyrazolopyrimidine derivatives as Hsp90 inhibitors.

Patent application WO 2008/044034 (Astex) claims hydroxybenzamide derivatives as Hsp90 inhibitors.

Patent application FR 2907453 (Sanofi-Aventis) claims a family of heterocyclic derivatives of fluorene, as Hsp90 inhibitors.

Patent application WO 2008/049105 (Wyeth) claims heterocycles containing a sulphamoyl residue, as anticancer agents which inhibit Hsp90.

Patent applications WO 2008/051416, WO 2008/057246, WO 2008/103353, WO 2008/112199 and WO 2008/021364 (Synta Pharma) claim families of triazoles as Hsp90 inhibitors.

Patent application WO 2008/053319 (Pfizer) claims amide derivatives of resorcinol as Hsp90 inhibitors.

Patent application WO 2008/056120 (Chroma Therapeutics) claims amino acid derivatives of adenine as Hsp90 inhibitors.

Patent application WO 2008/059368 (Pfizer) claims 2-aminopyridine derivatives as Hsp90 inhibitors.

Patent application WO 2008/073424 (Infinity) claims novel ansamycin analogues as orally active Hsp90 inhibitors.

Patent applications WO 2008/086857 and DE 102007002715 claim a family of triazolones as Hsp90 modulators.

Patent application WO 2008/093075 (Astra-Zeneca) claims tetrahydropteridine derivatives as Hsp90 inhibitors.

Patent application WO 2008/097640 (Synta Pharma) claims substituted phenyltriazole derivatives as Hsp90 inhibitors.

Patent application WO 2008/096218 (Pfizer) claims a family of 2-amino-5,7-dihydro-6H-pyrrolo[3,4-d]pyrimidines as Hsp90 inhibitors.

Patent application WO 2008/105526 (Chugai) claims novel macrocyclic compounds which inhibit Hsp90.

Patent application WO 2008/115262 (Curis) claims benzodioxolylpurine derivatives as Hsp90 inhibitors.

Patent application WO 2008/115719 (Curis) claims a family of imidazo[4,5-c]pyridines as Hsp90 inhibitors.

Patent application WO 2008/118391 (Synta Pharma) claims a family of phenylpyrimidinones as Hsp90 inhibitors.

Patent application WO 2008/130879 (Serenex) claims a family of tetrahydroindazoles as Hsp90 inhibitors.

Patent applications WO 2008/142720 and GB 2449293 (Dac) claim a family of 2-amino-7,8-dihydro-6H-quinazolin-5-one oximes as Hsp90 inhibitors.

Patent application WO 2008/150302 (Nexgenix Pharmaceuticals) claims novel macrocyclic compounds which are radicicol analogues, and which inhibit Hsp90.

Patent applications WO 2008/155001 and DE 102007028251 (Merck GmbH) claim a family of indazolamides as Hsp90 inhibitors.

Patent application WO 2009/004146 (Sanofi-Aventis) claims novel herbimycin A derivatives as Hsp90 inhibitors.

Patent application WO 2009/007399 (Crystax Pharmaceuticals) claims a family of 1H-imidazole-4-carboxamides as Hsp90 inhibitors,

Patent applications WO 2009/010139 and DE 102007032379 claim a family of quinazoline amides as Hsp90 modulators.

The present invention relates to indazole derivatives which are products of formula (I):

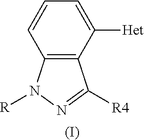

in which:
R4 represents H, CH₃, CH₂CH₃, CF₃, F, Cl, Br or I;
Het represents a monocyclic or bicyclic, aromatic or partially unsaturated heterocycle—of dihydro or tetrahydro type—, with from 5 to 11 ring members, containing from 1 to 4 heteroatoms chosen from N, O or S, optionally substituted with one or more radicals R1 or R′1, which may be identical or different, as described below,
R is chosen from the group constituted of

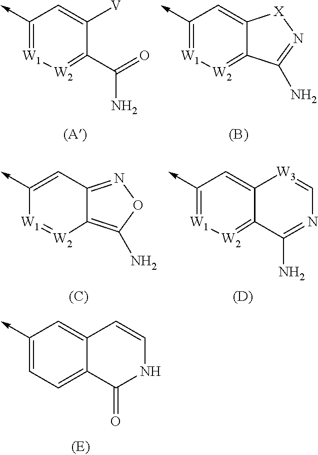

with R1 and/or R′1, which may be identical or different, chosen from the group constituted of H, halogen, CF₃, nitro, cyano, alkyl, hydroxyl, mercapto, amino, alkylamino, dialkylamino, alkoxy, phenylalkoxy, alkylthio, carboxyl in free form or esterified with an alkyl radical, carboxamide, CO—NH(alkyl), CON(alkyl)₂, NH—CO-alkyl, sulphonamide, NH—SO₂-alkyl, S(O)₂—NHalkyl and S(O₂)—N(alkyl)₂, all the alkyl, alkoxy and alkylthio radicals being themselves optionally substituted with one or more radicals, which may be identical or different, chosen from halogen, hydroxyl, alkoxy, amino, alkylamino and dialkylamino;
W1, W2 and W3 independently represent CH or N;
X represents an oxygen or sulphur atom, or an NR2, C(O), S(O) or S(O)₂ radical;
V represents a hydrogen atom or halogen atom or an —O—R2 radical or an —NH—R2 radical in which:
R2 represents a hydrogen atom or a C₁-C₆ alkyl radical, or a C₃-C₈ cycloalkyl radical or a C₃-C₁₀ heterocycloalkyl radical, which is monocyclic or bicyclic; these alkyl, cycloalkyl and heterocycloalkyl radicals being optionally substituted with one or more radicals, which may be identical or different, chosen from the radicals:
—O—PO₃H₂; —O—PO₃Na₂; —O—SO₃H₂; —O—SO₃Na₂; —O—CH₂—PO₃H₂; —O—CH₂—PO₃Na₂; —O—CO-alanine; —O—CO-glycine; —O—CO-serine; CO-lysine; —O—CO-arginine; —O—CO-glycine-lysine; —O—CO-alanine-lysine;
halogen; hydroxyl; mercapto; amino; carboxamide (CONH₂); carboxyl;
heterocycloalkyl; cycloalkyl; heteroaryl; carboxyl esterified with an alkyl radical; —CO—NH(alkyl); —O—CO-alkyl; —NH—CO-alkyl; alkyl; alkoxy; alkylthio; alkylamino; dialkylamino; in all the latter radicals, the alkyl, alkoxy and alkylthio radicals being themselves optionally substituted with one or more radicals, which may be identical or different, chosen from hydroxyl, mercapto, amino, alkylamino, dialkylamino, CO₂alkyl, NHCO₂alkyl and heterocycloalkyl radicals; in all these radicals, the cycloalkyl, heterocycloalkyl and heteroaryl radicals being themselves optionally substituted with one or more radicals, which may be identical or different, chosen from hydroxyl, alkyl, alkoxy, CH₂OH, amino, alkylamino, dialkylamino, CO₂alkyl or NHCO₂alkyl radicals;
   said products of formula (I) being in all the possible tautomeric and isomeric forms: racemic, enantiomeric and diastereoisomeric, and also as addition salts with inorganic and organic acids or with inorganic and organic bases of the products of formula (I), and also the prodrugs of the products of formula (I).

The present invention relates to indazole derivatives which are products of formula (I):

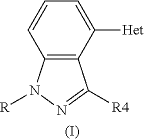

in which:
R4 represents H, CH₃, CH₂CH₃, CF₃, Cl or Br;
Het represents a monocyclic or bicyclic, aromatic or partially unsaturated heterocycle—of dihydro or tetrahydro type—, with from 5 to 11 ring members, containing from 1 to 4 heteroatoms chosen from N, O or S, optionally substituted with one or more radicals R1 or R′1, which may be identical or different, as described below,
R is chosen from the group constituted of

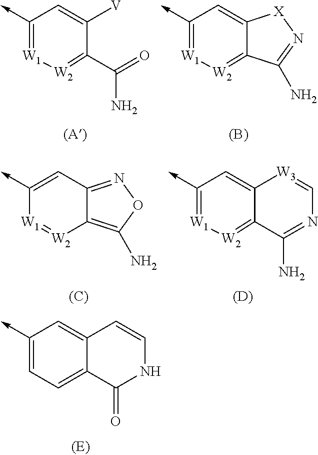

with R1 and/or R′1, which may be identical or different, chosen from the group constituted of H, halogen, CF₃, nitro, cyano, alkyl, hydroxyl, mercapto, amino, alkylamino, dialkylamino, alkoxy, phenylalkoxy, alkylthio, carboxyl in free form or esterified with an alkyl radical, carboxamide, CO—NH(alkyl), CON(alkyl)₂, NH—CO-alkyl, sulphonamide, NH—SO₂-alkyl, S(O)₂—NHalkyl and S(O₂)—N(alkyl)₂, all the alkyl, alkoxy and alkylthio radicals being themselves optionally substituted with one or more radicals, which may be identical or different, chosen from halogen, hydroxyl, alkoxy, amino, alkylamino and dialkylamino;
W1, W2 and W3 independently represent CH or N;
X represents an oxygen or sulphur atom, or an NR2, C(O), S(O) or S(O)₂ radical;
V represents a hydrogen atom or halogen atom or an —O—R2 radical or an —NH—R2 radical in which:
R2 represents a hydrogen atom or a C₁-C₆ alkyl radical, or a C₃-C₈ cycloalkyl radical or a C₃-C₁₀ heterocycloalkyl radical, which is monocyclic or bicyclic; these alkyl, cycloalkyl and heterocycloalkyl radicals being optionally substituted with one or more radicals, which may be identical or different, chosen from the radicals:
—O—PO₃H₂; —O—PO₃Na₂; —O—SO₃H₂; —O—SO₃Na₂; —O—CH₂—PO₃H₂; —O—CH₂—PO₃Na₂; —O—CO-alanine; —O—CO-glycine; —O—CO-serine; —O—CO-lysine; —O—CO-arginine; —O—CO-glycine-lysine; —O—CO-alanine-lysine;
halogen; hydroxyl; mercapto; amino; carboxamide (CONH₂); carboxyl;
heterocycloalkyl; cycloalkyl; heteroaryl; carboxyl esterified with an alkyl radical; —CO—NH(alkyl); —O—CO-alkyl; —NH—CO-alkyl; alkyl; alkoxy; alkylthio; alkylamino; dialkylamino; in all the latter radicals, the alkyl, alkoxy and alkylthio radicals being themselves optionally substituted with one or more radicals, which may be identical or different, chosen from hydroxyl, mercapto, amino, alkylamino, dialkylamino, CO₂alkyl, NHCO₂alkyl and heterocycloalkyl radicals; in all these radicals, the cycloalkyl, heterocycloalkyl and heteroaryl radicals being themselves optionally substituted with one or more radicals, which may be identical or different, chosen from hydroxyl, alkyl, alkoxy, CH₂OH, amino, alkylamino, dialkylamino, CO₂alkyl or NHCO₂alkyl radicals;
   said products of formula (I) being in all the possible tautomeric and isomeric forms; racemic, enantiomeric and diastereoisomeric, and also as addition salts with inorganic and organic acids or with inorganic and organic bases of the products of formula (I), and also the prodrugs of the products of formula (I).

In the products of formula (I) as defined above or below, it is the case that, without distinction, R4 represents H, CH₃, CH₂CH₃, CF₃, F, Cl, Br or I, or R4 represents H, CH₃, CH₂CH₃, CF₃, Cl or Br.

The present invention thus relates in particular to the products of formula (I) as defined above or below, in which;

R4 represents H, CH₃, CH₂CH₃, CF₃, F, Cl, Br or I;
Het is chosen from the group constituted of:

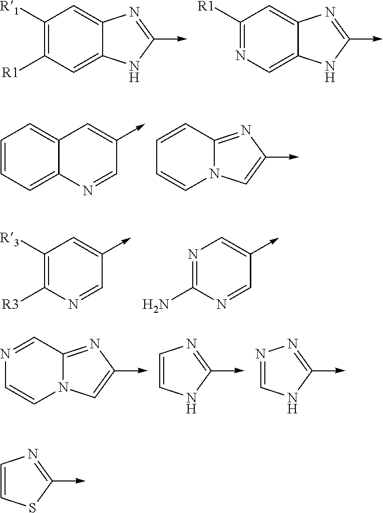

in which R′3 and R3 are such that one represents a hydrogen atom and the other is chosen from the values of R1 and R′1;
R1 and/or R′1, which may be identical or different, are chosen from the group constituted of H, halogen, CF₃, nitro, cyano, alkyl, hydroxyl, mercapto, amino, alkylamino, dialkylamino, alkoxy, phenylalkoxy, alkylthio, carboxyl in free form or esterified with an alkyl radical, carboxamide, CO—NH/alkyl), CON(alkyl)₂, NH—CO-alkyl, sulphonamide, NH—SO₂-alkyl, S(O)₂—NHalkyl and S(O₂)—N(alkyl)₂, all the alkyl, alkoxy and alkylthio radicals being themselves optionally substituted with one or more radicals, which may be identical or different, chosen from halogen, hydroxyl, alkoxy, amino, alkylamino and dialkylamino;
the values of the substituents R and R4 of said products of formula (I) being chosen from the values defined above or hereinafter,
said products of formula (I) being in all the possible tautomeric and isomeric forms: racemic, enantiomeric or diastereoisomeric, and also as addition salts with inorganic and organic acids or with inorganic and organic bases of the products of formula (I), and also the prodrugs of the products of formula (I).

The present invention thus relates in particular to the products of formula (I) as defined above or below, in which:

Het is chosen from the group constituted of;

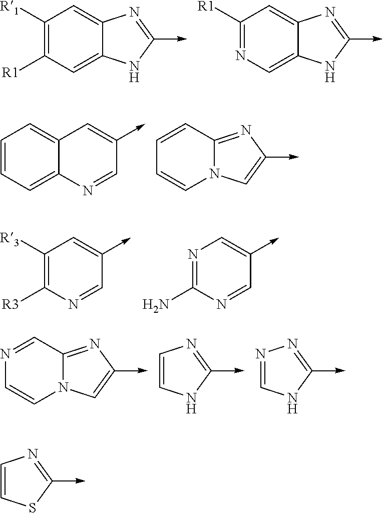

in which R′3 and R3 are such that one represents a hydrogen atom and the other is chosen from the radicals —NH₂, —CN, —CH₂—OH, —CF₃, —OH, —O—CH₂-phenyl, —O—CH₃ and —CO—NH₂;
R1 and/or R′1 are chosen from the group constituted of H, halogen, CF₃, nitro, cyano, alkyl, hydroxyl, mercapto, amino, alkylamino, dialkylamino, alkoxy, alkylthio, carboxyl in free form or esterified with an alkyl radical, carboxamide, CO—NH(alkyl), CON(alkyl)₂, NH—CO-alkyl, sulphonamide, NH—SO₂-alkyl, S(O)₂—NH(alkyl) and S(O)₂—N(alkyl)₂, or the alkyl, alkoxy and alkylthio radicals being themselves optionally substituted with one or more radicals, which may be identical or different, chosen from halogen, hydroxyl, alkoxy, amino, alkylamino and dialkylamino;
the values of the substituents R and R4 of said products of formula (I) being chosen from the values defined above or hereinafter,
said products of formula (I) being in all the possible tautomeric and isomeric forms: racemic, enantiomeric and diastereoisomeric, and also as addition salts with inorganic and organic acids or with inorganic and organic bases of the products of formula (I), and also the prodrugs of the products of formula (I)

The present invention thus relates in particular to the products of formula (I) as defined above or below, in which:

R4 represents H, CH₃, CH₂CH₃, CF₃, F, Cl, Br or I;
Het is chosen from the group constituted of:

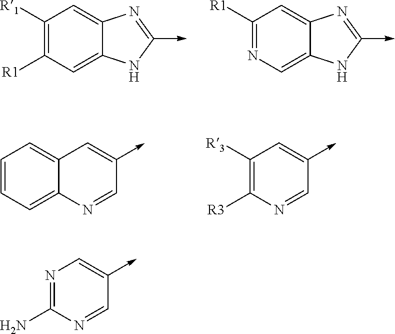

in which R′3 and R3 are such that one represents a hydrogen atom and the other is chosen from the radicals —NH₂, —CN, —CH₂—OH, —CF₃, —OH, —O—CH₂-phenyl, —O—CH₃ and —CO—NH₂;
R is chosen from the group constituted of:

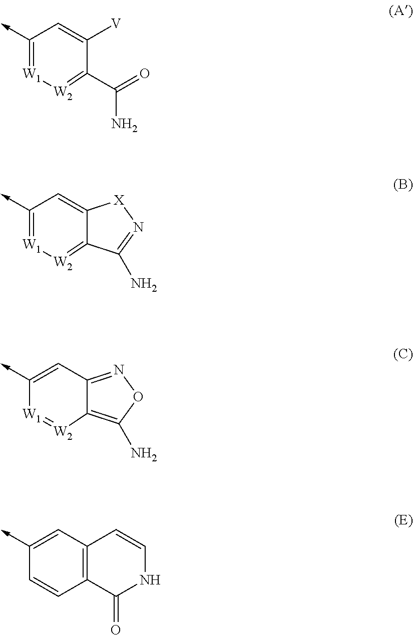

with R1 and/or R′1, which may be identical or different, chosen from the group constituted of H, halogen, CF₃, nitro, cyano, alkyl, hydroxyl, mercapto, amino, alkylamino, dialkylamino, alkoxy, —O—CH₂-phenyl, alkylthio, carboxyl in free form or esterified with an alkyl radical; carboxamide, CO—NH(alkyl), CON(alkyl)₂, NH—CO-alkyl, sulphonamide, NH—SO₂-alkyl, S(O)₂—NHalkyl and S(O₂)—N(alkyl)₂, all the alkyl, alkoxy and alkylthio radicals being themselves optionally substituted with one or more radicals, which may be identical or different, chosen from halogen, hydroxyl, alkoxy, amino, alkylamino and dialkylamino;
W1 and W2 independently represent CH or N,
X represents an oxygen or sulphur atom, or an NR2, C(O), S(O) or S(O)₂ radical;
V represents a hydrogen atom or a halogen atom or an —O—R2 radical or an NH—R2 radical in which:
R2 represents a hydrogen atom or a C₁-C₆ alkyl radical, or a C₃-C₈ cycloalkyl radical or a C₃-C₁₀ heterocycloalkyl radical, which is monocyclic or bicyclic; these alkyl, cycloalkyl and heterocycloalkyl radicals being optionally substituted with one or more radicals, which may be identical or different, chosen from the radicals:
halogen; hydroxyl; mercapto; amino; carboxamide (CONH₂); carboxyl;
heterocycloalkyl; cycloalkyl; heteroaryl; carboxyl esterified with an alkyl radical; CO—NH(alkyl); —O—CO-alkyl; —NH—CO-alkyl; alkyl; alkoxy; alkylthio; alkylamino, dialkylamino; in all the latter radicals, the alkyl, alkoxy and alkylthio radicals being themselves optionally substituted with one or more radicals, which may be identical or different, chosen from hydroxyl, mercapto, amino, alkylamino, dialkylamino, CO₂alkyl, NHCO₂alkyl and heterocycloalkyl radicals; in all these radicals, the cycloalkyl, heterocycloalkyl and heteroaryl radicals being themselves optionally substituted with one or more radicals, which may be identical or different, chosen from hydroxyl, alkyl, alkoxy, CH₂OH, amino, alkylamino, dialkylamino, CO₂alkyl or NHCO₂alkyl radicals;
   said products of formula (I) being in all the possible tautomeric and isomeric forms: racemic, enantiomeric and diastereoisomeric, and also as addition salts with inorganic and organic acids or with inorganic and organic bases of the products of formula (I), and also the prodrugs of the products of formula (I).

In the products of formula (I) and in the subsequent text, the terms indicated have the meanings which follow:

The term “halogen” denotes fluorine, chlorine, bromine or iodine atoms, and preferably fluorine, chlorine or bromine.

The term “alkyl radical” denotes a linear or branched radical containing at most 12 carbon atoms, chosen from methyl, ethyl, propyl, isopropyl, butyl, isobutyl, sec-butyl, tert-butyl, pentyl, isopentyl, sec-pentyl, tert-pentyl, neopentyl, hexyl, isohexyl, sec-hexyl, tert-hexyl and also heptyl, octyl, nonyl, decyl, undecyl and dodecyl radicals, and also the linear or branched positional isomers thereof. Mention may more particularly be made of alkyl radicals having at most 6 carbon atoms, and in particular the following radicals: methyl, ethyl, propyl, isopropyl, n-butyl, isobutyl, tert-butyl, pentyl, which may be linear or branched, and hexyl, which may be linear or branched.

The term “alkoxy radical” denotes a linear or branched radical containing at most 12 carbon atoms, and preferably 6 carbon atoms, chosen, for example, from the following radicals: methoxy, ethoxy, propoxy, isopropoxy, linear, secondary or tertiary butoxy, pentoxy, hexoxy or heptoxy, and also the linear or branched positional isomers thereof.

The term “alkylthio” or “alkyl-S-” denotes a linear or branched radical containing at most 12 carbon atoms and represents in particular methylthio, ethylthio, isopropylthio and heptylthio radicals. In the radicals containing a sulphur atom, the sulphur atom may be oxidized to an SO or S(O)₂ radical.

The term “carboxamide” denotes CONH₂,

The term “sulphonamide” denotes SO₂NH₂,

The term “acyl or NCO— radical” denotes a linear or branched radical containing at most 12 carbon atoms, in which the radical r represents a hydrogen atom or an alkyl, cycloalkyl, cycloalkenyl, heterocycloalkyl or aryl radical, these radicals having the values indicated above and being optionally substituted as indicated: mention is made, for example, of formyl, acetyl, propionyl, butyryl or benzoyl radicals, or else valeryl, hexanoyl, acryloyl, crotonoyl or carbamoyl radicals.

The term “cycloalkyl radical” denotes a monocyclic or bicyclic, carbocyclic radical containing from 3 to 10 ring members and denotes in particular cyclopropyl, cyclobutyl, cyclopentyl and cyclohexyl radicals.

The term “cycloalkylalkyl radical” denotes a radical in which cycloalkyl and alkyl are chosen from the values indicated above: this radical thus denotes, for example, cyclopropylmethyl, cyclopentylmethyl, cyclohexylmethyl and cycloheptylmethyl radicals.

The term “acyloxy radical” is intended to mean acyl-O— radicals in which acyl has the meaning indicated above: mention is made, for example, of acetoxy or propionyloxy radicals.

The term “acylamino radical” is intended to mean acyl-N— radicals in which acyl has the meaning indicated above.

The term “aryl radical” denotes carbocyclic unsaturated radicals which are monocyclic or consist of condensed rings. As examples of such an aryl radical, mention may be made of phenyl or naphthyl radicals.

The term “arylalkyl” is intended to mean radicals resulting from the combination of the alkyl radicals mentioned above, which are optionally substituted, and the aryl radicals also mentioned above, which are optionally substituted: mention is, for example, made of benzyl, phenylethyl, 2-phenethyl, triphenylmethyl or naphthalenemethyl radicals.

The term “heterocyclic radical” denotes a saturated (heterocycloalkyl) or partially or completely unsaturated (heteroaryl) carbocyclic radical consisting of 4 to 10 ring members interrupted with one or more heteroatoms, which may be identical or different, chosen from oxygen, nitrogen or sulphur atoms.

As heterocycloalkyl radicals, mention may in particular be made of aziridinyl, azetidinyl, oxetanyl, homopiperidinyl, homopiperazinyl, quinuclinidinyl, 7-oxabicyclo[2.2.1]heptanyl, dioxolanyl, dioxanyl, dithiolanyl, thiooxolanyl, thiooxanyl, oxiranyl, oxolanyl, dioxolanyl, piperazinyl, piperidyl, pyrrolidinyl, imidazolidinyl, imidazolidine-2,4-dione, pyrazolidinyl, morpholinyl or else tetrahydrofuryl, tetrahydropyranyl, tetrahydrothienyl, chromanyl, dihydrobenzofuranyl, indolinyl, perhydropyranyl, pyrindolinyl, tetrahydroquinolinyl, tetrahydroisoquinolinyl or thioazolidinyl radicals, all these radicals being optionally substituted as indicated above or below.

Among the heterocycloalkyl radicals, mention may in particular be made of 7-oxabicyclo[2.2.1]heptanyl, optionally substituted piperazinyl, N-methylpiperazinyl or piperidyl, which are optionally substituted, optionally substituted pyrrolidinyl, imidazolidinyl, pyrazolidinyl, morpholinyl, hexahydropyran or thiazolidinyl radicals.

The term “heterocycloalkylalkyl radical” is intended to mean radicals in which the heterocycloalkyl and alkyl residues have the meanings above.

Among the heteroaryl radicals with 5 ring members, mention may be made of furyl, pyrrolyl, tetrazolyl, thiazolyl, isothiazolyl, diazolyl, thiadiazolyl, thiatriazolyl, oxazolyl, oxadiazolyl, isoxazolyl, imidazolyl, pyrazolyl, thienyl and triazolyl radicals.

Among the heteroaryl radicals with 6 ring members, mention may in particular be made of pyridyl radicals such as 2-pyridyl, 3-pyridyl and 4-pyridyl, pyrimidyl radicals, pyridazinyl radicals and pyrazinyl radicals.

As condensed heteroaryl radicals containing at least one heteroatom chosen from sulphur, nitrogen and oxygen, mention may, for example, be made of benzothienyl, benzofuryl, benzopyrrolyl, benzothiazolyl, benzimidazolyl, imidazopyridyl, purinyl, pyrrolopyrimidinyl, pyrrolopyridinyl, benzoxazolyl, benzisoxazolyl, benzisothiazolyl, thionaphthyl, chromenyl, indolizinyl, quinazolinyl, quinoxalinyl, indolyl, indazolyl, purinyl, quinolyl, isoquinolyl and naphthyridinyl.

The term “alkylamino radical” is intended to mean radicals in which the alkyl radical is chosen from the alkyl radicals mentioned above. Preference is given to alkyl radicals having at most 4 carbon atoms, and mention may, for example, be made of methylamino, ethylamino, propylamino or linear or branched butylamino radicals.

The term “dialkylamino radical” is intended to mean radicals in which the alkyl radicals, which may be identical or different, are chosen from the alkyl radicals mentioned above. As above, preference is given to alkyl radicals having at most 4 carbon atoms, and mention may, for example, be made of dimethylamino radicals, diethylamino radicals or methylethylamino radicals, which may be linear or branched.

The term “patient” denotes human beings, but also other mammals.

The term “prodrug” denotes a product which can be converted in vivo, by metabolic mechanisms (such as hydrolysis), to a product of formula (I). For example, an ester of a product of formula (I) containing a hydroxyl group can be converted by hydrolysis, in vivo, to its parent molecule. Alternatively, an ester of a product of formula (I) containing a carboxyl group can be converted by hydrolysis, in vivo, to its parent molecule.

By way of examples, mention may be made of esters of products of formula (I) containing a hydroxyl group, such as acetates, citrates, lactates, tartrates, malonates, oxalates, salicylates, propionates, succinates, fumarates, maleates, methylene-bis-β-hydroxynaphthoates, gentisates, isethionates, di-p-toluoyl tartrates, methanesulphonates, ethanesulphonates, benzenesulphonates, p-toluenesulphonates, camphorsulphonates, cyclohexylsulphamates and quinates.

Particularly useful esters of products of formula (I) containing a hydroxyl group can be prepared from acid residues such as those described by Bundgaard et, al., J. Med, Chem., 1989, 32, page 2503-2507: these esters include, in particular, substituted (aminomethyl)benzoates, dialkylaminomethyl benzoates in which the two alkyl groups can be linked together or can be interrupted with an oxygen atom or with an optionally substituted nitrogen atom, i.e. an alkylated nitrogen atom, or else (morpholinomethyl)benzoates, e.g. 3- or 4-(morpholinomethyl)benzoates, and (4-alkylpiperazin-1-yl)benzoates, e.g. 3- or 4-(4-alkylpiperazin-1-yl)benzoates.

The carboxyl radical(s) of the products of formula (I) can be salified or esterified with the various groups known to those skilled in the art, among which mention may be made, by way of nonlimiting examples, of the following compounds:

among the salification compounds, inorganic bases such as, for example, an equivalent of sodium, of potassium, of lithium, of calcium, of magnesium or of ammonium, or organic bases such as, for example, methylamine, propylamine, trimethylamine, diethylamine, triethylamine, N,N-dimethylethanolamine, tris(hydroxymethyl)aminomethane, ethanolamine, pyridine, picoline, dicyclohexylamine, morpholine, benzylamine, procaine, lysine, arginine, histidine, or N-methylglucamine;

among the esterification compounds, alkyl radicals may form groups such as, for example, methoxycarbonyl, ethoxycarbonyl, tert-butoxycarbonyl or benzyloxycarbonyl, it being possible for these alkyl radicals to be substituted with radicals chosen, for example, from halogen atoms and hydroxyl, alkoxy, acyl, acyloxy, alkylthio, amino or aryl radicals, for instance from chloromethyl, hydroxypropyl, methoxymethyl, propionyloxymethyl, methylthiomethyl, dimethylaminoethyl, benzyl or phenethyl groups.

The term “esterified carboxyl” is intended to mean, for example, radicals such as alkyloxycarbonyl radicals, for example methoxycarbonyl, ethoxycarbonyl, propoxycarbonyl, butyl or tert-butyloxycarbonyl, cyclobutyloxycarbonyl, cyclopentyloxycarbonyl or cyclohexyloxycarbonyl radicals.

Mention may also be made of radicals formed with readily cleavable ester residues, such as methoxymethyl or ethoxymethyl radicals; acyloxyalkyl radicals such as pivaloyloxymethyl, pivaloyloxyethyl, acetoxymethyl or acetoxyethyl radicals; alkyloxycarbonyloxy alkyl radicals such as methoxycarbonyloxymethyl or methoxycarbonyloxyethyl radicals, isopropyloxycarbonyloxymethyl radicals or isopropyloxycarbonyloxyethyl radicals.

A list of such ester radicals can be found, for example, in European Patent EP 0 034 536.

The term “amidated carboxyl” is intended to mean radicals of the —CONH₂ type, the hydrogen atoms of which are optionally substituted with one or two alkyl radicals so as to form alkylamino or dialkylamino radicals, which are themselves optionally substituted as indicated above or below, it being possible for these radicals to also form, with the nitrogen atom to which they are attached, a cyclic amine as defined above.

The term “salified carboxyl” is intended to mean the salts formed, for example, with an equivalent of sodium, of potassium, of lithium, of calcium, of magnesium or of ammonium. Mention may also be made of the salts formed with organic bases such as methylamine, propylamine, trimethylamine, diethylamine or triethylamine.

The sodium salt is preferred.

When the products of formula (I) comprise an amino radical that can be salified with an acid, it is clearly understood that these acid salts are also part of the invention. Mention may be made of the salts provided with hydrochloric acid or methanesulphonic acid, for example.

The addition salts with inorganic or organic acids of the products of formula (I) may, for example, be the salts formed with hydrochloric acid, hydrobromic acid, hydriodic acid, nitric acid, sulphuric acid, phosphoric acid, propionic acid, acetic acid, trifluoroacetic acid, formic acid, benzoic acid, maleic acid, fumaric acid, succinic acid, tartaric acid, citric acid, oxalic acid, glyoxylic acid, aspartic acid, ascorbic acid, alkylmonosulphonic acids such as, for example, methanesulphonic acid, ethanesulphonic acid, propanesulphonic acid, alkoyldisulphonic acids such as, for example, methanedisulphonic acid, alpha, beta-ethanedisulphonic acid, arylmonosulphonic acids such as benzenesulphonic acid and aryldisulphonic acids.

It may be recalled that stereoisomerism can be defined, in its broad sense, as the isomerism of compounds having the same structural formulae, but the various groups of which are arranged differently in space, such as, in particular, in monosubstituted cyclohexanes in which the substituent can be in the axial or equatorial position, and the various possible rotational conformations of ethane derivatives. However, another type of stereoisomerism exists, due to the different spatial arrangements of attached substituents, either on double bonds or on rings, which is often referred to as geometric isomerism or cis-trans isomerism. The term “stereoisomer” is used, in the present application, in its broadest sense and therefore relates to all the compounds indicated above.

In particular, the present invention thus relates to the products of formula (I) as defined above, in which:

R4 represents H, CH₃, CH₂CH₃, CF₃, F, Cl, Br or I;
Het is chosen from the group constituted of:

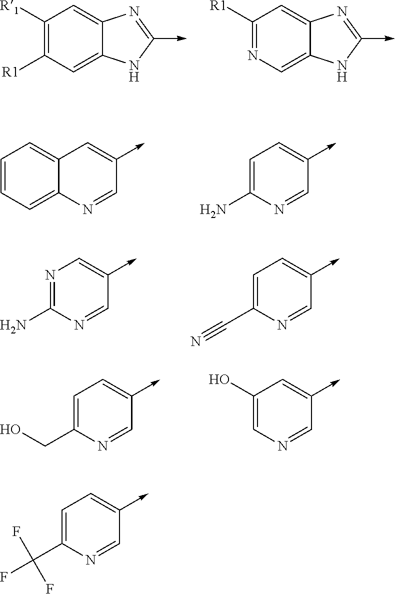

with
R1 and/or R′1 being chosen from the group constituted of H, halogen, CF₃, nitro, cyano, alkyl, hydroxyl, mercapto, amino, alkylamino, dialkylamino, alkoxy, alkylthio (methylthio), carboxyl in free form or esterified with an alkyl radical, carboxamide, CO—NH(alkyl), CON(alkyl)₂, NH—CO-alkyl, sulphonamide, NH—SO₂-alkyl, S(O)₂—NH(alkyl) and S(O)₂—N(alkyl)₂, all the alkyl, alkoxy and alkylthio radicals being themselves optionally substituted with one or more radicals, which may be identical or different, chosen from halogen, hydroxyl, alkoxy, amino, alkylamino and dialkylamino;
the substituent R of said products of formula (I) being chosen from the values defined above or hereinafter,
said products of formula (I) being in all the possible tautomeric and isomeric forms: racemic, enantiomeric and diastereoisomeric, and also as addition salts with inorganic and organic acids or with inorganic and organic bases of the products of formula (I), and also the prodrugs of the products of general formula (I).

In particular, R is chosen from the group constituted of:

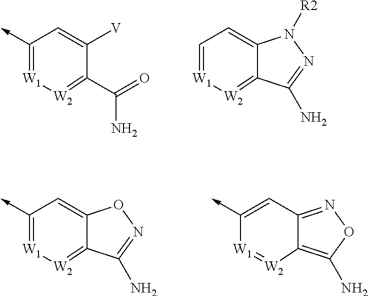

with W1, W2, V and R2 as defined above or hereinafter.

The present invention thus relates in particular to the products of formula (I) as defined above or hereinafter, in which:

R4 represents H, CH₃, CH₂CH₃, CF₃, F, Cl, Br or I;
Het is chosen from the group constituted of:

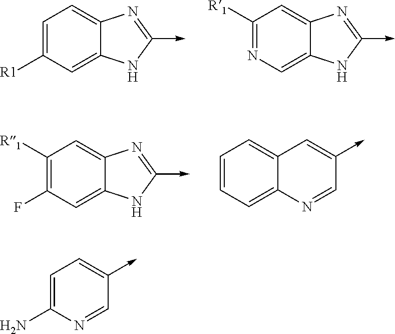

R is chosen from the group constituted of:

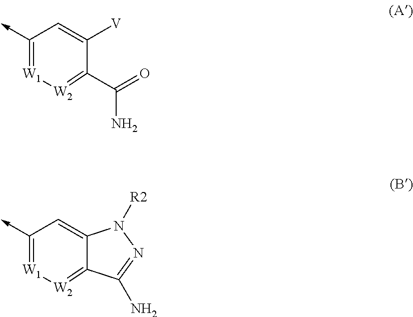

R1 is chosen from the group constituted of H, F, Cl, Br, CF₃, NO₂, ON, CH₃, OH, OCH₃, OCF₃, CO₂Me, CONH₂, CONHMe, CONH—(OH₂)₃—OMe, CONH—(CH₂)₃—N(Me)₂, NHC(O)Me, SO₂NH₂ and SO₂N(Me)₂;
R′1 is chosen from the group constituted of H, CONH₂, CONHMe and OMe;
R″1 is chosen from the group constituted of F, Cl, OH, OMe, CN, O—(CH₂)₃—OMe and O—(CH₂)₃—N(Me)₂;
W1 and W2, which may be identical or different, represent CH or N;
V represents a hydrogen atom or an —NH—R2 radical in which:
R2 represents a hydrogen atom or a C₁-C₆ alkyl, C₃-C₈ cycloalkyl or C₄-C₈ heterocycloalkyl radical, all these alkyl, cycloalkyl and heterocycloalkyl radicals being optionally substituted with one or more radicals, which may be identical or different, chosen from the radicals:
halogen; hydroxyl; amino; carboxamide; carboxyl;
7-oxabicyclo[2.2.1]hept-2-yl; azetidinyl; oxetanyl; tetrahydrofuranyl; tetrahydropyranyl; piperazinyl; alkylpiperazinyl; pyrrolidinyl; morpholinyl; homopiperidinyl; homopiperazinyl; quinuclidinyl; piperidinyl and pyridyl, all these cyclic radicals being themselves optionally substituted with one or more radicals chosen from hydroxyl and alkyl radicals;
carboxyl esterified with an alkyl radical, CO—NH(alkyl), O—CO-alkyl, NH—CO-alkyl, alkyl, alkoxy, methylthio, alkylamino, dialkylamino, all the latter alkyl and alkoxy radicals being themselves optionally substituted with a hydroxyl, mercapto, amino, alkylamino, dialkylamino, azetidino, oxetano, pyrrolidino, tetrahydrofuranyl, piperidino, tetrahydropyranyl, piperazino, morpholino, homopiperidino, homopiperazino or quinuclidino radical;
   said products of formula (I) being in all the possible tautomeric and isomeric forms: racemic, enantiomeric and diastereoisomeric, and also as addition salts with inorganic and organic acids or with inorganic and organic bases of the products of formula (I), and also the prodrugs of the products of general formula (I).

The present invention relates in particular to the products of formula (I) as defined above, in which R is chosen from the group constituted of:

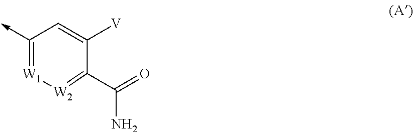

in which W1, W2, V and X have any one of the meanings indicated above.

It may be noted that R, which can represent (A′) as defined above, can in particular represent (A).

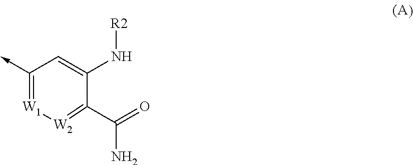

in which W1, W2 and R2 have any one of the meanings indicated above.

In particular, W1 and W2 may be such that W1 represents CH and W2 represents CH or N.

In particular, in the products of formula (I) according to the present invention, (A) can represent the following structures:

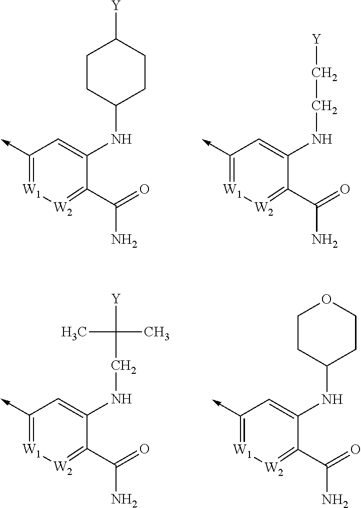

in which R2 represents a tetrahydropyranyl radical or a cyclohexyl, ethyl or 2,2-dimethylethyl radical substituted with Y, such that Y represents OH, O—PO₃H₂, O—PO₃Na₂, O—SO₃H₂, O—SO₃Na₂, O—OH₂—PO₃H₂, O—CH₂—PO₃Na₂, O—CO—CH₂—CO₂tBu, O—CO—CH₂—NH₂, O—CO-glycine, O—CO—OH₂—N(Me)₂, O—CO—OH₂—NHMe, O—CO-alanine, O—CO-serine, O—CO-lysine, O—CO-arginine, O—CO-glycine-lysine or O—CO-alanine-lysine,
said products of formula (I) being in all the possible isomeric forms: racemic, enantiomeric and diastereoisomeric, and also the addition salts with inorganic and organic acids or with inorganic and organic bases.

In the —O—CO-glycine, —O—CO—CH₂—N(Me)₂, —O—CO—CH₂—NHMe, —O—CO-alanine, —O—CO-serine, —O—CO-lysine, —O—CO-arginine, —O—CO-glycine-lysine and —O—CO-alanine-lysine radicals as defined above or hereinafter, the terms glycine, -alanine, -serine, -lysine and -arginine represent the amino acid residues as known and described in the customary manuals of those skilled in the art.

The subject of the invention is in particular the products of formula (I) as defined above, in which:

R4 represents H, CH₃, CF₃, Cl or Br;
Het is chosen from the amp constituted of:

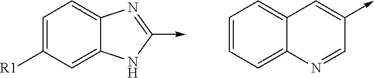

where R1 represents H, F, Cl, Br, CF₃, NO₂, CN, CH₃, OH, OCH₃, OCF₃, CO₂Me, CONH₂, CONHMe, CONH—(OH₂)₃—OMe, CONH—(CH₂)₃—N(Me)₂, NHC(O)Me, SO₂NH₂ or SO₂N(Me)₂;
R represents

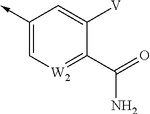

where W2 represents CH or N,
V represents a hydrogen atom or an —NH—R2 radical in which:
R2 represents a C₁-C₄ alkyl radical, a C₃-C₆ cycloalkyl radical or a C₅-C₇ heterocycloalkyl radical, all these alkyl, cycloalkyl and heterocycloalkyl radicals being optionally substituted with one or more radicals, which may be identical or different, chosen from the radicals:
halogen; hydroxyl; amino; carboxamide (CONH₂); carboxyl;
heterocycloalkyl such as tetrahydrofuranyl; piperidinyl; 7-oxabicyclo[2.2.1]hept-2-yl; tetrahydropyranyl; piperazinyl; alkylpiperazinyl; morpholinyl, homopiperidinyl; homopiperazinyl; quinuclidinyl; pyridyl; —O—CO-alkyl; alkyl; alkoxy; alkylamino; dialkylamino; in all these radicals, the alkyl radicals being themselves optionally substituted with one or more radicals, which may be identical or different, chosen from hydroxyl, amino, alkylamino and dialkylamino radicals; the piperidyl radical being itself optionally substituted with one or more radicals, which may be identical or different, chosen from hydroxyl, alkyl, alkoxy, CH₂OH, amino, alkylamino and dialkylamino radicals;
   and also the prodrugs thereof, said products of formula (I) being in all the possible isomeric forms: tautomeric, racemic, enantiomeric and diastereoisomeric, and also the addition salts with inorganic and organic acids or with inorganic and organic bases of said products of formula (I).

In the products of formula (I) above, R4 also represents H, CH₃, CF₃ or Cl; the substituents Het and R having any one of the definitions above.

In the products of formula (I) as defined above, when R2 represents a C₄-C₈ heterocycloalkyl radical. R2 may, for example, represent a piperidyl, morpholinyl, 7-oxabicyclo[2.2.1]hept-2-yl, tetrahydrofuranyl, tetrahydropyranyl, piperazinyl or quinuclidinyl radical, all optionally substituted as indicated above or hereinafter. The subject of the present invention is more particularly the products of formula (I) as defined above, which have the following names:
2-(trans-4-hydroxycyclohexylamino)-4-(3-methyl-4-quinolin-3-ylindazol-1-yl)benzamide.
4-(3-methyl-4-quinolin-3-ylindazol-1-yl)benzamide.
2-(3-hydroxypropylamino)-4-(3-methyl-4-quinolin-3-ylindazol-1-yl)benzamide.
2-[2-(4-hydroxy-1-methylpiperidin-4-yl)ethylamino]-4-(3-methyl-4-quinolin-3-ylindazol-1-yl)benzamide.
2-(2-hydroxy-2-methylpropylamino)-4-(3-methyl-4-quinolin-3-ylindazol-1-yl)benzamide.
4-(3-methyl-4-quinolin-3-ylindazol-1-yl)-2-(2,2,6,6-tetramethylpiperidin-4-ylamino)benzamide.
4-(3-methyl-4-quinolin-3-ylindazol-1-yl)-2-(tetrahydropyran-4-ylamino)benzamide.
2-(2-fluoroethylamino)-4-(3-methyl-4-quinolin-3-ylindazol-1-yl)benzamide.
3-(2-hydroxy-2-methylpropylamino)-5-(3-methyl-4-quinolin-3-ylindazol-1-yl)pyridine-2-carboxamide.
5-(3-methyl-4-quinolin-3-ylindazol-yl)-3-(tetrahydropyran-4-ylamino)pyridine-2-carboxamide.
trans-4-[2-carbamoyl-5-(3-methyl-4-quinolin-3-ylindazol-1-yl)phenyl-amino]cyclohexyl ester of aminoacetic add.
4-[4-(6-fluoro-1H-benzimidazol-2-yl)-3-methylindazol-1-yl]-2-(trans-4-hydroxy-cyclohexylamino)benzamide.
4-[4-[(6-fluoro-1H-benzimidazol-2-yl)-3-methylindazol-1-yl]-2-(2-hydroxy-2-methyl-propylamino)benzamide.
4-(3-methyl-4-quinolin-3-ylindazol-1H)-2-[exo-(7-oxabicyclo[2.2.1]hept-2-yl)amino]benzamide.
4-(3-methyl-4-quinolin-3-ylindazol-1-yl)-2-(1,2,2,6,6-pentamethylpiperidin-4-ylamino)benzamide.
3-(trans-4-hydroxycyclohexylamino)-5-(3-methyl-4-quinolin-3-ylindazol-1-yl)pyridine-2-carboxamide.
5-[3-methyl-4-quinolin-3-ylindazol-1-yl]-3-(1,2,2,6,6-pentamethylpiperidin-1-ylamino)pyridine-2-carboxamide.
5-[3-methyl-4-quinolin-3-ylindazol-1-yl]-3-[2-pyridin-2-ylethylamino]pyridine-2-carboxamide.
4-(3-methyl-4-quinolin-3-ylindazol-1-yl)-2-{[exo-1-(7-oxabicyclo[2.2.1]hept-2-yl)methyl]amino}benzamide,
4-(3-methyl-4-quinolin-3-ylindazol-1-yl)-2-{[endo-1-(7-oxabicyclo[2.2.1]hept-2-yl)methyl]amino}benzamide.
2-(trans-4-hydroxycyclohexylamino)-4-(4-quinolin-3-yl-3-trifluoromethylindazol-1-yl)benzamide.
4-[4-(6-fluoro-1H-benzimidazol-2-yl)-3-trifluoromethylindazol-1-yl]-2-(trans-4-hydroxycyclohexylamino)benzamide.
3-(trans-4-hydroxycyclohexylamino)-5-(4-quinolin-3-yl-3-trifluoromethylindazol-1-yl)pyridine-2-carboxamide.
2-(trans-4-hydroxycyclohexylamino)-4-(4-quinolin-3-ylindazol-1-yl)benzamide.
4-(4-quinolin-3-ylindazol-1-yl)benzamide.
5-(3-chloro-4-quinolin-3-ylindazol-1-O-3-(trans-4-hydroxycyclohexyl-amino)pyridine-2-carboxamide.
5-(3-bromo-4-quinolin-3-ylindazol-1-yl)-3-(2-hydroxy-2-methylpropyl-amino)pyridine-2-carboxamide.
   and also the addition salts with inorganic and organic acids or with inorganic and organic bases of said products of formula (I).

The products of formula (I) according to the present invention can be prepared according to the methods known to those skilled in the art and particularly according to the methods described hereinafter: the subject of the present invention is thus also the methods for synthesizing the products of formula (I) according to the present invention, and in particular the general methods of synthesis described in the schemes hereinafter.

### General Methods for Synthesizing the Compounds of General Formula (I):

The products of general formula (I) can be prepared from a 4-hydroxy-1H-indazole derivative, of general formula (II), by first introducing either the heterocycle Het so as to form a compound of general formula (III), or a precursor of the radical R so as to form a product of general formula (IV), according to general scheme (1) below:

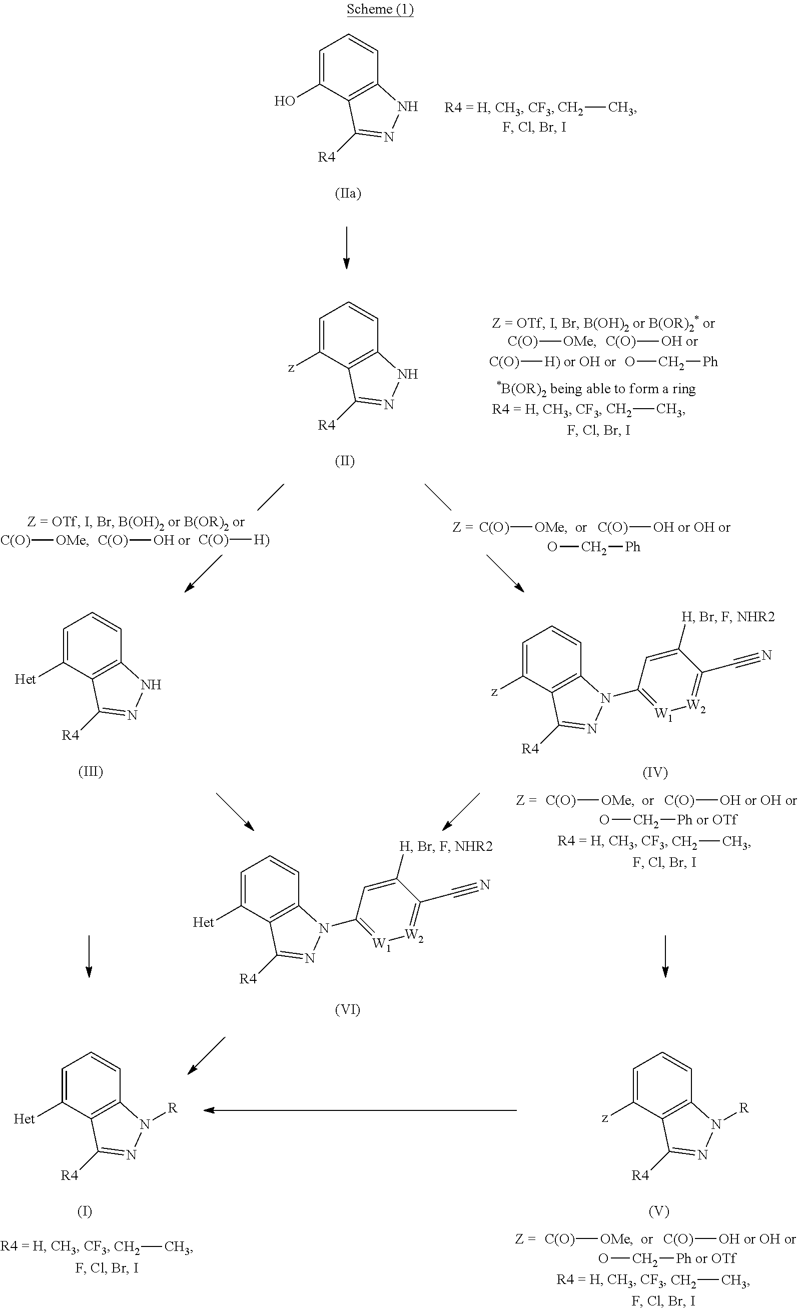

The subject of the present invention is thus in particular the process described in scheme (1) above for synthesizing the products of formula (I) as defined above and hereinafter.

The subject of the present invention is also, as novel industrial products, the synthesis intermediates of formulae (III), (IV), (V) and (VI) as defined above, in which the substituents Het, R, R2, R4, W1 and W2 have the meanings indicated above for the products of formula (I) as defined above, and Z has the meaning indicated above in scheme (1).

The subject of the present invention is also, as novel industrial products, the starting products of formula (IIa) as defined above and hereinafter:

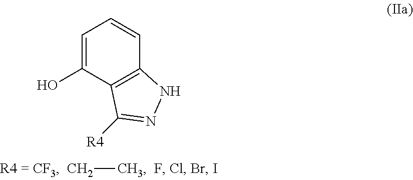

in which R4 represents CF₃, CH₂—CH₃, F, Cl, Br or I.

The subject of the present invention is also, as novel industrial products, the starting products or synthesis intermediates of formula (II) as defined above or hereinafter:

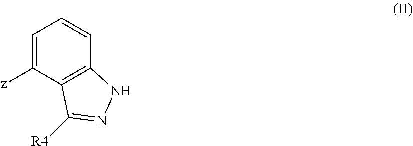

in which:

z represents OTf and R4 represents H, CF₃, CH₂—CH₃, F, Cl, Br or I;

z represents I and R4 represents CH₃, CF₃, CH₂—CH₃, F or Cl;

z represents Br and R4 represents CH₃, CF₃, CH₂—CH₃ or F;

z represents Bpinacol (among B(OR)₂) and R4 represents CH₃, CF₃, CH₂—CH₃, F, Cl, Br or I;

z represents CO₂Me and R4 represents CH₃, CF₃, CH₂—CH₃, F or Cl;

z represents CO₂H and R4 represents CH₃, CF₃, CH₂—CH₃, F, Cl or Br;

z represents CHO and R4 represents CH₃, CF₃, CH₂—CH₃, F, Cl or Br;

z represents OH (product of formula (IIa)) and R4 represents CF₃, CH₂—CH₃, F, Cl, Br or I;

z represents OCH₂-phenyl and R4 represents CF₃, CH₂—CH₃, F, Cl or Br.

More particularly, the subject of the present invention is also, as novel industrial products, the synthesis intermediates (III) for the products of formula (I) as defined above or hereinafter, in which R4 represents CF₃, F, Cl, Br or I, and Het has any one of the meanings indicated above or hereinafter.

More particularly, the subject of the present invention is also, as novel industrial products, the synthesis intermediates of formulae (IV), (V) and (VI) as defined in scheme (1) above and hereinafter:

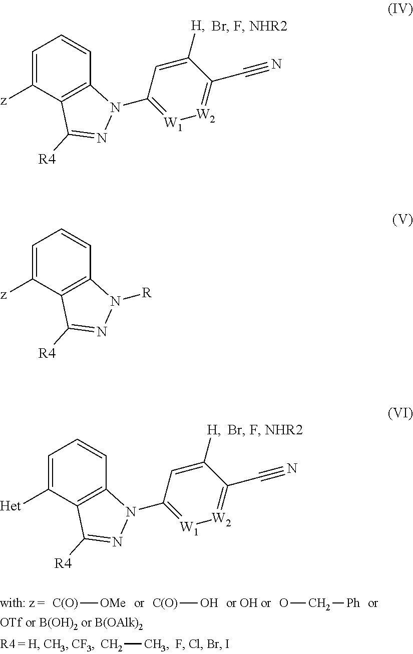

in which the substituents Het, z, R, R2, R4, W1 and W2 have the meanings indicated above for the products of formula (I) as defined above, and z has the meaning indicated above in scheme (1).

Such synthesis intermediates which are subjects of the present invention can be obtained in the course of scheme (1) or alternatively, where appropriate, in the course of one or more of the synthesis schemes (2) to (45) described hereinafter. The subject of the present invention is thus also, as novel industrial products, the synthesis intermediates (IV) for the products of formula (I):

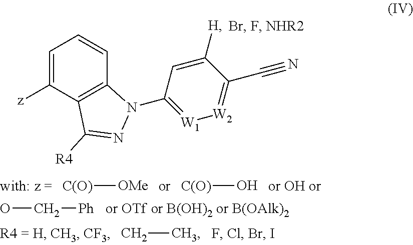

as defined above or hereinafter, in which z, R4, R2, W1 and W2 have any one of the meanings indicated above or hereinafter.

The subject of the present invention is thus also, as novel industrial products, the synthesis intermediates (V) for the products of formula (I):

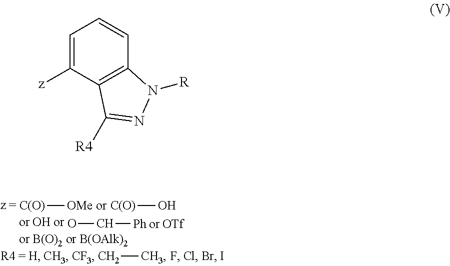

as defined above or hereinafter, in which Z. R4 and R have any one of the meanings indicated above or hereinafter.

The subject of the present invention is thus also, as novel industrial products, the synthesis intermediates (VI) for the products of formula (I):

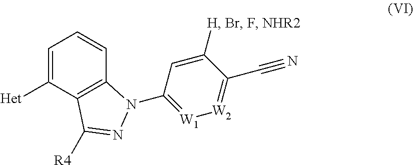

as defined above or hereinafter, in which Het, R2, R4, W1 and W2 have any one of the meanings indicated above or hereinafter.

The introduction of the group R4=Cl on the products (I) may be carried out by chlorination according to conventional methods (chlorine gas, N-chloro-succinimide, NaOCl, etc.) in the course of scheme (1) starting from the corresponding compound (I), (II), (IIa), (III), (IV), (V) or (VI) with R4=H.

The introduction of the R4=Br group on the products (I) may be carried out by bromination according to conventional methods (bromine, N-bromosuccinimide, NaOBr, pyridinium tribromide, etc.) in the course of scheme (1) starting from the corresponding compounds (I), (II), (IIa), (III), (IV), (V) or (VI) with R4=H.

The introduction of the group R4=F on the products (I) may be carried out by fluorination according to conventional methods (Selectfluor®, etc) in the course of scheme (1) starting from the corresponding compound (I), (II), (IIa), (III), (IV), (V) or (VI) with R4=H.

The introduction of the group R4=I on the products (I) may be carried out by iodination according to conventional methods (iodine in basic medium, N-iodosuccinimide, etc) in the course of scheme (1) starting from the corresponding compound (I), (II), (IIa), (III), (IV), (V) or (VI) with R4=H,

Preparation of the Compounds of General Formula (IIa)

The subject of the present invention is thus also the methods for synthesizing the products of formula (IIa), in which R4 represents the CF₃, CH₂CH₃, F, Cl, Br or I radical.

The product of general formula (IIa) in which R4 represents CH₃ can be obtained according to J. Med. Chem. 2000, 43(14), 2664 or patent WO 2004/039796.

The products of general formula (IIa) in which R4 represents H, CH₃, CF₃ or CH₂CH₃ can be obtained in two stages according to general scheme (2) below:

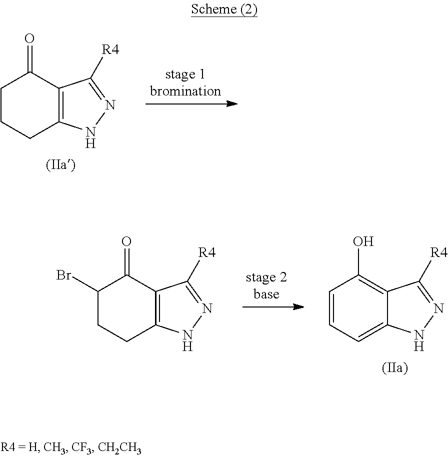

The subject of the present invention is thus in particular the process described in scheme (2) above for synthesizing the intermediates of formula (IIa) for the preparation of products of formula (I) as defined above.

The first stage, which is the bromination stage, is preferably carried out with cupric bromide in an organic solvent such as acetonitrile in the presence of lithium bromide. The second stage, which is the dehydrobromination stage, is carried out with a base and preferably lithium carbonate in the presence of lithium bromide in an organic solvent such as dimethylformamide.

The product of general formula (IIa) in which R4 represents H can also be obtained according to patent WO 2004/039796.

The product of general formula (IIa′) in which R4 represents H can be obtained according to Synthesis 2002, 12, 1669.

The products of general formula (IIa′) in which R4 represents CF₃ and CH₂CH₃ can be obtained in one stage according to scheme (3) below:

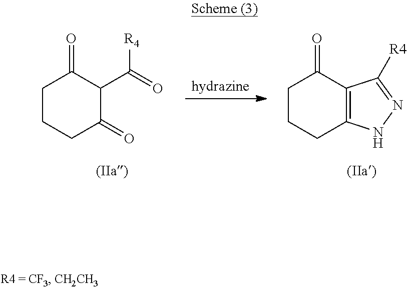

The subject of the present invention is thus in particular the process described in scheme (3) above for synthesizing the intermediates of formula (IIa′) for the preparation of products of formula (I) as defined above.

The cyclization is preferably carried out with hydrazine hydrate in an organic solvent such as ethanol.

The product of general formula (IIa″) in which R4 represents CF₃ can be obtained according to J. Fluorine Chem. 2006, 127, 1564.

The product of general formula (IIa″) in which R4 represents CH₂CH₃ can be obtained according to J. Org. Chem. 1999, 64 (19), 6984.

The product of general formula (IIa) in which R4 represents Cl can be obtained by chlorination of the compound of general formula (IIa) in which R4 represents H, by using a chlorinating reagent known to those skilled in the art, such as sodium hypochlorite in a basic aqueous medium (analogy with Bioorg. Med. Chem. 2007, 15 (6), 2441), chlorine gas in an acetic medium (analogy with J. Med. Chem. 2003, 46 (26), 5663) or preferably N-chlorosuccinimide in an organic solvent such as dimethylformamide (analogy with patent WO 1997/12884),

The product of general formula (IIa) in which R4 represents Br can be obtained by bromination of the compound of general formula (IIa) in which R4 represents H, using a brominating reagent known to those skilled in the art, such as sodium hypobromite in a basic aqueous medium (analogy with patent WO 2006/50006), bromine in an acetic medium (analogy with patent WO 2007/126841), pyridinium tribromide in an organic solvent such as methanol (analogy with patent US 2005/277638) or preferably N-bromosuccinimide in an organic solvent such as dimethylformamide (analogy with Bioorg. Med. Chem. 2008, 16 (11), 5962).

The product of general formula (IIa) in which R4 represents F can be obtained by fluorination of the compound of general formula (IIa) in which R4 represents H, using a fluorinating reagent known to those skilled in the art, such as, for example, Selectfluor® in an organic solvent such as acetonitrile as a mixture with acetic acid (analogy with patent WO 2009/147188).

The product of general formula (IIa) in which R4 represents I can be obtained by iodination of the compound of general formula (IIa) in which R4 represents H, using an iodinating reagent known to those skilled in the art, such as iodine in a basic aqueous medium (analogy with patent WO 2008/154241 or Synlett (20), 3216 (2008)), or N-iodosuccinimide in an organic solvent such as dimethylformamide.

Preparation of the Compounds of General Formula (II)

The subject of the present invention is thus also the methods for synthesizing the products of formula (II), in which Z represents the triflate radical, a boronic acid or a boronate, which is optionally cyclic, excluding the product of formula (II) with Z representing the triflate radical and R4 representing CH₃ (described in patent WO 2005/028445) and the product of formula (II) with Z representing pinacol boronate and R4 representing H (described in J. Med. Chem. 2008, 51 (18), 5522 and patent WO 2007/129161).

The products of general formula (II) in which Z represents the benzyloxy radical and R4 represents H, CH₃, CF₃, CH₂CH₃, F, Cl, Br or I can be obtained by alkylation with benzyl bromide starting from the corresponding compound (IIa), by analogy with hydroxycarbazole according to Bioorg. Med. Chem. 2005, 13 (13), 4279 or when R4 represents H and CH₃ according to patent WO 2008/107455.

The product of general formula (II) in which Z represents the benzyloxy radical and R4 represents Cl can also be obtained by chlorination of the compound of general formula (II) in which Z represents the benzyloxy radical and R4 represents H, by using a chlorinating reagent known to those skilled in the art, such as sodium hypochlorite in a basic aqueous medium (analogy with Bioorg. Med. Chem. 2007, 15 (6), 2441), chlorine gas in an acetic medium (analogy with J. Med. Chem. 2003, 46 (26), 5663) or preferably N-chlorosuccinimide in an organic solvent such as dimethylformamide (analogy with patent WO 1997/12884).

The product of general formula (II) in which Z represents the benzyloxy radical and R4 represents Br can be obtained by bromination of the compound of general formula (II) in which Z represents the benzyloxy radical and R4 represents H, by using a brominating reagent known to those skilled in the art, such as sodium hypobromite in a basic aqueous medium (analogy with patent WO 2006/50006), bromine in an acetic medium (analogy with patent WO 2007/126841), pyridinium tribromide in an organic solvent such as methanol (analogy with US patent 2005/277638) or preferably N-bromosuccinimide in an organic solvent such as dimethylformamide (analogy with Bioorg. Med. Chem. 2008, 16 (11), 5962).

The product of general formula (II) in which Z represents the benzyloxy radical and R4 represents F can also be obtained by fluorination of the compound of general formula (II) in which Z represents the benzyloxy radical and R4 represents H, by using a fluorinating reagent known to those skilled in the art, such as, for example, Selectfluor® in an organic solvent such as acetonitrile as a mixture with acetic acid (analogy with patent WO 2009/147188),

The product of general formula (II) in which Z represents the benzyloxy radical and R4 represents I is known in the literature (Registry Number=885962-49-2) and can also be obtained by iodination of the compound of general formula (II) in which Z represents the benzyloxy radical and R4 represents H, by using an iodinating reagent known to those skilled in the art, such as iodine in a basic aqueous medium (analogy with patent WO 2008/154241 or Synlett (20), 3216 (2008)), or N-iodosuccinimide in an organic solvent such as dimethylformamide.

The products of general formula (II) in which Z represents the trifluoro-methanesulphonyloxy radical (also referred to as “triflate” in the rest of the invention) and R4 represents H, CH₃, CF₃, CH₂CH₃, F, Cl, Br or I can be obtained by the action of a trifluoromethylsulphonating agent, such as N-phenyl-bis(trifluoromethanesulphonimide), in an organic solvent such as dichloromethane or tetrahydrofuran, in the presence of an organic base such as triethylamine, according to scheme (4) below:

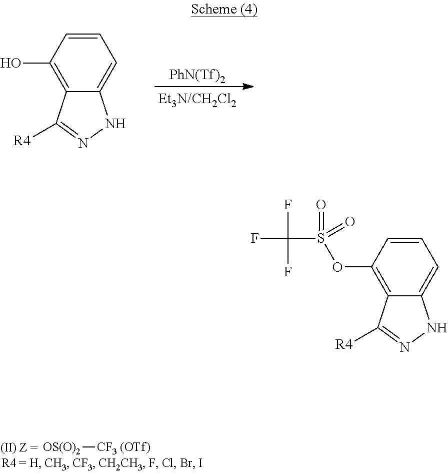

The subject of the present invention is thus in particular the process described in scheme (4) above for synthesizing the intermediates of formula (II) for the preparation of products of formula (I) as defined above.

By increasing the amount of trifluoromethylsulphonating reagent, it is possible to also obtain the ditriflate compounds according to scheme (4a). These ditriflates can then be converted directly into the compounds (III) according to scheme (8a), subsequently,

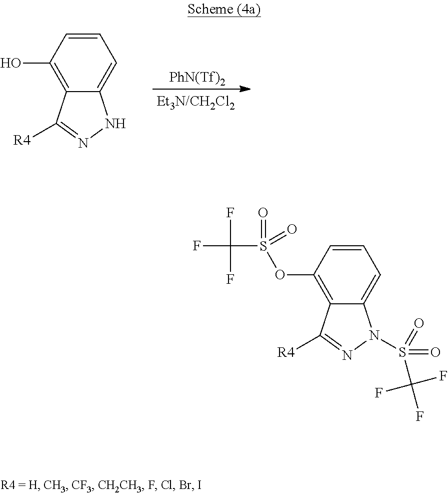

The subject of the present invention is thus in particular the process as described in scheme (4a) above.

The product of general formula (II) in which Z represents the trifluoromethanesulphonyloxy radical and R4 represents CH₃ can also be obtained according to patent WO 2005/028445.

The product of general formula (II) in which Z represents the trifluoro-methanesulphonyloxy radical and R4 represents Cl can be obtained by chlorination of the compound of general formula (II) in which Z represents the trifluoromethanesulphonyloxy radical and R4 represents H, by using a chlorinating reagent known to those skilled in the art, such as, for example, N-chloro-succinimide in an organic solvent such as dimethylformamide (analogy with patent WO 1997/12884).

The product of general formula (II) in which Z represents the trifluoro-methanesulphonyloxy radical and R4 represents Br can be obtained by bromination of the compound of general formula (II) in which Z represents the trifluoromethanesulphonyloxy radical and R4 represents H, by using a brominating reagent known to those skilled in the art, such as pyridinium tribromide in an organic solvent such as methanol (analogy with patent US 2005/277638) or preferably N-bromosuccinimide in an organic solvent such as dimethylformamide (analogy with Bioorg. Med. Chem. 2008, 16 (11), 5962).

The product of general formula (II) in which Z represents the trifluoromethanesulphonyloxy radical and R4 represents F can also be obtained by fluorination of the compound of general formula (II) in which Z represents the trifluoromethanesulphonyloxy radical and R4 represents H, by using a fluorinating reagent known to those skilled in the art, such as, for example, Selectfluor® in an organic solvent such as acetonitrile as a mixture with acetic acid (analogy with patent WO 2009/147188).

The product of general formula (II) in which Z represents the trifluoro-methanesulphonyloxy radical and R4 represents I can also be obtained by iodination of the compound of general formula (II) in which Z represents the trifluoromethanesulphonyloxy radical and R4 represents H, by using an iodinating reagent known to those skilled in the art, such as iodine in a basic aqueous medium (analogy with patent WO 2008/154241 or Synlett (20), 3216 (2008)), or N-iodosuccinimide in an organic solvent such as dimethylformamide.

The products of general formula (II) in which Z represents a methyl carboxylate radical and R4 represents H, CH₃, CF₃, CH₂CH₃, F, Cl or Br can be obtained by advantageously carrying out a carbonylation reaction in methanol, catalyzed by a palladium complex such as palladium acetate in the presence of a phosphine-type ligand such as 1,3-diphenylphosphinopropane, according to scheme (5) below:

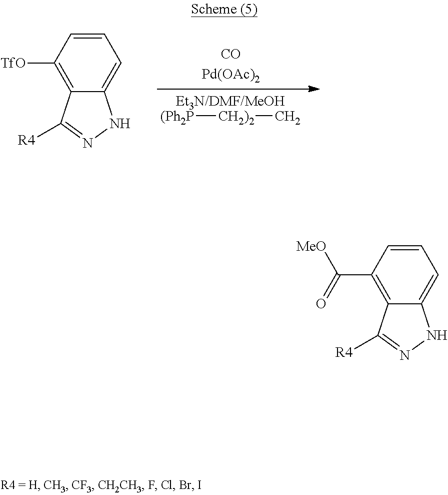

The product of general formula (II) in which Z represents the methyl carboxylate radical and R4 represents H can also be prepared according to patent WO 2005/028445.

The product of general formula (II) in which Z represents the methyl carboxylate radical and R4 represents I is known in the literature (Registry Number=885521-54-0).

The product of general formula (II) in which Z represents the methyl carboxylate radical and R4 represents Cl can also be obtained by chlorination of the compound of general formula (II) in which Z represents the methyl carboxylate radical and R4 represents H, by using a chlorinating reagent known to those skilled in the art, such as sodium hypochlorite in a basic aqueous medium (analogy with Bioorg. Med. Chem. 2007, 15 (6), 2441), chlorine gas in an acetic medium (analogy with J. Med. Chem. 2003, 46 (26), 5663) or preferably N-chloro-succinimide in an organic solvent such as dimethylformamide (analogy with patent WO 1997/12884).

The product of general formula (II) in which Z represents the methyl carboxylate radical and R4 represents Br can be obtained by bromination of the compound of general formula (II) in which Z represents the methyl carboxylate radical and R4 represents H, by using a brominating reagent known to those skilled in the art, such as sodium hypobromite in a basic aqueous medium (analogy with patent WO 2006/50006), bromine in an acetic medium (analogy with patent WO 2007/126841), pyridinium tribromide in an organic solvent such as methanol (analogy with patent US 2005/277638) or preferably N-bromo-succinimide in an organic solvent such as dimethylformamide (analogy with Bioorg. Med. Chem. 2008, 16 (11), 5962).

The product of general formula (II) in which Z represents the methyl carboxylate radical and R4 represents F can also be obtained by fluorination of the compound of general formula (II) in which Z represents the methyl carboxylate radical and R4 represents H, by using a fluorinating reagent known to those skilled in the art, such as, for example, Selectfluor® in an organic solvent such as acetonitrile as a mixture with acetic acid (analogy with patent WO 2009/147188).

The product of general formula (II) in which Z represents the methyl carboxylate radical and R4 represents I can also be obtained by iodination of the compound of general formula (II) in which Z represents the methyl carboxylate radical and R4 represents H, by using an iodinating reagent known to those skilled in the art, such as iodine in a basic aqueous medium (analogy with patent WO 2008/154241 or Synlett (20), 3216 (2008)), or N-iodosuccinimide in an organic solvent such as dimethylformamide.

The product of general formula (II) in which Z represents the carboxyl radical and R4 represents H can also be prepared according to patent WO 2005/028445.

The product of general formula (II) in which Z represents the carboxyl radical and R4 represents I is known in the literature (Registry Number=885520-80-9)

In general, the products of general formula (II) in which Z represents a carboxyl radical and R4 represents CH₃, CF₃, CH₂CH₃, F, Cl, Br or I can be obtained from the analogues in which Z represents a methyl carboxylate radical by saponification with aqueous sodium hydroxide in an organic medium and advantageously in methanol.

The product of general formula (II) in which Z represents the carboxyl radical and R4 represents Cl can also be obtained by chlorination of the compound of general formula (H) in which Z represents the carboxyl radical and R4 represents H, by using a chlorinating reagent known to those skilled in the art, such as sodium hypochlorite in a basic aqueous medium (analogy with Bioorg. Med. Chem. 2007, 15 (6), 2441), chlorine gas in an acetic medium (analogy with J. Med. Chem. 2003, 46 (26), 5663) or preferably N-chlorosuccinimide in an organic solvent such as dimethylformamide (analogy with patent WO 1997/12884).

The product of general formula (II) in which Z represents the carboxyl radical and R4 represents Br can be obtained by bromination of the compound of general formula (II) in which Z represents the carboxyl radical and R4 represents H, by using a brominating reagent known to those skilled in the art, such as sodium hypobromite in a basic aqueous medium (analogy with patent WO 2006/50006), bromine in an acetic medium (analogy with patent WO 2007/126841), pyridinium tribromide in an organic solvent such as methanol (analogy with patent US 2005/277638) or preferably N-bromosuccinimide in an organic solvent such as dimethylformamide (analogy with Bioorg. Med. Chem. 2008, 16 (11), 5962).

The product of general formula (II) in which Z represents the carboxyl radical and R4 represents F can also be obtained by fluorination of the compound of general formula (II) in which Z represents the carboxyl radical and R4 represents H, by using a fluorinating reagent known to those skilled in the art, such as, for example, Selectfluor® in an organic solvent such as acetonitrile as a mixture with acetic acid (analogy with patent WO 2009/147188).

The product of general formula (II) in which Z represents the carboxyl radical and R4 represents I can also be obtained by iodination of the compound of general formula (II) in which Z represents the carboxyl radical and R4 represents H, by using an iodinating reagent known to those skilled in the art, such as iodine in a basic aqueous medium (analogy with patent WO 2008/154241 or Synlett (20), 3216 (2008)), or N-iodosuccinimide in an organic solvent such as dimethylformamide.

The product of general formula (II) in which Z represents the formyl radical and R4 represents H can be prepared according to patent WO 2007/051062.

The product of general formula (II) in which Z represents the formyl radical and R4 represents I is known in the literature (Registry Number=944904-44-3),

In general, the products of general formula (H) in which Z represents a formyl radical and R4 represents CH₃, CF₃, CH₂CH₃, F, Cl, Br or I can be obtained by carrying out the process by analogy with carbazole according to the Journal of the Chemical Society (1957), 2210-5 or according to scheme (6) below:

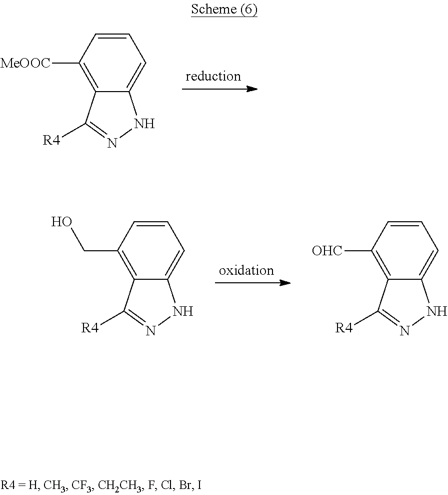

using reducing agents and oxidants known to those skilled in the art.

The product of general formula (II) in which Z represents the formyl radical and R4 represents Cl can also be obtained by chlorination of the compound of general formula (II) in which Z represents the formyl radical and R4 represents H, by using a chlorinating reagent known to those skilled in the art, such as sodium hypochlorite in a basic aqueous medium (analogy with Bioorg. Med. Chem. 2007, 15 (6), 2441), chlorine gas in an acetic medium (analogy with J. Med. Chem. 2003, 46 (26), 5663) or preferably N-chlorosuccinimide in an organic solvent such as dimethylformamide (analogy with patent WO 1997/12884).

The product of general formula (II) in which Z represents the formyl radical and R4 represents Br can be obtained by bromination of the compound of general formula (II) in which Z represents the formyl radical and R4 represents H, by using a brominating reagent known to those skilled in the art, such as sodium hypobromite in a basic aqueous medium (analogy with patent WO 2006/50006), bromine in an acetic medium (analogy with patent WO 2007/126841), pyridinium tribromide in an organic solvent such as methanol (analogy with patent US 2005/277638) or preferably N-bromosuccinimide in an organic solvent such as dimethylformamide (analogy with Bioorg. Med. Chem. 2008, 16 (11), 5962).

The product of general formula (II) in which Z represents the formyl radical and R4 represents F can also be obtained by fluorination of the compound of general formula (II) in which Z represents the formyl radical and R4 represents H, by using a fluorinating reagent known to those skilled in the art, such as, for example, Selectfluor® in an organic solvent such as acetonitrile as a mixture with acetic acid (analogy with patent WO 2009/147188).

The product of general formula (II) in which Z represents the formyl radical and R4 represents I can also be obtained by iodination of the compound of general formula (II) in which Z represents the formyl radical and R4 represents H, by using an iodinating reagent known to those skilled in the art, such as iodine in a basic aqueous medium (analogy with patent WO 2008/154241 or Synlett (20), 3216 (2008)), or N-iodosuccinimide in an organic solvent such as dimethylformamide.

The product of general formula (II) in which Z represents the bromine atom and R4 represents H can be obtained by carrying out the process according to J. Med. Chem. (2008), 51 (18), 5522.

The product of general formula (II) in which Z represents the bromine atom and R4 represents I is known in the literature (Registry Number=885521-72-2).

The product of general formula (II) in which Z represents the iodine atom and R4 represents H can be obtained by carrying out the process according to Bioorg. Med. Chem. Lett. (2007), 17 (11), 3177.

The product of general formula (II) in which Z represents the iodine atom and R4 represents I is known in the literature (Registry Number=885518-66-1).

The products of general formula (II) in which Z represents a boronic acid or a boronic ester, which is optionally cyclic, and R4 represents H, CH₃, CF₃, CH₂CH₃, F, Cl, Br or I can be advantageously prepared by the action of N-butyl-lithium and then of a borate, such as dimethyl borate, di-n-butyl borate, diisopropyl borate or pinacolyl borate, or of a diboronic ester, on the corresponding 4-bromo-1H-indazole at low temperature in an organic solvent such as tetrahydrofuran, or alternatively starting from the corresponding 4-iodo-1H-indazole derivative, or more advantageously starting from the corresponding 4-trifluoromethylsulphonyl-oxy derivative, in the presence of a palladium(0) catalyst, according to scheme (7).

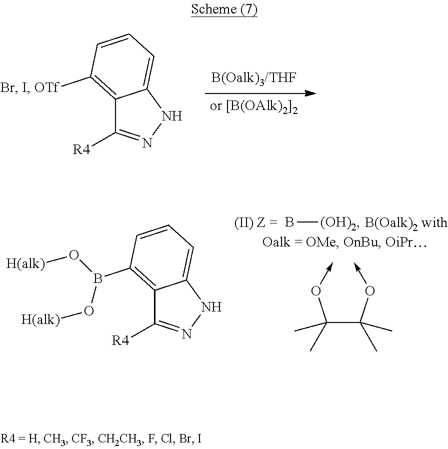

The product of general formula (II) in which Z represents the pinacolylboronic radical and R4 the H radical can also be prepared according to J. Med. Chem. (2008), 51 (18), 5522.

The product of general formula (II) in which Z represents a boronic acid or a boronic ester, which is optionally cyclic, and R4 represents Cl can also be obtained by chlorination of the compound of general formula (II) in which Z represents a boronic acid or a boronic ester, which is optionally cyclic, and R4 represents H, by using a chlorinating reagent known to those skilled in the art, and preferably N-chlorosuccinimide in an organic solvent such as dimethylformamide (analogy with patent WO 1997/12384).

The product of general formula (II) in which Z represents a boronic acid or a boronic ester, which is optionally cyclic, and R4 represents Br can be obtained by bromination of the compound of general formula (II) in which Z represents a boronic acid or a boronic ester, which is optionally cyclic, and R4 represents H, by using a brominating reagent known to those skilled in the art, such as pyridinium tribromide in an organic solvent such as methanol (analogy with patent US 2005/277638), or preferably N-bromosuccinimide in an organic solvent such as dimethylformamide (analogy with Bioorg. Med. Chem. 2008, 16 (11), 5962).

The product of general formula (II) in which Z represents a boronic acid or a boronic ester, which is optionally cyclic, and R4 represents F can also be obtained by fluorination of the compound of general formula (II) in which Z represents a boronic acid or a boronic ester, which is optionally cyclic, and R4 represents H, by using a fluorinating reagent known to those skilled in the art, such as, for example, Selectfluor® in an organic solvent such as acetonitrile as a mixture with acetic acid (analogy with patent WO 2009/147188).

The product of general formula (II) in which Z represents a boronic acid or a boronic ester, which is optionally cyclic, and R4 represents I can also be obtained by iodination of the compound of general formula (II) in which Z represents a boronic acid or a boronic ester, which is optionally cyclic, and R4 represents H, by using an iodinating reagent known to those skilled in the art, such as iodine in a basic aqueous medium (analogy with patent WO 2008/154241 or Synlett (20), 3216 (2008)), or N-iodosuccinimide in an organic solvent such as dimethylformamide.

Preparation of the Compounds of General Formula (III)

The subject of the present invention is thus also the methods for synthesizing the products of formula (III), in which, R1 and/or R′1 being as defined above, R4 represents H, CH₃, CF₃, CH₂CH₃, F, Cl, Br or I, and Het is in the group constituted of:

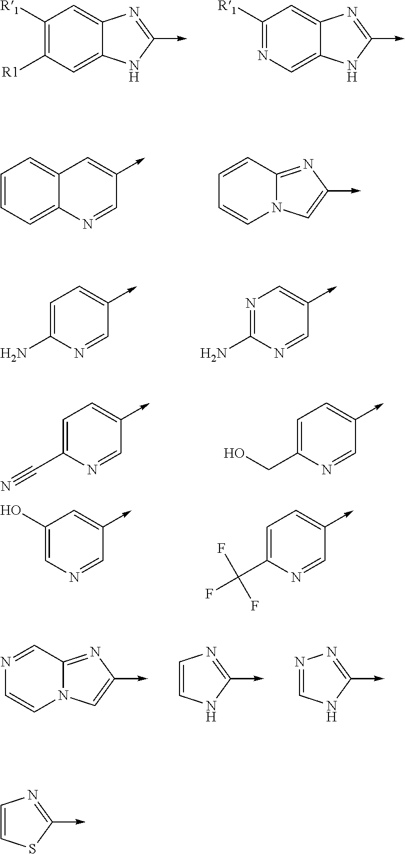

More particularly, when Het does not represent a heterocycle of imidazol-2-yl, triazol-3-yl, benzimidazol-2-yl or azabenzimidazol-2-yl type, and is optionally substituted with one or more R1 radicals, as defined above, it is particularly advantageous, according to the invention, to prepare the compounds of general formula (III)
either by coupling the corresponding 4-bromoindazole, 4-iodo-indazole or 4-trifluoromethylsulphonyloxyindazole with a heterocyclic boronic derivative, which may be an acid or an ester,
or by coupling the corresponding indazol-4-boronic acid, or an ester thereof, such as the methyl, n-butyl, isopropyl or pinacol ester, with a bromo or an iodo heterocycle,
   under the Suzuki reaction conditions, in the presence of a palladium(0) derivative as catalyst, the process being carried out according to scheme (8):

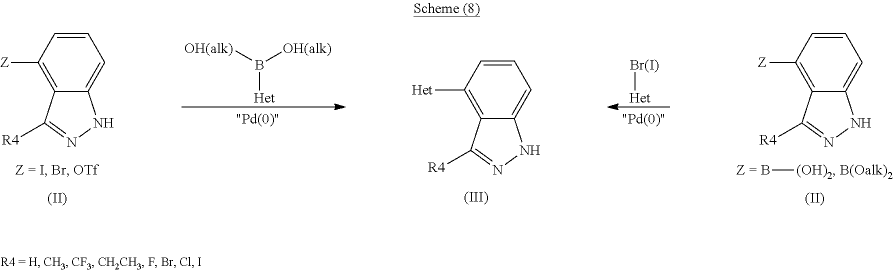

It is also possible to directly obtain the compounds (III) according to scheme (8a) starting from the ditriflate compounds prepared according to scheme (4a):

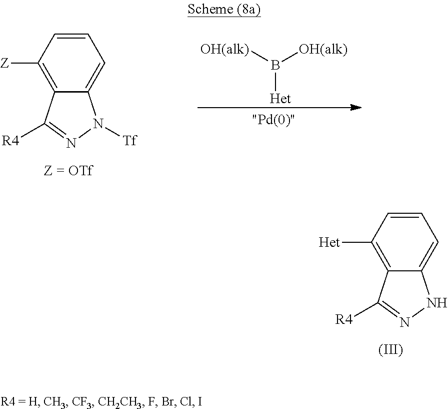

More particularly, when the heterocycle Het is of the type benzimidazole or azabenzimidazole—or alternatively the type benzoxazole or azabenzoxazole, or benzothiazole or azabenzothiazole, linked via its 2-position to position 4 of the indazole, it is particularly advantageous to form said heterocycle by coupling a derivative of ortho-phenylenediamine or of diaminopyridine—or alternatively of ortho-aminophenol, of ortho-aminothiophenol or of aminohydroxypyridine or of aminomercaptopyridine, which is ortho-disubstituted, —with an acid, an acid chloride, a methyl or ethyl ester or an aldehyde at position 4 of the corresponding indazole N-protected with a protective group such as a tert-butyloxycarbonyl (Boc) radical or a tert-butyldimethylsilyl (TBDMS) radical or a 2-(trimethylsilyl)ethoxy-methyl (SEM) radical, followed by cyclization in an acid medium, which enables cleavage of the Boc, TBDMS or SEM protective group borne by the nitrogen atom of the corresponding indazole, the process being carried out according to scheme (9):

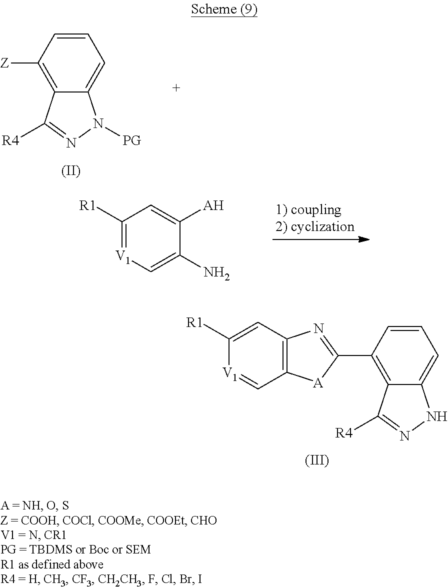

In the context of the invention, it is advantageous to protect the nitrogen of an indazole derivative bearing an acid, ester or aldehyde radical at position 4, with a tert-butyloxycarbonyl (Boc) group—by the action of Boc₂O, BocCl or BocON in an organic solvent such as dichloromethane or tetrahydrofuran in the presence of an organic or inorganic base—or with a tert-butyldimethylsilyl (TBDMS) group—by the action of tert-butyldimethylsilane chloride (TBDMSCl) in an organic solvent such as dichloromethane or tetrahydrofuran in the presence of an organic or inorganic base—or with a 2-(trimethylsilyl)ethoxymethyl (SEM) group—by the action of 2-(trimethylsilyl)ethoxymethyl chloride (SEMCl) in an organic solvent such as dichloromethane or tetrahydrofuran in the presence of an organic or inorganic base.

When an N-protected derivative of indazole-4-carboxylic acid is used, it is particularly advantageous to activate this acid using a coupling agent known to those skilled in the art, such as 1-(3-dimethylaminopropyl)-3-ethylcarbodiimide hydrochloride (EDCl) in the presence of 1-hydroxybenzotriazole (HOBT), or of O-(ethoxycarbonyl)cyanomethyleneamino)-N,N,N′,N′-tetramethyluronium tetrafluoroborate (TOTU).

When an N-protected derivative of indazole-4-carboxylic acid methyl or ethyl ester is used, it is advantageous, in the context of the invention, to carry out the process in the presence of trimethylaluminium in a halogenated organic solvent such as dichloromethane or dichloroethane.

When an N-protected derivative of indazole-4-carboxaldehyde is used, it is advantageous, in the context of the invention, to carry out the process:

either by microwave heating in the presence of silica, according to Tetrahedron Lett. 1998, 39, 4481-84;

or in the presence of dichlorodicyanobenzoquinone (DDQ), according to Tetrahedron 1995, 51, 5813-18;

or in the presence of a mixture of thionyl chloride and of pyridine, according to EP 511187;

or in the presence of ferric chloride, according to Eur. J. Med. Chem., 2006, 31, 635-42.

Various conditions for cyclization of the mixture of intermediate amides can be used in the context of the invention, such as acetic acid or a mixture of trifluoroacetic acid and trifluoroacetic anhydride. It is also particularly advantageous, in the context of the invention, to perform this type of thermal cyclization in an acid medium by heating in a microwave reactor.

More particularly, when said heterocycle is of imidazole, oxazole or thiazole type, linked via its 2-position to position 4 of the indazole, it is particularly advantageous to form said heterocycle using an acid, an acid chloride, an ester or an aldehyde at position 4 of an N-protected indazole derivative, the process being carried out according to scheme (10):

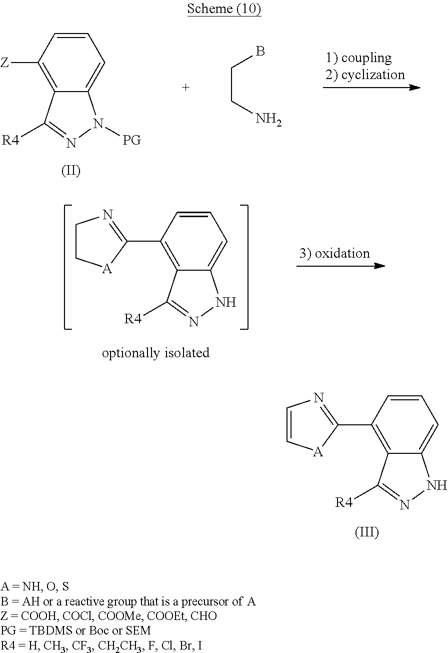

In the context of the invention, it is advantageous to protect the nitrogen of an indazole derivative bearing an acid, ester or aldehyde radical at position 4, with a tert-butyloxycarbonyl (Boc) group—by the action of Boc₂O, BocCl or BocON in an organic solvent such as dichloromethane or tetrahydrofuran in the presence of an organic or inorganic base—or with a tert-butyldimethylsilyl (TBDMS) group—by the action of tert-butyldimethylsilane chloride (TBDMSCl) in an organic solvent such as dichloromethane or tetrahydrofuran in the presence of an organic or inorganic base—or with a 2-(trimethylsilyl)ethoxymethyl (SEM) group—by the action of 2-(trimethylsilyl)ethoxymethyl chloride (SEMCl) in an organic solvent such as dichloromethane or tetrahydrofuran in the presence of an organic or inorganic base.

In the context of the invention, it is particularly advantageous to carry out the process:

1. when said heterocycle is an imidazole or an imidazoline:
using a 2-azidoethylamine, according to Tetrahedron, 47(38), 1991, 8177-94,
an ethylenediamine, according to Bioorg. Med. Chem. Lett. 12(3), 2002, 471-75,
glyoxal and aqueous ammonia, according to J. Med, Chem., 46(25), 2003, 5416-27;

2. when said heterocycle is an oxazole or an oxazoline:
using a 2-azidoethanol, according to J. Org. Chem., 61(7), 1996, 2487-96,
a 2-aminoethanol, according to J. Med. Chem. 47(8), 2004, 1969-86 or Khim. Geterosikl. Soed. 1984(7), 881-4,
2-aminoacetaldehyde diethyl acetal, according to Heterocycles, 39(2), 1994, 767-78;

3. when said heterocycle is a thiazole or a thiazoline:
using a 2-chloroethylamine and Lawesson's reagent, according to Helv. Chim. Acta, 88(2), 2005, 187-95,
a 2-aminoethanethiol, according to J. Org. Chem. 69(3), 2004, 811-4, or Tetrahedron Lett., 41(18), 2000, 3381-4.

The products of general formula (III) in which R4 represents Cl can also be obtained by chlorination of the corresponding compound of general formula (III) in which R4 represents H, by using a chlorinating reagent known to those skilled in the art, such as sodium hypochlorite in a basic aqueous medium (analogy with Bioorg. Med. Chem. 2007, 15 (6), 2441), chlorine gas in an acetic medium (analogy with J. Med. Chem. 2003, 46 (26), 5663) or preferably N-chlorosuccinimide in an organic solvent such as dimethylformamide (analogy with patent WO 1997/12884).

The products of general formula (III) in which R4 represents Br can also be obtained by bromination of the corresponding compound of general formula (III) in which R4 represents H, by using a brominating reagent known to those skilled in the art, such as sodium hypobromite in a basic aqueous medium (analogy with patent WO 2006/50006), bromine in an acetic medium (analogy with patent WO 2007/126841), pyridinium tribromide in an organic solvent such as methanol (analogy with patent US 2005/277638) or preferably N-bromosuccinimide in an organic solvent such as dimethylformamide (analogy with Bioorg. Med. Chem. 2008, 16 (11), 5962),

The products of general formula (III) in which R4 represents F can also be obtained by fluorination of the corresponding compound of general formula (III) in which R4 represents H, by using a fluorinating reagent known to those skilled in the art, such as, for example, Selectfluor® in an organic solvent such as acetonitrile as a mixture with acetic acid (analogy with patent WO 2009/147188).

The products of general formula (III) in which R4 represents I can also be obtained by iodination of the corresponding compound of general formula (III) in which R4 represents H, by using an iodinating reagent known to those skilled in the art, such as iodine in a basic aqueous medium (analogy with patent WO 2008/154241 or Synlett (20), 3216 (2008)), or N-iodosuccinimide in an organic solvent such as dimethylformamide.

More generally, it is advantageous, in the context of the invention, to form the heterocycle of a product of general formula (III) using a triflate, a brominated or iodinated derivative, a boronic acid or ester, a carboxylic acid, an acid chloride, a carboxylic acid ester or an aldehyde, at position 4 of an indazole, by any one of the methods of synthesis known to those skilled in the art, such as those described in Comprehensive Organic Chemistry, by D. H. R. Barton et al. (Pergamon Press) or Advances in Heterocyclic Chemistry (Academic Press) or Heterocyclic Compounds (Wiley Intersciences).

Preparation of the Compounds of General Formula (IV)

The subject of the present invention is thus also the methods of synthesizing the products of formula (IV), in which Z represents a carboxylic ester group, in particular methyl or ethyl ester, or a benzyloxy radical, and R4 represents H, CH₃, CF₃, CH₂CH₃, F, Cl, Br or I.

The products of general formula (IV) in which Z represents a carboxylic ester or a benzyloxy radical can be advantageously prepared, in the context of the present invention, by reacting a product of general formula (II), in which Z represents a carboxylic ester or a benzyloxy radical and R4 represents H, CH₃, CF₃, CH₂CH₃, F, Cl, Br or I,

1) either by carrying out the process according to scheme (11) or (11a):
by means of a reaction of aromatic nucleophilic substitution of 2-bromo-4-fluorobenzonitrile, or of 4-bromo-5-cyano-2-fluoropyridine or of 3-bromo-2-cyano-5-fluoropyridine, in a solvent such as dimethylformamide (DMF), dimethyl sulphoxide (DMSO) or N-methylpyrrolidone (NMP), after having pretreated the indazole derivative of general formula (II) with a strong base, for instance sodium hydride,
followed by Buchwald-Hartwig amination with an amine R₂—NH₂, in which R2 is as defined above, in the presence of a base such as, for example, potassium tert-butoxide or cesium carbonate and of a palladium(0) derivative, formed from palladium acetate and from a ligand such as 1,1′-bis(diphenylphosphino)ferrocene or 4,5-bis(diphenylphosphino)-9,9-dimethylxanthene, in a solvent such as, for example, toluene or dioxane.

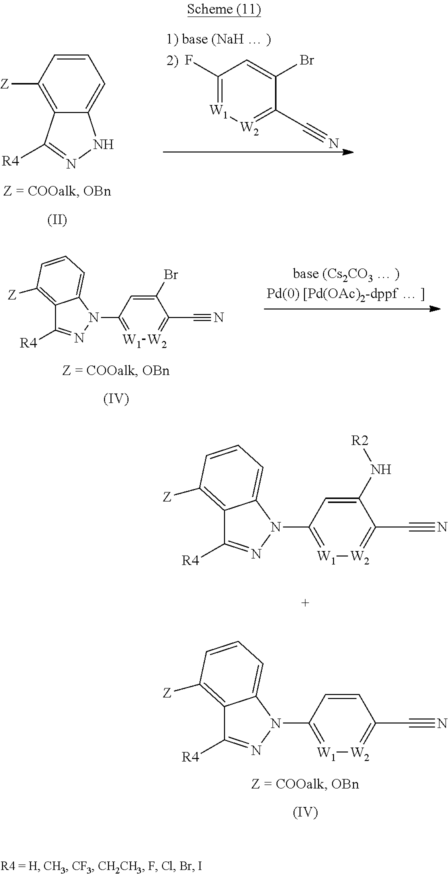

Generally, the compound resulting from bromine hydrogenolysis can also be obtained during this Buchwald-Hartwig step, it being possible for this compound to also be obtained directly from the compounds (II) with Z=COOAlk or OBn according to scheme (11a).

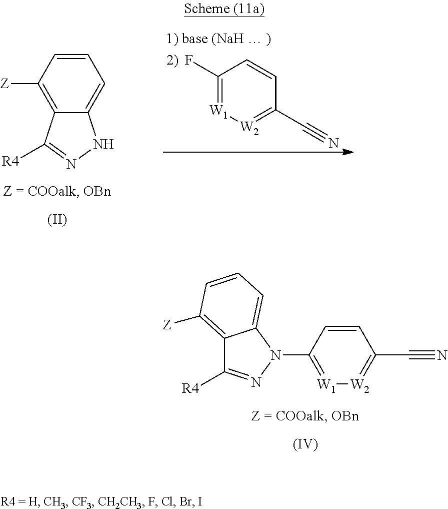

2) or by carrying out the process according to scheme (12) or (12a):
by means of a Buchwald-Hartwig reaction between 4-bromo-2-fluorobenzonitrile or 2-bromo-5-cyano-4-fluoropyridine or 5-bromo-2-cyano-3-fluoropyridine, and an indazole of general formula (II), in the presence of a base such as cesium carbonate and a palladium(0) derivative, such as “Palladium-Xanthphos” formed from palladium acetate and from 4,5-bis(diphenylphosphino)-9,9-dimethylxanthene, in a solvent such as dioxane,
followed by a reaction of aromatic nucleophilic substitution with an amine R2—NH₂, in which R2 is as defined above, in the presence of a base such as potassium carbonate, in a solvent such as DMSO.

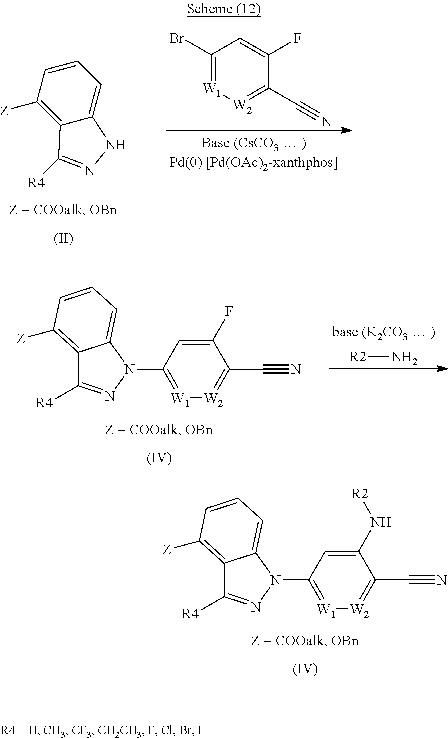

The compounds (IV) in which Z represents a trifluoromethanesulphonyloxy or COOAlk radical and R4 represents CH₃, CF₃ or CH₂CH₃ can also be prepared according to scheme (12a) directly from the compounds (IIa″):
by reaction with the corresponding hydrazines, which can be prepared according to the described methods known to those skilled in the art for forming hydrazones and preferably in a hydroxylated solvent such as ethanol;
then cyclization in an acid medium, preferably with microwave irradiation, so as to obtain the corresponding compounds (IIb′);
then bromination and treatment with a base by analogy with scheme (2), so as to obtain the products (IV) in which Z represents OH;
then introduction of an amine R2—NH₂ by means of a Buchwald-Hartwig coupling reaction (analogy with scheme (11)) or by means of an aromatic nucleophilic substitution (analogy with scheme (12)), so as to obtain the aminated products (IV) in which Z represents OH;
then trifluoromethanesulphonation by analogy with scheme (4), so as to obtain the products (IV) in which Z represents OTf;
finally, carbonylation as in scheme (5), so as to obtain the products (IV) in which Z represents the COOAlk group.

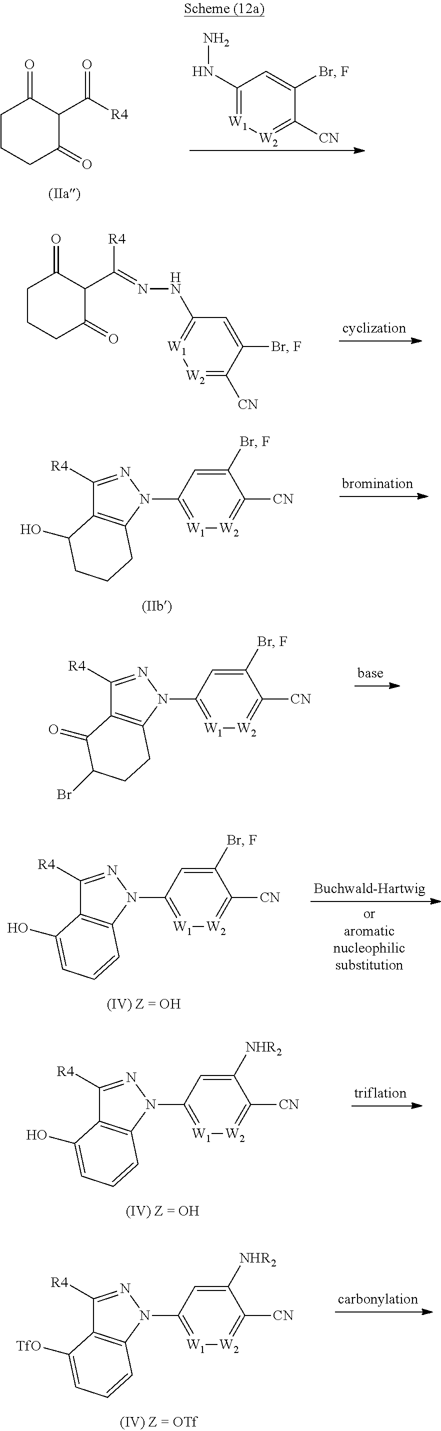

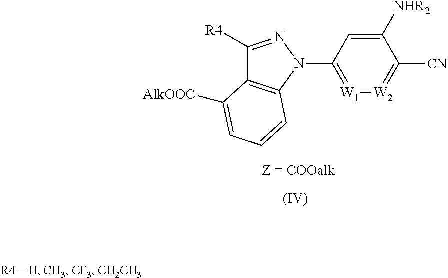

The products of general formula (IV) in which Z represents a carboxylic acid or a hydroxyl radical and R4 represents H, CH₃, CF₃, CH₂CH₃, F, Cl, Br or I can be respectively prepared by alkaline hydrolysis of the corresponding esters or by hydrogenolysis of the corresponding benzyloxy derivatives, according to the conventional methods known to those skilled in the art.

The products of general formula (IV) in which Z represents a trifluoromethanesulphonyloxy radical and R4 represents H, CH₃, CF₃, CH₂CH₃, F, Cl, Br or I can be obtained as described above in scheme (4), by the action of N-phenylbis(trifluoromethanesulphonimide), in an organic solvent such as dichloromethane, in the presence of an organic base such as triethylamine, on a product of general formula (IV) in which Z represents a hydroxyl radical and R4 represents H, CH₃, CF₃, CH₂CH₃, F, Cl, Br or I.

The products of general formula (IV) in which R4 represents Cl can also be obtained by chlorination of the corresponding compound of general formula (IV) in which R4 represents H, by using a chlorinating reagent known to those skilled in the art, such as sodium hypochlorite in a basic aqueous medium (analogy with Bioorg. Med. Chem. 2007, 15 (6), 2441), chlorine gas in an acetic medium (analogy with J. Med. Chem. 2003, 46 (26), 5663) or preferably N-chlorosuccinimide in an organic solvent such as dimethylformamide (analogy with patent WO 1997/12884).

The products of general formula (IV) in which R4 represents Br can also be obtained by bromination of the corresponding compound of general formula (IV) in which R4 represents H, by using a brominating reagent known to those skilled in the art, such as sodium hypobromite in a basic aqueous medium (analogy with patent WO 2006/50006), bromine in an acetic medium (analogy with patent WO 2007/126841), pyridinium tribromide in an organic solvent such as methanol (analogy with patent US 2005/277638) or preferably N-bromosuccinimide in an organic solvent such as dimethylformamide (analogy with Bioorg. Med. Chem. 2008, 16 (11), 5962).

The products of general formula (IV) in which R4 represents F can also be obtained by fluorination of the corresponding compound of general formula (IV) in which R4 represents H, by using a fluorinating reagent known to those skilled in the art, such as, for example, Selectfluor® in an organic solvent such as acetonitrile as a mixture with acetic acid (analogy with patent WO 2009/147188).

The products of general formula (IV) in which R4 represents I can also be obtained by iodination of the corresponding compound of general formula (IV) in which R4 represents H, by using an iodinating reagent known to those skilled in the art, such as iodine in a basic aqueous medium (analogy with patent WO 2008/154241 or Synlett (20), 3216 (2008)), or N-iodosuccinimide in an organic solvent such as dimethylformamide.

The products of general formula (IV) in which Z represents COOMe and R4 represents H, CH₃, CF₃ or CH₂CH₃ can also be obtained starting from the corresponding compounds (IV) in which Z represents OTf, by carbonylation in the presence of a catalyst such as palladium, by analogy with the reaction of scheme (5).

Preparation of the Compounds of General Formula (V)

The subject of the present invention is thus also the methods for synthesizing the products of formula (V), in which Z represents a carboxyl radical, a carboxylic ester group, in particular methyl or ethyl ester, a hydroxyl radical or a benzyloxy radical and R4 represents H, CH₃, CF₃, CH₂CH₃, F, Cl, Br or I.

The compounds of general formula (V) in which R is of type A can be prepared by hydrolysis of the cyano radical of a compound of general formula (IV). This hydrolysis can be carried out, advantageously in the context of the invention, by the action of an aqueous solution of hydrogen peroxide, according to scheme (13):

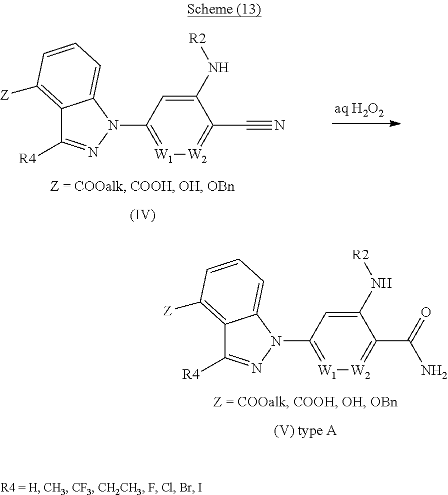

The compounds of general formula (V) in which R is of type B and X is an NH radical can be prepared, advantageously in the context of the invention, by means of an aromatic nucleophilic substitution reaction, followed by intramolecular cyclization, by the action of hydrazine hydrate in a polar solvent, such as n-butanol, on a nitrile of general formula (IV), ortho-substituted with a halogen atom, very preferably a fluorine atom, according to scheme (14):

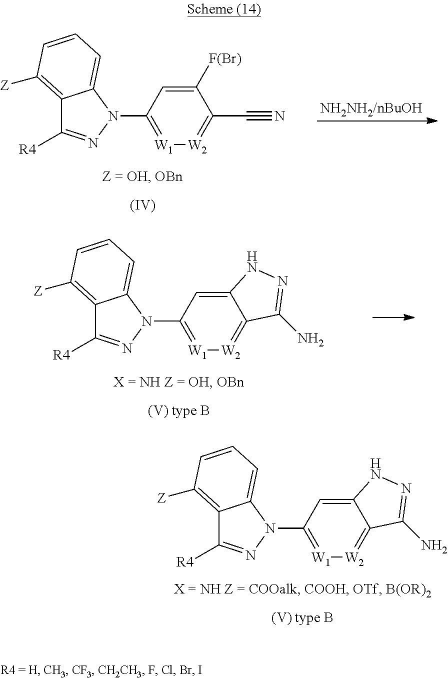

The compounds of general formula (V), in which R is of type B and X is an NR2 radical, with R2 as defined above, can be prepared according to scheme (15):
either, advantageously in the context of the invention, by the action of a hydrazine monosubstituted with an R2 radical, in a polar solvent, such as n-butanol, on a nitrile of general formula (IV), ortho-substituted with a halogen atom, very preferably a fluorine atom,
or by N-alkylation of a product of general formula (V) of type B with X=NH. This alkylation can be carried out according to the methods known to those skilled in the art, in particular by treatment with a base such as sodium hydride, followed by the action of a halogenated derivative R2—Hal.

When the process is carried out in this way, a mixture of N1-N2-alkylated regio-isomers is generally obtained, it being possible for these regioisomers to be separated using the conventional methods known to those skilled in the art.

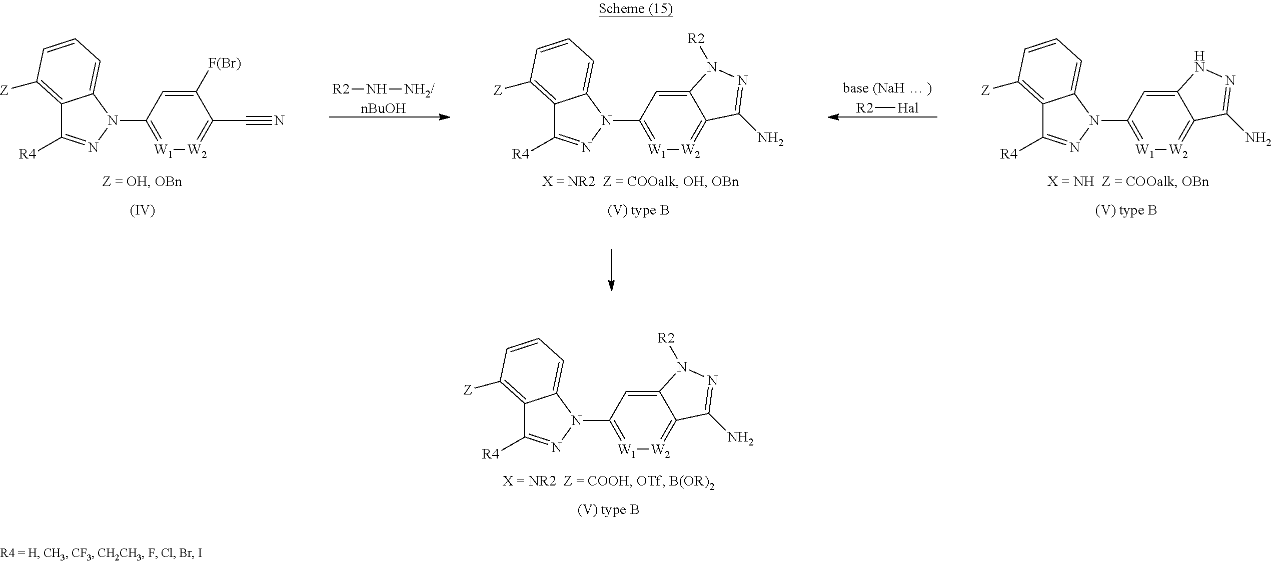

The compounds of general formula (V) in which R is of type B and X is an oxygen atom can be prepared, advantageously in the context of the invention, by the action of an N-protected hydroxylamine, such as N-tert-butyloxycarbonyl-hydroxylamine, in the presence of a strong base, such as potassium tert-butoxide, on a nitrile of general formula (IV), ortho-substituted with a halogen atom, very preferably a fluorine atom, in a solvent such as DMF, by carrying out the process according to scheme (16):

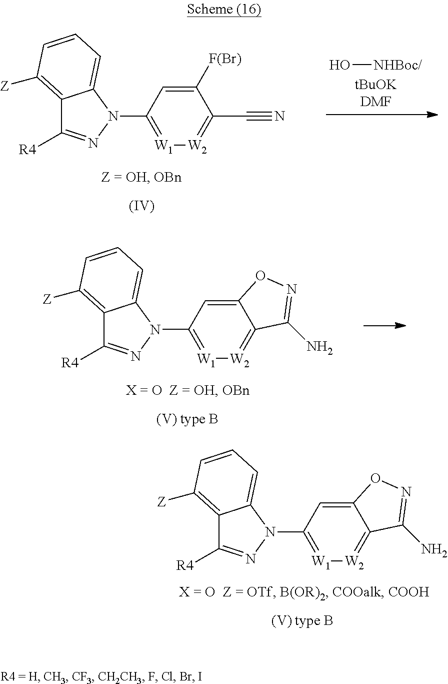

The compounds of general formula (V) in which R is of type B and X is a sulphur atom can be prepared, advantageously in the context of the invention, by the action of sodium sulphide in a solvent such as DMSO, on a nitrile of general formula (IV), ortho-substituted with a halogen atom, very preferably a fluorine atom, followed by the action of aqueous ammonia in the presence of sodium hypochlorite, by carrying out the process according to scheme (17), in particular under the conditions described in Bioorg. Med. Chem. Lett. (2007), 17(6), 4568:

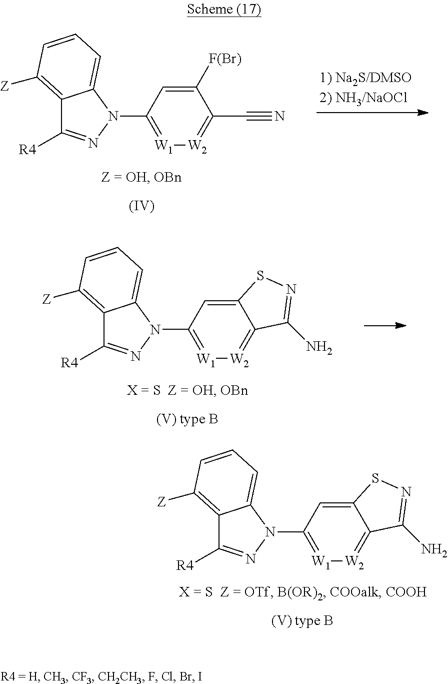

The compounds of general formula (V) in which R is of type C can be prepared, advantageously in the context of the invention, by the action of hydroxylamine hydrochloride on a nitrile of general formula (IV), ortho-substituted with a halogen atom, very preferably a fluorine atom, by carrying out the process according to scheme (18), in particular under the conditions described in Zeitschrift für Chemie (1984), 24(7), 254:

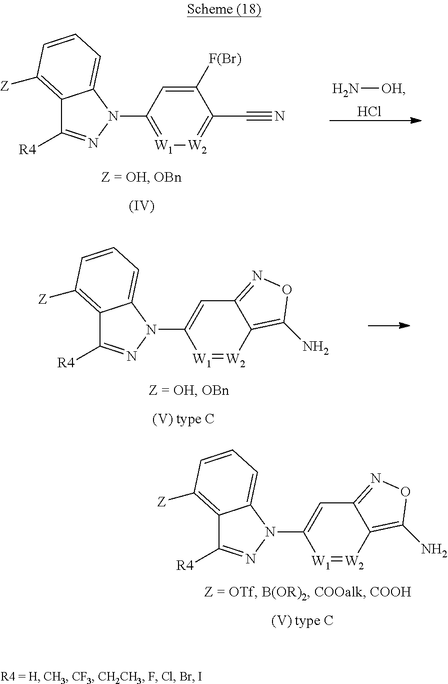

The compounds of general formula (V) in which R is of type D, with W3 being a nitrogen atom, can be prepared, advantageously in the context of the invention, by the action of aqueous ammonia on a nitrile of general formula (IV), ortho-substituted with a halogen atom, very preferably a fluorine atom, followed by the action of a mixture of ethyl orthoformate and ammonium acetate, by carrying out the process according to scheme (19), in particular under the conditions described in J. Het. Chem. (2006), 43(4), 913:

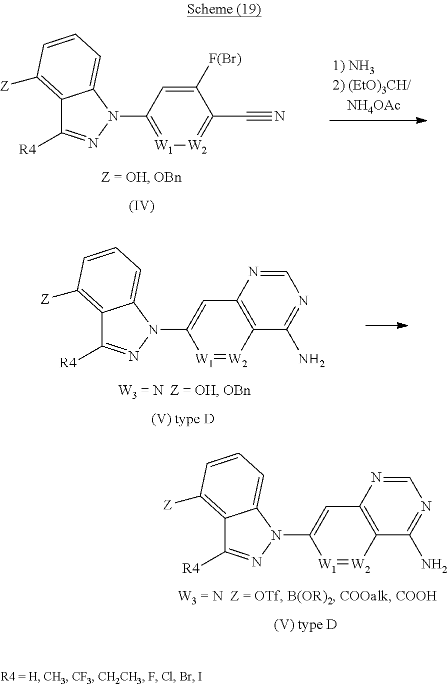

The compounds of general formula (V) in which R is of type E can be prepared, advantageously in the context of the invention, by the action of trimethylsilylacetylene, in the presence of a base, such as triethylamine or n-butylamine, in the presence of cuprous iodide and of tetrakis(triphenylphosphine)palladium, on a compound of general formula (IV), ortho-substituted with a bromine atom, so as to give an acetylenic intermediate, which is then successively treated with sodium ethoxide in ethanol, and then with a solution of hydrogen peroxide in an alkali medium and, finally, heated in the presence of para-toluenesulphonic acid, by carrying out the process according to general scheme (20), in particular under the conditions described in Chem. Pharm. Bull. (1986), 34, 2760.

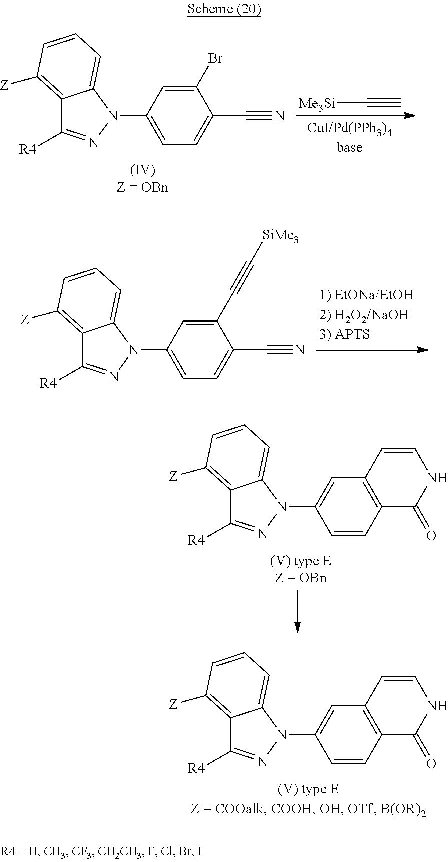

The compounds of general formula (V) in which R is of type D, with W₁, W₂ and W₃=CH, can be prepared, advantageously in the context of the invention, by the action of phosphorus trichloride and then of acetamide, at a temperature close to 180° C. in the presence of a base such as potassium carbonate, on a product of general formula (V) of type E, by carrying out the process according to scheme (21), in particular under the conditions described in Bioorg. Med. Chem. (2006), 14(20), 6832.

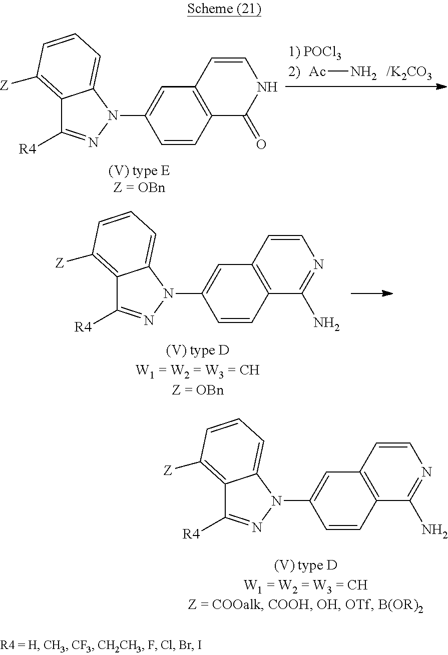

The products of general formula (V) in which R4 represents Cl can also be obtained by chlorination of the corresponding compound of general formula (V) in which R4 represents H, by using a chlorinating reagent known to those skilled in the art, such as sodium hypochlorite in a basic aqueous medium (analogy with Bioorg. Med. Chem. 2007, 15 (6), 2441), chlorine gas in an acetic medium (analogy with J. Med. Chem. 2003, 46 (26), 5663) or preferably N-chlorosuccinimide in an organic solvent such as dimethylformamide (analogy with patent WO 1997/12884).

The products of general formula (V) in which R4 represents Br can also be obtained by bromination of the corresponding compound of general formula (V) in which R4 represents H, by using a brominating reagent known to those skilled in the art, such as sodium hypobromite in a basic aqueous medium (analogy with patent WO 2006/50006), bromine in an acetic medium (analogy with patent WO 2007/126841), pyridinium tribromide in an organic solvent such as methanol (analogy with patent US 2005/277638) or preferably N-bromosuccinimide in an organic solvent such as dimethylformamide (analogy with Bioorg. Med. Chem. 2008, 16 (11), 5962).

The products of general formula (V) in which R4 represents F can also be obtained by fluorination of the corresponding compound of general formula (V) in which R4 represents H, by using a fluorinating reagent known to those skilled in the art, such as, for example, Selectfluor® in an organic solvent such as acetonitrile as a mixture with acetic acid (analogy with patent WO 2009/147188).

The products of general formula (V) in which R4 represents I can also be obtained by iodination of the corresponding compound of general formula (V) in which R4 represents H, by using an iodinating reagent known to those skilled in the art, such as iodine in a basic aqueous medium (analogy with patent WO 2008/154241 or Synlett (20), 3216 (2008)), or N-iodosuccinimide in an organic solvent such as dimethylformamide.

Preparation of the Compounds of General Formula (VI)

The subject of the present invention is thus also the methods for synthesizing the products of formula (VI).

A) Starting from the Product of General Formula (IV)

More particularly, when Het does not represent a heterocycle of imidazol-2-yl, triazol-3-yl, benzimidazol-2-yl or azabenzimidazol-2-yl type, and is optionally substituted with one or more R1 radicals, as defined above, it is particularly advantageous, according to the invention, to prepare the compounds of general formula (VI) by coupling:

either a product of general formula (IV), in which Z represents a trifluoromethyl-sulphonyloxy radical, with a heterocyclic boronic derivative, which may be an acid or an ester, such as the methyl, n-butyl, isopropyl or pinacol ester, under the Suzuki reaction conditions, in the presence of a palladium(0) derivative as catalyst,

or a product of general formula (IV), in which Z represents a boronic derivative, which may be an acid or an ester, such as the methyl, n-butyl, isopropyl or pinacol ester, itself prepared by coupling, in the presence of palladium(0) as catalyst, with a brominated or iodinated heterocyclic derivative, by carrying out the process according to scheme (22):

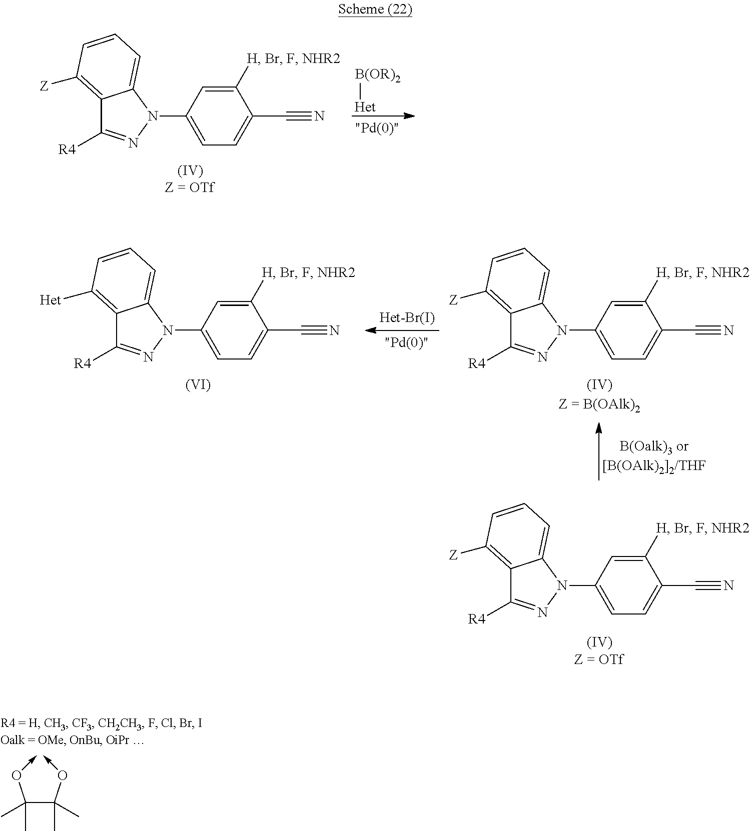

More particularly, when Het is a heterocycle of benzimidazole or azabenzimidazole type—or alternatively of benzoxazole or azabenzoxazole, benzothiazole or azabenzothiazole type, linked via its 2-position to position 4 of the indazole, it is particularly advantageous to form said heterocycle by coupling a derivative of ortho-phenylenediamine or of diaminopyridine—or alternatively of ortho-aminophenol, of ortho-aminothiophenol or of aminohydroxypyridine or of aminomercaptopyridine, which is ortho-disubstituted, —with a derivative of general formula (IV) in which Z represents an acid or an ester, in particular a methyl or ethyl ester, by carrying out the process according to scheme (23):

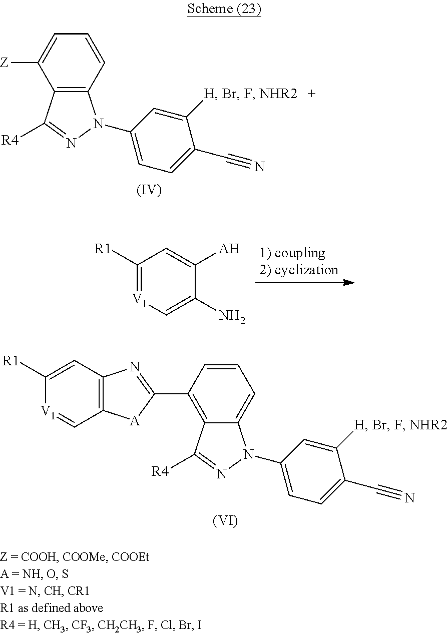

When a product of general formula (IV) in which Z is an acid is used, it is particularly advantageous to activate this acid using a coupling agent known to those skilled in the art, such as 1-(3-dimethylaminopropyl)-3-ethylcarbodiimide hydrochloride (EDCl) in the presence of 1-hydroxybenzotriazole (HOBT), or of O-((ethoxycarbonyl)cyanomethyleneamino)-N,N,N′,N′-tetramethyluronium tetrafluoroborate (TOTU).

When a product of general formula (IV) in which Z is a methyl or ethyl ester is used, it is advantageous, in the context of the invention, to carry out the process in the presence of trimethylaluminium in a halogenated organic solvent such as dichloromethane or dichloroethane.

Various conditions for cyclization of the mixture of intermediate amides can be used in the context of the invention, such as acetic acid or a mixture of trifluoroacetic acid and trifluoroacetic anhydride. It is also particularly advantageous, in the context of the invention, to carry out this type of thermal cyclization in an acid medium by heating in a microwave reactor.

More particularly, when said heterocycle is of imidazole, oxazole or thiazole type, linked via its 2-position to position 4 of the indazole, it is particularly advantageous to form said heterocycle using an acid or an ester, by carrying out the process according to scheme (24):

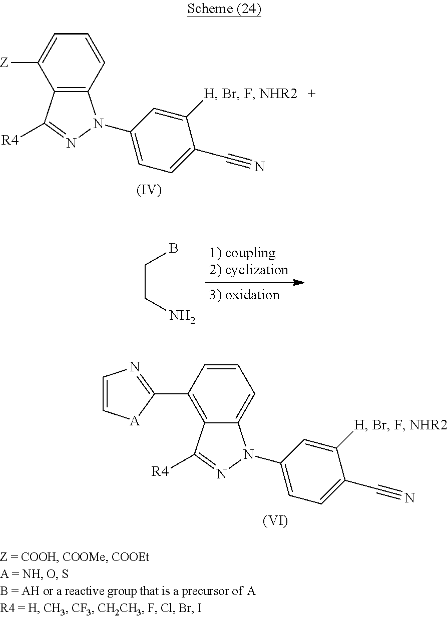

In the context of the invention, it is particulaly advantageous to carry out the process:

1. when said heterocycle is an imidazole or an imidazoline:
using a 2-azidoethylamine, according to Tetrahedron, 47(38), 1991, 8177-94,
an ethylenediamine, according to Bioorg. Med. Chem. Lett. 12(3), 2002, 471-75,
glyoxal and aqueous ammonia, according to J. Med. Chem., 46(25), 2003, 5416-27;

2. when said heterocycle is an oxazole or an oxazoline:
using a 2-azidoethanol, according to J. Org. Chem., 61(7), 1996, 2487-96,
a 2-aminoethanol, according to J. Med. Chem. 47(8), 2004, 1969-86 or Khim. Geterosikl, Soed. 1984(7), 881-4,
2-aminoacetaldehyde diethyl acetal, according to Heterocycles, 39(2), 1994, 767-78;

3. when said heterocycle is a thiazole or a thiazoline:
using a 2-chloroethylamine and Lawesson's reagent, according to Helv. Chim. Acta, 88(2), 2005, 187-95,
a 2-aminoethanethiol, according to J. Org, Chem. 69(3), 2004, 811-4, or Tetrahedron Lett., 41(18), 2000, 3381-4.

More generally, it is advantageous, in the context of the invention, to form the heterocycle of a product of general formula (VI) using a triflate, a carboxylic acid or a carboxylic acid ester, by any one of the methods of synthesis known to those skilled in the art, such as those described in Comprehensive Organic Chemistry, by D. H. R. Barton et al. (Pergamon Press) or Advances in Heterocyclic Chemistry (Academic Press) or Heterocyclic Compounds (Wiley Intersciences).

The products of general formula (VI) in which R4 represents Cl can also be obtained by chlorination of the corresponding compound of general formula (VI) in which R4 represents H, by using a chlorinating reagent known to those skilled in the art, such as sodium hypochlorite in a basic aqueous medium (analogy with Bioorg. Med. Chem. 2007, 15 (6), 2441), chlorine gas in an acetic medium (analogy with J. Med. Chem. 2003, 46 (26), 5663) or preferably N-chlorosuccinimide in an organic solvent such as dimethylformamide (analogy with patent WO 1997/12884).

The products of general formula (VI) in which R4 represents Br can also be obtained by bromination of the corresponding compound of general formula (VI) in which R4 represents H, by using a brominating reagent known to those skilled in the art, such as sodium hypobromite in a basic aqueous medium (analogy with patent WO 2006/50006), bromine in an acetic medium (analogy with patent WO 2007/126841), pyridinium tribromide in an organic solvent such as methanol (analogy with patent US 2005/277638) or preferably N-bromosuccinimide in an organic solvent such as dimethylformamide (analogy with Bioorg. Med. Chem. 2008, 16 (11), 5962).

The products of general formula (VI) in which R4 represents F can also be obtained by fluorination of the corresponding compound of general formula (VI) in which R4 represents H, by using a fluorinating reagent known to those skilled in the art, such as, for example, Selectfluor® in an organic solvent such as acetonitrile as a mixture with acetic acid (analogy with patent WO 2009/147188).

The products of general formula (VI) in which R4 represents I can also be obtained by iodination of the corresponding compound of general formula (VI) in which R4 represents H, by using an iodinating reagent known to those skilled in the art, such as iodine in a basic aqueous medium (analogy with patent WO 2008/154241 or Synlett (20), 3216 (2008)), or N-iodosuccinimide in an organic solvent such as dimethylformamide.

B) Starting from a Product of General Formula (III)

More particularly, when Het does not represent a heterocycle of imidazol-2-yl, triazol-3-yl, benzimidazol-2-yl or azabenzimidazol-2-yl type, and is optionally substituted with one or more R1 radicals, as defined above, it is particularly advantageous, according to the invention, to prepare the compounds of general formula (VI) starting from the products of general formula (III):

1) either by carrying out the process according to scheme (25):
by means of a reaction of aromatic nucleophilic substitution of 2-bromo-4-fluorobenzonitrile, or of 4-bromo-5-cyano-2-fluoropyridine or of 3-bromo-2-cyano-5-fluoropyridine, in a solvent such as dimethylformamide (DMF), dimethyl sulphoxide (DMSO) or N-methylpyrrolidone (NMP), after having pretreated the indazole derivative of general formula (III) with a strong base, for instance sodium hydride,
optionally followed by a Buchwald-Hartwig amination with an amine R2-NH₂, in which R2 is as defined above, in the presence of a base such as potassium tert-butoxide and of a palladium(0) derivative, such as “palladium-dppf”, formed from palladium acetate and 1,1′-bis(diphenylphosphino)ferrocene, in a solvent such as toluene.

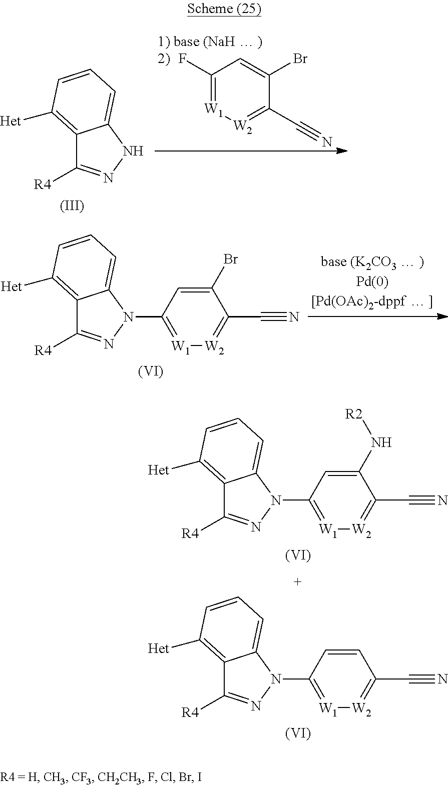

In general, the compound resulting from bromine hydrogenolysis is also obtained during this Buckhwald-Hartwig step, it being possible for this compound to also be obtained directly starting from the compounds (III) according to scheme (25a).

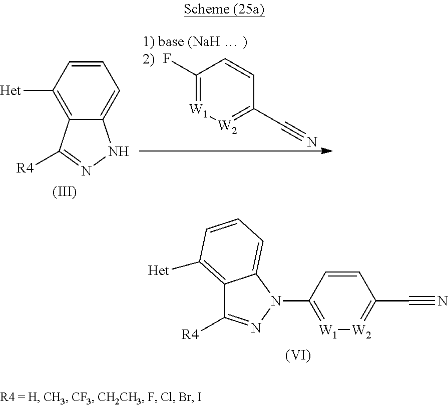

2) or by carrying out the process according to scheme (26)
by means of a Buchwald-Hartwig reaction between 4-bromo-2-fluorobenzonitrile, or 2-bromo-5-cyano-4-fluoropyridine or 5-bromo-2-cyano-3-fluoropyridine, and an indazole of general formula (III), in the presence of a base such as cesium carbonate and a palladium(0) derivative, such as “palladium-xanthphos” formed from palladium acetate and 4,5-bis(diphenylphosphino)-9,9-dimethylxanthene, in a solvent such as dioxane,
optionally followed by a reaction of aromatic nucleophilic substitution with an amine R2-NH₂, in which R2 is as defined above, in the presence of a base such as potassium carbonate, in a solvent such as DMSO.

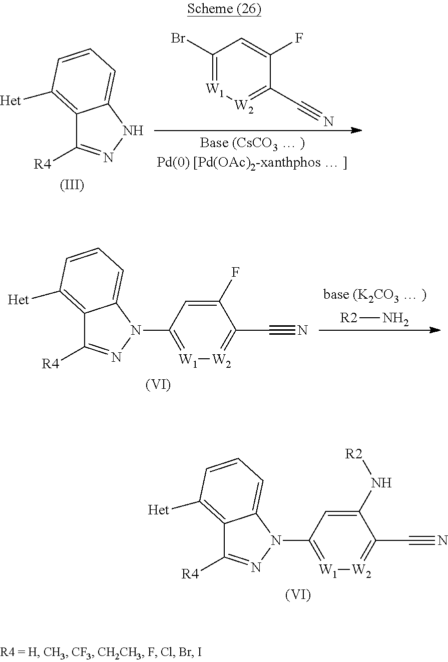

3) or by carrying out the process according to scheme (26a)
by means of a reaction of aromatic nucleophilic monosubstitution with an amine R2-NH₂, in which R2 is as defined above, in the presence of a base such as potassium carbonate, in a solvent such as DMSO, on a dihalopyridine, advantageously a difluoropyridine, and preferably thermally or with microwave irradiation,
followed by a reaction of aromatic nucleophilic substitution of the second halogen in a solvent such as dimethylformamide (DMF), dimethyl sulphoxide (DMSO) or N-methylpyrrolidone (NMP), after having pretreated the indazole derivative of general formula (III) with a strong base, for instance sodium hydride,

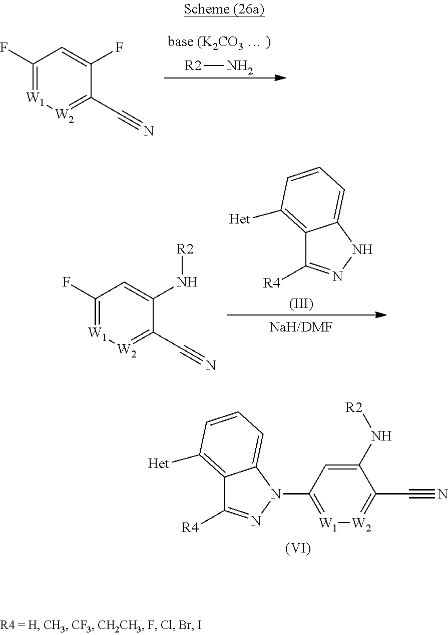

When Het represents a heterocycle of imidazol-2-yl, triazol-3-yl, benzimidazol-2-yl or azabenzimidazol-2yl type, and is optionally substituted with one or more R1 radicals, as defined above, it is also advantageous, according to the invention, to prepare the compounds of general formula (VI), starting from the products of general formula (III), by carrying out the process according to the methods described previously in schemes (25), (26) and (26a). However, in these cases, it is appropriate, prior to the Buchwald-Hartwig and/or aromatic nucleophilic substitution reaction, to protect the nitrogen of NH type, of the heterocycle Het, with a protective group such as a Boc or TBDMS or SEM radical, according to any one of the methods previously described or known to those skilled in the art. Said protective group will be either spontaneously cleaved during the Buchwald-Hartwig and/or aromatic nucleophilic substitution reactions, or cleaved after these reactions, using any one of the methods known to those skilled in the art.

### Preparation of the Compounds of General Formula (I)

The subject of the present invention is thus also the methods for synthesizing the products of formula (I).

A) Starting from the Products of General Formula (III)

According to the invention, it is particularly advantageous to prepare the compounds of general formula (I), starting from the products of general formula (III), by means of a Buchwald-Hartwig reaction between a heterocyclic derivative of indazole of general formula (III) and an aromatic derivative R—Br, R—I or R—OTf, in which R is as described above. The process is then carried out according to scheme (27), in the presence of a base such as cesium carbonate and a palladium(0) derivative, such as “palladium-xanthphos”, formed from palladium acetate and 4,5-bis(diphenylphosphino)-9,9-dimethylxanthene, in a solvent such as dioxane:

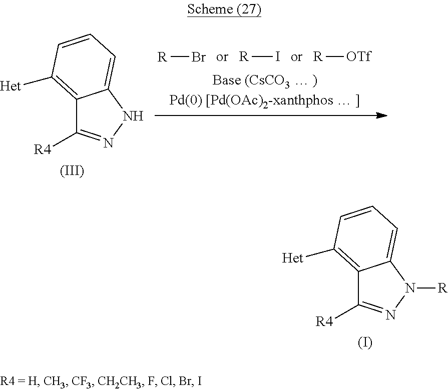

It is also possible to isolate the compounds (I) directly by aromatic nucleophilic substitution on a halogenated carbonitrile compound, itself prepared by a method described or known to those skilled in the art, according to scheme (27a):

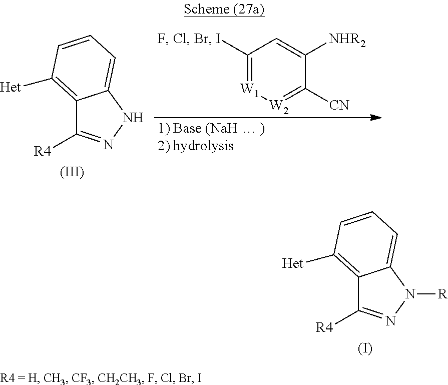

B) Starting from the Products of General Formula (V)

More particularly, when Het does not represent a heterocycle of imidazol-2-yl, triazol-3-yl, benzimidazol-2-yl or azabenzimidazol-2-yl type, and is optionally substituted with one or more R1 radicals, as defined above, it is particularly advantageous, according to the invention, to prepare the compounds of general formula (I) by coupling:

either a product of general formula (V), in which Z represents a trifluoromethylsulphonyloxy radical, with a heterocyclic boronic derivative, which may be an acid or an ester, such as the methyl, n-butyl, isopropyl or pinacol ester, under the Suzuki reaction conditions, in the presence of a palladium(0) derivative as catalyst,

or a product of general formula (V), in which Z represents a boronic radical, which may be an acid or an ester, such as the methyl, n-butyl, isopropyl or pinacol ester, itself prepared by coupling, with a brominated or iodinated heterocyclic derivative, by carrying out the process according to scheme (28):

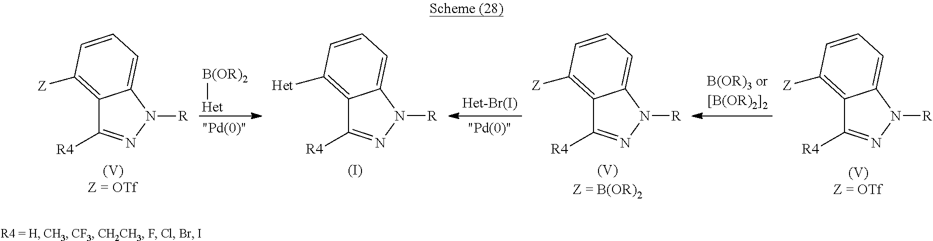

More particularly, when the heterocycle Het is of benzimidazole or azabenzimidazole type—or alternatively of benzoxazole or azabenzoxazole, or benzothiazole or azabenzothiazole type, linked via its 2-position to position 4 of the indazole, it is particularly advantageous to form said heterocycle by coupling a derivative of ortho-phenylenediamine or of diaminopyridine—or alternatively of ortho-aminophenol, of ortho-aminothiophenol or of aminohydroxypyridine or of aminomercaptopyridine, which is ortho-disubstituted—with a derivative of general formula (V) in which Z represents an acid or an ester, in particular a methyl or ethyl ester, by carrying out the process according to scheme (29):

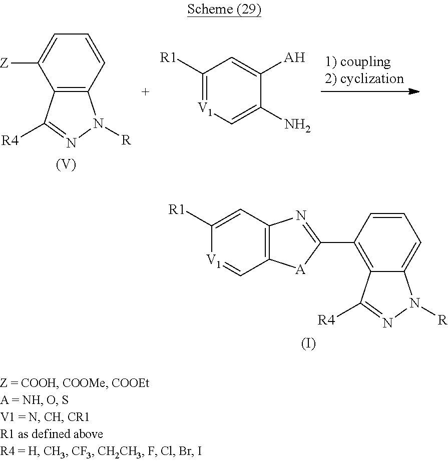

When a product of general formula (V) in which Z is an acid is used, it is particularly advantageous to activate this acid using a coupling agent known to those skilled in the art, such as 1-(3-dimethylaminopropyl)-3-ethylcarbodiimide hydrochloride (EDCl) in the presence of 1-hydroxybenzotriazole (HOBT) or of O-((ethoxycarbonyl)cyanomethyleneamino)-N,N,N′,N′-tetramethyluronium tetra-fluoroborate (TOTU).

When a product of general formula (V) in which Z is a methyl or ethyl ester is used, it is advantageous, in the context of the invention, to carry out the process in the presence of trimethylaluminium in a halogenated organic solvent, such as dichloromethane or dichloroethane.

Various conditions for cyclization of the mixture of intermediate amides can be used in the context of the invention, such as acetic acid or a mixture of trifluoroacetic acid and trifluoroacetic anhydride. It is also particularly advantageous, in the context of the invention, to carry out this type of thermal cyclization in an acid medium by heating in a microwave reactor.

More particularly, when the heterocycle Het is of the type imidazole, oxazole or thiazole, linked via its 2-position to position 4 of the indazole, it is particularly advantageous to form said heterocycle using an acid or an ester, by carrying out the process according to scheme (30):

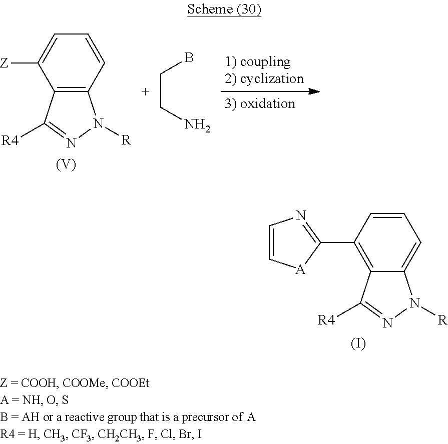

In the context of the invention, it is particularly advantageous to carry out the process:

1. when said heterocycle is an imidazole or an imidazoline:
using a 2-azidoethylamine, according to Tetrahedron, 47(38), 1991, 8177-94,
an ethylenediamine, according to Bioorg. Med. Chem. Lett, 12(3), 2002, 471-75,
glyoxal and aqueous ammonia, according to J. Med. Chem., 46(25), 2003, 5416-27;

2. when said heterocycle is an oxazole or an oxazoline:
using a 2-azidoethanol, according to J. Org. Chem., 61(7), 1996, 2487-96,
a 2-aminoethanol, according to J. Med. Chem., 47(8), 2004, 1969-86 or Khim. Geterosikl, Soed. 1984(7), 881-4,
2-aminoacetaldehyde diethyl acetal, according to Heterocycles, 39(2), 1994, 767-78;

3. when said heterocycle is a thiazole or a thiazoline:
using a 2-chloroethylamine and Lawesson's reagent, according to Helv. Chim. Acta, 88(2), 2005, 187-95,
a 2-aminoethanethiol, according to J. Org. Chem. 69(3), 2004, 811-4, or Tetrahedron Lett., 41(18), 2000, 3381-4.

More generally, it is advantageous, in the context of the invention, to form the heterocycle of a product of general formula (I) using a triflate, a carboxylic acid or a carboxylic acid ester, by any one of the methods of synthesis known to those skilled in the art, such as those described in Comprehensive Organic Chemistry, by D. H. R. Barton et al. (Pergamon Press) or Advances in Heterocyclic Chemistry (Academic Press) or Heterocyclic Compounds (Wiley Intersciences).

C) Starting from a Product of General Formula (VI)

The compounds of general formula (I) in which R is of type A can be prepared by hydrolysis of the cyano radical of a compound of general formula (VI). This hydrolysis can be carried out, advantageously in the context of the invention, by the action of an aqueous solution of hydrogen peroxide in an alkali medium in a mixture of DMSO and ethanol, according to scheme (31):

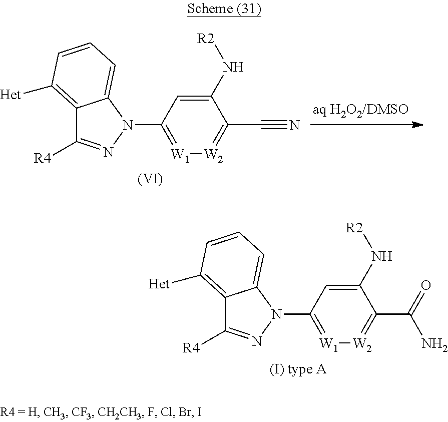

The compounds of general formula (I) in which R is of type B and X is an NH radical can be prepared, advantageously in the context of the invention, by means of an aromatic nucleophilic substitution reaction, followed by intramolecular cyclization, by the action of hydrazine hydrate in a polar solvent, such as n-butanol, on a nitrile of general formula (VI), ortho-substituted with a halogen atom, very preferably a fluorine atom, according to scheme (32):

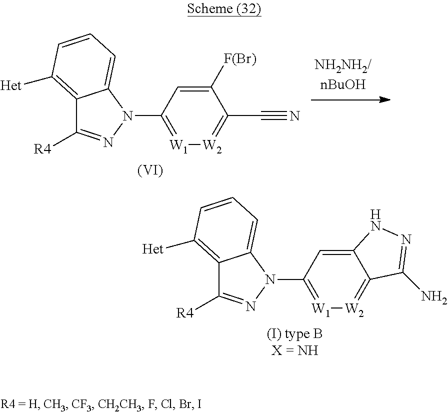

The compounds of general formula (I), in which R is of type B and X is an NR2 radical, with R2 as defined above, can be prepared, advantageously in the context of the invention, according to scheme (33), by the action of a hydrazine monosubstituted with an R2 radical, in a polar solvent, such as n-butanol, on a nitrile of general formula (VI), ortho-substituted with a halogen atom, very preferably a fluorine atom.

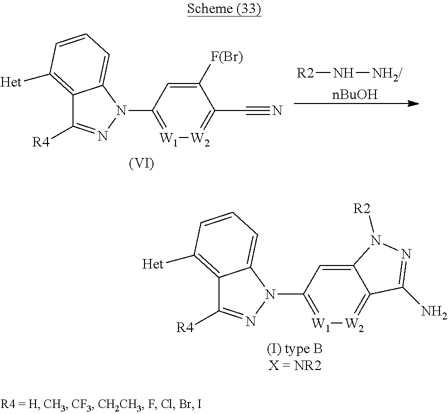

The compounds of general formula (I), in which R is of type B and X is an oxygen atom, can be prepared, advantageously in the context of the invention, by the action of an N-protected hydroxylamine, such as N-tert-butyloxycarbonyl-hydroxylamine, in the presence of a strong base, such as potassium tert-butoxide, on a nitrile of general formula (VI), ortho-substituted with a halogen atom, very preferably a fluorine atom, in a solvent such as DMF, by carrying out the process according to scheme (34):

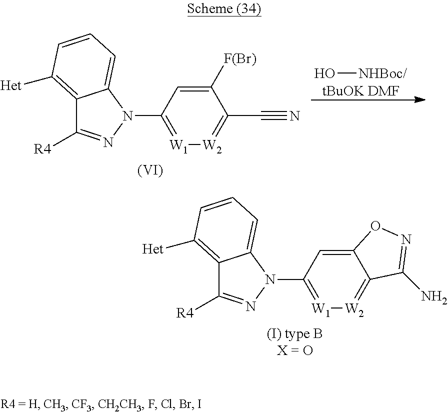

The compounds of general formula (I), in which R is of type B and X is a sulphur atom, can be prepared, advantageously in the context of the invention, by the action of sodium sulphide in a solvent such as DMSO, on a nitrile of general formula (VI), ortho-substituted with a halogen atom, very preferably a fluorine atom, followed by the action of aqueous ammonia in the presence of sodium hypochlorite, by carrying out the process according to scheme (35), in particular under the conditions described in Bioorg. Med. Chem. Lett. (2007), 17(6), 4568:

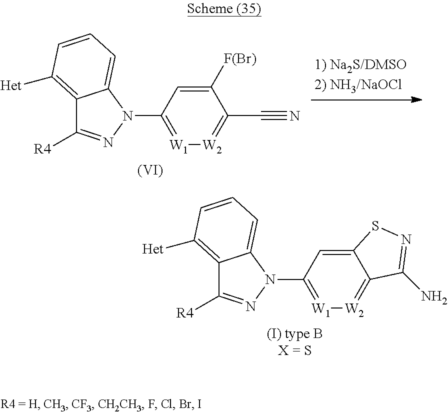

The compounds of general formula (I), in which R is of type C, can be prepared, advantageously in the context of the invention, by the action of hydroxylamine hydrochloride on a nitrile of general formula (VI), ortho-substituted with a halogen atom, very preferably a fluorine atom, by carrying out the process according to scheme (36), in particular under the conditions described in Zeitschrift für Chemie (1984), 24(7), 254:

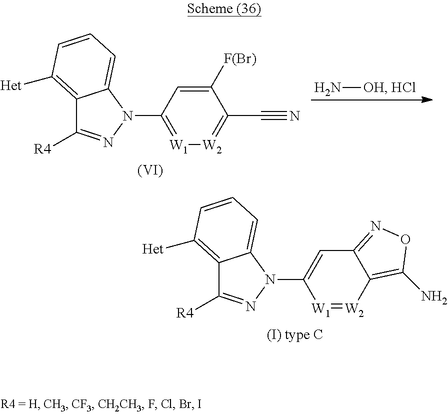

The compounds of general formula (I), in which R is of type D, with W3a nitrogen atom, can be prepared, advantageously in the context of the invention, by the action of aqueous ammonia on a nitrile of general formula (VI), ortho-substituted with a halogen atom, very preferably a fluorine atom, followed by the action of a mixture of ethyl orthoformate and ammonium acetate, by carrying out the process according to scheme (37), in particular under the conditions described in J. Het. Chem. (2006), 43(4), 913:

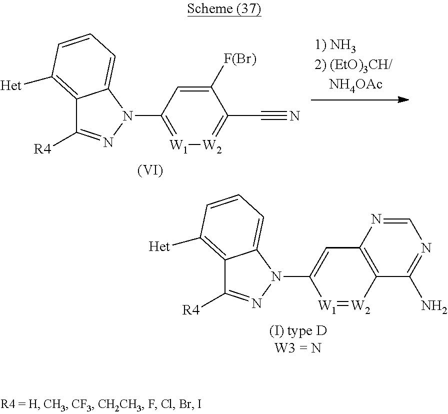

The compounds of general formula (I), in which R is of type E, can be prepared, advantageously in the context of the invention, by the action of trimethylsilylacetylene, in the presence of a base such as triethylamine or n-butylamine, in the presence of cuprous iodide and of tetrakis(triphenylphosphine)palladium, on a compound of general formula (VI), ortho-substituted with a bromine atom, so as to give an acetylenic intermediate, which is then successively treated with sodium ethoxide in ethanol, and then with a solution of hydrogen peroxide in an alkaline medium and, finally, heated in the presence of para-toluenesulphonic acid, by carrying out the process according to general scheme (38), in particular under the conditions described in Chem. Pharm. Bull. (1986), 34, 2760.

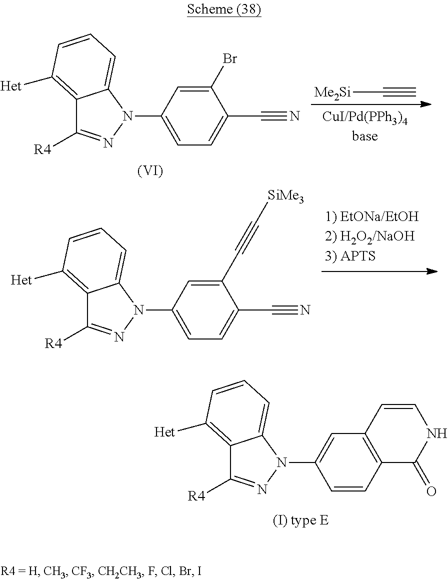

D) Starting from Products of General Formula (I)

The compounds of general formula (I), in which R is of type B and X is an NR2 radical, with R2 as defined above, and in which Het does not represent a heterocycle of imidazol-2-yl, triazol-3-yl, benzimidazol-2-yl or azabenzimidazol-2-yl type, can be prepared according to scheme (39) by N-alkylation of a product of general formula (I) of type B with X=NH. This alkylation can be carried out according to the methods known to those skilled in the art, in particular by a treatment with a base such as sodium hydride, followed by the action of a halogenated derivative R2-Hal. By carrying out the process in this way, a mixture of N1- and N2-alkylated regioisomers is generally obtained, it being possible for these regioisomers to be separated using the conventional methods known to those skilled in the art.

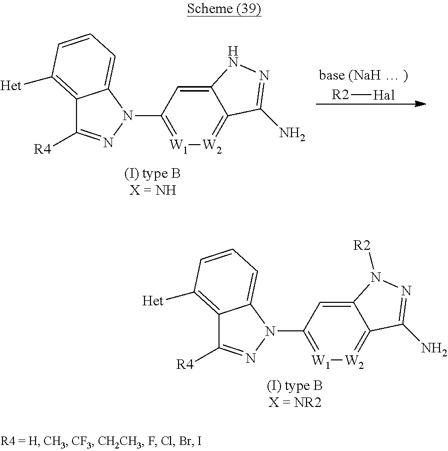

The compounds of general formula (I), in which R is of type A, and in which Y represents O—PO₃H₂, O—PO₃Na₂, O—SO₃H₂, O—SO₃Na₂, O—CH₂—PO₃H₂, O—CH₂—PO₃Na₂, O—CO-alkyl, including in particular O—CO—CH₂—CO₂tBu, O—CO—CH₂—NHMe, O—CO—CH₂—N(Me)₂ and the derivatives which are esters of amino acids of the natural or unnatural series and the derivatives which are esters of dipeptides or of tripeptides, and more particularly O—CO-glycine, O—CO-alanine, O—CO-serine, O—CO-lysine, O—CO-arginine, O—CO-glycine-lysine and O—CO-alanine-lysine, can be prepared starting from the compounds of general formula (I) in which R is of type A with Y representing OH, by carrying out the process according to scheme (40).

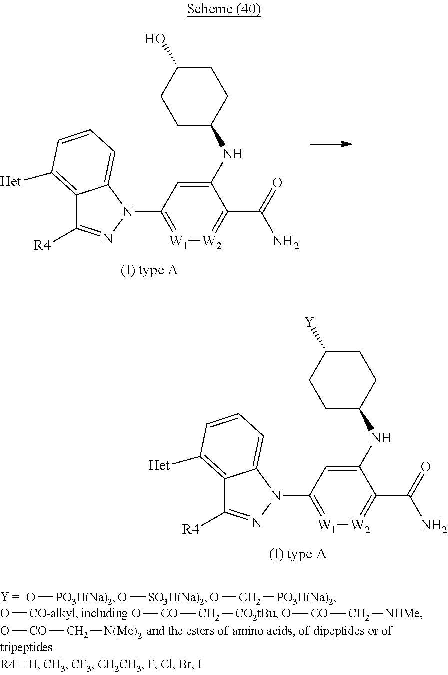

The compounds of general formula (I), in which R is of type B′, and in which Y represents O—PO₃H₂, O—PO₃Na₂, O—SO₃H₂, O—SO₃Na₂, O—CH₂—PO₃H₂, O—CH₂—PO₃Na₂, O—CO-alkyl, including in particular O—CO—CH₂—CO₂tBu, O—CO—CH₂—NHMe, O—CO—CH₂—N(Me)₂ and the derivatives which are esters of amino acids of the natural or unnatural series and the derivatives which are esters of dipeptides or of tripeptides, and more particularly O—CO-glycine, O—CO-alanine, O—CO-serine, O—CO-lysine, O—CO-arginine, O—CO-glycine-lysine and O—CO-alanine-lysine and n represents 2 or 3, can be prepared starting from the compounds of general formula (I) in which R is of type B′ with Y representing OH, by carrying out the process according to scheme (41).

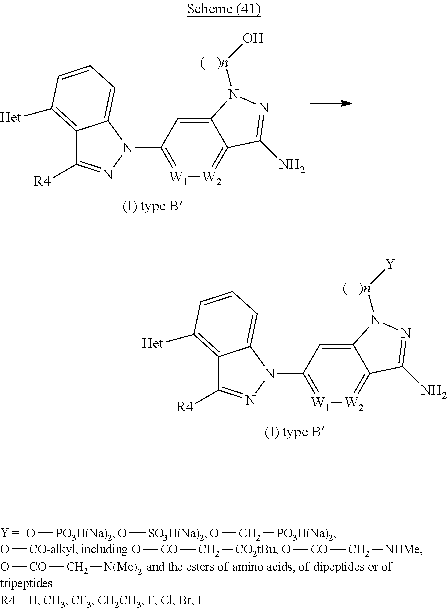

More particularly, when Y represents a phosphate radical, in acid or salified form, the process is generally carried out by the action of di-O-benzylphosphoric or di-O-phenylphosphoric acid chloride on a derivative of general formula (I) of type A or B′ in which Y is OH, in a solvent such as pyridine, followed by hydrogenolysis in the presence of a palladium catalyst (palladium-on-charcoal or palladium hydroxide). When the heterocycle Het is of the type benzimidazole, azabenzimidazole or imidazole, linked via its 2-position to position 4 of the indazole, it may be advantageous, in the context of the invention, to protect the NH of the heterocycle in the form of N-Boc, N-TBDMS or N-SEM.

More particularly, when Y represents a sulphate radical, in acid or salified form, the process is generally carried out by the action of sulphuric anhydride—or sulphur trioxide—or of oleum—a mixture of sulphuric acid and of sulphuric anhydride—on a derivative of general formula (I) of type A or B′ in which Y is OH, in a solvent such as pyridine. When the heterocycle Het is of the type benzimidazole or azabenzimidazole or imidazole, linked via its 2-position to position 4 of the indazole, it may be advantageous, in the context of the invention, to protect the NH of the heterocycle in the form of N-Boc, N-TBDMS or N-SEM.

More particularly, when Y represents a phosphonyloxymethyloxy radical, the process is generally carried out by the action of a strong base, such as sodium hydride, and then of phosphoric acid di-tert-butyl ester or of phosphoric acid chloromethyl ester on a derivative of general formula (I) of type A or B′ in which Y is OH, in a solvent such as DMF, followed by hydrolysis in an acidic medium, such as a 4N solution of hydrochloric acid. When the heterocycle Het is of the type benzimidazole or azabenzimidazole or imidazole type, linked via its 2-position to position 4 of the indazole, it may be advantageous, in the context of the invention, to protect the NH of the heterocycle in the form of N-Boc, N-TBDMS or N-SEM.

More particularly, when Y represents a carboxylic ester radical, the process is generally carried out by the action of a carboxylic acid, in the presence of an agent for activating the acid function, such as 1-(3-dimethylaminopropyl)-3-ethylcarbodiimide hydrochloride (EDCl), and of a base, such as 4-dimethylaminopyridine (DMAP), or of O-((ethoxycarbonyl)cyanomethylene-amino)-N,N,N′,N′-tetramethyluronium tetrafluoroborate (TOTU), in a solvent such as dichloromethane. When said ester is an amino acid-derived, dipeptide-derived or tripeptide-derived ester, it is advantageous, in the context of the invention, to use an amino acid or a dipeptide-derived or tripeptide-derived acid, the amino and/or hydroxyl residue(s) of which is (are) protected, for example in NH-Boc, NH-Fmoc or O-Su form.

The compounds of general formula (I), in which R is of type D, with W₁, W₂ and W₃=CH, can be prepared, advantageously in the context of the invention, by the action of phosphorus trichloride and then of acetamide, at a temperature close to 180°, in the presence of a base such as potassium carbonate, on a product of general formula (I) of type E, by carrying out the process according to scheme (42), in particular under the conditions described in Bioorg. Med. Chem. (2006), 14(20), 6832. When the heterocycle Het is of the type benzimidazole or azabenzimidazole or imidazole, linked via its 2-position to position 4 of the indazole, it may be advantageous, in the context of the invention, to protect the NH of the heterocycle in the form of N-Boc, N-TBDMS or N-SEM.

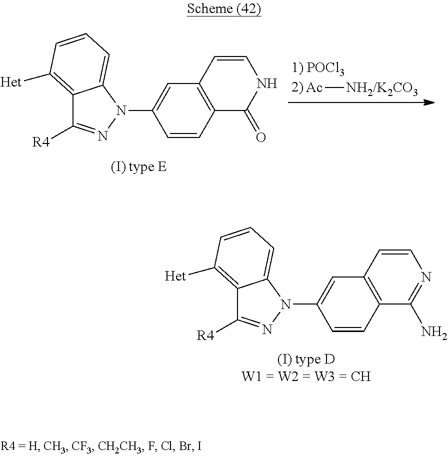

The products of general formula (I), in which R4 represents Cl, can also be obtained by chlorination of the corresponding compound of general formula (I) in which R4 represents H, by using a chlorinating reagent known to those skilled in the art, such as sodium hypochlorite in a basic aqueous medium (analogy with Bioorg. Med. Chem. 2007, 15 (6), 2441), chlorine gas in an acetic medium (analogy with J. Med. Chem. 2003, 46 (26), 5663) or preferably N-chlorosuccinimide in an organic solvent such as dimethylformamide (analogy with patent WO 1997/12884).

The products of general formula (I), in which R4 represents Br, can also be obtained by bromination of the corresponding compound of general formula (I) in which R4 represents H, by using a brominating reagent known to those skilled in the art, such as sodium hypobromite in a basic aqueous medium (analogy with patent WO 2006/50006), bromine in an acetic medium (analogy with patent WO 2007/126841), pyridinium tribromide in an organic solvent such as methanol (analogy with patent US 2005/277638) or preferably N-bromosuccinimide in an organic solvent such as dimethylformamide (analogy with Bioorg. Med. Chem. 2008, 16 (11), 5962).

The products of general formula (I), in which R4 represents F, can also be obtained by fluorination of the corresponding compound of general formula (I) in which R4 represents H, by using a fluorinating reagent known to those skilled in the art, such as, for example, Selectfluor® in an organic solvent such as acetonitrile as a mixture with acetic acid (analogy with patent WO 2009/147188).

The products of general formula (I), in which R4 represents I, can also be obtained by iodination of the corresponding compound of general formula (I) in which R4 represents H, by using an iodinating reagent known to those skilled in the art, such as iodine in a basic aqueous medium (analogy with patent WO 2008/154241 or Synlett (20), 3216 (2008)), or N-iodosuccinimide in an organic solvent such as dimethylformamide.

More particularly, the compounds of general formula (IA), in which R represents the group below:

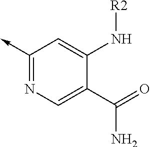

can be advantageously prepared starting from 4,6-dichloronicotinamide by carrying out the process according to scheme (43) or scheme (44)

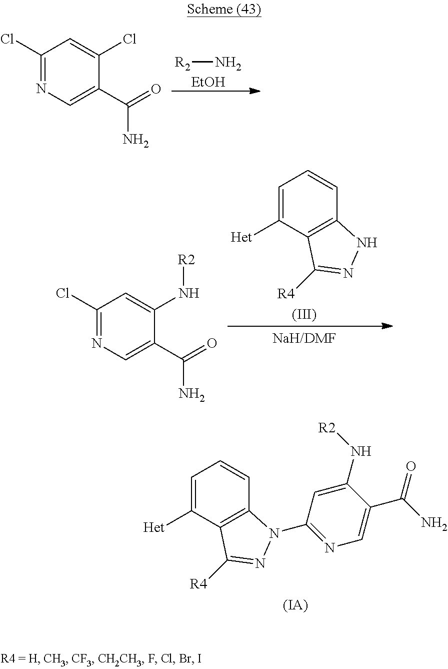

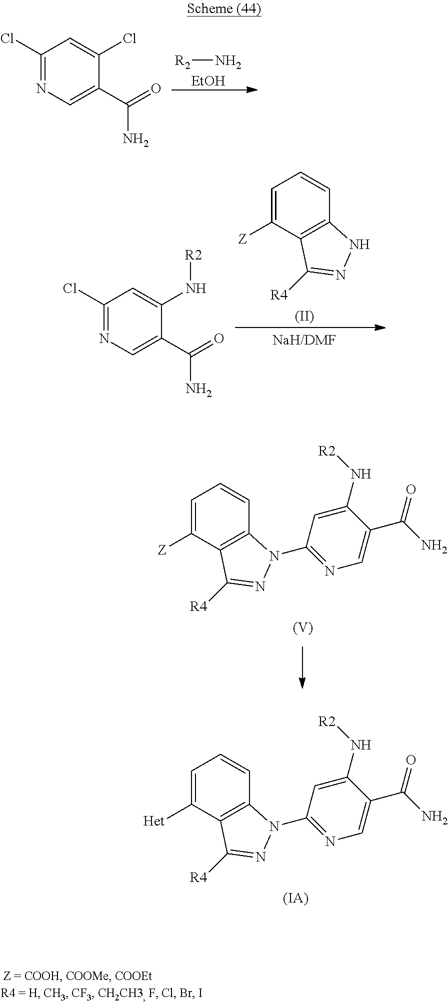

More particularly, the compounds of general formula (I) where Het represents the group below:

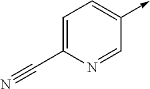

can be advantageously prepared starting from the compounds of general formula (III) by carrying out the process according to scheme (45), implementing the following successive reactions:
Buchwald-Hartwig reaction with the tert-butyl ester of 4-bromo-2-fluorobenzoic acid in the presence of a base such as cesium carbonate and a palladium(0) derivative, such as “palladium-xanthphos”, formed from palladium acetate and 4,5-bis(diphenylphosphino)-9,9-dimethylxanthene, in a solvent such as dioxane,
then an aromatic nucleophilic substitution reaction with an amine R2-NH₂, in which R2 is as defined above, in the presence of a base such as potassium carbonate, in a solvent such as DMSO,
then hydrolysis of the ester to give the acid, by reaction with hydrochloric acid in a solvent such as dioxane, at a temperature close to 100° C.,
and, finally, formation of the carbamoyl radical by coupling of the acid, pre-activated with (1H-benzotriazol-1-yloxy)[tris(dimethylamino)]phosphonium hexafluorophosphate (BOP) and hydroxybenzotriazole (HOBT), with ammonium chloride in the presence of a base such as diisopropylethylamine, in a solvent such as N, N-dimethylformamide.

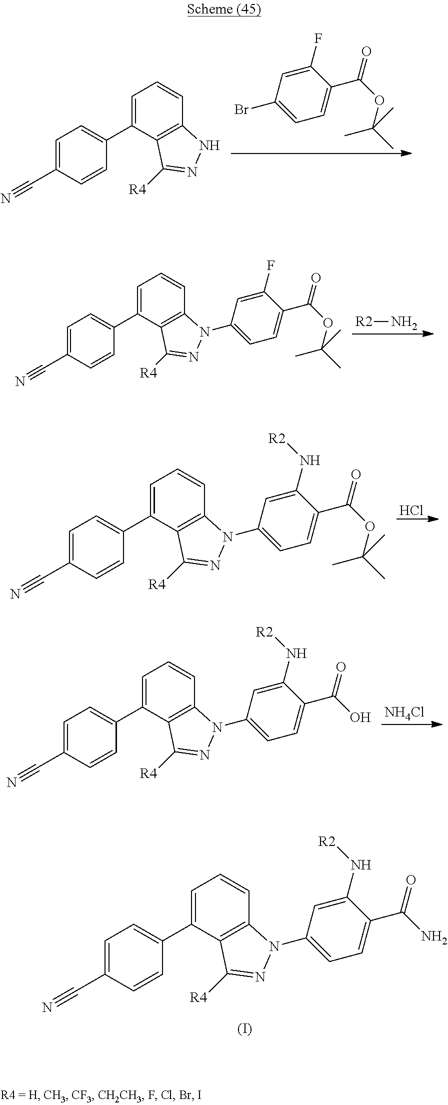

The reactions described above can be carried out according to the conditions described in the preparation of the examples hereinafter and also according to the general methods known to those skilled in the art, in particular those described in: Comprehensive Organic Chemistry, by D. H. R. Barton et al. (Pergamon Press); Advanced Organic Chemistry, by J. Marsh (Wiley Interscience).

The subject of the present invention is thus in particular the processes described above in schemes 1 to 45, which can thus be used for the synthesis of the products of formula (I) as defined above.

The products which are subjects of the present invention have advantageous pharmacological properties: it has been observed that they in particular possess inhibitory properties on the activities of chaperone proteins, and in particular on their ATPase activities.

Among these chaperone proteins, mention is in particular made of the human chaperone Hsp90.

The products corresponding to general formula (I) as defined above thus have a considerable inhibitory activity on the Hsp90 chaperone.

Tests given in the experimental section hereinafter illustrate the inhibitory activity of products of the present invention with respect to such chaperone proteins.

These properties thus mean that the products of general formula (I) of the present invention can be used in medicaments in the treatment of malignant tumours.

The products of formula (I) can also be used in the veterinary field.

The subject of the invention is therefore the use, as medicaments, of the products of formula (I) as defined above.

The subject of the present invention is, in particular, as medicaments, the products of formula (I) as defined above:

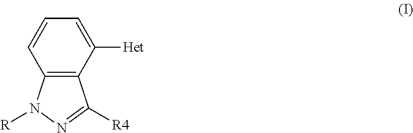

in which:
R4 represents H, CH₃, CH₂CH₃, CF₃, F, Cl, Br or I;
Het represents a monocyclic or bicyclic, aromatic or partially unsaturated heterocycle—of dihydro or tetrahydro type —, with from 5 to 11 ring members, containing from 1 to 4 heteroatoms chosen from N, O or S, optionally substituted with one or more radicals R1 or R′1, which may be identical or different, as described below,
R is chosen from the group constituted of

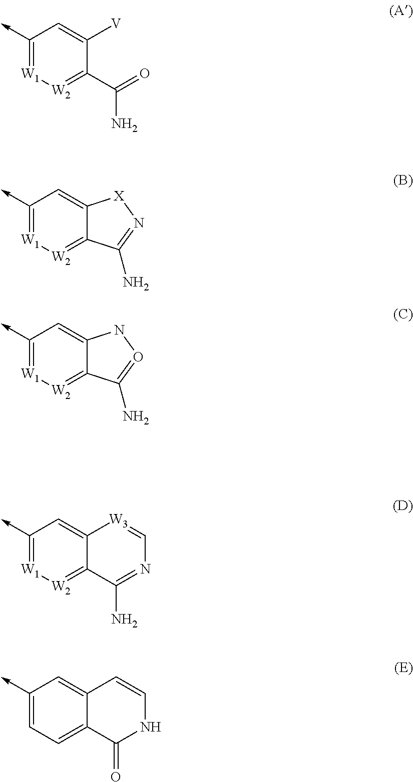

with R1 and/or R′1, which may be identical or different, chosen from the group constituted of H, halogen, CF₃, nitro, cyano, alkyl, hydroxyl, mercapto, amino, alkylamino, dialkylamino, alkoxy, phenylalkoxy, alkylthio, carboxyl in free form or esterified with an alkyl radical, carboxamide, CO—NH(alkyl), CON(alkyl)₂, NH—CO-alkyl, sulphonamide, NH—SO₂-alkyl, S(O)₂—NHalkyl and S(O₂)—N(alkyl)₂, all the alkyl, alkoxy and alkylthio radicals being themselves optionally substituted with one or more radicals, which may be identical or different, chosen from halogen, hydroxyl, alkoxy, amino, alkylamino and dialkylamino;
W1, W2 and W3 independently represent CH or N;
X represents an oxygen or sulphur atom, or an NR2, C(O), S(O) or S(O)₂ radical;
V represents a hydrogen atom or halogen atom or an —O—R2 radical or an —NH—R2 radical in which:
R2 represents a hydrogen atom or a C₁-C₆ alkyl radical, or a C₃-C₈ cycloalkyl radical or a C₃-C₁₀ heterocycloalkyl radical, which is monocyclic or bicyclic; these alkyl, cycloalkyl and heterocycloalkyl radicals being optionally substituted with one or more radicals, which may be identical or different, chosen from the radicals:
—O—PO₃H₂; —O—PO₃Na₂; —O—CH₂; —O—SO₃Na₂; —O—CH₂—PO₃H₂; —O—CH₂—PO₃Na₂; —O—CO-alanine; —O—CO-glycine; —O—CO-serine; —O—CO-lysine; —O—CO-arginine; —O—CO-glycine-lysine; —O—CO-alanine-lysine;
halogen; hydroxyl; mercapto; amino; carboxamide (CONH₂); carboxyl;
heterocycloalkyl; cycloalkyl; heteroaryl; carboxyl esterified with an alkyl radical; —CO—NH(alkyl); —O—CO-alkyl; —NH—CO-alkyl; alkyl; alkoxy; alkylthio; alkylamino; dialkylamino; in all the latter radicals, the alkyl, alkoxy and alkylthio radicals being themselves optionally substituted with one or more radicals, which may be identical or different, chosen from hydroxyl, mercapto, amino, alkylamino, dialkylamino, CO₂alkyl, NHCO₂alkyl and heterocycloalkyl radicals; in all these radicals, the cycloalkyl, heterocycloalkyl and heteroaryl radicals being themselves optionally substituted with one or more radicals, which may be identical or different, chosen from hydroxyl, alkyl, alkoxy, CH₂OH, amino, alkylamino, dialkylamino. CO₂alkyl or NHCO₂alkyl radicals;
   said products of formula (I) being in all the possible tautomeric and isomeric forms: racemic, enantiomeric and diastereoisomeric, and also as pharmaceutically acceptable addition salts with inorganic and organic acids or with inorganic and organic bases of the products of formula (I), and also the prodrug of the products of formula (I).

The subject of the invention is in particular the use, as medicaments, of the products of formula (I) as defined above, which have the following names:
2-(trans-4-hydroxycyclohexylamino)-4-(3-methyl-4-quinolin-3-ylindazol-1-yl)benzamide.
4-(3-methyl-4-quinolin-3-ylindazol-1-yl)benzamide.
2-(3-hydroxypropylamino)-4-(3-methyl-4-quinolin-3-ylindazol-1-yl)benzamide.
2-[2-(4-hydroxy-1-methylpiperidin-4-yl)ethylamino]-4-(3-methyl-4-quinolin-3-ylindazol-1-yl)benzamide.
2-(2-hydroxy-2-methylpropylamino)-4-(3-methyl-4-quinolin-3-ylindazol-1-yl)benzamide.
4-(3-methyl-4-guinolin-3-ylindazol-1-yl)-2-(2,2,6,6-tetramethylpiperidin-4-ylamino)benzamide.
4-(3-methyl-4-quinolin-3-ylindazol-1-yl)-2-(tetrahydropyran-4-ylamino)benzamide.
2-(2-fluoroethylamino)-4-(3-methyl-4-quinolin-3-ylindazol-1-yl)benzamide.
3-(2-hydroxy-2-methylpropylamino)-5-(3-methyl-4-quinolin-3-ylindazol-1-yl)pyridine-2-carboxamide.
5-(3-methyl-4-quinolin-3-ylindazol-1-yl)-3-(tetrahydropyran-4-ylamino)pyridine-2-carboxamide,
trans-4-[2-carbamoyl-5-(3-methyl-4-quinolin-3-ylindazol-1-yl)phenyl-amino]cyclohexyl ester of aminoacetic acid.
4-[4-(6-fluoro-1H-benzimidazol-2-yl)-3-methylindazol-1-yl]-2-(trans-4-hydroxy-cyclohexylamino)benzamide.
4-[4-(6-fluoro-1H-benzimidazol-2-yl)-3-methylindazol-1-yl]-2-(2-hydroxy-2-methyl-propylamino)benzamide,
4-(3-methyl-4-quinolin-3-ylindazol-1-yl)-2-[exo-(7-oxabicyclo[2.2.1]hept-2-yl)amino]benzamide.
4-(3-methyl-4-quinolin-3-ylindazol-1-yl)-2-(1,2,2,6,6-pentamethylpiperidin-4-ylamino)benzamide.
3-(trans-4-hydroxycyclohexylamino)-5-(3-methyl-4-quinolin-3-ylindazol-1-yl)pyridine-2-carboxamide.
5-[3-methyl-4-quinolin-3-ylindazol-1-yl]-3-(1,2,2,6,6-pentamethylpiperidin-1-ylamino)pyridine-2-carboxamide.
5-[3-methyl-4-quinolin-3-ylindazol-1-yl]-3-[2-pyridin-2-ylethylamino]pyridine-2-carboxamide,
4-(3-methyl-4-quinolin-3-ylindazol-1-yl)-2-{[exo-1-(7-oxabicyclo[2.2.1]hept-2-yl)methyl]amino}benzamide.
4-(3-methyl-4-quinolin-3-ylindazol-1-yl)-2-{[endo-1-(7-oxabicyclo[2.2.1]hept-2-yl)methyl]amino}benzamide.
2-(trans-4-hydroxycyclohexylamino)-4-(4-quinolin-3-yl-3-trifluoromethylindazol-1-yl)benzamide.
4-[4-(6-fluoro-1H-benzimidazol-2-yl)-3-trifluoromethylindazol-1-yl]-2-(trans-4-hydroxycyclohexylamino)benzamide,
3-(trans-4-hydroxycyclohexylamino)-5-(4-quinolin-3-yl-3-trifluoromethylindazol-1-yl)pyridine-2-carboxamide.
2-(trans-4-hydroxycyclohexylamino)-4-(4-quinolin-3-ylindazol-1-yl)benzamide.
4-(4-quinolin-3-ylindazol-1-yl)benzamide.
5-(3-chloro-4-quinolin-3-ylindazol-1-yl)-3-(trans-4-hydroxycyclohexylamino)pyridine-2-carboxamide.
5-(3-bromo-4-quinolin-3-ylindazol-1-yl)-3-(2-hydroxy-2-methylpropylamino)pyridine-2-carboxamide.
   and also the pharmaceutically acceptable addition salts with inorganic and organic acids or with inorganic and organic bases of said products of formula (I).

The products can be administered parenterally, orally, perlingually, rectally or topically.

The subject of the invention is also the pharmaceutical compositions, characterized in that they contain, as active ingredient, at least one of the medicaments of general formula (I).

These compositions can be provided in the form of injectable solutions or suspensions, tablets, coated tablets, capsules, syrups, suppositories, creams, ointments and lotions. These pharmaceutical forms are prepared according to the usual methods. The active ingredient can be incorporated into excipients normally used in these compositions, such as aqueous or nonaqueous carriers, talc, gum arabic, lactose, starch, magnesium stearate, cocoa butter, fatty substances of animal or plant origin, paraffin derivatives, glycols, various wetting agents, dispersants or emulsifiers, or preserving agents.

The usual dose, which can vary according to the individual treated and the condition in question, can be, for example, from 10 mg to 500 mg per day in humans, orally.

The present invention thus relates to the use of products of formula (I) as defined above or of pharmaceutically acceptable salts of said products of formula (I), for the preparation of medicaments for inhibiting the activity of chaperone proteins, and in particular of Hsp90.

The present invention thus relates in particular to the use of products of formula (I) as defined above or of pharmaceutically acceptable salts of said products of formula (I), in which the chaperone protein is Hsp90.

The present invention thus relates to the use of products of formula (I) as defined above or of pharmaceutically acceptable salts of said products of formula (I), for the preparation of a medicament for preventing or treating a disease characterized by a disturbance of the activity of a chaperone protein of Hsp90 type, and in particular such a disease in a mammal.

The present invention relates to the use of products of formula (I) as defined above or of pharmaceutically acceptable salts of said products of formula (I), for the preparation of a medicament for preventing or treating a disease belonging to the following group: neurodegenerative diseases such as Huntington's disease, Parkinson's disease, focal cerebral ischaemia, Alzheimer's disease, multiple sclerosis and amyotrophic lateral sclerosis, malaria, Brugia filariasis, Bancroft's filariasis, toxoplasmosis, treatment-resistant mycoses, hepatitis B, hepatitis C, the herpesvirus, dengue (or tropical flu), spinal and bulbar muscular atrophy, mesangial cell proliferation disorders, thromboses, retinopathies, psoriasis, muscle degeneration, diseases in oncology, and cancers.

The present invention thus relates to the use of products of formula (I) as defined above or of pharmaceutically acceptable salts of said products of formula (I), for the preparation of a medicament for treating diseases in oncology.

The present invention relates in particular to the use of products of formula (I) as defined above or of pharmaceutically acceptable salts of said products of formula (I), for the preparation of a medicament for treating cancers.

Among these cancers, the present invention focuses most particularly on the treatment of solid tumours and on the treatment of cancers resistant to cytotoxic agents.

The present invention thus relates in particular to the use of products of formula (I) as defined in any one of the preceding claims or of pharmaceutically acceptable salts of said products of formula (I), for the preparation of a medicament for treating cancers, among which are lung cancer, breast cancer and ovarian cancer, glioblastomas, chronic myeloid leukaemias, acute lymphoblastic leukaemias, prostate cancer, pancreatic cancer and colon cancer, metastatic melanomas, thyroid tumours and renal carcinomas.

Thus, among the main potential indications of Hsp90 inhibitors, mention may, by way of nonlimiting example, be made of:

“non small cell” lung cancers, breast cancers, ovarian cancers and glioblastomas which overexpress EGF-R or HER2;

metastatic melanomas and thyroid tumours which overexpress the mutated form of the B-Raf protein;

breast, prostate, lung, pancreatic, colon or ovarian cancers which overexpress Akt;

chronic myeloid leukaemias which overexpress Bcr-Abl;

acute lymphoblastic leukaemias which overexpress Flt-3;

androgen-dependent and androgen-independent prostate cancers;

oestrogen-dependent and oestrogen-independent breast cancers;

renal carcinomas which overexpress H IF-1a or the mutated C-Met protein.

The present invention focuses even more particularly on the treatment of breast cancer, colon cancer and lung cancer.

The present invention also relates to the use of products of formula (I) as defined above or of pharmaceutically acceptable salts of said products of formula (I), for the preparation of a medicament for use in cancer chemotherapy.

As medicaments according to the present invention for use in cancer chemotherapy, the products of formula (I) according to the present invention can be used alone or in combination with chemotherapy or radiotherapy, or alternatively in combination with other therapeutic agents.

The present invention thus relates in particular to the pharmaceutical compositions as defined above containing, in addition to the active ingredients, other medicaments for anti-cancer chemotherapy.

Such therapeutic agents may be anti-tumour agents that are commonly used.

As examples of known protein kinase inhibitors, mention may in particular be made of butyrolactone, flavopiridol, 2-(2-hydroxyethylamino)-6-benzylamino-9-methylpurine, olomucine, Glivec and Iressa.

The products of formula (I) according to the present invention may thus also be advantageously used in combination with anti-proliferative agents: by way of examples of such anti-proliferative agents, but without however being limited to this list, mention may be made of aromatase inhibitors, anti-oestrogens, topoisomerase I inhibitors, topoisomerase II inhibitors, agents that are active on microtubules, alkylating agents, histone deacetylase inhibitors, farnesyl transferase inhibitors, COX-2 inhibitors, MMP inhibitors, mTOR inhibitors, antineoplastic antimetabolites, platinum compounds, proteasome inhibitors, such as Bortezomib, inhibitors of Histone Deactylase (HDACs), such as SAHA, and in particular inhibitors of HDAC6, compounds which bring about a reduction in protein kinase activity and also anti-angiogenic compounds, gonadorelin agonists, anti-androgens, bengamides, biphosphonates and trastuzumab.

By way of examples, mention may thus be made of anti-microtubule agents, such as taxoids, epothilones, or vinka-alkaloids, alkylating agents such as cyclophosphamide, DNA-intercalating agents such as cis-platinum and oxaliplatin, topoisomerase-interactive agents such as camptothecin and derivatives, anthracyclines such as adriamycin, antimetabolites such as 5-fluorouracil and derivatives and analogues.

The present invention therefore relates to products of formula (I) as Hsp90 chaperone inhibitors, said products of formula (I) being in all the possible isomeric forms: tautomeric, racemic, enantiomeric and diastereoisomeric, and also the pharmaceutically acceptable addition salts with inorganic and organic acids or with inorganic and organic bases of said products of formula (I), and the prodrugs thereof.

The present invention relates in particular to products of formula (I) as defined above, as Hsp90 inhibitors.

The products of formula (I) according to the present invention can be prepared by application or adaptation of known methods, and in particular of the methods described in the literature, for instance those described by R. C. Larock in: Comprehensive Organic Transformations, VCH publishers, 1989.

In the reactions described hereinafter, it may be necessary to protect reactive functional groups such as, for example, hydroxyl, amino, imino, thio or carboxyl groups, when the latter are desired in the final product but when their participation is not desired in the reactions for synthesizing the products of formula (I). Conventional protective groups can be used in accordance with the usual standard practices, such as those described, for example, by T. W. Greene and P. G. M. Wuts in “Protective Groups in Organic Chemistry”, John Wiley and Sons, 1991.

The experimental section hereinafter gives non-limiting examples of starting products: other starting products can be found commercially or can be prepared according to the usual methods known to those skilled in the art.

Examples illustrating the invention: The examples of which the preparation follows illustrate the present invention without, however, limiting it.

All the examples described were characterized by proton NMR spectroscopy and/or by mass spectroscopy, the majority of these examples were also characterized by infrared spectroscopy.

Unless different conditions are specifically described, the LC/MS mass spectra, reported in the description of the various examples below, were carried out under the following liquid chromatography conditions:

### Method A:

### Column: ACQUITY BEH C₁₈ 1.7 μm 2.1×50 mm

Solvent: A: H₂O (0.1% formic acid) B; CH₃CN (0.1% formic acid)
Column temperature: 70° C.
Flow rate: 0.7 ml/min
Gradient (11 min): from 5% to 100% B in 9 min; 9.3 min: 5% of B

### Method B:

### Column: XBridge C₁₋₁₈ 2.5 μm 3×50 mm

Solvent: A: H₂O (0.1% formic acid) B: CH₃CN (0.1% formic acid)
Column temperature: 70° C.
Flow rate: 0.9 ml/min
Gradient (7 min): from 5% to 100% of B in 5.3 min; 5.5 min: 100% of B; 6.3 min: 5% of B

### Method C:

### Column: ACQUITY BEH C₁₈ 1.7 μm 2.1×50 mm

Solvent: A: H₂O (0.1% formic acid) B: CH₃CN (0.1% formic acid)
Column temperature: 50° C.
Flow rate: 1.0 ml/min
Gradient (2 min): from 5% to 50% of B in 0.8 min; 1.2 min: 100% of B; 1.85 min: 100% of B; 1.95 min; 5% of B.

### EXAMPLE 1

### Synthesis of 2-(trans-4-hydroxycyclohexylamino)-4-(3-methyl-4-quinolin-3-ylindazol-1-yl)benzamide

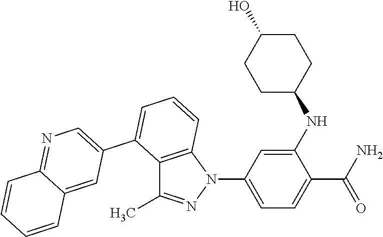

Stage 1: In a 50 ml three-necked flask under argon, 1,326 g of N-phenyl-bis(trifluoromethanesulphonimide) are added, at ambient temperature, to a suspension of 500 mg of 3-methyl-1H-indazol-4-ol [which can be prepared according to J. Med. Chem. 2000, 43(14), 2664] in 24 ml of dichloromethane. After stirring for 5 minutes, 518 μl of triethylamine and then 2 ml of tetrahydrofuran are added and the resulting mixture is left to stir overnight. The following day, the reaction medium is diluted with dichloromethane, and the organic phase is washed with distilled water, dried over magnesium sulphate and evaporated to dryness under vacuum. The residue is chromatographed on silica gel (15-40 μm), elution being carried out with a mixture of ethyl acetate and cyclohexane (50:50 v/v), 487 mg of trifluoromethanesulphonic acid 3-methyl-1H-indazol-4-yl ester are obtained in the form of a solid, the characteristics of which are the following (identical to WO 2005/028445 description 39 page 39 of Merck):

¹H NMR spectrum (400 MHz, δ in ppm, DMSO-d6): 2.60 (s, 3H); 7.12 (d, J=7.6 Hz, 1H); 7.44 (t, J=7.9 Hz, 1H); 7.60 (d, J=8.3 Hz, 1H); 13.24 (broad s, 1H).

Mass spectrum (LC/MS method A): Retention time Tr (min)=4.18; [M+H]+: m/z=281; [M−H]−: m/z=279.

Stage 2: In a 500 ml three-necked flask, a mixture of 9.3 g of trifluoromethanesulphonic acid 3-methyl-1H-indazol-4-yl ester obtained according to the preceding stage, 8.6 g of quinolin-3-boronic acid, 10.55 g of sodium carbonate and 3.84 g of tetrakis(triphenylphosphine)palladium(0) in 180 ml of toluene and 180 ml of ethanol and also 2.7 ml of distilled water is heated for one hour at 90° C. under argon. The reaction medium is evaporated to dryness under vacuum and the residue is taken up in 250 ml of ethyl acetate and washed with 100 ml of distilled water and then with a saturated solution of sodium chloride. The organic phase is dried over magnesium sulphate and evaporated to dryness under vacuum. The residue is chromatographed on silica gel (15-40 μm), elution being carried out with a mixture of methanol and dichloromethane (5:95 v/v), A mixture that contains the expected product is obtained, which is repurified by silica gel chromatography, elution being carried out with a mixture of ethyl acetate and cyclohexane (50:50 v/v). 6.43 g of 3-(3-methyl-1H-indazol-4-yl)quinoline are obtained in the form of a yellow solid, the characteristics of which are the following:

¹H NMR spectrum (400 MHz, δ in ppm, DMSO-d6): 2.10 (s, 3H); 7.11 (d, J=6.8 Hz, 1H); 7.45 (dd, J=8.4 and 7.0 Hz, 1H); 7.58 (d, J=8.3 Hz, 1H); 7.68 (td, J=7.6 and 1.0 Hz, 1H); 7.82 (ddd, J=8.4 and 7.0 and 1.2 Hz, 1H); 8.06 to 8.16 (m, 2H); 8.48 (d, J=1.7 Hz, 1H); 9.03 (d, J=2.2 Hz, 1H); 12.91 (broad s, 1H).

Mass spectrum (LC/MS method B): Retention time Tr (min)=3.21; [M+H]₊: m/z=260; [M−H]−: m/z=258.

Stage 3: In a 500 ml round-bottomed flask, 695 mg of sodium hydride as a dispersion at 60% in oil are added, in small portions, under argon at ambient temperature, to a mixture of 3.0 g of 3-(3-methyl-1H-indazol-4-yl)quinoline obtained according to the preceding stage and 2.55 g of 2-bromo-4-fluorobenzonitrile in 100 ml of anhydrous dimethylformamide. After stirring at ambient temperature for 1 hour, the reaction mixture is diluted with 500 ml of ethyl acetate and 30 ml of distilled water. The solid in suspension is filtered off and the filtrate is decanted. The organic phase is washed with 100 ml of a saturated solution of sodium chloride, dried over magnesium sulphate and evaporated to dryness under vacuum. The residue combined with the solid previously filtered off is triturated from isopropyl ether and then filtered. The solid is washed with 3 times 80 ml of isopropyl ether and then dried under vacuum. 4.12 g of 2-bromo-4-(3-methyl-4-quinolin-3-ylindazol-1-yl)benzonitrile are obtained in the form of a solid, the characteristics of which are the following:

¹H NMR spectrum (400 MHz, δ in ppm, DMSO-d6): 2.15 (s, 3H); 7.38 (d, J=6.8 Hz, 1H); 7.58 to 7.77 (m, 2H); 7.86 (t, J=7.6 Hz, 1H); 8.00 to 8.21 (m, 5H); 8.28 (s, 1H); 8.54 (s, 1H); 9.05 (s, 1H).

Mass spectrum (LC/MS method C): Retention time Tr (min)=1.18; [M+H]₊: m/z=439.

Stage 4: 286 mg of 2-bromo-4-(3-methyl-4-quinolin-3-ylindazol-1-yl)benzonitrile obtained according to the preceding stage, 300 mg of trans-4-aminocyclohexanol, 29 mg of palladium acetate, 125 mg of sodium tert-butoxide and 72 mg of 1,1′-bis(diphenylphosphino)ferrocene in 15 ml of toluene are respectively charged, under argon, to seven 20 ml microwave reactors. After stirring for 30 seconds at ambient temperature, the reaction medium is heated at 115° C. for 25 minutes with stirring. After cooling, the seven reaction media are combined and the resulting mixture is evaporated to dryness under vacuum. The residue is chromatographed on silica gel (15-40 μm), elution being carried out with a mixture of methanol and dichloromethane (5:95 v/v). Two compounds are obtained:

800 mg of 4-(3-methyl-4-quinolin-3-ylindazol-1-yl)benzonitrile in the form of a solid, the characteristics of which are the following:

¹H NMR spectrum (400 MHz, δ in ppm, DMSO-d6): 2.15 (s, 3H); 7.35 (d, J=7.1 Hz, 1H); 7.56 to 7.76 (m, 2H); 7.85 (t, J=7.6 Hz, 1H); 7.98 to 8.09 (m, 5H); 8.13 (t, J=7.9 Hz, 2H); 8.54 (s, 1H); 9.06 (d, J=2.2 Hz, 1H).

Mass spectrum (LC/MS method C): Retention time Tr (min)=1.09; [M+H]₊: m/z=361;

and 654 mg of 2-(trans-4-hydroxycyclohexylamino)-4-(3-methyl-4-quinolin-3-ylindazol-1-yl)benzonitrile in the form of a solid, the characteristics of which are the following:

¹H NMR spectrum (400 MHz, δ in ppm, DMSO-d6): 1.23 to 1.50 (m, 4H); 1.86 (d, J=12.0 Hz, 2H); 1.97 (d, J=9.0 Hz, 2H); 2.14 (s, 3H); 3.39 to 3.57 (m, 2 H); 4.54 (d, J=4.2 Hz, 1H); 5.86 (d, J=8.1 Hz, 1H); 7.09 (dd, J=8.4 and 1.8 Hz, 1H); 7.15 (d, J=2.0 Hz, 1H); 7.32 (d, J=7.1 Hz, 1H); 7.61 to 7.68 (m, 2H); 7.71 (dd, J=8.1 and 7.1 Hz, 1H); 7.85 (ddd, J=8.4 and 6.9 and 1.3 Hz, 1H); 7.95 (d, J=8.1 Hz, 1H); 8.10 to 8.15 (m, 2H); 8.53 (d, J=2.2 Hz, 1H); 9.06 (d, J=2.4 Hz, 1H).

Mass spectrum (LC/MS method C): Retention time Tr (min)=1.05; [M+H]+m/z=474.

Stage 5: In a 100 ml round-bottomed flask at ambient temperature under argon, 12.4 ml of ethanol and then 2.03 ml of 1M sodium hydroxide and finally 1.88 ml of aqueous hydrogen peroxide at 30% are added successively to a mixture of 480 mg of 2-(trans-4-hydroxycyclohexylamino)-4-(3-methyl-4-quinolin-3-ylindazol-1-yl)benzonitrile obtained according to the preceding stage, in 5.15 ml of dimethyl sulphoxide. After stirring at ambient temperature for 0.75 hour, the reaction mixture is poured into 100 ml of distilled water. After extraction with twice 200 ml of ethyl acetate, with the aqueous phase being saturated with sodium chloride, the combined organic phases are washed with twice 100 ml of distilled water and once with a saturated solution of sodium chloride, dried over magnesium sulphate and evaporated to dryness under vacuum. The solid is washed with 10 ml of ethyl ether and 10 ml of diisopropyl ether and then dried under vacuum. 437 mg of 2-(trans-4-hydroxycyclohexylamino)-4-(3-methyl-4-quinolin-3-ylindazol-1-yl)benzamide are obtained in the form of a beige solid, the characteristics of which are the following:

¹H NMR spectrum (400 MHz, δ in ppm, DMSO-d6):118 to 1.41 (m, 4H); 1.74 to 1.88 (m, 2H); 1.99 to 2.08 (m, 2H); 2.15 (s, 3H); 3.34 to 3.44 (m, 1H); 3.45 to 3.56 (m, 1H); 4.53 (d, J=4.2 Hz, 1H); 6.89 (dd, J=8.3 and 2.0 Hz, 1H); 6.98 (d, J=1.7 Hz, 1H); 7.18 (broad s, 1H); 7.29 (d, J=6.8 Hz, 1H); 7.62 (dd, J=8.4 and 7.2 Hz, 1H); 7.71 (t, J=7.7 Hz, 1H); 7.77 to 7.95 (m, 4H); 8.13 (t, J=7.2 Hz, 2H); 8.49 (d, J=7.6 Hz, 1H); 8.54 (d, J=2.2 Hz, 1H); 9.07 (d, J=2.2 Hz, 1H).

Mass spectrum (LC/MS method C): Retention time Tr (min)=0.86; [M+H]₊: m/z=492.

### EXAMPLE 2

### Synthesis of 4-(3-methyl-4-quinolin-3-ylindazol-1-yl)benzamide

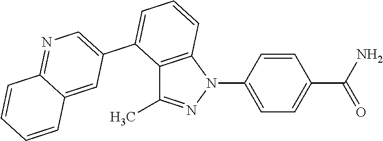

In a 100 ml round-bottomed flask at ambient temperature under argon, 18.6 ml of ethanol and then 3.05 ml of 1M sodium hydroxide and finally 2.84 ml of aqueous hydrogen peroxide at 30% are added successively to a mixture of 550 mg of 4-(3-methyl-4-quinolin-3-ylindazol-1-yl)benzonitrile obtained according to stage 4 of Example 1, dissolved in 7.75 ml of dimethyl sulphoxide. After stirring at ambient temperature for 0.5 hour, the reaction medium is poured into 100 ml of distilled water. After extraction with twice 200 ml of ethyl acetate, with the aqueous phase being saturated with sodium chloride, the combined organic phases are washed with distilled water and a saturated solution of sodium chloride, dried over magnesium sulphate and evaporated to dryness under vacuum. The solid is washed with diisopropyl ether and then dried under vacuum. 493 mg of 4-(3-methyl-4-quinolin-3-ylindazol-1-yl)benzamide are obtained in the form of a beige solid, the characteristics of which are the following:

¹H NMR spectrum (400 MHz, δ in ppm, DMSO-d6): 2.16 (s, 3H); 7.32 (d, J=7.1 Hz, 1H); 7.41 (broad s, 1H); 7.64 (dd, J=8.6 and 7.1 Hz, 1H); 7.71 (t, J=7.1 Hz, 1H); 7.81 to 7.91 (m, 3H); 8.00 (d, J=8.6 Hz, 1H); 8.04 to 8.17 (m, 5H); 8.55 (d, J=2.2 Hz, 1H); 9.07 (d, J=2.2 Hz, 1H).

Mass spectrum (LC/MS method B): Retention time Tr (min)=3.65; [M+H]₊: m/z=379; [M−H]−+HCOOH: m/z=423.

### EXAMPLE 3

### Synthesis of 2-(3-hydroxypropylamino)-4-(3-methyl-4-quinolin-3-ylindazol-1-yl)benzamide

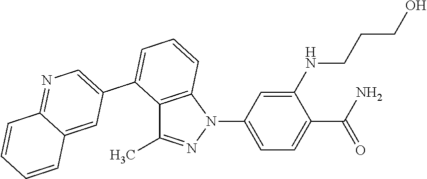

Stage 1: In a 250 ml round-bottomed flask, a mixture of 2.0 g of 2-bromo-4-(3-methyl-4-quinolin-3-ylindazol-1-yl)benzonitrile obtained according to stage 3 of Example 1, 684 mg of 3-aminopropanol, 4.45 g of cesium carbonate, 316 mg of 4,5-bis(diphenylphosphino)-9,9-dimethylxanthene and 102 mg of palladium acetate in 150 ml of dioxane is heated under argon at 90° C. for 3 hours. The reaction medium is then evaporated to dryness under vacuum and the residue is chromatographed on silica gel (15-40 μm), elution being carried out with a mixture of methanol and dichloromethane (5:95 v/v). 1.1 g of 2-(3-hydroxypropylamino)-4-(3-methyl-4-quinolin-3-ylindazol-1-yl)benzonitrile are obtained in the form of a beige solid, the characteristics of which are the following:

¹H NMR spectrum (400 MHz, δ in ppm, DMSO-d6): 1.79 (quin, J=6.4 Hz, 2H); 2.14 (s, 3H); 3.31 to 3.39 (m, 2H); 3.52 to 3.58 (m, 2H); 4.65 (t, J=5.0 Hz, 1H); 6.42 (t, J=5.3 Hz, 1H); 7.05 to 7.12 (m, 2H); 7.32 (dd, J=7.1 and 0.5 Hz, 1H); 7.61 to 7.68 (m, 2H); 7.68 to 7.73 (m, 1H); 7.85 (ddd, J=8.4 and 7.0 and 1.5 Hz, 1H); 8.00 (d, J=8.6 Hz, 1H); 8.12 (t, J=8.1 Hz, 2H); 8.53 (d, J=2.0 Hz, 1H); 9.06 (d, J=2.2 Hz, 1H).

Mass spectrum (LC/MS method B): Retention time Tr (min)=4.26; [M+H]₊: m/z=434; [M−H]−: m/z=432.

Stage 2: In a 50 ml round-bottomed flask at ambient temperature under argon, 14 ml of ethanol and then 2.31 ml of 1M sodium hydroxide and finally 2.15 ml of aqueous hydrogen peroxide at 30% are added successively to a mixture of 500 mg of 2-(3-hydroxypropylamino)-4-(3-methyl-4-quinolin-3-ylindazol-1-yl)benzonitrile obtained according to the preceding stage, in 5.85 ml of dimethyl sulphoxide. After stirring at ambient temperature for 0.75 hour, the reaction mixture is diluted with 150 ml of distilled water. After extraction with twice 150 ml of ethyl acetate, with the aqueous phase being saturated with sodium chloride, the combined organic phases are washed with twice 100 ml of distilled water and then a saturated solution of sodium chloride, dried over magnesium sulphate and evaporated to dryness under vacuum. The solid is washed with diisopropyl ether and ethyl ether and then dried under vacuum. 500 mg of 2-(3-hydroxypropylamino)-4-(3-methyl-4-quinolin-3-ylindazol-1-yl)benzamide are obtained in the form of a beige solid, the characteristics of which are the following:

¹H NMR spectrum (400 MHz, δ in ppm, DMSO-d6): 1.78 (quin, J=6.5 Hz, 2H); 2.14 (s, 3H); 3.21 to 3.29 (m, 2H); 3.50 to 3.58 (m, 2H); 4.55 (t, J=5.0 Hz, 1H); 6.93 (dd, J=8.3 and 2.0 Hz, 1H); 6.99 (d, J=2.0 Hz, 1H); 7.17 (broad s, 1H); 7.29 (d, J=6.8 Hz, 1H); 7.62 (dd, J=8.6 and 7.1 Hz, 1H); 7.71 (t, J=7.9 Hz, 1H); 7.76 to 8.01 (m, 4H); 8.13 (t, J=7.3 Hz, 2H); 8.47 (t, J=5.1 Hz, 1H); 8.54 (d, J=2.0 Hz, 1H); 9.06 (d, J=2.2 Hz, 1H).

Mass spectrum (LC/MS method C): Retention time Tr (min)=0.83; [M+H]₊: m/z=452.

### EXAMPLE 4

### Synthesis of 2-[2-(4-hydroxy-1-methylpiperidin-4-yl)ethylamino]-4-(3-methyl-4-quinolin-3-ylindazol-1-yl)benzamide

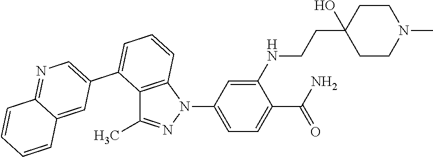

Stage 1: In a 100 ml three-necked flask, a mixture of 080 g of 2-bromo-4-(3-methyl-4-quinolin-3-ylindazol-1-yl)benzonitrile obtained according to stage 3 of Example 1, 576 mg of 4-(2-aminoethyl)-1-methyl-4-piperidinol, 1.78 g of cesium carbonate, 126 mg of 4,5-bis(diphenylphosphino)-9,9-dimethylxanthene and 41 mg of palladium acetate in 60 ml of dioxane is heated under argon at 90° C. for 2.5 hours. The reaction medium is diluted with ethyl acetate and filtered through clarcel. The filtrate is evaporated to dryness under vacuum and the residue is chromatographed on silica gel (15-40 μm), elution being carried out with a gradient of methanol and dichloromethane (from 10:90 to 20:80 v/v). 817 mg of 2-[2-(4-hydroxy-1-methylpiperidin-4-yl)ethylamino]-4-(3-methyl-4-quinolin-3-ylindazol-1-yl)benzonitrile are obtained in the form of a beige solid which is used, without further characterization, in the next stage.

Stage 2: In a 100 ml round-bottomed flask at ambient temperature under argon, 19.2 ml of ethanol and then 3.16 ml of 1M sodium hydroxide and finally 2.95 ml of aqueous hydrogen peroxide at 30% are added successively to a mixture of 817 mg of 2-[2-(4-hydroxy-1-methylpiperidin-4-yl)ethylamino]-4-(3-methyl-4-quinolin-3-ylindazol-1-yl)benzonitrile obtained according to the preceding stage, in 8.0 ml of dimethyl sulphoxide. After stirring at ambient temperature for 0.75 hour, the reaction mixture is diluted with 150 ml of distilled water. After extraction with twice 200 ml of ethyl acetate, with the aqueous phase being saturated with sodium chloride, the combined organic phases are washed with twice 150 ml of distilled water and then a saturated solution of sodium chloride, dried over magnesium sulphate and evaporated to dryness under vacuum. The residue is chromatographed on silica gel (15-40 μm), elution being carried out with a mixture of 7N ammoniacal methanol and dichloromethane (10:90 v/v). 444 mg of 2-[2-(4-hydroxy-1-methylpiperidin-4-yl)ethylamino]-4-(3-methyl-4-quinolin-3-ylindazol-1-yl)benzamide are obtained in the form of a white solid, the characteristics of which are the following:

¹H NMR spectrum (400 MHz, δ in ppm, DMSO-d6) 1.54 (t, J=5.1 Hz, 4 H); 1.66 to 1.78 (m, 2H); 2.14 (s, 3H); 2.15 (s, 3H); 2.21 to 2.32 (m, 2H); 2.33 to 2.44 (m, 2H); 3.22 to 3.29 (m, 2H); 4.20 (s, 1H); 6.92 (dd, J=8.6 and 2.0 Hz, 1H); 6.99 (d, J=2.0 Hz, 1H); 7.17 (broad s, 1H); 7.29 (d, J=6.6 Hz, 1H); 7.61 (dd, J=8.6 and 7.1 Hz, 1H); 7.71 (t, J=7.5 Hz, 1H); 7.78 to 7.93 (m, 3H); 7.97 (d, J=8.6 Hz, 1H); 8.13 (t, J=7.2 Hz, 2H); 8.40 (t, J=5.0 Hz, 1H); 8.53 (d, J=2.2 Hz, 1 H); 9.06 (d, J=2.2 Hz, 1H).

Mass spectrum (LC/MS method C): Retention time Tr (min)=0.65; [M+H]₊: m/z=535; [M−H]−: m/z=533.

### EXAMPLE 5

### Synthesis of 2-(2-hydroxy-2-methylpropylamino)-4-(3-methyl-4-quinolin-3-ylindazol-1-yl)benzamide

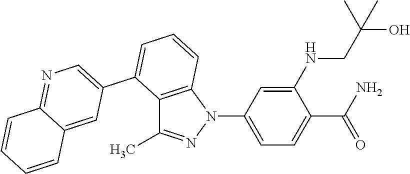

Stage 1: In a 100 ml round-bottomed flask, a mixture of 350 mg of 2-bromo-4-(3-methyl-4-quinolin-3-ylindazol-1-yl)benzonitrile obtained according to stage 3 of Example 1, 142 mg of 1-amino-2-methylpropan-2-ol, 779 mg of cesium carbonate, 55 mg of 4,5-bis(diphenylphosphino)-9,9-dimethylxanthene and 18 mg of palladium acetate in 28 ml of dioxane is heated under argon at 90° C. for 3.5 hours. The reaction medium is diluted with 150 ml of ethyl acetate and filtered through clarcel. The filtrate is evaporated to dryness under vacuum and the residue is chromatographed on silica gel (15-40 μm), elution being carried out with a mixture of methanol and dichloromethane (5:95 v/v), 300 mg of 2-(2-hydroxy-2-methylpropylamino)-4-(3-methyl-4-quinolin-3-ylindazol-1-yl)benzonitrile are obtained in the form of a solid, the characteristics of which are the following:

¹H NMR spectrum (400 MHz, δ in ppm, DMSO-d6): 1.21 (s, 6H); 2.14 (s, 3 H); 3.22 (d, J=5.6 Hz, 2H); 4.80 (s, 1H); 5.80 (t, J=5.4 Hz, 1H); 7.09 (dd, J=8.3 and 2.0 Hz, 1H); 7.24 (d, J=1.7 Hz, 1H); 7.32 (d, J=6.8 Hz, 1H); 7.57 to 7.76 (m, 3H); 7.85 (ddd, J=8.4 and 7.0 and 1.5 Hz, 1H); 8.01 (d, J=8.6 Hz, 1H); 8.12 (t, J=7.8 Hz, 2H); 8.53 (d, J=2.2 Hz, 1H); 9.05 (d, J=2.4 Hz, 1H).

Mass spectrum (LC/MS method C): Retention time Tr (min)=1.07; [M+H]₊: m/z=448; [M−H]−: m/z=446.

Stage 2: In a 50 ml round-bottomed flask at ambient temperature under argon, 7.7 ml of ethanol and then 1.26 ml of 1M sodium hydroxide and finally 1.17 ml of aqueous hydrogen peroxide at 30% are added successively to a mixture of 281 mg of 2-(2-hydroxy-2-methylpropylamino)-4-(3-methyl-4-quinolin-3-ylindazol-1-yl)benzonitrile obtained according to the preceding stage, in 6.0 ml of dimethyl sulphoxide. After stirring at ambient temperature for 0.75 hour, the reaction medium is diluted with 150 ml of distilled water. After extraction with twice 200 ml of ethyl acetate, with the aqueous phase being saturated with sodium chloride, the combined organic phases are washed with twice 150 ml of distilled water and then a saturated solution of sodium chloride, dried over magnesium sulphate and evaporated to dryness under vacuum. The residual solid is triturated from isopropyl ether, filtered, washed with ethyl ether and dried under vacuum. 285 mg of 2-(2-hydroxy-2-methylpropylamino)-4-(3-methyl-4-quinolin-3-ylindazol-1-yl)benzamide are obtained in the form of a beige solid, the characteristics of which are the following:

¹H NMR spectrum (400 MHz, δ in ppm, DMSO-d6): 1.21 (s, 6H); 2.14 (s, 3H); 3.10 (d, J=5.1 Hz, 2H); 4.58 (s, 1H); 6.89 (dd, J=8.3 and 2.0 Hz, 1H); 6.99 (d, J=2.0 Hz, 1H); 7.13 (broad s, 1H); 7.29 (d, J=7.1 Hz, 1H); 7.56 to 7.66 (m, 1H); 7.71 (t, J=7.5 Hz, 1H); 7.75 to 7.92 (m, 3H); 7.95 (d, J=8.6 Hz, 1H); 8.13 (t, J=7.3 Hz, 2H); 8.53 (d, J=2.0 Hz, 1H); 8.64 (t, J=5.0 Hz, 1H); 9.06 (d, J=2.2 Hz, 1H).

Mass spectrum (LC/MS method C): Retention time Tr (min)=0.90; [M+H]₊: m/z=466; [M−H]−: m/z=464.

### EXAMPLE 6

### Synthesis of 4-(3-methyl-4-quinolin-3-ylindazol-1-yl)-2-(2,2,6,6-tetramethylpiperidin-4-ylamino)benzamide

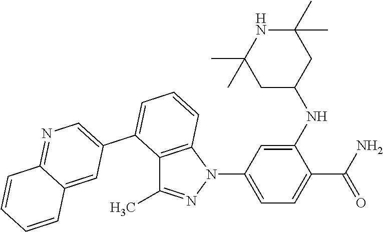

Stage 1: In a 100 ml round-bottomed flask, a mixture of 350 mg of 2-bromo-4-(3-methyl-4-quinolin-3-ylindazol-1-yl)benzonitrile obtained according to stage 3 of Example 1, 273 mg of 4-amino-2,2,6,6-tetramethylpiperidine, 779 mg of cesium carbonate, 55 mg of 4,5-bis(diphenylphosphino)-9,9-dimethylxanthene and 18 mg of palladium acetate in 26 ml of dioxane is heated under argon at 90° C. for 5 hours. The reaction medium is diluted with 150 ml of ethyl acetate and filtered through clarcel. The filtrate is evaporated to dryness under vacuum and the residue is chromatographed on silica gel (15-40 μm), elution being carried out with a mixture of 7N ammoniacal methanol and dichloromethane (5:95 v/v). 368 mg of 4-(3-methyl-4-quinolin-3-ylindazol-1-yl)-2-(2,2,6,6-tetramethylpiperidin-4-ylamino)benzonitrile are obtained in the form of a solid, the characteristics of which are the following:

¹H NMR spectrum (400 MHz, δ in ppm, DMSO-d6): 1.08 (s, 6H); 1.15 to 1.21 (m, 2H); 1.22 (s, 6H); 1.91 (dd, J=12.1 and 3.1 Hz, 2H); 2.13 (s, 3H); 3.92 to 4.06 (m, 1H); 5.84 (d, J=7:1 Hz, 1H); 7.12 (dd, J=8.4 and 1.8 Hz, 1H); 7.25 (d, J=1.7 Hz, 1H); 7.32 (d, J=6.6 Hz, 1H); 7.63 (dd, J=8.6 and 7.1 Hz, 1H); 7.67 (d, J=8.3 Hz, 1H); 7.71 (ddd, J=8.1 and 7.0 and 1.0 Hz, 1H); 7.85 (ddd, J=8.5 and 6.9 and 1.5 Hz, 1H); 8.00 (d, J=8.3 Hz, 1H); 8.12 (t, J=7.5 Hz, 2H); 8.54 (d, J=2.0 Hz, 1H); 9.06 (d, J=2.4 Hz, 1H).

Mass spectrum (LC/MS method C): Retention time Tr (min)=0.84; [M−H]₊: m/z=515.

Stage 2: In a 50 ml round-bottomed flask at ambient temperature under argon, 10 ml of ethanol and then 1.32 ml of 1M sodium hydroxide and finally 1.24 ml of aqueous hydrogen peroxide at 30% are added successively to a mixture of 338 mg of 4-(3-methyl-4-quinolin-3-ylindazol-1-yl)-2-(2,2,6,6-tetramethylpiperidin-4-ylamino)benzonitrile obtained according to the preceding stage, in 10 ml of dimethyl sulphoxide. After stirring at ambient temperature for 1.5 hours, the reaction medium is diluted with 100 ml of distilled water. After extraction with twice 200 ml of ethyl acetate, with the aqueous phase being saturated with sodium chloride, the combined organic phases are washed with twice 150 ml of distilled water and then a saturated solution of sodium chloride, dried over magnesium sulphate and evaporated to dryness under vacuum. 320 mg of 4-(3-methyl-4-quinolin-3-ylindazol-1-yl)-2-(2,2,6,6-tetramethylpiperidin-4-ylamino)benzamide are obtained in the form of a white solid, the characteristics of which are the following:

¹H NMR spectrum (400 MHz, δ in ppm, DMSO-d6): 1.03 (t, J=12.0 Hz, 2 H); 1.08 (s, 6H); 1.23 (s, 6H); 1.98 (dd, J=12.2 and 2.9 Hz, 2H); 2.14 (s, 3H); 3.78 to 3.88 (m, 1H); 6.92 (dd, J=8.4 and 2.1 Hz, 1H); 7.11 (d, J=1.7 Hz, 1H); 7.18 (broad s, 1H); 7.29 (d, J=6.6 Hz, 1H); 7.60 (dd, J=8.6 and 7.1 Hz, 1H); 7.67 to 7.74 (m, 1H); 7.79 to 7.92 (m, 3H); 7.97 (d, J=8.3 Hz, 1H); 8.13 (t, J=7.2 Hz, 2 H); 8.41 (d, J=7.3 Hz, 1H); 8.54 (d, J=2.2 Hz, 1H); 9.06 (d, J=2.4 Hz, 1H).

Mass spectrum (LC/MS method C): Retention time Tr (min)=0.72; [M+H]₊: m/z=533.

### EXAMPLE 7

### Synthesis of 4-(3-methyl-4-quinolin-3-ylindazol-1-yl)-2-(tetrahydropyran-4-ylamino)benzamide

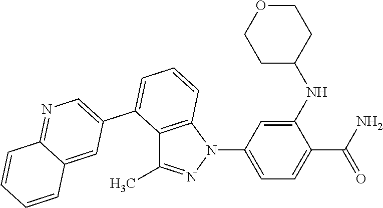

Stage 1: In a 50 ml three-necked flask, a mixture of 250 mg of 2-bromo-4-(3-methyl-4-quinolin-3-ylindazol-1-yl)benzonitrile obtained according to stage 3 of Example 1, 115 mg of 4-aminotetrahydropyran, 556 mg of cesium carbonate, 40 mg of 4,5-bis(diphenylphosphino)-9,9-dimethylxanthene and 13 mg of palladium acetate in 20 ml of dioxane is heated under argon at 90° C. for 4 hours. The reaction medium is diluted with ethyl acetate and filtered through clarcel. The filtrate is washed with distilled water and then a saturated solution of sodium chloride, dried over magnesium sulphate and evaporated to dryness under vacuum. The residue is chromatographed on silica gel (15-40 μm), elution being carried out with a mixture of methanol and dichloromethane (2:98 v/v). 185 mg of 4-(3-methyl-4-quinolin-3-ylindazol-1-yl)-2-(tetrahydropyran-4-ylamino)benzonitrile are obtained in the form of a solid which is used, without further characterization, in the next stage.

Stage 2: In a 50 ml round-bottomed flask at ambient temperature under argon, 4.8 ml of ethanol and then 0.81 ml of 1M sodium hydroxide and finally 0.74 ml of aqueous hydrogen peroxide at 30% are added successively to a mixture of 185 mg of 4-(3-methyl-4-quinolin-3-ylindazol-1-yl)-2-(tetrahydropyran-4-ylamino)benzonitrile obtained according to the preceding stage, in 2 ml of dimethyl sulphoxide. After stirring at ambient temperature for 0.5 hour, the reaction medium is diluted with 100 ml of distilled water. After extraction with ethyl acetate, the combined organic phases are washed with distilled water and then a saturated solution of sodium chloride, dried over magnesium sulphate and evaporated to dryness under vacuum. The residue is chromatographed on silica gel (15-40 μm), elution being carried out with a mixture of ethyl acetate and heptane (90:10 v/v). 150 mg of 4-(3-methyl-4-quinolin-3-ylindazol-1-yl)-2-(tetrahydropyran-4-ylamino)benzamide are obtained in the form of a white solid, the characteristics of which are the following:

¹H NMR spectrum (400 MHz, δ in ppm, DMSO-d6): 1.36 to 1.52 (m, 2H); 1.92 to 2.04 (m, 2H); 2.14 (s, 3H); 3.41 to 3.55 (m, 2H); 3.61 to 3.77 (m, 1H); 3.84 (dt, J=11.7 and 3.9 Hz, 2H); 6.91 (dd, J=8.4 and 2.1 Hz, 1H); 7.04 (d, J=2.0 Hz, 1H); 7.22 (broad s, 1H); 7.29 (d, J=7.1 Hz, 1H); 7.62 (dd, J=8.6 and 7.1 Hz, 1H); 7.71 (t, J=7.9 Hz, 1H); 7.78 to 8.01 (m, 4H); 8.06 to 8.18 (m, 2H); 8.53 (d, J=2.2 Hz, 1H); 8.62 (d, J=7.6 Hz, 1H); 9.06 (d, J=2.2 Hz, 1H).

Mass spectrum (LC/MS method C); Retention time Tr (min)=0.94; [M+H]+: m/z=478.

### EXAMPLE 8

### Synthesis of 2-(2-fluoroethylamino)-4-(3-methyl-4-quinolin-3-ylindazol-1-yl)benzamide

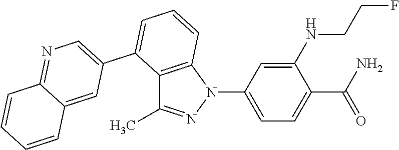

Stage 1: In a 50 ml three-necked flask, a mixture of 250 mg of 2-bromo-4-(3-methyl-4-quinolin-3-ylindazol-1-yl)benzonitrile obtained according to stage 3 of Example 1, 113 mg of 2-fluoroethylamine hydrochloride, 556 mg of cesium carbonate, 40 mg of 4,5-bis(diphenylphosphino)-9,9-dimethylxanthene, 160 μl of triethylamine and 13 mg of palladium acetate in 20 ml of dioxane is heated under argon at 100° C. for 20 hours. The reaction medium is poured into 100 ml of distilled water and extracted with three times 50 ml of ethyl acetate. The combined organic phases are washed with 50 ml of a saturated solution of sodium chloride, dried over magnesium sulphate and evaporated to dryness under vacuum. The residue is chromatographed on silica gel (40-63 μm), elution being carried out with a mixture of ethanol and dichloromethane (1:99 v/v). 160 mg of 2-(2-fluoroethylamino)-4-(3-methyl-4-quinolin-3-ylindazol-1-yl)benzonitrile are obtained in the form of an amorphous solid which is used, without further characterization, in the next stage.

Stage 2: In a 25 ml three-necked flask at ambient temperature under argon, 4.0 ml of ethanol and then 0.72 ml of 1M sodium hydroxide and finally 0.70 ml of aqueous hydrogen peroxide at 30% are added successively to a mixture of 160 mg of 2-(2-fluoroethylamino)-4-(3-methyl-4-quinolin-3-ylindazol-1-yl)benzonitrile obtained according to the preceding stage, in 2.5 ml of dimethyl sulphoxide. After stirring at ambient temperature for 0.25 hour, the reaction medium is diluted with 100 ml of distilled water. After extraction with three times 50 ml of ethyl acetate, the combined organic phases are washed with 50 ml of a saturated solution of sodium chloride, dried over magnesium sulphate and evaporated to dryness under vacuum. The residue is chromatographed on silica gel (40-63 μm), elution being carried out with a mixture of ethanol and dichloromethane (4:96 v/v). 125 mg of 2-(2-fluoroethylamino)-4-(3-methyl-4-quinolin-3-ylindazol-1-yl)benzamide are obtained in the form of an amorphous beige solid, the characteristics of which are the following:

¹H NMR spectrum (400 MHz, δ in ppm, DMSO-d6); 2.14 (s, 3H); 3.56 (dt, J=32.5 and 4.6 Hz, 2H); 4.67 (dt, J=47.7 and 4.6 Hz, 2H); 6.97 (d, J=7.1 Hz, 1H); 7.05 (s, 1H); 7.25 (broad s, 1H); 7.29 (d, J=6.8 Hz, 1H); 7.61 (t, J=7.8 Hz, 1H); 7.71 (t, J=7.5 Hz, 1H); 7.76 to 7.89 (m, 2H); 7.90 to 8.04 (m, 2H); 8.13 (t, J=7.2 Hz, 2H); 8.53 (d, J=1.2 Hz, 1H); 8.70 (t, J=5.4 Hz, 1H); 9.06 (d, J=2.0 Hz, 1H).

Mass spectrum (LC/MS method C): Retention time Tr (min)=0.94; [M+H]₊: m/z=440; [M−H]−: m/z=438.

### EXAMPLE 9

### Synthesis of 3-(2-hydroxy-2-methylpropylamino)-5-(3-methyl-4-quinolin-3-ylindazol-1-yl)pyridine-2-carboxamide

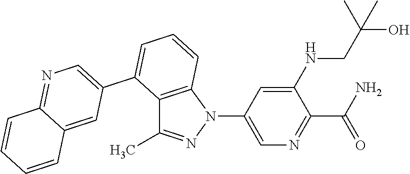

Stage 1: 600 mg of 2-cyano-3,5-difluoropyridine, 475 mg of 2-amino-2-methylpropan-2-ol and 1.18 g of potassium carbonate in 9 ml of dimethyl sulphoxide are respectively charged, under argon, to three 20 ml microwave reactors. After stirring for 30 seconds at ambient temperature, the reaction medium is heated at 115° C. for 1 hour with stirring. After cooling, the three reactions are combined, the resulting mixture is diluted with 200 ml of ethyl acetate and the organic phase is washed with 100 ml of distilled water. The aqueous phase is re-extracted with 200 ml of ethyl acetate. The combined organic phases are washed with twice 150 ml of distilled water and then with a saturated solution of sodium chloride, dried over magnesium sulphate and evaporated to dryness under vacuum. The residue is chromatographed on silica gel (15-40 μm), elution being carried out with a mixture of cyclohexane and ethyl acetate (50:50 v/v). 768 mg of 5-fluoro-3-(2-hydroxy-2-methylpropylamino)pyridine-2-carbonitrile are obtained in the form of a solid, the characteristics of which are the following:

¹H NMR spectrum (400 MHz, δ in ppm, DMSO-d6): 1.14 (s, 6H); 3.16 (d, J=5.9 Hz, 2H); 4.67 (s, 1H); 6.21 (broad s, 1H); 7.36 (dd, J=12.0 and 2.4 Hz, 1H); 7.86 (d, J=2.4 Hz, 1H),

Mass spectrum (LC/MS method C): Retention time Tr (min)=0.58; [M+H]₊: m/z=210; [M−H]−: m/z=208.

Stage 2: In a 50 ml round-bottomed flask under argon, 58 mg of a dispersion of sodium hydride at 60% in oil are added, at ambient temperature in small portions, to a solution of 250 mg of 3-(3-methyl-1H-indazol-4-yl)quinoline obtained according to stage 2 of Example 1, in 10 ml of anhydrous dimethylformamide. After stirring at ambient temperature for 0.5 hour, 222 mg of 5-fluoro-3-(2-hydroxy-2-methylpropylamino)pyridine-2-carbonitrile obtained according to the preceding stage are added and the reaction medium is kept at ambient temperature for a further 0.25 hour and then heated at 50° C. for 2 hours. The reaction medium, after cooling to ambient temperature, is diluted with 200 ml of ethyl acetate and the organic phase is washed with twice 100 ml of distilled water and then with a saturated solution of sodium chloride, dried over magnesium sulphate and evaporated to dryness under vacuum. The solid residue is triturated from isopropyl ether, filtered, washed with 10 ml of ethyl ether and then 20 ml of isopropyl ether and dried under vacuum. 360 mg of 3-(2-hydroxy-2-methylpropylamino)-5-(3-methyl-4-quinolin-3-ylindazol-1-yl)pyridine-2-carbonitrile are obtained in the form of a yellow solid, the characteristics of which are the following:

¹H NMR spectrum (400 MHz, δ in ppm, DMSO-d6): 1.21 (s, 6H); 2.15 (s, 3H); 3.25 to 3.29 (m, 2H); 4.81 (s, 1H); 6.30 (t, J=5.7 Hz, 1H); 7.35 (d, J=7.1 Hz, 1H); 7.61 to 7.75 (m, 2H); 7.78 (d, J=2.0 Hz, 1H); 7.85 (td, J=7.6 and 1.3 Hz, 1H); 8.07 (d, J=8.3 Hz, 1H); 8.13 (t, J=7.5 Hz, 2H); 8.36 (d, J=2.0 Hz, 1H); 8.54 (d, J=2.0 Hz, 1H); 9.05 (d, J=2.2 Hz, 1H).

Mass spectrum (LC/MS method C): Retention time Tr (min)=1.01; [M+H]₊: m/z=449; [M−H]−: m/z=447.

Stage 3: In a 50 ml round-bottomed flask at ambient temperature under argon, 10 ml of ethanol and then 1.6 ml of 1M sodium hydroxide and finally 1.5 ml of aqueous hydrogen peroxide at 30% are added successively to a mixture of 355 mg of 3-(2-hydroxy-2-methylpropylamino)-5-(3-methyl-4-quinolin-3-ylindazol-1-yl)pyridine-2-carbonitrile obtained according to the preceding stage, in 4.2 ml of dimethyl sulphoxide. After stirring at ambient temperature for 0.75 hour, the reaction medium is diluted with 150 ml of distilled water and saturated with solid sodium chloride. After extraction with twice 200 ml of ethyl acetate, the combined organic phases are washed with twice 100 ml of distilled water and then with a saturated solution of sodium chloride, dried over magnesium sulphate and evaporated to dryness under vacuum. The solid residue is triturated from 10 ml of isopropyl ether, filtered, washed with 10 ml of ethyl ether and dried under vacuum. 300 mg of 3-(2-hydroxy-2-methylpropylamino)-5-(3-methyl-4-quinolin-3-ylindazol-1-yl)pyridine-2-carboxamide are obtained in the form of a yellow solid, the characteristics of which are the following:

¹H NMR spectrum (400 MHz, δ in ppm, DMSO-d6): 1.21 (s, 6H); 2.16 (s, 3H); 3.13 to 3.21 (m, 2H); 4.64 (s, 1H); 7.33 (d, J=6.8 Hz, 1H); 7.41 (broad s, 1 H); 7.51 (s, 1H); 7.65 (t, J=7.7 Hz, 1H); 7.71 (t, J=7.6 Hz, 1H); 7.85 (t, J=7.6 Hz, 1H); 8.00 (d, J=8.1 Hz, 2H); 8.13 (d, J=7.6 Hz, 2H); 8.19 (s, 1H); 8.54 (s, 1H); 8.92 (t, J=5.4 Hz, 1H); 9.06 (s, 1H).

Mass spectrum (LC/MS method B): Retention time Tr (min)=4.02; [M+H]₊: m/z=467.

### EXAMPLE 10

### Synthesis of 5-(3-methyl-4-quinolin-3-ylindazol-1-yl)-3-(tetrahydro-pyran-4-ylamino)pyridine-2-carboxamide

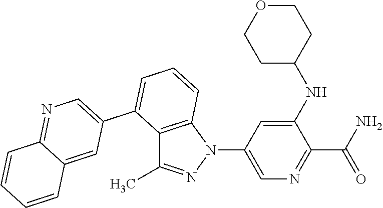

Stage 1: 410 mg of 2-cyano-3,5-difluoropyridine, 483 mg of 4-aminotetrahydropyran hydrochloride, 809 mg of potassium carbonate and 490 μl of triethylamine in 6.1 ml of dimethyl sulphoxide are charged successively, under argon, to a 20 ml microwave reactor. After stirring for 30 seconds at ambient temperature, the reaction medium is heated at 115° C. for 1 hour with stirring. After cooling, the reaction medium is diluted with ethyl acetate and the organic phase is washed with distilled water and then with a saturated solution of sodium chloride, dried over magnesium sulphate and evaporated to dryness under vacuum. The residue is chromatographed on silica gel (15-40 μm), elution being carried out with a mixture of heptane and ethyl acetate (60:40 v/v), 160 mg of 5-fluoro-3-(tetrahydropyran-4-ylamino)pyridine-2-carbonitrile are obtained in the form of a solid, the characteristics of which are the following:

¹H NMR spectrum (500 MHz, δ in ppm, DMSO-d6): 1.55 to 1.68 (m, 2H); 1.78 (dd, J=12.5 and 2.2 Hz, 2H); 3.40 (td, J=11.7 and 1.5 Hz, 2H); 3.56 to 3.69 (m, 1H); 3.87 (dd, J=11.5 and 2.7 Hz, 2H); 6.48 (d, J=7.8 Hz, 1H); 7.40 (dd, J=12.0 and 2.2 Hz, 1H); 7.89 (d, J=2.4 Hz, 1H).

Stage 2: In a 50 ml three-necked flask under argon, 43 mg of a dispersion of sodium hydride at 60% in oil are added, at ambient temperature, to a mixture of 185 mg of 3-(3-methyl-1H-indazol-4-yl)quinoline obtained according to stage 2 of Example 1, in 10 ml of anhydrous dimethylformamide. After stirring at ambient temperature for 0.5 hour at 30° C., 158 mg of 5-fluoro-3-(tetrahydropyran-4-ylamino)pyridine-2-carbonitrile obtained according to the preceding stage are added at this temperature, and then the reaction medium is heated at 50° C. overnight. After the reaction medium has cooled to ambient temperature, a small amount of ethanol and ethyl acetate are added and the whole mixture is evaporated to dryness under vacuum. 325 mg of 5-(3-methyl-4-quinolin-3-ylindazol-1-yl)-3-(tetrahydropyran-4-ylamino)pyridine-2-carbonitrile are obtained in the form of a solid which is used as it is for the next stage without further purification and characterization.

Stage 3: In a 50 ml round-bottomed flask at ambient temperature under argon, 8.5 ml of ethanol and then 1.4 ml of 1M sodium hydroxide and finally 1.3 ml of aqueous hydrogen peroxide at 30% are added successively to a mixture of 325 mg of 5-(3-methyl-4-quinolin-3-ylindazol-1-yl)-3-(tetrahydropyran-4-ylamino)pyridine-2-carbonitrile obtained according to the preceding stage, in 3.5 ml of dimethyl sulphoxide. After stirring at ambient temperature for 0.75 hour, the reaction medium is diluted with distilled water. The resulting mixture is extracted with ethyl acetate, and the organic phase is washed with distilled water and then with a saturated solution of sodium chloride, dried over magnesium sulphate and evaporated to dryness under vacuum. The residue is chromatographed on silica gel (15-40 μm), elution being carried out with a mixture of ethyl acetate and heptane (70:30 v/v). 217 mg of 5-(3-methyl-4-quinolin-3-ylindazol-1-O-3-(tetrahydropyran-4-ylamino)pyridine-2-carboxamide are obtained in the form of a white solid, the characteristics of which are the following:

¹H NMR spectrum (400 MHz, δ in ppm, DMSO-d6): 1.39 to 1.54 (m, 2H); 1.99 (dd, J=13.4 and 2.4 Hz, 2H); 2.16 (s, 3H); 3.51 (dd, J=12.5 and 2.2 Hz, 2H); 3.72 to 3.82 (m, 1H); 3.85 (dt, J=11.7 and 3.8 Hz, 2H); 7.33 (d, J=7.1 Hz, 1H); 7.50 (broad s, 1H); 7.53 (d, J=2.2 Hz, 1H); 7.66 (dd, J=8.6 and 7.1 Hz, 1H); 7.71 (td, J=7.5 and 1.1 Hz, 1H); 7.86 (ddd, J=8.5 and 6.9 and 1.5 Hz, 1H); 7.95 (d, J=8.6 Hz, 1H); 8.06 (broad s, 1H); 8.09 to 8.16 (m, 2H); 8.22 (d, J=2.0 Hz, 1H); 8.53 (d, J=1.7 Hz, 1H); 8.85 (d, J=8.1 Hz, 1H); 9.06 (d, J=2.2 Hz, 1H).

Mass spectrum (LC/MS method B): Retention time Tr (min)=4.16; [M+H]₊: m/z=479; [M−H]−+HCOOH: m/z=523.

### EXAMPLE 11

### Synthesis of aminoacetic acid trans-4-[2-carbamoyl-5-(3-methyl-4-quinolin-3-ylindazol-1-yl)phenylamino]cyclohexyl ester

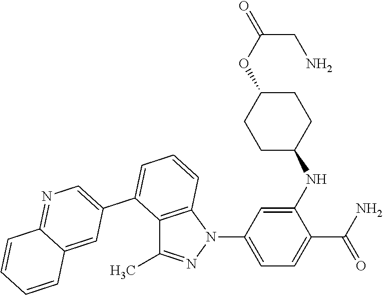

Stage 1: In a 500 ml three-necked flask, a mixture of 175 mg of 2-(trans-4-hydroxycyclohexylamino)-4-(3-methyl-4-quinolin-3-ylindazol-1-yl)benzamide obtained according to Example 1, 125 mg of tert-butoxycarbonylaminoacetic acid, 87 mg of 4-dimethylaminopyridine, 124 μl of N,N-diisopropylethylamine and 234 mg of O-[(ethoxycarbonyl)cyanomethyleneamino]-N,N,N′,N′-tetramethyluronium tetrafluoroborate (TOTU) in 20 ml of dichloromethane and 2 ml of anhydrous dimethylformamide is stirred under argon at ambient temperature for 20 hours. The reaction medium is evaporated to dryness under vacuum. 25 ml of distilled water are added dropwise to the residue, with vigorous stirring, and the resulting mixture is then extracted with three times 50 ml of dichloromethane. The combined organic phases are washed with three times 25 ml of distilled water and three times 25 ml of a saturated solution of sodium bicarbonate, dried over magnesium sulphate and evaporated to dryness under vacuum. The residue is chromatographed on silica gel (40-63 μm), elution being carried out with a gradient of ethanol and dichloromethane (from 1:99 to 2:98 v/v). 190 mg of tert-butoxycarbonylaminoacetic acid trans-4-[2-carbamoyl-5-(3-methyl-4-quinolin-3-ylindazol-1-yl)phenylamino]cyclohexyl ester are obtained in the form of an amorphous orangey solid which is used in the next stage without further characterization.

Stage 2: In a 25 ml three-necked flask, 2.5 ml of trifluoroacetic acid are added, dropwise at 0° C. under argon, to a mixture of 190 mg of tert-butoxycarbonylaminoacetic acid trans-4-[2-carbamoyl-5-(3-methyl-4-quinolin-3′ ylindazol-1-yl)phenylamino]cyclohexyl ester obtained according to the preceding stage, in 5 ml of dichloromethane. After stirring at 0° C. for 30 minutes, the reaction medium is left to return to ambient temperature and stirred for a further 1 hour. The reaction medium is evaporated to dryness under vacuum and 10 ml of distilled water are added dropwise to the residue, with vigorous stirring. The aqueous phase is brought to pH 7-8 with a saturated solution of sodium bicarbonate and extracted with twice 25 ml of ethyl acetate. The combined organic phases are washed with twice 10 ml of distilled water and 10 ml of a saturated solution of sodium chloride, dried over magnesium sulphate and evaporated to dryness under vacuum. The solid residue is triturated from 5 ml of isopropyl ether, filtered, washed with isopropyl ether and dried under vacuum. 140 mg of aminoacetic acid trans-4-[2-carbamoyl-5-(3-methyl-4-quinolin-3-ylindazol-1-yl)phenylamino]cyclo-hexyl ester are obtained in the form of a beige solid, the characteristics of which are the following:

¹H NMR spectrum (400 MHz, δ in ppm, DMSO-d6): 1.30 to 1.48 (m, 2H); 1.47 to 1.62 (m, 2H); 1.73 (broad s, 2H); 1.93 (d, J=9.3 Hz, 2H); 2.07 (d, J=11.7 Hz, 2H); 2.14 (s, 3H); 3.24 (broad s, 2H); 3.41 to 3.57 (m, 1H); 4.75 (t, J=8.8 Hz, 1H); 6.90 (d, J=8.3 Hz, 1H); 7.01 (broad s, 1H); 7.19 (broad s, 1H); 7.29 (d, J=6.8 Hz, 1H); 7.62 (t, J=7.7 Hz, 1H); 7.71 (t, J=7.3 Hz, 1H); 7.75 to 8.02 (m, 4 H); 8.13 (t, J=6.8 Hz, 2H); 8.43 to 8.62 (m, 2H); 9.06 (s, 1H).

Mass spectrum (LC/MS method C): Retention time Tr (min)=0.73; [M+H]₊: m/z=549.

### EXAMPLE 12

### Synthesis of 4-[4-(6-fluoro-1H-benzimidazol-2-yl)-3-methylindazol-1-yl]-2-(trans-4-hydroxycyclohexylamino)benzamide

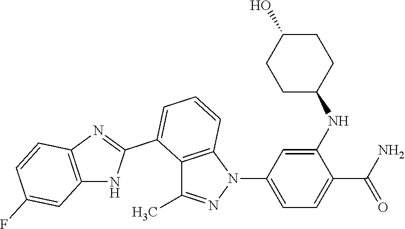

Stage 1: In an autoclave, a mixture of 7.045 g of trifluoromethanesulphonic acid 3-methyl-1H-indazol-4-yl ester obtained according to stage 1 of Example 1, 1.129 g of palladium acetate, 2.074 g of 1,3-bis(diphenylphosphino)propane and 3.51 ml of triethylamine in 34 ml of methanol and 78 ml of dimethylformamide is maintained at 50° C. for 16 hours at 2 bar of carbon monoxide pressure. After flushing with argon, the reaction medium is evaporated to dryness under vacuum. The residue is taken up in 200 ml of dichloromethane. The organic phase is washed with twice 100 ml of distilled water, dried over magnesium sulphate and evaporated to dryness under vacuum. The residue is chromatographed on silica gel (15-40 μm), elution being carried out with a mixture of methanol and dichloromethane (5:95 v/v). The product obtained is rechromatographed on silica gel (15-40 μm), elution being carried out with a mixture of ethyl acetate and cyclohexane (40:60 v/v). 3.33 g of 3-methyl-1H-indazole-4-carboxylic acid methyl ester are obtained in the form of a solid, the characteristics of which are the following:

¹H NMR spectrum (400 MHz, δ in ppm, DMSO-d6): 2.59 (s, 3H); 3.91 (s, 3 H); 7.40 (t, J=7.7 Hz, 1H); 7.61 (d, J=7.1 Hz, 1H); 7.73 (d, J=8.3 Hz, 1H); 13.02 (broad s, 1H).

Mass spectrum (LC/MS Method B): Retention time Tr (min)=2.99; [M+H]+: m/z=191; [M−H]−: m/z=189.

Stage 2: In a 250 ml round-bottomed flask, 1.05 g of a dispersion of sodium hydride at 60% in oil are added, in small portions under argon at ambient temperature, to a mixture of 3.32 g of 3-methyl-1H-indazole-4-carboxylic acid methyl ester obtained according to the preceding stage and 3.84 g of 2-bromo-4-fluorobenzonitrile in 120 ml of anhydrous dimethylformamide. The reaction medium is left to stir for 1.5 hours and is then diluted with 500 ml of ethyl acetate, and then 20 ml of distilled water are added. After separation by settling out, the aqueous phase is re-extracted with 500 ml of ethyl acetate. The combined organic phases are washed twice with distilled water and then with a saturated solution of sodium chloride, dried over magnesium sulphate and evaporated to dryness under vacuum. The solid obtained is filtered off and washed with four times 100 ml of isopropyl ether and dried under vacuum. 4.4 g of 1-(3-bromo-4-cyanophenyl)-3-methyl-1H-indazole-4-carboxylic acid methyl ester are obtained in the form of a solid, the characteristics of which are the following:

¹H NMR spectrum (400 MHz, δ in ppm, DMSO-d6): 2.66 (s, 3H); 3.95 (s, 3H); 7.65 (t, J=7.8 Hz, 1H); 7.79 (d, J=7.1 Hz, 1H); 8.02 (d, J=8.3 Hz, 1H); 8.13 (d, J=8.3 Hz, 1H); 8.21 (d, J=8.6 Hz, 1H); 8.25 (s, 1H).

Mass spectrum (LC/MS method C): Retention time Tr (min)=1.13; [M+H]₊: m/z=370.

Stage 3: In a 250 ml round-bottomed flask, a mixture of 2.0 g of 1-(3-bromo-4-cyanophenyl)-3-methyl-1H-indazole-4-carboxylic acid methyl ester obtained according to the preceding stage, 1.244 g of trans-4-aminocyclohexanol, 5.28 g of cesium carbonate, 375 mg of 4,5-bis(diphenylphosphino)-9,9-dimethylxanthene and 121 mg of palladium acetate in 150 ml of dioxane is heated under argon at 90° C. for 5 hours. The reaction medium is diluted with ethyl acetate and filtered through clarcel. The filtrate is evaporated to dryness under vacuum and the residue is chromatographed on silica gel (15-40 μm), elution being carried out with a mixture of methanol and dichloromethane (5:95 v/v). 445 mg of 1-[4-cyano-3-(trans-4-hydroxycyclohexylamino)phenyl]-3-methyl-1H-indazole-4-carboxylic acid methyl ester are obtained in the form of a resin, the characteristics of which are the following:

¹H NMR spectrum (400 MHz, δ in ppm, DMSO-d6): 1.16 to 1.49 (m, 4H); 184 (d, J=13.7 Hz, 2H); 1.94 (d, J=12.2 Hz, 2H); 2.66 (s, 3H); 3.35 to 3.58 (m, 2 H); 3.95 (s, 3H); 4.52 (d, J=4.6 Hz, 1H); 5.85 (d, J=8.1 Hz, 1H); 7.01 (dd, J=8.3 and 2.0 Hz, 1H); 7.10 (d, J=1.7 Hz, 1H); 7.59 (dd, J=8.4 and 7.2 Hz, 1H); 7.64 (d, J=8.3 Hz, 1H); 7.74 (d, J=6.6 Hz, 1H); 8.05 (d, J=8.6 Hz, 1H).

Mass spectrum (LC/MS method B): Retention time Tr (min)=4.21; [M+H]+: m/z=405; [M−H]−: m/z=403.

Stage 4: In a 50 ml round-bottomed flask, 438 mg of 1-[4-cyano-3-(trans-4-hydroxycyclohexylamino)phenyl]-3-methyl-1H-indazole-4-carboxylic acid methyl ester obtained according to the preceding stage and 3.3 ml of 1M sodium hydroxide in a mixture of 11 ml of dioxane, 5 ml of methanol and 3 ml of distilled water are stirred under argon at ambient temperature for 4 hours. The reaction medium is evaporated to dryness under vacuum and the residue is taken up in 10 ml of distilled water and acidified with 7 ml of 1N hydrochloric acid. The solution is saturated with solid sodium chloride, and extracted with twice 50 ml of ethyl acetate. The combined organic phases are washed with a saturated solution of sodium chloride, dried over magnesium sulphate and evaporated to dryness under vacuum. The solid is filtered off, washed with isopropyl ether and dried under vacuum. 380 mg of 1-[4-cyano-3-(trans-4-hydroxycyclohexylamino)phenyl]-3-methyl-1H-indazole-4-carboxylic acid are obtained in the form of a solid, the characteristics of which are the following:

¹H NMR spectrum (400 MHz, δ in ppm, DMSO-d6): 1.20 to 1.50 (m, 4H); 1.85 (d, J=11.2 Hz, 2H); 1.95 (d, J=11.5 Hz, 2H); 2.68 (s, 3H); 3.38 to 3.55 (m, 2 H); 4.53 (d, J=4.2 Hz, 1H); 5.84 (d, J=8.3 Hz, 1H); 7.01 (dd, J=8.4 and 1.8 Hz, 1H); 7.10 (d, J=1.5 Hz, 1H); 7.57 (dd, J=8.4 and 7.2 Hz, 1H); 7.63 (d, J=8.3 Hz, 1H); 7.71 (d, J=6.8 Hz; 1H); 8.01 (d, J=8.3 Hz, 1H); 13.26 (broad s, 1H).

Mass spectrum (LC/MS method B): Retention time Tr (min)=3.59; [M+H]₊: m/z=391; [M−H]−: m/z=389.

Stage 5: In a 100 ml round-bottomed flask, a mixture of 376 mg of 1-[4-cyano-3-(trans-4-hydroxycyclohexylamino)phenyl]-3-methyl-1H-indazole-4-carboxylic acid obtained according to the preceding stage, 128 mg of 4-fluoro-O-phenylenediamine, 347 mg of O-((ethoxycarbonyl)cyanomethyleneamino)-N,N,N′,N′-tetramethyluronium tetrafluoroborate (TOTU) and 185 μl of diisopropylethylamine in 50 ml of anhydrous dimethylformamide is stirred overnight at ambient temperature under an argon atmosphere. The reaction medium is evaporated to dryness under vacuum and the residue is taken up with 200 ml of ethyl acetate. The organic phase is washed with twice 50 ml of distilled water and with a saturated solution of sodium chloride, dried over magnesium sulphate and evaporated to dryness under vacuum. The residue is chromatographed on silica gel (15-40 μm), elution being carried out with a mixture of ethyl acetate and cyclohexane (80:20 v/v). 253 mg of 1-[4-cyano-3-(trans-4-hydroxycyclohexylamino)phenyl]-3-methyl-1H-indazole-4-carboxylic acid (2-amino-4-fluorophenyl)amide are obtained in the form of a solid, the characteristics of which are the following:

¹H NMR spectrum (400 MHz, δ in ppm, DMSO-d6): 1.21 to 1.48 (m, 4H); 1.85 (d, J=12.7 Hz, 2H); 1.96 (d, J=12.2 Hz, 2H); 2.58 (s, 3H); 3.37 to 3.58 (m, 2H); 4.54 (d, J=4.2 Hz, 1H); 5.29 (broad s, 2H); 5.87 (d, J=8.3 Hz, 1H); 6.41 (td, J=8.4 and 2.9 Hz, 1H); 6.57 (dd, J=11.2 and 2.9 Hz, 1H); 7.05 (dd, J=8.4 and 1.8 Hz, 1H); 7.11 (d, J=1.5 Hz, 1H); 7.33 (dd, J=8.6 and 6.4 Hz, 1H); 7.50 to 7.67 (m, 3H); 7.96 (d, J=9.0 Hz, 1H); 9.73 (s, 1H),

Mass spectrum (LC/MS method C): Retention time Tr (min)=0.88; [M+H]₊: m/z=499; [M−H]−: m/z=497.

Stage 6: 120 mg of 1-[4-cyano-3-(trans-4-hydroxycyclohexylamino)phenyl]-3-methyl-1H-indazole-4-carboxylic acid (2-amino-4-fluorophenyl)amide obtained according to the preceding stage in 12 ml of glacial acetic acid are respectively charged, under argon, to two 20 ml microwave reactors. After stirring for 30 seconds at ambient temperature, the reaction medium is heated at 115° C. for 1 hour with stirring. After cooling, the two reactions are combined, the reaction medium is diluted with 40 ml of methanol and 5 ml of 1M sodium hydroxide are added. After stirring for 30 minutes at ambient temperature, the mixture is evaporated to dryness under vacuum and the residue is taken up in ethyl acetate. The organic phase is washed with distilled water and then with a saturated solution of sodium chloride, dried over magnesium sulphate and evaporated to dryness under vacuum. The residue is triturated from isopropyl ether, filtered, washed with isopropyl ether and dried under vacuum. 214 mg of 4-[4-(6-fluoro-1H-benzimidazol-2-O-3-methylindazol-1-yl]-2-(trans-4-hydroxycyclohexylamino)-benzonitrile are obtained in the form of a solid, the characteristics of which are the following:

¹H NMR spectrum (400 MHz, δ in ppm, DMSO-d6): 1.21 to 1.50 (m, 4H); 1.86 (d, J=13.9 Hz, 2H); 1.96 (d, J=11.0 Hz, 2H); 2.47 (s, 3H); 3.37 to 3.58 (m, 2H); 4.59 (d, J=4.4 Hz, 1H); 5.94 (d, J=8.1 Hz, 1H); 7.01 to 7.20 (m, 3H); 7.34 to 7.83 (m, 5H); 8.01 (d, J=8.3 Hz, 1H); 13.09 (broad s, 1H).

Mass spectrum (LC/MS method B): Retention time Tr (min)=3.67; [M+H]₊: m/z=481; [M−H]−: m/z=479.

Stage 7: In a 50 ml round-bottomed flask at ambient temperature under argon, 7 ml of ethanol and then 0.89 ml of 1M sodium hydroxide and finally 0.85 ml of aqueous hydrogen peroxide at 30% are successively added to a mixture of 212 mg of 4-[(4-(6-fluoro-1H-benzimidazol-2-yl)-3-methylindazol-1-yl]-2-(trans-4-hydroxycyclohexylamino)benzonitrile obtained according to the preceding stage, in 3.5 ml of dimethyl sulphoxide. After stirring at ambient temperature for 0.75 hour, the reaction medium is diluted with 100 ml of distilled water. The resulting mixture is extracted twice with 200 ml of ethyl acetate after saturation of the aqueous phase with solid sodium chloride, and the combined organic phases are washed with twice 100 ml of distilled water and then with a saturated solution of sodium chloride, dried over magnesium sulphate and evaporated to dryness under vacuum. The residue is triturated from isopropyl ether, filtered and washed with 10 ml of isopropyl ether. 210 mg of 4-[4-(6-fluoro-1H-benzimidazol-2-yl)-3-methylindazol-1-yl]-2-(trans-4-hydroxycyclohexylamino)benzamide are obtained in the form of an amber solid, the characteristics of which are the following:

¹H NMR spectrum (400 MHz, δ in ppm, DMSO-d6): 1.20 to 1.43 (m, 4H); 1.78 to 1.89 (m, 2H); 2.00 to 2.07 (m, 2H); 2.49 (masked s, 3H); 3.34 to 3.45 (m, 1H); 3.45 to 3.57 (m, 1H); 4.53 (d, J=3.9 Hz, 1H); 6.88 (d, J=8.3 Hz, 1H); 6.98 (s, 1H); 7.02 to 7.30 (m, 2H); 7.38 (broad s, 1H); 7.56 (d, J=7.3 Hz, 1H); 7.57 to 7.61 (m, 1H); 7.64 (t, J=7.8 Hz, 1H); 7.82 (d, J=8.3 Hz, 1H); 7.89 (broad s, 1H); 7.96 (d, J=8.3 Hz, 1H); 8.49 (d, J=7.3 Hz, 1H); 13.02 (broad s, 1H).
Mass spectrum (LC/MS method C): Retention time Tr (min)=0.67; [M+H]₊: m/z=499; [M−H]−: m/z=497.

### EXAMPLE 13

### Synthesis of 4-[4-(6-fluoro-1H-benzimidazol-2-yl)-3-methylindazol-1-yl]-2-(2-hydroxy-2-methylpropylamino)benzamide

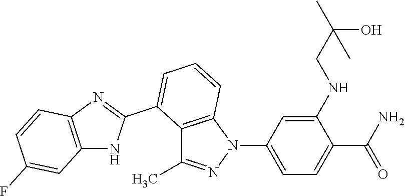

Stage 1: In a 250 ml round-bottomed flask, a mixture of 2.0 g of 1-(3-bromo-4-cyanophenyl)-3-methyl-1H-indazole-4-carboxylic acid methyl ester obtained according to stage 2 of Example 12, 963 mg of 1-amino-2-methylpropan-2-ol, 5.28 g of cesium carbonate, 375 mg of 4,5-bis(diphenylphosphino)-9,9-dimethylxanthene and 121 mg of palladium acetate in 150 ml of dioxane is heated under argon at 90° C. for 3.5 hours. The reaction medium, after cooling, is diluted with 200 ml of ethyl acetate and filtered through clarcel. The filtrate is evaporated to dryness under vacuum and the residue is chromatographed on silica gel (15-40 μm), elution being carried out with a mixture of ethyl acetate and cyclohexane (40:60 v/v). 1.4 g of 1-[4-cyano-3-(2-hydroxy-2-methylpropylamino)phenyl]-3-methyl-1H-indazole-4-carboxylic acid methyl ester are obtained in the form of a solid, the characteristics of which are the following:

¹H NMR spectrum (400 MHz, δ in ppm, DMSO-d6): 1.20 (s, 6H); 2.65 (s, 3 H); 3.20 (d, J=5.6 Hz, 2H); 3.95 (s, 3H); 4.79 (s, 1H); 5.80 (t, J=5.6 Hz, 1H); 7.02 (dd, J=1.9 and 8.5 Hz, 1H); 7.19 (d, J=1.9 Hz, 1H); 7.58 (dd, J=7.3 and 8.6 Hz, 1H); 7.66 (d, J=8.5 Hz, 1H); 7.74 (dd, J=0.7 and 7.3 Hz, 1H); 8.12 (dd, J=0.7 and 8.6 Hz, 1H).

Mass spectrum (LC/MS method C): Retention time r (min)=1.02; [M+H]+: m/z=379; [M−H]−: m/z=377.

Stage 2: In a 250 ml round-bottomed flask, 1.39 g of 1-[4-cyano-3-(2-hydroxy-2-methylpropylamino)phenyl]-3-methyl-1H-indazole-4-carboxylic acid methyl ester obtained according to the preceding stage, dissolved in a mixture of 30 ml of dioxane, 18 ml of methanol and 10.2 ml of distilled water with 11.25 ml of 1M sodium hydroxide are stirred for 4 hours at ambient temperature under argon. The reaction medium is evaporated to dryness under vacuum and the residue taken up with 50 ml of distilled water is acidified with 20 ml of 1N hydrochloric acid. The aqueous phase is extracted with 200 ml of ethyl acetate and the organic phase is washed with twice 50 ml of distilled water, dried over magnesium sulphate and evaporated to dryness under vacuum.

1.3 g of 1-[4-cyano-3-(2-hydroxy-2-methylpropylamino)phenyl]-3-methyl-1H-indazole-4-carboxylic acid are obtained in the form of a solid, the characteristics of which are the following:

¹H NMR spectrum (400 MHz, δ in ppm, DMSO-d6): 1.20 (s, 6H); 2.68 (s, 3H); 3.20 (d, J=5.5 Hz, 2H); 4.79 (s, 1H); 5.78 (t, J=5.5 Hz, 1H); 7.03 (dd, J=1.9 and 8.4 Hz, 1H); 7.19 (d, J=1.9 Hz, 1H); 7.56 (dd, J=7.2 and 8.5 Hz, 1H); 7.66 (d, J=8.4 Hz, 1H); 7.71 (d, J=7.2 Hz, 1H); 8.07 (d, J=8.5 Hz, 1H); 13.28 (broad s, 1H).

Mass spectrum (LC/MS method C): Retention time Tr (min)=0.80; [M+H]₊: m/z=365; [M−H]₋: m/z=363.

Stage 3: In a 250 ml round-bottomed flask, a mixture of 1.3 g of 1-[4-cyano-3-(2-hydroxy-2-methylpropylamino)phenyl]-3-methyl-1H-indazole-4-carboxylic acid obtained according to the preceding stage, 472 mg of 4-fluoro-O-phenylenediamine, 1.287 g of O-((ethoxycarbonyl)cyanomethyleneamino)-N,N,N′,N′-tetramethyluronium tetrafluoroborate (TOTU) and 685 μl of diisopropylethylamine in 150 ml of anhydrous dimethylformamide is stirred overnight at ambient temperature under an argon atmosphere. The reaction medium is evaporated to dryness under vacuum and the residue is taken up with 200 ml of ethyl acetate. The organic phase is washed with twice 50 ml of distilled water and with a saturated solution of sodium chloride, dried over magnesium sulphate and evaporated to dryness under vacuum. The residue is chromatographed on silica gel (15-40 μm), elution being carried out with a mixture of ethyl acetate and cyclohexane (80:20 v/v). 1.33 g of 1-[4-cyano-3-(2-hydroxy-2-methylpropylamino)phenyl]-3-methyl-1H-indazole-4-carboxylic acid (2-amino-4-fluorophenyl)amide are obtained in the form of a solid, the characteristics of which are the following:

¹H NMR spectrum (400 MHz, δ in ppm, DMSO-d6): 1.21 (s, 6H); 2.58 (s, 3H); 3.21 (d, J=5.6 Hz, 2H); 4.80 (s, 1H); 5.29 (broad s, 2H); 5.79 (t, J=5.6 Hz, 1H); 6.41 (td, J=2.8 and 8.5 Hz, 1H); 6.57 (dd, J=2.8 and 11.1 Hz, 1H); 7.06 (dd, J=1.8 and 8.4 Hz, 1H); 7.21 (d, J=1.8 Hz, 1H); 7.33 (dd, J=6.4 and 8.8 Hz, 1H); 7.53 to 7.62 (m, 2H); 7.66 (d, J=8.4 Hz, 1H); 8.03 (dd, J=1.5 and 7.8 Hz, 1H); 9.73 (s, 1H).

Mass spectrum (LC/MS method C): Retention time Tr (min)=0.90; [M+H]₊: m/z=473; [M−H]₋: m/z=471.

Stage 4: 114 mg of 1-[4-cyano-3-(2-hydroxy-2-methylpropylamino)phenyl]-3-methyl-1H-indazole-4-carboxylic acid (2-amino-4-fluorophenyl)amide obtained according to the preceding stage, in 13 ml of glacial acetic acid, are charged respectively, under argon, to ten 20 ml microwave reactors. After stirring for 30 seconds at ambient temperature, the reaction medium is heated at 115° C. for 45 minutes with stirring. After cooling, the ten reactions are combined and evaporated to dryness under vacuum. The residue is chromatographed on silica gel, elution being carried out with a mixture of ethyl acetate and cyclohexane (70:30 v/v). 966 mg of 4-[4-(6-fluoro-1H-benzimidazol-2-yl)-3-methylindazol-1-yl]-2-(2-hydroxy-2-methylpropylamino)benzonitrile are obtained in the form of a solid, the characteristics of which are the following:

¹H NMR spectrum (400 MHz, δ in ppm, DMSO-d6): 1.21 (s, 6H); 2.49 (s, 3H); 3.22 (d, J=5.6 Hz, 2H); 4.80 (s, 1H); 5.80 (t, J=5.6 Hz, 1H); 7.08 (dd, J=1.9 and 8.5 Hz, 1H); 7.10 to 7.16 (m, 1H); 7.24 (d, J=1.9 Hz, 1H); 7.41 to 7.50 (m, 1H); 7.58 (d, J=7.2 Hz, 1H); 7.62 to 7.70 (m, 3H); 8.06 (d, J=8.4 Hz, 1H); 13.04 (broad s, 1H).

Mass spectrum (LC/MS method C): Retention time Tr (min)=0.86; [M+H]₊: m/z=455; [M−H]₋: m/z=453.

Stage 5: In a 100 ml round-bottomed flask at ambient temperature under argon, 20 ml of ethanol and then 4.2 ml of 1M sodium hydroxide and finally 4.0 ml of aqueous hydrogen peroxide at 30% are successively added to a mixture of 955 mg of 4-[4-(6-fluoro-1H-benzimidazol-2-yl)-3-methylindazol-1-yl]-2-(2-hydroxy-2-methylpropylamino)benzonitrile obtained according to the preceding stage, in 20 ml of dimethyl sulphoxide. After stirring at ambient temperature for 0.75 hour, the reaction medium is diluted with 100 ml of distilled water. The resulting mixture is extracted twice with 250 ml of ethyl acetate after saturation of the aqueous phase with solid sodium chloride, and the combined organic phases are washed with twice 100 ml of distilled water and then with 50 ml of a saturated solution of sodium chloride, dried over magnesium sulphate and evaporated to dryness under vacuum. The residue is chromatographed on silica gel (15-40 μm), elution being carried out with a mixture of methanol and dichloromethane (10:90 v/v). 834 mg of 4-[4-(6-fluoro-1H-benzimidazol-2-yl)-3-methylindazol-1-yl]-2-(2-hydroxy-2-methylpropylamino)benzamide are obtained in the form of a white solid, the characteristics of which are the following:

¹H NMR spectrum (400 MHz, δ in ppm, DMSO-d6): 1.21 (s, 6H); 2.50 (s, 3 H); 3.10 (d, J=5.1 Hz, 2H); 4.57 (s, 1H); 6.88 (dd, J=2.0 and 8.5 Hz, 1H); 6.98 (d, J=2.0 Hz, 1H); 7.08 to 7.17 (m, 1H); 7.46 (broad s, 1H); 7.55 (d, J=7.2 Hz, 1H); 7.63 (dd, J=7.2 and 8.4 Hz, 1H); 7.64 (broad s, 1H); 7.72 to 7.99 (m, 2H); 7.81 (d, J=8.5 Hz, 1H); 8.01 (d, J=8.4 Hz, 1H); 8.64 (t, J=5.1 Hz, 1H); 13.03 (broad s, 1H).

Mass spectrum (LC/MS method C): Retention time Tr (min)=0.71; [M+H]₊: m/z=473; [M−H]₋: m/z=471.

### EXAMPLE 14

### Synthesis of 4-(3-methyl-4-quinolin-3-ylindazol-1-yl)-2-[exo-(7-oxabicyclo[2.2.1]hept-2-yl)amino]benzamide

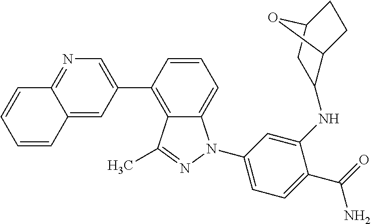

Stage 1: The benzyl ester of exo-(7-oxabicyclo[2.2.1]hept-2-yl)carbamic acid is prepared by carrying out the process as described by P. Spurr et al., WO2008/0154043 for the synthesis of the ethyl ester of exo-(7-oxabicyclo[2.2.1]hept-2-yl)carbamic acid, replacing the ethanol with benzyl alcohol in the Curtius reaction used in the final stage. 3.21 g of exo-(7-oxabicyclo[2.2.1]hept-2-yl)carbamic acid benzyl ester are thus obtained in the form of a thick, dark yellow oil, the characteristics of which are the following:

Mass spectrum (LC/MS method B): Retention time Tr (min)=3.42; [M+H]₊: m/z=248.

Stage 2: 3.81 g of exo-(7-oxabicyclo[2.2.1]hept-2-yl)carbamic acid benzyl ester obtained according to the preceding stage, 0.82 g of palladium-on-charcoal at 10% and 40 ml of ethanol are successively charged to an autoclave, and then the reaction medium is hydrogenated at 2 bar at 25° C. for 16 h with stirring. The mixture is subsequently filtered through clarcel and the solid is washed with ethanol. The filtrate is concentrated to dryness under reduced pressure and the residue obtained is chromatographed on silica gel (15-40 μm), elution being carried out with a mixture of chloroform, methanol and aqueous ammonia at 28% (55:6:1 v/v/v). 647 mg of 2-exo-7-oxabicyclo[2.2.1]hept-2-ylamine are thus obtained in the form of a yellow liquid, the characteristics of which are the following:

Mass spectrum (LC/MS method C): Retention time Tr (min)=0.11; [M+H]₊: m/z=114.

Stage 3: A solution of 235 mg of 2-exo-7-oxabicyclo[2.2.1]heptanamine in 10 ml of dioxane, 72 mg of 4,5-bis(diphenylphosphino)-9,9-dimethylxanthene, 23 mg of palladium(II) acetate and 1.02 g of cesium carbonate are successively added to a solution of 456 mg of 2-bromo-4-(3-methyl-4-quinolin-3-ylindazol-1-yl)benzonitrile, obtained according to stage 3 of Example 1, in 25 ml of dioxane under argon. The reaction medium is subsequently heated at 90° C. with stirring and under argon for 24 hours. After cooling, the reaction mixture is diluted with 200 ml of ethyl acetate and then filtered through clarcel. The filtrate is concentrated to dryness under reduced pressure. The crude residue obtained is chromatographed on silica gel (15-40 μm), elution being carried out with a mixture of ethanol and dichloromethane (3:97 v/v). The pure fractions are combined and then concentrated to dryness under reduced pressure, and the residue is triturated from diisopropyl ether. A first batch of 88 mg of a white powder is thus obtained. The impure fractions are combined and then concentrated to dryness under reduced pressure. The residue is repurified by chromatography on silica gel (15-40 μm), elution being carried out with a mixture of acetonitrile and dichloromethane (10:90 v/v). The pure fractions are combined and then concentrated to dryness under reduced pressure, and the residue is triturated from diisopropyl ether. A second batch of product is thus obtained in the form of a white powder. The two batches are combined and 260 mg of 4-(3-methyl-4-quinolin-3-ylindazol-1-yl)-2-[exo-(7-oxabicyclo[2.2.1]hept-2-yl)amino]benzonitrile, are thus obtained in the form of a white powder, the characteristics of which are the following:

Melting point (Kofler bench)=213-15° C.

Mass spectrum (LC/MS method C): Retention time Tr (min)=1.13; [M+H]+: m/z=472.

Stage 4: 2.8 ml of dimethyl sulphoxide, 1.1 ml of a 1N aqueous solution of sodium hydroxide, and then 1.0 ml of an aqueous solution of hydrogen peroxide at 30% are successively added to a suspension of 260 mg of 4-(3-methyl-4-quinolin-3-ylindazol-1-yl)-2-[exo-(7-oxabicyclo[2.2.1]hept-2-yl)amino]benzonitrile obtained according to the preceding stage, in 6.8 ml of absolute ethanol, under argon. The reaction mixture is subsequently stirred at 25° C. for 15 minutes, and is then poured into 20 ml of water. After extraction with 3 times 25 ml of ethyl acetate, the organic extracts are combined, washed with a saturated solution of brine, dried over magnesium sulphate, filtered, and then concentrated to dryness under reduced pressure. The residue is chromatographed on silica gel (15-40 μm), elution being carried out with a mixture of ethanol and dichloromethane (3:97 v/v). The pure fractions are combined and then concentrated to dryness under reduced pressure. A first batch of 75 mg of white solid is thus obtained. The impure fractions are combined and then concentrated to dryness under reduced pressure. The residue is repurified by chromatography on silica gel (15-40 μm), elution being carried out with a mixture of ethanol and dichloromethane (3:97 v/v). The pure fractions are combined and then concentrated to dryness under reduced pressure. A second batch of product is thus obtained in the form of a white powder. The two batches are combined, triturated from diisopropyl ether, filtered, washed with diisopropyl ether and spin-filter-dried. After drying under reduced pressure at 40° C., 223 mg of 4-(3-methyl-4-quinolin-3-ylindazol-1-yl)-2-[exo-(7-oxabicyclo[2.2.1]hept-2-yl)amino]benzamide are obtained in the form of a white powder, the characteristics of which are the following:

Melting point (Kofler bench)=170-2° C.

¹H NMR spectrum (400 MHz, δ in ppm, DMSO-d6): 1.36 to 1.64 (m, 5H); 2.10 (dd, J=7.5 and 12.3 Hz, 1H); 2.15 (s, 3H); 3.68 (td, J=2.6 and 6.9 Hz, 1H); 4.40 (d, J=4.6 Hz, 1H); 4.61 (t, J=4.4 Hz, 1H); 6.91 to 6.97 (m, 2H); 7.21 (broad s, 1H); 7.29 (d, J=7.1 Hz, 1H); 7.63 (t, J=7.8 Hz, 1H); 7.71 (dd, J=7.1 and 8.1 Hz, 1H); 7.81 to 7.88 (m, 2H); 7.90 (broad s, 1H); 7.95 (d, J=8.6 Hz, 1H); 8.10 to 8.16 (m, 2H); 8.47 (d, J=6.8 Hz, 1H); 8.53 (s, 1H); 9.06 (d, J=2.2 Hz, 1H).

Mass spectrum (LC/MS method C): Retention time Tr (min)=0.99; [M+H]+: m/z=490.

### EXAMPLE 15

### Synthesis of 4-(3-methyl-4-quinolin-3-ylindazol-1-yl)-2-(1,2,2,6,6-pentamethylpiperidin-4-ylamino)benzamide

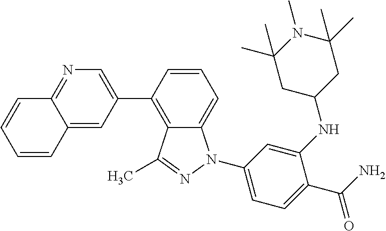

Stage 1: In a 50 ml three-necked flask, a mixture of 250 mg of 2-bromo-4-(3-methyl-4-quinolin-3-ylindazol-1-yl)benzonitrile obtained according to stage 3 of Example 1, 170 mg of 4-amino-1,2,2,6,6-pentamethylpiperidine, 489 mg of cesium carbonate, 35 mg of 4,5-bis(diphenylphosphino)-9,9-dimethylxanthene and 11 mg of palladium acetate in 16 ml of dioxane is heated under argon at 90° C. for 2.5 hours. The reaction medium is poured into 50 ml of distilled water and extracted with 3 times 30 ml of ethyl acetate. The combined organic phases are washed with 30 ml of a saturated solution of sodium chloride, dried over magnesium sulphate and evaporated to dryness under vacuum. The residue is chromatographed on silica gel (15-40 μm), elution being carried out with a mixture of 7M ammonia in methanol and of dichloromethane (5:95 v/v). 230 mg of 4-(3-methyl-4-quinolin-3-ylindazol-1-yl)-2-(1,2,2,6,6-pentamethylpiperidin-4-ylamino)benzonitrile are obtained in the form of a beige foam which is used, without further characterization, in the next stage.

Stage 2: In a 100 ml single-necked flask at ambient temperature under argon, 5 ml of ethanol and then 0.87 ml of 1M sodium hydroxide and finally 0.8 ml of aqueous hydrogen peroxide at 30% are successively added to a mixture of 230 mg of 4-(3-methyl-4-quinolin-3-ylindazol-1-yl)-2-(1,2,2,6,6-pentamethyl-piperidin-4-ylamino)benzonitrile obtained according to the preceding stage, in 3 ml of dimethyl sulphoxide. After stirring at ambient temperature for 2 hours, the reaction medium is diluted with 100 ml of distilled water. After extraction with 3 times 50 ml of ethyl acetate, the combined organic phases are washed with 50 ml of a saturated solution of sodium chloride, dried over magnesium sulphate and evaporated to dryness under vacuum. The residue is chromatographed on silica gel (15-40 μm), elution being carried out with a mixture of 7M ammonia in methanol and of dichloromethane (5:95 v/v). 47 mg of 4-(3-methyl-4-quinolin-3-ylindazol-1-yl)-2-(1,2,2,6,6-pentamethylpiperidin-4-ylamino)benzamide are obtained in the form of an amorphous white solid, the characteristics of which are the following:

¹H NMR spectrum (400 MHz, δ in ppm, DMSO-d6): 1.10 (s, 6H); 1.13 (s, 6 H); 1.28 (t, J=12.1 Hz, 2H); 1.98 (dd, J=2.7 and 12.2 Hz, 2H); 2.14 (s, 3H); 2.20 (s, 3H); 3.64 to 3.79 (m, 1H); 6.93 (dd, J=2.0 and 8.4 Hz, 1H); 7.10 (d, J=2.0 Hz, 1H); 7.18 (broad s, 1H); 7.29 (d, J=7.1 Hz, 1H); 7.60 (dd, J=7.1 and 8.6 Hz, 1H); 7.66 to 7.75 (m, 1H); 7.80 to 7.88 (m, 2H); 7.92 (broad s, 1H); 7.99 (d, J=8.3 Hz, 1H); 8.13 (t, J=7.1 Hz, 2H); 8.40 (d, J=7.3 Hz, 1H); 8.54 (d, J=2.2 Hz, 1H); 9.06 (d, J=2.2 Hz, 1H).

Mass spectrum (LC/MS method C): Retention time Tr (min)=0.76; [M+H]₊: m/z=547.

### EXAMPLE 16

### Synthesis of 3-(trans-4-hydroxycyclohexylamino)-5-(3-methyl-4-quinolin-3-ylindazol-1-yl)pyridine-2-carboxamide

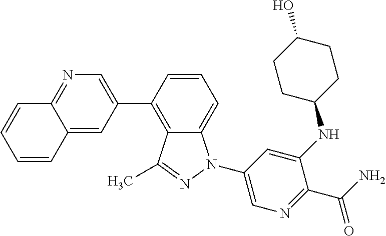

Stage 1: 500 mg of 2-cyano-3,5-difluoropyridine, 493 mg of trans-4-aminocyclohexanol and 987 mg of potassium carbonate in 7.5 ml of dimethyl sulphoxide are charged to a 20 ml microwave tube-reactor. The mixture is then microwave-heated for 1 hour at 115° C. The reaction medium is run into 100 ml of water and 100 ml of ethyl acetate. The aqueous phase is re-extracted twice with 50 ml of ethyl acetate. The combined organic phases are washed with water and then with a saturated aqueous solution of sodium chloride, dried over sodium sulphate and concentrated under reduced pressure. After flash chromatography on silica gel (40-63 μm), elution being carried out with a mixture of ethyl acetate and cyclohexane (50:50 v/v), with the first eluted product being collected, 309 mg of 2-cyano-5-fluoro-3-(trans-4-hydroxycyclohexylamino)pyridine are obtained in the form of a white powder, the characteristics of which are the following:

TLC on silica gel: Rf=0.20 (50/50 ethyl acetate/cyclohexane).

¹H NMR spectrum (400 MHz, δ in ppm, DMSO-d6): 1.20 to 1.47 (m, 4H); 1.75 to 1.89 (m, 4H); 3.32 to 3.45 (m, 2H); 4.54 (d, J=4.4 Hz, 1H); 6.23 (d, J=8.1 Hz, 1H); 7.30 (dd, J=12.1 and 2.3 Hz, 1H); 7.85 (d, J=2.4 Hz, 1H).

Stage 2: In a 50 ml three-necked flask under an argon atmosphere, 150 mg of 3-(3-methyl-1H-indazol-4-yl)quinoline obtained according to stage 2 of Example 1 are dissolved in 10 ml of dimethylformamide. 35 mg of sodium hydride at 60% in oil are then added and the mixture is stirred for 30 minutes at ambient temperature and then 30 minutes at 50° C. 150 mg of 2-cyano-5-fluoro-3-(trans-4-hydroxycyclohexylamino)pyridine obtained according to the preceding stage are added at 50° C., and the mixture is heated at 80° C. for 1.5 hours. The reaction medium is run into 50 ml of water and 50 ml of ethyl acetate. The aqueous phase is re-extracted twice with 25 ml of ethyl acetate. The combined organic phases are washed with water and then with a saturated aqueous solution of sodium chloride, dried over sodium sulphate and concentrated under reduced pressure. 280 mg of a mixture containing very predominantly 3-(trans-4-hydroxycyclohex-1-ylamino)-5-[3-methyl-4-quinolin-3-ylindazol-1-yl]pyridine-2-carbonitrile are thus obtained in the form of a beige powder which is used as it is in the next stage.

Stage 3: 280 mg of 3-(trans-4-hydroxycyclohex-1-ylamino)-5-[3-methyl-4-quinolin-3-ylindazol-1-yl]pyridine-2-carbonitrile obtained according to the preceding stage are dissolved in 3 ml of dimethyl sulphoxide and 7.5 ml of ethanol, and then 1.16 ml of a 1M aqueous solution of sodium hydroxide and 1.06 ml of a 30% aqueous solution of hydrogen peroxide are successively added. After stirring for 5 minutes at ambient temperature, the reaction medium is run into 100 ml of water and 100 ml of ethyl acetate. The aqueous phase is re-extracted three times with 25 ml of ethyl acetate. The combined organic phases are washed with water, dried over sodium sulphate and concentrated under reduced pressure. After flash chromatography on silica gel (40-63 μm), elution being carried out with a mixture of dichloromethane and ethanol (95:5 v/v), 156 mg of 3-(trans-4-hydroxycyclohexyl)amino]-5-[3-methyl-4-quinolin-3-ylindazol-1-yl]pyridine-2-carboxamide are obtained in the form of a pale yellow powder, the characteristics of which are the following:

¹H NMR spectrum (400 MHz, δ in ppm, DMSO-d6): 1.20 to 1.45 (m, 4H); 1.84 (d, J=10.8 Hz, 2H); 2.03 (d, J=10.8 Hz, 2H); 2.16 (s, 3H); 3.46 to 3.56 (m, 2 H); 4.55 (d, J=4.2 Hz, 1H); 7.33 (d, J=6.8 Hz, 1H); 7.46 (d, J=2.0 Hz, 2H); 7.61 to 7.75 (m, 2H); 7.86 (ddd, J=1.5 and 6.9 and 8.5 Hz, 1H); 7.95 (d, J=8.6 Hz, 1H); 8.02 (broad s, 1H); 8.08 to 8.17 (m, 2H); 8.19 (d, J=2.0 Hz, 1H); 8.54 (d, J=2.0 Hz, 1H); 8.72 (d, J=7.8 Hz, 1H); 9.06 (d, J=2.4 Hz, 1H).

Mass spectrum (LC/MS method B): Retention time Tr (min)=3.93; [M+H]₊: m/z=493; [M−H]₋+HCOOH: m/z=537.

### EXAMPLE 17

### Synthesis of 5-[3-methyl-4-quinolin-3-ylindazol-1-yl]-3-(1,2,2,6,6-pentamethylpiperidin-1-ylamino)pyridine-2-carboxamide

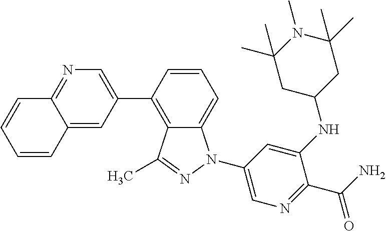

Stage 1: 682 mg of 2-cyano-3,5-difluoropyridine, 995 mg of 4-amino-1,2,2,6,6-pentamethylpiperidine and 1.346 g of potassium carbonate in 10 ml of dimethyl sulphoxide are charged to a 20 ml microwave tube-reactor. The mixture is then microwave-heated for 1 hour at 115° C. The reaction medium is run into 100 ml of water and 100 ml of ethyl acetate. The aqueous phase is re-extracted twice with 50 ml of ethyl acetate. The combined organic phases are washed with water and then with a saturated aqueous solution of sodium chloride, dried over sodium sulphate and concentrated under reduced pressure. After flash chromatography on silica gel (15-40 μm), elution being carried out with a mixture of dichloromethane, methanol and 4N aqueous ammonia (99:1:0.8 v/v/v), with the first eluted product being collected, 290 mg of 2-cyano-5-fluoro-3-(1,2,2,6,6-pentamethylpiperidin-4-ylamino)pyridine are obtained in the form of an ecru powder, the characteristics of which are the following:

¹H NMR spectrum (400 MHz, δ in ppm, DMSO-d6): 1.07 (s, 6H); 1.08 (s, 6H); 1.46 (t, J=12.1 Hz, 2H); 1.73 (dd, J=12.5 and 3.5 Hz, 2H); 2.18 (s, 3H); 3.70 to 3.82 (m, 1H); 627 (d, J=8.6 Hz, 1H); 7.18 (dd, J=11.8 and 2.4 Hz, 1H); 7.89 (d, J=2.4 Hz, 1H).

Stage 2: In a 50 ml three-necked flask under an argon atmosphere, 136 mg of 3-(3-methyl-1H-indazol-4-yl)quinoline obtained according to stage 2 of Example 1 are dissolved in 2.5 ml of dimethylformamide. 32 mg of sodium hydride at 60% in oil and 1 ml of dimethylformamide are added and the mixture is stirred for 30 minutes at ambient temperature. 168 mg of 2-cyano-5-fluoro-3-(1,2,2,6,6-pentamethylpiperidin-1-ylamino)pyridine obtained according to the preceding stage are subsequently added and the mixture is heated at 50-55° C. for 2 hours. The reaction medium is run into 50 ml of a saturated aqueous solution of sodium chloride and 50 ml of ethyl acetate. The aqueous phase is re-extracted twice with 25 ml of ethyl acetate. The combined organic phases are washed four times with 5 ml of water, dried over sodium sulphate and concentrated under reduced pressure. 335 mg of a mixture containing very predominantly 5-[3-methyl-4-quinolin-3-ylindazol-1-yl]-3-(1,2,2,6,6-pentamethylpiperidin-1-ylamino)pyridine-2-carbonitrile are thus obtained in the form of a yellow powder which is used as it is in the next stage.

Stage 3: 307 mg of 5-[3-methyl-4-quinolin-3-ylindazol-1-yl]-3-(1,2,2,6,6-pentamethylpiperidin-1-ylamino)pyridine-2-carbonitrile obtained according to the preceding stage are dissolved in 3 ml of dimethyl sulphoxide and 3 ml of ethanol, and then 0.89 ml of a 1M aqueous solution of sodium hydroxide and 0.87 ml of a 30% aqueous solution of hydrogen peroxide are successively added. After stirring for 6 hours at ambient temperature, the insoluble material formed is filter-dried through sintered glass and then washed four times with 5 ml of water. After flash chromatography on silica gel (15-40 μm), elution being carried out with a mixture of dichloromethane and 7M ammonia in methanol (95:5 v/v), 141 mg of 5-[3-methyl-4-quinolin-3-ylindazol-1-yl]-3-(1,2,2,6,6-pentamethylpiperidin-1-ylamino)pyridine-2-carboxamide are obtained in the form of a pale yellow powder, the characteristics of which are the following:

¹H NMR spectrum (400 MHz, δ in ppm, DMSO-d6): 1.11 (s, 6H); 1.13 (s, 6H); 1.30 (t, J=11.9 Hz, 2H); 1.98 (dd, J=2.1 and 11.9 Hz, 2H); 2.15 (s, 3H); 2.21 (s, 3H); 3.75 to 3.88 (m, 1H); 7.33 (d, J=7.1 Hz, 1H); 7.47 (broad s, 1H); 7.56 (d, J=2.0 Hz, 1H); 7.63 (dd, J=7.2 and 8.4 Hz, 1H); 7.68 to 7.74 (m, 1H); 7.85 (ddd, J=1.3 and 7.0 and 8.5 Hz, 1H); 7.98 to 8.05 (m, 2H); 8.13 (t, J=7.0 Hz, 2H); 8.24 (d, J=2.0 Hz, 1H); 8.54 (d, J=2.0 Hz, 1H); 8.60 (d, J=7.3 Hz, 1H); 9.06 (d, J=2.2 Hz, 1H).

Mass spectrum (LC/MS method B): Retention time Tr (min)=3.26; [M+H]₊: m/z=548.

### EXAMPLE 18

### Synthesis of 5-[3-methyl-4-quinolin-3-ylindazol-1-yl]-3-[2-pyridin-2-ylethylamino]pyridine-2-carboxamide

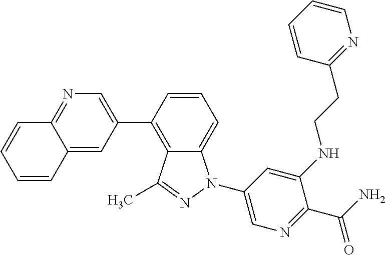

Stage 1: 841 mg of 2-cyano-3,5-difluoropyridine, 880 mg of 2-(2-aminoethyl)pyridine and 1.658 g of potassium carbonate in 12.5 ml of dimethyl sulphoxide are charged to a 20 ml microwave tube-reactor. The mixture is then microwave-heated for 1.5 hours at 115° C. The reaction medium is run into 100 ml of water and 100 ml of ethyl acetate. The aqueous phase is re-extracted twice with 50 ml of ethyl acetate. The combined organic phases are washed with water and then with a saturated aqueous solution of sodium chloride, dried over sodium sulphate and concentrated under reduced pressure. After flash chromatography on silica gel (40-63 μm), elution being carried out with a mixture of ethyl acetate and cyclohexane (40:60 v/v), with the first eluted product being collected, 549 mg of 2-cyano-5-fluoro-3-(2-pyridin-2-ylethylamino)pyridine are obtained in the form of a beige powder, the characteristics of which are the following:

¹H NMR spectrum (400 MHz, δ in ppm, DMSO-d6): 3.02 (t, J=7.1 Hz, 2H); 3.57 (q, J=6.8 Hz, 2H); 6.94 (broad s, 1H); 7.15 to 7.27 (m, 2H); 7.33 (d, J=7.8 Hz, 1H); 7.71 (td, J=7.6 and 1.8 Hz, 1H); 7.86 (d, J=2.4 Hz, 1H); 8.51 (d, J=4.9 Hz, 1H).

Stage 2: In a 50 ml three-necked flask under an argon atmosphere, 143 mg of 3-(3-methyl-1H-indazol-4-yl)quinoline obtained according to stage 2 of Example 1 are dissolved in 14 ml of dimethylformamide, 33 mg of sodium hydride at 60% in oil are added and the mixture is stirred for 30 minutes at ambient temperature and then for 30 minutes at 50° C. 147 mg of 2-cyano-5-fluoro-3-(2-pyridin-2-ylethylamino)pyridine obtained according to the preceding stage are then added at 50° C., and the mixture is heated at 80° C. for 1.5 hours. The reaction medium is run into 50 ml of water and 50 ml of ethyl acetate. The aqueous phase is re-extracted twice with 25 ml of ethyl acetate. The combined organic phases are washed with water and then with a saturated aqueous solution of sodium chloride, dried over sodium sulphate and concentrated under reduced pressure. 300 mg of a mixture containing very predominantly 5-[3-methyl-4-quinolin-3-ylindazol-1-yl]-3-[2-(pyridin-2-yl)aminoethyl]pyridine-2-carbonitrile are obtained in the form of a beige powder which is used as it is in the next stage.

Stage 3: 300 mg of 5-[3-methyl-4-quinolin-3-ylindazol-1-yl]-3-[2-(pyridin-2-yl)aminoethyl]pyridine-2-carbonitrile obtained according to the preceding stage are dissolved in 3.2 ml of dimethyl sulphoxide and 8.1 ml of ethanol, and then 1.25 ml of a 1M aqueous solution of sodium hydroxide and 1.15 ml of a 30% aqueous solution of hydrogen peroxide are successively added. After stirring for 15 minutes at ambient temperature, the reaction medium is run into 100 ml of water and 100 ml of ethyl acetate. The aqueous phase is re-extracted three times with 25 ml of ethyl acetate. The combined organic phases are washed with water, dried over sodium sulphate and concentrated under reduced pressure. After flash chromatography on silica gel (40-63 μm), elution being carried out with a mixture of dichloromethane and ethanol (96:4 v/v), 128 mg of 5-[3-methyl-4-quinolin-3-ylindazol-1-yl]-3-[2-(pyridin-2-yl)aminoethyl]pyridine-2-carboxamide are obtained in the form of an off-white powder, the characteristics of which are the following:

¹H NMR spectrum (400 MHz, δ in ppm, DMSO-d6): 2.16 (s, 3H); 3.11 (t, J=6.4 Hz, 2H); 3.68 (q, J=6.5 Hz, 2H); 7.09 to 7.28 (m, 1H); 7.35 (t, J=7.3 Hz, 2H); 7.42 (broad s, 1H); 7.52 (broad s, 1H); 7.59 to 7.77 (m, 3H); 7.86 (t, J=7.3 Hz, 1H); 7.93 to 8.08 (m, 2H); 8.13 (t, J=7.0 Hz, 2H); 8.24 (broad s, 1H); 8.51 (d, J=4.2 Hz, 1H); 8.55 (broad s, 1H); 8.79 (broad s, 1H); 9.07 (broad s, 1H).

Mass spectrum (LC/MS method C): Retention time Tr (min)=0.76; [M+H]₊: m/z=500.

### EXAMPLE 19

### Synthesis of 4-(3-methyl-4-quinolin-3-ylindazol-1-yl)-2-{[exo-1-(7-oxabicyclo[2.2.1]hept-2-yl)methyl]amino}benzamide

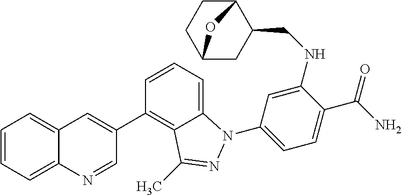

Stage 1: 2.84 g of lithium aluminium hydride and 26 ml of anhydrous diethyl ether are successively charged to a 250 ml three-necked flask under argon. A solution of 2.30 g of 7-oxabicyclo[2.2.1]heptane-2-carbonitrile obtained according to P. Spurr et al., WO2008/0154043, in 84 ml of anhydrous diethyl ether, is subsequently added dropwise with stirring. The resulting grey suspension is stirred at 25° C. under argon for 16 hours, and is then cooled in an ice bath and treated successively with 5 ml of water, 11 ml of an aqueous 30% solution of sodium hydroxide and 13 ml of water. After stirring for 30 minutes, the reaction mixture is filtered through sintered glass and the solid is washed twice with diethyl ether. The filtrate is concentrated to dryness under reduced pressure and the residue obtained is chromatographed on silica gel (15-40 μm), elution being carried out with a mixture of dichloromethane and methanol (90/10 v/v). 0.92 g of (7-oxabicyclo[2.2.1]hept-2-yl)methylamine is obtained in the form of a yellow oil, the characteristics of which are the following:

¹H NMR spectrum (400 MHz, δ in ppm, DMSO-d6): 0.86 (dd, J=5.1 and 11.7 Hz, 0.5H); 1.03 to 1.11 (m, 0.5H); 1.27 to 1.79 (m, 5.5H); 1.93 to 2.04 (m, 0.5H); 2.23 (dd, J=6.1 and 12.2 Hz, 0.5H); 2.35 (dd, J=8.8 and 12.2 Hz_{;} 0.5H); 2.45 to 2.50 (m, 0.5H); 2.63 (dd, J=7.1 and 12.2 Hz, 0.5H); 4.31 to 4.44 (m, 2H) 50/50 mixture of diastereoisomers.

Stage 2: A solution of 392 mg of (7-oxabicyclo[2.2.1]hept-2-yl)methylamine obtained according to the preceding stage, in 10 ml of dioxane, 109 mg of 4,5-bis(diphenylphosphino)-9,9-dimethylxanthene, 35 mg of palladium acetate and 1.51 g of cesium carbonate are successively added to a solution of 0.68 g of 2-bromo-4-(3-methyl-4-quinolin-3-ylindazol-1-yl)benzonitrile, obtained according to stage 3 of Example 1, in 40 ml of dioxane under argon. The reaction medium is heated at 90° C. with stirring and under argon for 3 hours. After cooling, the reaction mixture is diluted with 200 ml of ethyl acetate and then filtered through clarcel. The filtrate is concentrated to dryness under reduced pressure. The residue obtained is chromatographed on silica gel (15-40 μm), elution being carried out with a mixture of dichloromethane and methanol (98:2 v/v), and then rechromatographed on silica gel (15-40 μm), elution being carried out with a mixture of ethyl acetate and cyclohexane (30:70 v/v). 97 mg of diastereoisomer A are obtained in the form of a white lacquer and 236 mg of diastereoisomer B are obtained in the form of a white lacquer. The mixture fractions are combined and rechromatographed on silica gel (15-40 μm), elution being carried out with a mixture of ethyl acetate and cyclohexane (20:80 v/v). 44 mg of diastereoisomer A are thus obtained in the form of a white solid and 156 mg of diastereoisomer B are thus obtained in the form of a white solid. In total, 141 mg of 4-(3-methyl-4-quinolin-3-ylindazol-1-yl)-2-{[exo-1-(7-oxabicyclo[2.2.1]hept-2-yl)methyl]amino}benzonitrile (diastereoisomer A) are obtained in the form of a white powder, the characteristics of which are the following: ¹H NMR spectrum (400 MHz, δ in ppm, DMSO-d6): 1.26 to 1.35 (m, 1H); 1.41 to 1.73 (m, 5H); 2.14 (s, 3H); 2.15 to 2.21 (m, 1H); 2.96 to 3.05 (m, 1H); 3.11 to 3.20 (m, 1H); 4.40 (d, J=4.9 Hz, 1H); 4.53 (t, J=4.9 Hz, 1H); 6.56 (t, J=5.0 Hz, 1H); 7.06 to 7.11 (m, 2H); 7.32 (d, J=7.1 Hz, 1H); 7.61 to 7.73 (m, 3H); 7.85 (ddd, J=1.6 and 6.9 and 8.4 Hz, 1H); 7.98 (d, J=8.1 Hz, 1H); 8.09 to 8.16 (m, 2 H); 8.53 (d, J=2.1 Hz, 1H); 9.05 (d, J=2.1 Hz, 1H);

and 392 mg of 4-(3-methyl-4-quinolin-3-ylindazol-1-yl)-2-{[endo-1-(7-oxabicyclo[2.2.1]hept-2-yl)methyl]amino}benzonitrile (diastereoisomer B) are obtained in the form of a white foam, the characteristics of which are the following:

¹H NMR spectrum (400 MHz, δ in ppm, DMSO-d6): 1.11 (dd, J=5.3 and 11.6 Hz, 1H); 1.40 to 1.72 (m, 3H); 1.82 to 1.91 (m, 2H); 2.14 (s, 3H); 2.40 to 2.48 (m, 1H); 3.24 to 3.39 (m, 2H); 4.44 to 4.50 (m, 2H); 6.32 (t, J=5.0 Hz, 1H); 7.11 (dd, J=1.8 and 8.4 Hz, 1H); 7.14 (d, J=1.8 Hz, 1H); 7.32 (d, J=7.1 Hz, 1H); 7.62 to 7.74 (m, 3H); 7.85 (ddd, J=1.3 and 6.9 and 8.4 Hz, 1H); 7.97 (d, J=8.6 Hz, 1H); 8.10 to 8.16 (m, 2H); 8.53 (d, J=2.2 Hz, 1H); 9.05 (d, J=2.2 Hz, 1H).

Stage 3: 3.6 ml of absolute ethanol, 0.53 ml of an aqueous solution of hydrogen peroxide at 30%, and then 0.58 ml of a 1N aqueous solution of sodium hydroxide are successively added to a suspension of 141 mg of 4-(3-methyl-4-quinolin-3-ylindazol-1-yl)-2-[exo-1-(7-oxabicyclo[2.2.1]hept-2-yl)amino]benzonitrile in 1.5 ml of dimethyl sulphoxide. The reaction mixture is stirred at 25° C. for 2 hours and is then poured into 10 ml of water. After extraction with 3 times 13 ml of ethyl acetate, the organic extracts are combined, washed with 10 ml of saturated brine, dried over magnesium sulphate, filtered, and then concentrated to dryness under reduced pressure. The residue is chromatographed on silica gel (15-40 μm), elution being carried out with a gradient of dichloromethane and methanol (100:0 then 99:1 then 98:2 v/v). The pure fractions are combined and then concentrated to dryness under reduced pressure. The residue is triturated from diethyl ether and then dried under reduced pressure at 40° C. 94 mg of 4-(3-methyl-4-quinolin-3-ylindazol-1-yl)-2-{[exo-1-(7-oxabicyclo[2.2.1]hept-2-yl)methyl]amino}benzamide are obtained in the form of a white powder, the characteristics of which are the following:

¹H NMR spectrum (400 MHz, δ in ppm, DMSO-d6): 1.24 to 1.32 (m, 1H); 1.42 to 1.70 (m, 5H); 2.04 to 2.11 (m, 1H); 2.14 (s, 3H); 2.87 to 2.95 (m, 1H); 3.04 to 3.13 (m, 1H); 4.33 (d, J=5.1 Hz, 1H); 4.53 (t, J=4.9 Hz, 1H); 6.93 (dd, J=2.0 and 8.2 Hz, 1H); 6.97 (d, J=2.0 Hz, 1H); 7.19 (broad s, 1H); 7.29 (d, J=7.0 Hz, 1H); 7.62 (dd, J=7.0 and 8.6 Hz, 1H); 7.71 (t, J=8.1 Hz, 1H); 7.81 to 7.88 (m, 2H); 7.91 (broad s, 1H); 7.95 (d, J=8.2 Hz, 1H); 8.10 to 8.16 (m, 2H); 8.53 (d, J=2.1 Hz, 1H); 8.59 (t, J=5.0 Hz, 1H); 9.06 (d, J=2.1 Hz, 1H).

Mass spectrum (LC/MS method C): Retention time Tr (min)=1.02; [M+H]₊: m/z=504.

### EXAMPLE 20

### Synthesis of 4-(3-methyl-4-quinolin-3-ylindazol-1-yl)-2-{[endo-1-(7-oxabicyclo[2.2.1]hept-2-yl)methyl]amino}benzamide

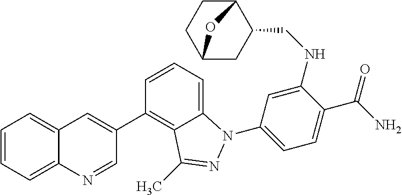

10 ml of absolute ethanol, 1.48 ml of an aqueous solution of hydrogen peroxide at 30% and then 1.61 ml of a 1N aqueous solution of sodium hydroxide are successively added to a suspension of 392 mg of 4-(3-methyl-4-quinolin-3-ylindazol-1-yl)-2-{[endo-1-(7-oxabicyclo[2.2.1]hept-2-yl)-methyl]amino}benzonitrile obtained according to stage 2 of Example 19, in 4.2 ml of dimethyl sulphoxide. The reaction mixture is stirred at 25° C. for 2 hours and is then poured into 30 ml of water. After extraction with 3 times 37 ml of ethyl acetate, the organic extracts are combined, washed with 25 ml of saturated brine, dried over magnesium sulphate, filtered and then concentrated to dryness under reduced pressure. The residue is chromatographed on silica gel (15-40 μm), elution being carried out with a gradient of dichloromethane and methanol (100:0 then 98:2 v/v). The pure fractions are combined and then concentrated to dryness under reduced pressure. The residue is taken up in 10 ml of water and then extracted with 3 times 12 ml of ethyl acetate. The organic extracts are combined, washed with 10 ml of water, dried over magnesium sulphate, filtered, and then concentrated to dryness under reduced pressure. The residue is triturated from diisopropyl ether and then dried under reduced pressure at 40° C. 163 mg of 4-(3-methyl-4-quinolin-3-ylindazol-1-yl)-2-{[endo-1-(7-oxabicyclo[2.2.1]hept-2-yl)methyl]amino}benzamide are obtained in the form of a white powder, the characteristics of which are the following:

¹H NMR spectrum (400 MHz, δ in ppm, DMSO-d6): 1.10 (dd, J=5.1 and 11.7 Hz, 1H); 1.43 to 1.64 (m, 3H); 1.74 to 1.82 (m, 1H); 1.84 to 1.95 (m, 1H); 2.15 (s, 3H); 2.30 to 2.38 (m, 1H); 3.10 to 3.39 (m, 2H); 4.45 to 4.51 (m, 2H); 6.95 (dd, J=2.0 and 8.6 Hz, 1H); 7.00 (d, J=2.0 Hz, 1H); 7.20 (broad s, 1H); 7.29 (d, J=7.2 Hz, 1H); 7.62 (dd, J=7.2 and 8.5 Hz, 1H); 7.71 (t, J=7.8 Hz, 1H); 7.82 to 7.88 (m, 2H); 7.91 (broad s, 1H); 7.96 (d, J=8.5 Hz, 1H); 8.10 to 8.16 (m, 2H); 8.52 to 8.56 (m, 2H); 9.06 (d, J=2.2 Hz, 1H).

Mass spectrum (LC/MS method C): Retention time Tr (min)=1.00; [M+H]₊: m/z=504.

### EXAMPLE 21

### Synthesis of 2-(trans-4-hydroxycyclohexylamino)-4-(4-quinolin-3-trifluoromethylindazol-1-yl)benzamide

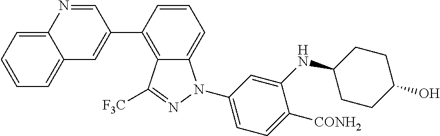

Stage 1: In a 250 ml round-bottomed flask, a mixture of 4.65 g of 2-(2,2,2-trifluoroacetyl)cyclohexane-1,3-dione [which can be prepared according to J. Fluorine Chem. 127 (2006), 1564] and 1.15 ml of hydrazine hydrate in 150 ml of absolute ethanol is refluxed under argon. After 2.5 hours, the reaction medium is left to return to ambient temperature and is evaporated to dryness under vacuum. The residue is chromatographed on silica gel (15-40 μm), elution being carried out with a mixture of methanol and dichloromethane (3:97 v/v then 6:94 v/v). 3.43 g of 3-trifluoromethyl-1,5,6,7-tetrahydroindazol-4-one are obtained in the form of a pale yellow solid, the characteristics of which are the following:

¹H NMR spectrum (400 MHz, δ in ppm, DMSO-d6): 2.08 (quin, J=6.4 Hz, 2H); 2.45 (m, 2H); 2.90 (t, J=6.2 Hz, 2H); 13.77 (broad s, 1H).

Mass spectrum (LC/MS method C):

Retention time Tr (min)=0.49; [M+H]₊: m/z 205; [M−H]₋: m/z 203.

Stage 2: In a 250 ml round-bottomed flask, a mixture of 2.41 g of 3-trifluoromethyl-1,5,6,7-tetrahydroindazol-4-one obtained according to the preceding stage, 5.27 g of cupric bromide and 1.02 g of lithium bromide in 120 ml of acetonitrile is refluxed. After 5 hours, the reaction medium is evaporated to dryness under vacuum. The residue is taken up with 100 ml of a saturated solution of sodium chloride and the aqueous phase is extracted with 3 times 100 ml of ethyl acetate. The combined organic phases are washed with twice 50 ml of a saturated solution of sodium chloride, dried over magnesium sulphate and evaporated to dryness under vacuum. The residual brown oil is chromatographed on silica gel (40-63 μm), elution being carried out with a mixture of ethyl acetate and cyclohexane (30:70 v/v). 2.36 g of 5-bromo-3-trifluoromethyl-1,5,6,7-tetrahydro-indazol-4-one are obtained in the form of a yellowish solid, the characteristics of which are the following:

¹H NMR spectrum (400 MHz, δ in ppm, DMSO-d6): 2.35 to 2.45 (m, 1H); 2.55 to 2.66 (m, 1H); 2.94 to 3.02 (m, 2H); 4.85 (dd, J=3.4 and 5.1 Hz, 1H); 14.11 (broad s, 1H).

Mass spectrum (LC/MS method C): Retention time Tr (min)=0.68; [M+H]+m/z 283; [M−H]−: m/z 281.

Stage 3: In a 250 ml round-bottomed flask, a mixture of 2.35 g of 5-bromo-3-trifluoromethyl-1,5,6,7-tetrahydroindazol-4-one obtained according to the preceding stage, 1.23 g of lithium carbonate and 721 mg of lithium bromide in 120 ml of anhydrous dimethylformamide is heated at 150° C. under argon. After 1 hour, the reaction medium is allowed to return to ambient temperature and is evaporated to dryness under vacuum. The black residue is taken up with 100 ml of ethyl acetate and 100 ml of distilled water. After separation by settling out, the aqueous phase is re-extracted with 3 times 100 ml of ethyl acetate, the aqueous phase being salted out with sodium chloride. The combined organic phases are washed with 100 ml of a saturated solution of sodium chloride, dried over magnesium sulphate and evaporated to dryness under vacuum. The oily black residue is chromatographed on silica gel (15-40 μm), elution being carried out with a mixture of ethyl acetate and cyclohexane (20:80 v/v). 413 mg of 4-hydroxy-3-trifluoromethyl-1H-indazole are obtained in the form of a yellowish solid, the characteristics of which are the following:

¹H NMR spectrum (400 MHz, δ in ppm, DMSO-d6): 6.57 (d, J=7.6 Hz, 1H); 7.03 (d, J=8.3 Hz, 1H); 7.25 (t, J=8.6 Hz, 1H); 10.32 (broad s, 1H); 13.62 (broad s, 1H).

Mass spectrum (LC/MS method C): Retention time Tr (min)=0.55; [M+H]+; m/z 203; [M−H]−: m/z 201.

Stage 4: In a 100 ml round-bottomed flask under argon, 1.7 ml of diisopropylethylamine are added to a mixture of 987 mg of 4-hydroxy-3-trifluoromethyl-1H-indazole obtained according to the preceding stage and 3.48 g of N-phenylbis(trifluoromethanesulphonimide) in 30 ml of dichloromethane and the mixture is then stirred at ambient temperature. After stirring for 7 hours, a further 3.48 g of N-phenylbis(trifluoromethanesulphonimide) and 1.7 ml of diisopropylethylamine are added to the reaction medium and the resulting mixture is left to stir for 24 hours. The reaction medium is poured into a saturated solution of sodium chloride and the aqueous phase is extracted twice with dichloromethane. The combined organic phases are dried over magnesium sulphate and evaporated to dryness under vacuum. The residue is chromatographed on silica gel (15-40 μm), elution being carried out with a mixture of dichloromethane and n-heptane (10:90 v/v). 1.58 g of trifluoromethanesulphonic acid 1-trifluoromethanesulphonyl-3-trifluoromethyl-1H-indazol-4-yl ester are obtained in the form of a colourless solid, the characteristics of which are the following:

¹H NMR spectrum (400 MHz, δ in ppm, DMSO-ds): 7.89 (d, J=8.5 Hz, 1H); 8.10 (t, J=8.5 Hz, 1H); 8.21 (d, J=8.5 Hz, 1H).

Mass spectrum (LC/MS method C): Retention time Tr (min)=1.21; [M−H]₋: m/z 465.

Stage 5: In a 100 ml round-bottomed flask under argon, a mixture of 510 mg of trifluoromethanesulphonic acid 1-trifluoromethanesulphonyl-3-trifluoromethyl-1H-indazol-4-yl ester obtained according to the preceding stage, 284 mg of 3-quinolineboronic acid, 348 mg of sodium carbonate and 190 mg of tetrakis(triphenylphosphine)palladium(0) in a mixture of 20.5 ml of toluene, 20.5 ml of ethanol and 320 μl of water is heated at 90° C. overnight. The following day, the reaction medium is evaporated to dryness under vacuum. The residue is taken up with ethyl acetate and the organic phase is washed successively with water and a saturated solution of sodium chloride, and then dried over magnesium sulphate. After evaporation to dryness under vacuum, the residue is chromatographed on silica gel (15-40 μm), elution being carried out with a mixture of ethyl acetate and n-heptane (35:65 v/v). 76.5 mg of 3-(3-trifluoromethyl-1H-indazol-4-yl)quinoline are obtained in the form of a white solid, the characteristics of which are the following:

¹H NMR spectrum (400 MHz, δ in ppm, DMSO-d6): 7.30 (d, J=6.8 Hz, 1H); 7.57 to 7.72 (m, 2H); 7.78 to 7.87 (m, 2H); 8.04 (d, J=8.6 Hz, 1H); 8.11 (d, J=8.3 Hz, 1H); 8.40 (s, 1H); 8.91 (s, 1H).

Mass spectrum (LC/MS method C): Retention time Tr (min)=0.84; [M+H]₊: m/z 314; [M−H]−: m/z 312.

Stage 6: In a 20 ml round-bottomed flask under argon, 26 mg of sodium hydride as a dispersion at 60% in oil are added, at ambient temperature, to a mixture of 147 mg of 3-(3-trifluoromethyl-1H-indazol-4-yl)quinoline obtained according to the preceding stage and 96 mg of 2-bromo-4-fluorobenzonitrile in 4 ml of anhydrous dimethylformamide. The reaction medium is then heated at 50° C. for 1 hour under argon and is then poured into a saturated solution of sodium chloride. The aqueous phase is extracted twice with ethyl acetate. The combined organic phases are washed with a saturated solution of sodium chloride, dried over magnesium sulphate and evaporated to dryness under vacuum. The residue is chromatographed on silica gel (15-40 μm), elution being carried out with dichloromethane. 128 mg of 2-bromo-4-(4-quinolin-3-yl-3-trifluoromethylindazol-1-yl)benzonitrile are obtained in the form of a beige solid, the characteristics of which are the following:

¹H NMR spectrum (400 MHz, δ in ppm, DMSO-d6): 7.54 (d, J=6.8 Hz, 1H); 7.71 (t, J=8.2 Hz, 1H); 7.80 to 7.89 (m, 2H); 8.07 (d, J=8.1 Hz, 1H); 8.10 to 8.19 (m, 3H); 8.25 (d, J=8.6 Hz, 1H); 8.39 (d, J=2.2 Hz, 1H); 8.45 (s, 1H); 8.94 (s, 1H).

Mass spectrum (LC/MS method B): Retention time Tr (min)=5.11; [M+H]+: m/z 493; [M−H]−+[HCOOH]: m/z 537.

Stage 7: In a 25 ml round-bottomed flask under argon, a mixture of 120 mg of 2-bromo-4-(4-quinolin-3-yl-3-trifluoromethylindazol-1-yl)benzonitrile obtained according to the preceding stage, 56 mg of trans-4-aminocyclohexanol, 238 mg of cesium carbonate, 17 mg of 4,5-bis(diphenylphosphino)-9,9-dimethylxanthene and 5 mg of palladium acetate in 9 ml of dioxane is heated at 95° C. After 5 hours, the reaction medium is evaporated to dryness under vacuum and the residue is chromatographed on silica gel (15-40 μm), elution being carried out with a gradient of ethyl acetate and n-heptane (20:80 then 30:70 then 40:60 then 50:50 v/v). 14 mg of 2-(trans-4-hydroxycyclohexylamino)-4-(4-quinolin-3-yl-3-trifluoromethyl-indazol-1-yl)benzonitrile are obtained in the form of a white solid, the characteristics of which are the following:

¹H NMR spectrum (400 MHz, δ in ppm, DMSO-d6): 1.25 to 1.51 (m, 4H); 1.84 (d, J=13.9 Hz, 2H); 1.96 (d, J=12.7 Hz, 2H); 3.39 to 3.59 (m, 2H); 4.52 (d, J=4.4 Hz, 1H); 6.05 (d, J=7.8 Hz, 1H); 7.06 (dd, J=2.0 and 8.3 Hz, 1H); 7.23 (d, J=2.0 Hz, 1H); 7.49 (d, J=6.8 Hz, 1H); 7.67 to 7.82 (m, 3H); 7.86 (ddd, J=1.5 and 6.9 and 8.5 Hz, 1H); 8.01 (d, J=8.6 Hz, 1H); 8.07 (d, J=8.3 Hz, 1H); 8.13 (d, J=8.3 Hz, 1H); 8.45 (d, J=1.7 Hz, 1H); 8.95 (d, J=1.7 Hz, 1H).

Mass spectrum (LC/MS method C): Retention time Tr (min)=1.10; [M+H]+: m/z 528; [M−H]−: m/z 526.

Stage 8: In a 5 ml round-bottomed flask under argon, 50 μl of 1M sodium hydroxide then 50 μl of a 30% solution of hydrogen peroxide are successively added, at ambient temperature, to a mixture of 14 mg of 2-(trans-4-hydroxycyclohexylamino)-4-(4-quinolin-3-yl-3-trifluoromethylindazol-1-yl)benzonitrile obtained according to the preceding stage, in 0.3 ml of dimethyl sulphoxide and 0.1 ml of ethanol. After stirring for 30 minutes, water is added and then the resulting mixture is extracted twice with ethyl acetate. The combined organic phases are washed with a saturated solution of sodium chloride, dried over magnesium sulphate and then evaporated to dryness under vacuum. The residual solid is triturated from isopropyl ether, filtered and washed with ethyl ether then pentane, and dried under vacuum. 6.7 mg of 2-(trans-4-hydroxycyclohexylamino)-4-(4-quinolin-3-yl-3-trifluoromethylindazol-1-yl)benzamide are obtained in the form of a white solid, the characteristics of which are as follows:

¹H NMR spectrum (400 MHz, δ in ppm, DMSO-d6): 1.14 to 1.43 (m, 4H); 1.75 to 1.89 (m, J=12.7 Hz, 2H); 1.95 to 2.10 (m, 2H); 3.37 to 3.55 (m, 2H); 4.51 (d, J=4.4 Hz, 1H); 6.88 (dd, J=2.1 and 8.4 Hz, 1H); 7.05 (d, J=1.5 Hz, 1H); 7.29 (broad s, 1H), 7.47 (d, J=7.1 Hz, 1H); 7.71 (t, J=8.3 Hz, 1H); 7.78 (dd, J=7.1 and 8.8 Hz, 1H); 7.82 to 7.90 (m, 2H); 7.93 to 8.02 (m, J=8.6 Hz, 2H); 8.07 (d, J=8.6 Hz, 1H); 8.13 (d, J=8.8 Hz, 1H); 8.45 (s, 1H); 8.49 (d, J=7.6 Hz, 1H); 8.96 (s, 1H).

Mass spectrum (LC/MS method C): Retention time Tr (min)=0.95; [M+H]+: m/z 546; [M−H]−: m/z 544.

### EXAMPLE 22

### Synthesis of 4-[4-(6-fluoro-1H-benzimidazol-2-yl)-3-trifluoromethylindazol-1-yl]-2-(trans-4-hydroxycyclohexylamino)benzamide

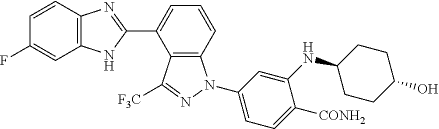

Stage 1: In a 500 ml round-bottomed flask under argon, a mixture of 6.1 g of 2-(2,2,2-trifluoroacetyl)cyclohexane-1,3-dione (which can be prepared according to J. Fluorine Chem. 2007, 127, 1564) and 6.2 g of 2-bromo-4-hydrazinobenzo-nitrile (which can be prepared according to WO 2007/101 156) in 180 ml of ethanol is heated at 50-60° C. After 15 minutes, the reaction medium is allowed to return to ambient temperature and is evaporated to dryness under vacuum. The off-white solid obtained is triturated from isopropyl ether, filtered and washed twice with pentane. After drying under vacuum, 8.38 g of 2-bromo-4-{N′-[2,2,2-trifluoro-1-(2-hydroxy-6-oxocyclohex-1-enyl)ethylidene]hydrazino}benzonitrile are obtained in the form of a pinkish solid, the characteristics of which are the following:

¹H NMR spectrum (400 MHz, δ in ppm, DMSO-d6): 1.85 to 2.09 (m, 2H); 2.38 to 2.47 (m, 4H); 7.26 (dd, J=2.1 and 8.7 Hz, 1H); 7.51 (d, J=2.1 Hz, 1H); 7.76 (d, J=8.6 Hz, 1H); 9.93 (s, 1H); 11.95 (broad s, 1H).

Mass spectrum (LC/MS method C): Retention time Tr (min)=0.89; [M+H]+: m/z 402; [M−H]−: m/z 400.

Stage 2: In eight 20 ml reactors, a mixture of 1 g of 2-bromo-4-{N′-[2,2,2-trifluoro-1-(2-hydroxy-6-oxocyclohex-1-enyl)ethylidene]hydrazino}benzonitrile obtained according to the preceding stage and 1.7 ml of acetic acid in 13 ml of ethanol is each time irradiated with microwaves at 150° C. for 15 minutes. The combined eight reactions are evaporated to dryness under vacuum. The residue is taken up in ethyl acetate, and washed with water and then a saturated solution of sodium chloride. The organic phase dried over magnesium sulphate is evaporated to dryness under vacuum. The solid residue is triturated from isopropyl ether, filtered and washed with pentane. After drying under vacuum, 7.25 g of 2-bromo-4-(4-oxo-3-trifluoromethyl-4,5,6,7-tetrahydroindazol-1-yl)benzonitrile are obtained in the form of a pinkish solid, the characteristics of which are the following:

¹H NMR spectrum (400 MHz, δ in ppm, DMSO-d₆): 2.11 (quin, J=6.4 Hz, 2 H); 2.51 to 2.56 (m, 2H); 3.10 (t, J=6.1 Hz, 2H); 7.91 (dd, J=2.1 and 8.4 Hz, 1H); 8.17 to 8.23 (m, 2H).

Mass spectrum (LC/MS method C): Retention time Tr (min)=1.02; [M+H]+: m/z 384; [M−H]−: m/z 382.

Stage 3: In a 500 ml round-bottomed flask under argon, a mixture of 7.25 g of 2-bromo-4-(4-oxo-3-trifluoromethyl-4,5,6,7-tetrahydroindazol-1-yl)benzonitrile obtained according to the preceding stage, 8.4 g of cupric bromide and 1.6 g of lithium bromide in 300 ml of acetonitrile is brought to reflux for 1.5 hours. After cooling to ambient temperature, the reaction medium is evaporated to dryness under vacuum. Distilled water, ethyl acetate and clarcel are added to the residue and the mixture is filtered, the solid being washed with ethyl acetate. The filtrate is separated by settling out and the organic phase is washed successively with water and twice with a saturated solution of sodium chloride, dried over magnesium sulphate and evaporated to dryness under vacuum. 8.36 g of 2-bromo-4-(5-bromo-4-oxo-3-trifluoromethyl-4,5,6,7-tetrahydroindazol-1-yl)benzonitrile are obtained in the form of a brown solid which is used as it is, without characterization, in the next stage.

Stage 4: In a 1 l round-bottomed flask under argon, a mixture of 8.36 g of 2-bromo-4-(5-bromo-4-oxo-3-trifluoromethyl-4,5,6,7-tetrahydroindazol-1-yl)benzonitrile, 2.67 g of lithium carbonate and 1.57 g of lithium bromide in 400 ml of anhydrous dimethylformamide is heated at 140° C. for 1 hour. After cooling, the reaction medium is carefully poured into a 1N solution of hydrochloric acid and extracted twice with ethyl acetate. The combined organic phases are washed twice with a saturated solution of sodium chloride, dried over sodium sulphate and evaporated to dryness under vacuum. The residue is chromatographed on silica gel (15-40 μm), elution being carried out with a gradient of ethyl acetate and n-heptane [20:80 (15 min); 30:70 (10 min); 40:60 (15 min) v/v]. 4.2 g of 2-bromo-4-(4-hydroxy-3-trifluoromethylindazol-1-yl)benzonitrile are obtained in the form of a beige solid, the characteristics of which are the following:

¹H NMR spectrum (400 MHz, δ in ppm, DMSO-d6): 6.79 (d, J=7.6 Hz, 1H); 7.40 (d, J=8.0 Hz, 1H); 7.48 (t, J=8.6 Hz, 1H); 8.05 (dd, J=2.2 and 8.6 Hz, 1H); 8.17 (d, J=8.6 Hz, 1H); 8.28 (d, J=2.2 Hz, 1H); 10.89 (broad s, 1H).

Mass spectrum (LC/MS method C): Retention time Tr (min)=1.08; [M−H]−: m/z 380.

Stage 5: In a 500 ml round-bottomed flask, argon is bubbled into a mixture of 2.0 g of 2-bromo-4-(4-hydroxy-3-trifluoromethylindazol-1-yl)benzonitrile obtained according to the preceding stage, in 200 ml of dioxane. 1.2 g of trans-4-aminocyclohexanol, 6.8 g of cesium carbonate, 360 mg of 4,5-bis(diphenylphosphino)-9,9-dimethylxanthene and 117 mg of palladium acetate are successively added. The mixture is heated at 95° C. under argon for 24 hours. The reaction medium, after cooling, is carefully poured into 400 ml of a 1N solution of hydrochloric acid. The aqueous phase is extracted twice with ethyl acetate and the combined organic phases are washed with a saturated solution of sodium chloride, dried over magnesium sulphate and evaporated to dryness under vacuum. The residue is chromatographed on silica gel (15-40 μm), elution being carried out with a gradient of ethyl acetate and n-heptane [50:50 (20 min); 60:40 (20 min) v/v]. 340 mg of 2-(trans-4-hydroxycyclohexylamino)-4-(4-hydroxy-3-trifluoromethylindazol-1-yl)benzonitrile are obtained in the form of a beige solid, the characteristics of which are the following:

¹H NMR spectrum (400 MHz, δ in ppm, DMSO-d6): 1.22 to 1.33 (m, 2H); 1.35 to 1.49 (m, 2H); 1.80 to 1.88 (m, 2H); 1.90 to 1.97 (m, 2H); 3.37 to 3.57 (m, 2H); 4.52 (d, J=4.4 Hz, 1H); 5.98 (d, J=8.3 Hz, 1H); 6.75 (d, J=7.6 Hz, 1H); 7.00 (dd, J=2.2 and 8.3 Hz, 1H); 7.14 (d, J=2.2 Hz, 1H); 7.26 (d, J=8.6 Hz, 1H); 7.43 (t, J=7.5 Hz, 1H); 7.68 (d, J=8.6 Hz, 1H); 10.76 (broad s, 1H).

Mass spectrum (LC/MS method C): Retention time Tr (min)=0.96; [M−H]−: m/z 415.

Stage 6: In a 30 ml round-bottomed flask, a mixture of 340 mg of 2-(trans-4-hydroxycyclohexylamino)-4-(4-hydroxy-3-trifluoromethylindazol-1-yl)benzonitrile obtained according to the preceding stage and 584 mg of N-phenylbis(trifluoro-methanesulphonimide) in 5 ml of dichloromethane and 2 ml of tetrahydrofuran and 285 μl of diisopropylethylamine is stirred at ambient temperature under argon. After 7 hours, a further 500 mg of N-phenylbis(trifluoromethanesulphonimide) and 500 μl of diisopropylethylamine are added and the stirring is continued under argon at ambient temperature for 24 hours. The reaction medium is poured into a saturated solution of sodium chloride and the aqueous phase is extracted twice with methylene chloride. The combined organic phases are dried over magnesium sulphate and evaporated to dryness under vacuum. The residue is chromatographed on silica gel (15-40 μm), elution being carried out with a gradient of ethyl acetate and n-heptane [30:70 (5 min); 50:50 (20 min) v/v]. 335 mg of trifluoromethanesulphonic acid 1-[4-cyano-3-(trans-4-hydroxycyclohexylamino)-phenyl]-3-trifluoromethyl-1H-indazol-4-yl ester are obtained in the form of an amber solid, the characteristics of which are the following:

¹H NMR spectrum (400 MHz, δ in ppm, DMSO-d6): 1.20 to 1.33 (m, 2H); 1.35 to 1.49 (m, 2H); 1.82 (d, J=12.0 Hz, 2H); 1.93 (d, J=12.7 Hz, 2H); 3.36 to 3.59 (m, 2H); 4.51 (broad s, 1H); 6.03 (d, J=8.1 Hz, 1H); 7.01 (dd, J=2.0 and 8.3 Hz, 1H); 7.23 (d, J=2.2 Hz, 1H); 7.62 (d, J=7.8 Hz, 1H); 7.73 (d, J=8.3 Hz, 1H); 7.80 (t, J=8.3 Hz, 1H); 7.99 (d, J=8.8 Hz, 1H).

Mass spectrum (LC/MS method C): Retention time T1 (min)=1.17; [M−H]−: m/z 547.

Stage 7: In an autoclave, a mixture of 390 mg of trifluoromethanesulphonic acid 1-[4-cyano-3-(trans-4-hydroxycyclohexylamino)phenyl]-3-trifluoromethyl-1H-indazol-4-yl ester obtained according to the preceding stage, 32 mg of palladium acetate, 57 mg of 1,3-bis(diphenylphosphino)propane and 0.1 ml of triethylamine in 2 ml of methanol and 5 ml of dimethylformamide is maintained at 50° C. for 16 hours under a carbon monoxide pressure of 2 bar. After flushing with argon, the reaction medium is taken up in distilled water and ethyl acetate. After separation by settling out, the aqueous phase is re-extracted with ethyl acetate. The combined organic phases are washed with a saturated solution of sodium chloride, dried over magnesium sulphate and evaporated to dryness under vacuum. The residue is chromatographed on silica gel (15-40 μm), elution being carried out with a mixture of ethyl acetate and n-heptane (50:50 v/v). 260 mg of 1-[4-cyano-3-(trans-4-hydroxycyclohexylamino)phenyl]-3-trifluoromethyl-1H-indazole-4-carboxylic acid methyl ester are obtained in the form of a beige solid, the characteristics of which are the following:

¹H NMR spectrum (400 MHz, δ in ppm, DMSO-d6): 1.16 to 1.49 (m, 4H); 1.78 to 1.86 (m, 2H); 1.88 to 1.99 (m, 2H); 3.34 to 3.57 (m, 2H); 3.92 (s, 3H); 4.51 (d, J=4.4 Hz, 1H); 6.03 (d, J=8.3 Hz, 1H); 7.00 (dd, J=2.0 and 8.3 Hz, 1H); 7.20 (d, J=2.4 Hz, 1H); 7.67 to 7.80 (m, 2H); 7.86 (d, J=6.8 Hz, 1H); 8.09 (d, J=8.8 Hz, 1H).

Mass spectrum (LC/MS method B): Retention time Tr (min)=4.47; [M+H]+: m/z 459; [M−H]−: m/z 457.

Stage 8: in a 50 ml round-bottomed flask, a mixture of 260 mg of 1-[4-cyano-3-(trans-4-hydroxycyclohexylamino)phenyl]-3-trifluoromethyl-1H-indazole-4-carboxylic acid methyl ester obtained according to the preceding stage and 2.3 ml of 1M sodium hydroxide in 7 ml of dioxane, 1 ml of methanol and 2 ml of distilled water is stirred at ambient temperature for 5 hours. 15 ml of a 1N solution of hydrochloric acid are then carefully added and the resulting mixture is extracted twice with ethyl acetate. The combined organic phases are washed with a saturated solution of sodium chloride, dried over magnesium sulphate and evaporated to dryness under vacuum. The residue is triturated from isopropyl ether and the whole is again evaporated to dryness under vacuum. 244 mg of 1-[4-cyano-3-(trans-4-hydroxycyclohexylamino)phenyl]-3-trifluoromethyl-1H-indazole-4-carboxylic acid are obtained in the form of a beige solid, the characteristics of which are the following:

¹H NMR spectrum (400 MHz, δ in ppm, DMSO-d6): 1.16 to 1.51 (m, 4H); 1.83 (d, J=15.4 Hz, 2H); 1.93 (d, J=11.0 Hz, 2H); 3.36 to 3.55 (m, 2H); 4.51 (d, J=4.4 Hz, 1H); 6.01 (d, J=8.1 Hz, 1H); 7.00 (dd, J=2.0 and 8.3 Hz, 1H); 7.19 (d, J=2.2 Hz, 1H); 7.72 (d, J=8.3 Hz, 2H); 7.80 (d, J=7.3 Hz, 1H); 8.01 (d, J=9.0 Hz, 1H); 13.48 (s, 1H).

Mass spectrum (LC/MS method C): Retention time T1 (min)=0.88; [M+H]+: m/z 445; [M−H]−: m/z 443.

Stage 9: In a 50 ml round-bottomed flask under argon, a mixture of 244 mg of 1-[4-cyano-3-(trans-4-hydroxycyclohexylamino)phenyl]-3-trifluoromethyl-1H-indazole-4-carboxylic acid obtained according to the preceding stage, 73 mg of 1,2-diamino-4-fluorobenzene, 198 mg of O-((ethoxycarbonyl)cyanomethyleneamino)-N,N,N′,N′-tetramethyluronium tetrafluoroborate (TOTU) and 105 μl of diisopropylethylamine in 10 ml of anhydrous dimethylformamide is stirred at ambient temperature for 4 hours. The reaction medium is poured into a saturated solution of sodium chloride. A small amount of distilled water is added and the resulting mixture is extracted twice with ethyl acetate. The combined organic phases are washed with a saturated solution of sodium chloride, dried over magnesium sulphate and evaporated to dryness under vacuum. 360 mg of 1-[4-cyano-3-(trans-4-hydroxycyclohexylamino)phenyl]-3-trifluoromethyl-1H-indazole-4-carboxylic acid (2-amino-4-fluorophenyl)amide are obtained in the form of a brown solid which is used as it is in the next stage without further characterization.

Stage 10: In a 20 ml reactor, a mixture of 303 mg of 1-[4-cyano-3-(trans-4-hydroxycyclohexylamino)phenyl]-3-trifluoromethyl-1H-indazole-4-carboxylic acid (2-amino-4-fluorophenyl)amide obtained according to the preceding stage, in 15 ml of acetic acid, is irradiated with microwaves at 115° C. for 60 minutes. The reaction medium is evaporated to dryness under vacuum and the residue is taken up, with vigorous stirring, with 30 ml of methanol and 3 ml of 1N sodium hydroxide. The resulting product is poured into distilled water and acidified to pH=1 with a 1N solution of hydrochloric acid. A small amount of saturated solution of sodium chloride is added and the resulting mixture is extracted twice with ethyl acetate. The combined organic phases are washed with a saturated solution of sodium chloride, dried over sodium sulphate and evaporated to dryness under vacuum. The residue is chromatographed on silica gel (15-40 μm), elution being carried out with a gradient of ethyl acetate in n-heptane [60:40 (10 min); 70:30 (10 min); 80:20 (10 min)]. 88 mg of a beige solid containing predominantly 4-[4-(6-fluoro-1H-benzimidazol-2-yl)-3-trifluoromethylindazol-1-yl]-2-(trans-4-hydroxycyclohexylamino)benzonitrile are obtained, the characteristics of which are the following:

¹H NMR spectrum (400 MHz, δ in ppm, DMSO-d6): 1.20 to 1.50 (m, 4H); 1.84 (d, J=9.3 Hz, 2H); 1.95 (d, J=12.2 Hz, 2H); 3.38 to 3.58 (m, 2H); 4.52 (d, J=4.6 Hz, 1H); 6.06 (d, J=8.3 Hz, 1H); 7.05 (dd, J=2.1 and 8.4 Hz, 1H); 7.11 (t, J=7.0 Hz, 1H) 7.22 (s, 1H); 7.42 (broad s, 1H); 7.62 (broad s, 1H); 7.68 to 7.76 (m, 2H); 7.80 (t, J=7.0 Hz, 1H); 8.06 (d, J=8.6 Hz, 1H); 12.97 (broad s, 1H).

Mass spectrum (LC/MS method C): Retention time Tr (min)=0.94; [M+H]+m/z 535; [M−H]−: m/z 533.

Stage 11: In a 10 ml round-bottomed flask under argon, 300 μl of 1M sodium hydroxide and then 300 μl of a solution of hydrogen peroxide at 30% are successively added, at ambient temperature, to a mixture of 85 mg of 4-[4-(6-fluoro-1H-benzimidazol-2-yl)-3-trifluoromethylindazol-1-yl]-2-(trans-4-hydroxycyclohexylamino)benzonitrile obtained according to the preceding stage, in 1.8 ml of dimethyl sulphoxide and 0.6 ml of ethanol. After stirring for 40 minutes, a saturated solution of sodium chloride is added and the resulting mixture is then extracted twice with ethyl acetate. The combined organic phases are washed with a saturated solution of sodium chloride, dried over magnesium sulphate and then evaporated to dryness under vacuum. The solid residue is triturated from isopropyl ether, filtered, washed with isopropyl ether and then pentane, and dried at 40° C. under vacuum. 77 mg of 4-[4-(6-fluoro-1H-benzimidazol-2-yl)-3-trifluoromethylindazol-1-yl]-2-(trans-4-hydroxycyclohexylamino)benzamide are obtained in the form of a beige solid, the characteristics of which are the following:

¹H NMR spectrum (400 MHz, δ in ppm, DMSO-d₆); 1.16 to 1.41 (m, 4H); 1.82 (d, J=12.2 Hz, 2H); 2.02 (d, J=13.0 Hz, 2H); 3.36 to 3.54 (m, 2H); 4.51 (d, J=4.2 Hz, 1H); 6.41 (broad s, 1H); 6.88 (dd, J=2.1 and 8.4 Hz, 1H); 7.04 (d, J=1.7 Hz, 1H); 7.11 (broad s, 1H); 7.21 to 7.62 (broad m, 3H); 7.69 (d, J=7.1 Hz, 1H); 7.78 (dd, J=7.1 and 8.8 Hz, 1H); 7.87 (d, J=8.3 Hz, 1H); 8.02 (d, J=8.3 Hz, 1H); 8.49 (d, J=7.6 Hz, 1H); 12.96 (broad s, 1H).

Mass spectrum (LC/MS method B): Retention time Tr (min)=3.42; [M+H]+: m/z 553; [M−H]−: m/z 551.

### EXAMPLE 23

### Synthesis of 3-(trans-4-hydroxycyclohexylamino)-5-(4-quinolin-3-yl-3-trifluoromethylindazol-1-yl)pyridine-2-carboxamide

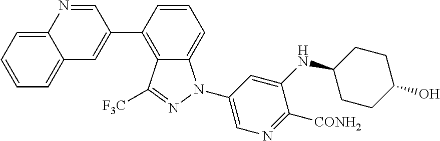

In a 50 ml three-necked flask, 15 mg of sodium hydride as a dispersion at 60% in petroleum jelly are added, in one step under argon at 50° C., to a mixture of 76 mg of 3-(3-trifluoromethyl-1H-indazol-4-yl)quinoline obtained according to stage 5 of Example 21, in 5 ml of anhydrous dimethylformamide. The mixture is maintained at 50° C. for 20 minutes and then a solution of 63 mg of 2-cyano-5-fluoro-3-(trans-4-hydroxycyclohexylamino)pyridine obtained according to stage 1 of Example 16, in 2 ml of anhydrous dimethylformamide, is added. The reaction medium is heated at 80° C. under argon for 8 hours and then allowed to return to 35° C. 15 mg of sodium hydride as a dispersion at 60% in petroleum jelly are again added and the resulting mixture is heated at 50° C. for 1 hour and then at 80° C. overnight. The following day, the reaction medium is allowed to cool to ambient temperature, a few drops of ethanol are added, and the resulting mixture is evaporated to dryness under vacuum. The residue is taken up with ethyl acetate and the organic phase is washed with distilled water, dried over magnesium sulphate and evaporated to dryness under vacuum. The residue is chromatographed on silica gel (15-40 μm), elution being carried out with a mixture of ethyl acetate and n-heptane (30:70 v/v). The interesting fractions are combined and evaporated to dryness under vacuum and the solid obtained is taken up in a mixture of methanol and dichloromethane (10:90 v/v) and evaporated to dryness under vacuum. 9 mg of 3-(trans-4-hydroxycyclohexylamino)-5-(4-quinolin-3-yl-3-trifluoromethylindazol-1-yl)pyridine-2-carboxamide are obtained in the form of a pale yellow solid, the characteristics of which are the following:

¹H NMR spectrum (400 MHz, δ in ppm, DMSO-d6): 1.18 to 1.42 (m, 4H); 1.78 to 1.86 (m, 2H); 1.97 to 2.06 (m, 2H); 3.44 to 3.54 (m, 2H); 4.54 (d, J=4.4 Hz, 1H); 7.50 (d, J=6.8 Hz, 1H); 7.58 (broad s, 1H); 7.63 (d, J=2.4 Hz, 1H); 7.71 (t, J=8.1 Hz 1H); 7.80 (t, J=7.3 Hz, 1H); 7.86 (t, J=8.1 Hz, 1H); 7.99 (d, J=8.8 Hz, 1H); 8.08 (d, J=8.3 Hz, 1H); 8.11 to 8.16 (m, 3H); 8.45 (s, 1H); 8.78 (d, J=7.8 Hz, 1H); 8.95 (s, 1H).

Mass spectrum (LC/MS method C): Retention time Tr (min)=1; [M+H]+: m/z 547.

### EXAMPLE 24

### Synthesis of 2-(trans-4-hydroxycyclohexylamino)-4-(4-quinolin-3-ylindazol-1-yl)benzamide

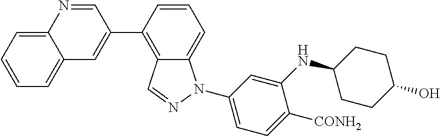

Stage 1: In a 500 ml round-bottomed flask, a mixture of 2.5 g of 1,5,6,7-tetrahydroindazol-4-one (which can be prepared according to Synthesis 2002, 12, 1669), 8.2 g of cupric bromide and 1.59 g of lithium bromide in 400 ml of acetonitrile is refluxed under argon for 3 hours. The reaction medium is allowed to cool and is evaporated to approximately 50 ml. 200 ml of distilled water and 200 ml of ethyl acetate are added. After separation by settling out, the aqueous phase is re-extracted with 200 ml of ethyl acetate and then the combined organic phases are washed twice with 100 ml of a saturated solution of sodium chloride and then once with 100 ml of distilled water. After drying over magnesium sulphate, the resulting product is evaporated to dryness under vacuum. 3.5 g of 5-bromo-1,5,6,7-tetrahydroindazol-4-one are obtained in the form of a greenish solid, the characteristics of which are the following:

¹H NMR spectrum (400 MHz, δ in ppm, DMSO-d6) tautomeric mixture: 2.26 to 2.43 (m, 1H); 2.51 to 2.59 (m, 1H); 2.74 to 3.01 (m, 2H); 4.78 (m, 1H); 7.89 (s, 0.6; H); 8.39 (broad s, 0.4; H); 13.38 (broad s, 0.6; H) 13.49 (broad s, 0.4; H).

Mass spectrum (LC/MS method C): Retention time Tr (min)=0.40; [M+H]+: m/z 215; [M−H]−: m/z 213.

Stage 2: In a 500 ml round-bottomed flask, a mixture of 2.5 g of 5-bromo-1,5,6,7-tetrahydroindazol-4-one obtained according to the preceding stage, 1.72 g of lithium carbonate and 1.0 g of lithium bromide in 125 ml of anhydrous dimethylformamide is heated under argon at 150° C. for 1 hour. The reaction medium is allowed to return to ambient temperature and is then evaporated to dryness under vacuum. The black residue is taken up with 100 ml of ethyl acetate and 100 ml of a saturated solution of sodium chloride and carefully with 60 ml of 1N hydrochloric acid. After separation by settling out, the aqueous phase is re-extracted with 3 times 100 ml of ethyl acetate. The combined organic phases are washed with twice 100 ml of a saturated solution of sodium chloride, dried over magnesium sulphate and evaporated to dryness under vacuum. The residue is chromatographed on silica gel (15-40 μm), elution being carried out with a mixture of ethyl acetate and cyclohexane (30:70 v/v). 455 mg of 1H-indazol-4-ol are obtained in the form of an off-white solid, the characteristics of which are the following:

¹H NMR spectrum (400 MHz, δ in ppm, DMSO-d6); 6.38 (d, J=7.3 Hz, 1H); 6.91 (d, J=8.3 Hz, 1H); 7.10 (t, J=7.9 Hz, 1H); 8.02 (s, 1H); 9.99 (broad s, 1H); 12.83 (broad s, 1H).

Mass spectrum (LC/MS method B): Retention time Tr (min)=1.06; [M+H]+: m/z 135; [M−H]−: m/z

Stage 3: In a 250 ml round-bottomed flask, a mixture of 645 mg of 1H-indazol-4-ol obtained according to the preceding stage, 1.08 g of N-phenylbis(trifluoromethanesulphonimide) and 1.24 ml of diisopropylethylamine in 40 ml of tetrahydrofuran is stirred under argon at ambient temperature. After 3 hours, 0.9 g of N-phenylbis(trifluoromethanesulphonimide) and 0.6 ml of diisopropylethylamine are added and the stirring is continued overnight. The following day, the reaction medium is evaporated to dryness under vacuum. The residue is chromatographed on silica gel (15-40 μm), elution being carried out with a mixture of ethyl acetate and cyclohexane (20:80 v/v). 863 mg of trifluoromethanesulphonic acid 1H-indazol-4-yl ester are obtained in the form of a white solid, the characteristics of which are the following:

¹H NMR spectrum (400 MHz, δ in ppm, DMSO-d₆): 7.24 (d, J=7.6 Hz, 1H); 7.49 (t, J=7.9 Hz, 1H); 7.70 (d, J=8.3 Hz, 1H); 8.17 (s, 1H); 13.68 (broad s, 1H).

Mass spectrum (LC/MS method C): Retention time Tr (min)=0.90; [M+H]+: m/z 267; [M−H]−: m/z 265.

Stage 4: In a 250 ml round-bottomed flask under argon, a mixture, degassed beforehand with argon, of 255 mg of trifluoromethanesulphonic acid 1H-indazol-4-yl ester obtained according to the preceding stage, 249 mg of 3-quinolineboronic acid, 305 mg of sodium carbonate and 166 mg of tetrakis(triphenylphosphine)palladium(0) in a mixture of 10 ml of ethanol, 10 ml of toluene and 1.3 ml of distilled water is heated at 95° C. for 1.25 hours. After cooling to ambient temperature, the reaction medium is evaporated to dryness under vacuum. The residue is taken up with 50 ml of a saturated solution of sodium chloride and extracted four times with 50 ml of ethyl acetate. The combined organic phases are washed with 50 ml of a saturated solution of sodium chloride, dried over magnesium sulphate and evaporated to dryness under vacuum. The residue is chromatographed on silica gel (15-40 μm), elution being carried out with a mixture of ethyl acetate and n-heptane (30:70 v/v). 137 mg of 3-(1H-indazol-4-yl)quinoline are obtained in the form of a white solid, the characteristics of which are the following:

¹H NMR spectrum (400 MHz, δ in ppm, DMSO-d₆); 7.46 (d, J=6.8 Hz, 1H); 7.53 (t, J=7.6 Hz, 1H); 7.61 to 7.72 (m, 2H); 7.82 (ddd, J=1.5 and 6.9 and 8.5 Hz, 1H); 8.10 (d, J=7.8 Hz, 1H); 8.15 (d, J=8.1 Hz, 1H); 8.34 (s, 1H); 8.74 (dd, J=0.5 and 2.0 Hz, 1H); 9.29 (d, J=2.4 Hz, 1H); 13.33 (broad s, 1H).

Mass spectrum (LC/MS method C): Retention time Tr (min)=0.64; [M+H]+: m/z 246; m/z 244.

Stage 5: In a 100 ml round-bottomed flask, 10.4 mg of sodium hydride as a dispersion at 60% in petroleum jelly are added, at ambient temperature under argon, to a mixture of 58 mg of 3-(1H-indazol-4-yl)quinoline obtained according to the preceding stage and 47.3 mg of 2-bromo-4-fluorobenzonitrile in 5 ml of anhydrous dimethylformamide, and the resulting mixture is then stirred at ambient temperature for 2.5 hours. The reaction medium is evaporated to dryness under vacuum and the residue is chromatographed on silica gel (15-40 μm), elution being carried out with a mixture of ethyl acetate and cyclohexane (30:70 v/v). 24 mg of 2-bromo-4-(4-quinolin-3-ylindazol-1-yl)benzonitrile are obtained in the form of an off-white solid, the characteristics of which are the following;

¹H NMR spectrum (400 MHz, δ in ppm, DMSO-d6): 7.64 to 7.80 (m, 3H); 7.85 (ddd, J=1.6 and 6.9 and 8.4 Hz, 1H); 8.03 to 8.22 (m, 5H); 8.35 (d, J=2.0 Hz, 1H); 8.70 to 8.83 (m, 2H); 9.29 (d, J=2.4 Hz, 1H).

Mass spectrum (LC/MS method C): Retention time Tr (min)=1.14; [M+H]+: m/z 425.

10 mg of 2-bromo-4-(4-quinolin-3-ylindazol-2-yl)benzonitrile are also obtained in the form of an off-white solid, the characteristics of which are the following:

¹H NMR spectrum (400 MHz, δ in ppm, DMSO-d₆); 7.49 to 7.59 (m, 2H); 7.71 (ddd, J=1.3 and 6.8 and 8.2 Hz, 1H); 7.75 to 7.91 (m, 2H); 8.10 to 8.20 (m, 3 H); 8.47 (dd, J=2.2 and 8.6 Hz, 1H); 8.78 (d, J=2.0 Hz, 1H); 8.80 (d, J=1.8 Hz, 1 H) 9.34 (d, J=2.2 Hz, 1H); 9.62 (s, 1H).

Mass spectrum (LC/MS method C): Retention time Tr (min)=1.14; [M+H]+: m/z 425; [M−H]−+HCOOH: m/z 469.

Stage 6: In a 100 ml round-bottomed flask, a mixture, degassed beforehand with argon, of 223 mg of 2-bromo-4-(4-quinolin-3-ylindazol-1-yl)benzonitrile obtained according to the preceding stage, 121 mg of trans-4-aminocyclohexanol, 512 mg of cesium carbonate, 34 mg of 4,5-bis(diphenylphosphino)-9,9-dimethylxanthene and 12 mg of palladium acetate in 18 ml of dioxane is heated for 4 hours at 95° C. under argon. The reaction medium is evaporated to dryness under vacuum and the residue is chromatographed on silica gel (15-40 μm), elution being carried out with a mixture of ethyl acetate and cyclohexane (30:70 then 50:50 then 70:30 v/v). 61 mg of 4-(4-quinolin-3-ylindazol-1-yl)benzonitrile are obtained in the form of a white solid, the characteristics of which are the following:

¹H NMR spectrum (400 MHz, δ in ppm, DMSO-d6): 7.65 to 7.78 (m, 3H); 7.85 (ddd, J=1.6 and 6.8 and 8.4 Hz, 1H); 8.03 to 8.20 (m, 7H); 8.73 (s, 1H); 8.79 (d, J=2.2 Hz, 1H); 9.30 (d, J=2.4 Hz, 1H).

Mass spectrum (LC/MS method B): Retention time Tr (min)=4.42; [M+H]+: m/z 347.

57 mg of 2-(trans-4-hydroxycyclohexylamino)-4-(4-quinolin-3-ylindazol-1-yl)benzonitrile are also obtained in the form of a yellowish foam, the characteristics of which are the following:

¹H NMR spectrum (400 MHz, δ in ppm, DMSO-d6): 1.22 to 1.50 (m, 4H); 1.86 (d, J=12.7 Hz, 2H); 1.94 to 2.00 (m, 2H); 3.38 to 3.58 (m, 2H); 4.55 (d, J=4.6 Hz, 1H); 5.90 (d, J=7.8 Hz, 1H); 7.13 (dd, J=2.0 and 8.3 Hz, 1H); 7.20 (d, J=2.2 Hz, 1H); 7.63 (d, J=6.8 Hz, 1H); 7.67 to 7.76 (m, 3H); 7.85 (ddd, J=1.5 and 6.9 and 8.5 Hz, 1H); 7.98 (d, J=8.6 Hz, 1H); 8.12 (d, J=8.0 Hz, 1H); 8.16 (d, J=8.0 Hz, 1H); 8.67 (s, 1H); 8.78 (d, J=2.2 Hz, 1H); 9.30 (d, J=2.4 Hz, 1H).

Mass spectrum (LC/MS method B): Retention time Tr (min)=4.31; [M+H]₊: m/z 460.

Stage 7: In a 100 ml round-bottomed flask, 236 μl of 1M sodium hydroxide and then 230 μl of a solution of hydrogen peroxide at 30% are successively added, at ambient temperature, to a mixture of 57 mg of 2-(trans-4-hydroxycyclohexylamino)-4-(4-quinolin-3-ylindazol-1-yl)benzonitrile obtained according to the preceding stage, in 2.0 ml of dimethyl sulphoxide and 5.0 ml of ethanol. After stirring for 30 minutes, 40 ml of distilled water are added and the mixture is then extracted three times with 40 ml of ethyl acetate, salting out with sodium chloride. The combined organic phases are washed twice with 40 ml of a saturated solution of sodium chloride, dried over magnesium sulphate and then evaporated to dryness under vacuum. The solid residue is triturated from 10 ml of isopropyl ether, filtered, washed with isopropyl ether and then pentane, and dried at 40° C. under vacuum. 41 mg of 2-(trans-4-hydroxycyclohexylamino)-4-(4-quinolin-3-ylindazol-1-yl)benzamide are obtained in the form of a yellowish solid, the characteristics of which are the following:

¹H NMR spectrum (400 MHz, δ in ppm, DMSO-d₆): 1.21 to 1.41 (m, 4H); 1.84 (d, J=11.7 Hz, 2H); 2.04 (d, J=12.7 Hz, 2H); 3.36 to 3.55 (m, 2H); 4.53 (d, J=4.4 Hz, 1H); 6.93 (dd, J=2.1 and 8.4 Hz, 1H); 7.03 (d, J=2.2 Hz, 1H); 7.20 (broad s, 1H); 7.61 (d, J=6.8 Hz, 1H); 7.65 to 7.75 (m, 2H); 7.79 to 7.97 (m, 4H); 8.09 to 8.19 (m, 2H); 8.49 (d, J=7.6 Hz, 1H); 8.62 (s, 1H); 8.78 (d, J=2.4 Hz, 1 H); 9.31 (d, J=2.4 Hz, 1H).

Mass spectrum (LC/MS method C): Retention time Tr (min)=0.83; [M+H]+: m/z 478.

### EXAMPLE 25

### Synthesis of 4-(4-quinolin-3-ylindazol-1-yl)benzamide

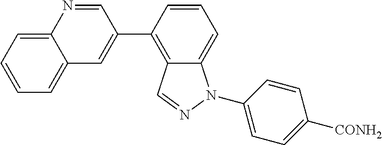

In a 100 ml round-bottomed flask, 335 μl of 1M sodium hydroxide and then 330 μl of a solution of hydrogen peroxide at 30% are successively added, at ambient temperature, to a mixture of 61 mg of 4-(4-quinolin-3-ylindazol-1-yl)benzonitrile obtained according to stage 6 of Example 24, in 3.0 ml of dimethyl sulphoxide and 10 ml of ethanol. After stirring for 30 minutes, 50 ml of distilled water and 50 ml of ethyl acetate are added. After separation by settling out, the aqueous phase is re-extracted three times with 50 ml of ethyl acetate. The combined organic phases are washed with 50 ml of a saturated solution of sodium chloride, dried over magnesium sulphate and then evaporated to dryness under vacuum. 25 mg of 4-(4-quinolin-3-ylindazol-1-yl)benzamide are obtained in the form of an off-white solid, the characteristics of which are the following:

¹H NMR spectrum (400 MHz, δ in ppm, DMSO-d6): 7.44 (broad s, 1H); 7.64 (d, J=6.6 Hz, 1H); 7.71 (t, J=7.8 Hz, 2H); 7.85 (ddd, J=1.6 and 6.8 and 8.4 Hz, 1H); 7.94 (d, J=8.8 Hz, 2H); 8.04 (d, J=8.6 Hz, 1H); 8.07 to 8.21 (m, 5H); 8.67 (s, 1H); 8.79 (d, J=2.2 Hz, 1H); 9.31 (d, J=2.4 Hz, 1H).

Mass spectrum (LC/MS method C): Retention time Tr (min)=0.79; [M+H]+: m/z 365.

### EXAMPLE 26

### Synthesis of 5-(3-chloro-4-quinolin-3-ylindazol-1-yl)-3-(trans-4-hydroxycyclohexylamino)pyridine-2-carboxamide

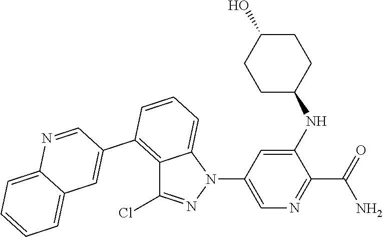

Stage 1: In a 10 ml round-bottomed flask, a mixture of 88 mg of trifluoromethanesulphonic acid 1H-indazol-4-yl ester obtained according to stage 3 of Example 24 and 46 mg of N-chlorosuccinimide in 3 ml of dimethylformamide is heated for 15 minutes at 150° C. under argon. After cooling to ambient temperature, the reaction medium is evaporated to dryness under vacuum and the residue is chromatographed on silica gel (15-40 μm), elution being carried out with a mixture of ethyl acetate and n-heptane (20:80 v/v). 67 mg of trifluoromethanesulphonic acid 3-chloro-1H-indazol-4-yl ester are obtained in the form of a white solid, the characteristics of which are the following:

¹H NMR spectrum (400 MHz, δ in ppm, DMSO-d6): 7.28 (d, J=7.3 Hz, 1H); 7.55 (t, J=8.3 Hz, 1H); 7.72 (d, J=8.8 Hz, 1H).

Mass spectrum (LC/MS method C): Retention time Tr (min)=1.08; [M+H]+m/z 301; [M−H]−: m/z 299.

Stage 2:

Method A: In a 250 ml round-bottomed flask, argon is bubbled, for 5 minutes, into a mixture of 67 mg of trifluoromethanesulphonic acid 3-chloro-1H-indazol-4-yl ester obtained according to the preceding stage, 58 mg of 3-quinolineboronic acid and 71 mg of sodium carbonate in a mixture of 2.5 ml of ethanol, 2.5 ml of toluene and 0.5 ml of distilled water. 39 mg of tetrakis(triphenylphosphine)palladium(0) are then added and the mixture is heated at 95° C. under argon for 4.5 hours, then 10 mg of tetrakis(triphenylphosphine)palladium(0) and 10 mg of 3-quinolineboronic acid are added and then the heating is continued for 8.5 hours. After cooling to ambient temperature, the reaction medium is evaporated to dryness under vacuum. The residue is taken up with 25 ml of a saturated solution of sodium chloride and extracted with four times 20 ml of ethyl acetate. The combined organic phases are washed with 25 ml of a saturated solution of sodium chloride, dried over magnesium sulphate and evaporated to dryness under vacuum. The residue is chromatographed on silica gel (15-40 μm), elution being carried out with a mixture of ethyl acetate and n-heptane (20:80 v/v). 18 mg of 3-(3-chloro-1H-indazol-4-yl)quinoline are obtained in the form of a beige solid, the characteristics of which are the following:

¹H NMR spectrum (400 MHz, δ in ppm, DMSO-d6): 7.27 (d, J=6.8 Hz, 1H); 7.57 (dd, J=7.1 and 8.6 Hz, 1H); 7.64 to 7.71 (m, 2H); 7.83 (ddd, J=1.6 and 6.8 and 8.4 Hz, 1H); 8.09 (t, J=8.2 Hz, 2H); 8.49 (d, 1H); 9.03 (d, J=2.2 Hz, 1H); 13.53 (1H).

Mass spectrum (LC/MS method C): Retention time Tr (min)=0.87; [M+H]+: m/z 280; [M−H]−: m/z 278.

Method B: In a 20 ml round-bottomed flask, a mixture of 160 mg of 3-(1H-indazol-4-yl)quinoline obtained according to stage 4 of Example 24 and 92 mg of N-chlorosuccinimide in 5 ml of anhydrous dimethylformamide is heated at 150° C. under argon for 45 minutes. After cooling to ambient temperature, the reaction medium is evaporated to dryness under vacuum and the residue is chromatographed on silica gel (15-40 μm), elution being carried out with a mixture of ethyl acetate and n-heptane (20:80 v/v). 62 mg of 3-(3-chloro-1H-indazol-4-yl)quinoline are obtained in the form of a beige solid, the characteristics of which are the same as those described for method A above.

Stage 3: In a 100 ml round-bottomed flask under argon, 18 mg of sodium hydride dispersed at 60% in petroleum jelly are added to a solution of 80 mg of 3-(3-chloro-1H-indazol-4-yl)quinoline obtained according to the preceding stage, in 5 ml of anhydrous dimethylformamide. This mixture is heated at 50° C. for 10 minutes, then 74 mg of 2-cyano-5-fluoro-3-(trans-4-hydroxycyclohexylamino)pyridine obtained according to stage 1 of Example 16 are added, at this temperature, then the temperature is increased to 80° C. and this temperature is maintained for 4 hours. After cooling to ambient temperature, the reaction medium is evaporated to dryness under vacuum and the residue is chromatographed on silica gel (15-40 μm), elution being carried out with a mixture of ethyl acetate and n-heptane (30:70 then 50:50 v/v). 50 mg of 5-(3-chloro-4-quinolin-3-ylindazol-1-yl)-3-(trans-4-hydroxycyclohexylamino)pyridine-2-carbonitrile are obtained in the form of a beige foam, the characteristics of which are the following:

¹H NMR spectrum (400 MHz, δ in ppm, DMSO-d6): 1.31 (m, J=12.7 Hz, 2H); 1.46 (q, J=13.4 Hz, 2H); 1.86 (d, J=11.5 Hz, 2H); 1.94 (d, J=12.2 Hz, 2H); 3.43 (m, 1H); 3.58 (m, 1H); 4.55 (d, J=4.4 Hz, 1H); 6.36 (d, J=8.3 Hz, 1H); 7.49 (d, J=7.1 Hz, 1H); 7.66 (d, J=2.2 Hz, 1H); 7.71 (ddd, J=1.2 and 6.8 and 8.1 Hz, 1H); 7.78 (dd, J=7.1 and 8.8 Hz, 1H); 7.86 (ddd, J=1.6 and 6.8 and 8.4 Hz, 1H); 8.05 (d, J=8.6 Hz, 1H); 8.12 (t, J=7.8 Hz, 1H); 8.35 (d, J=2.2 Hz, 1H); 8.55 (d, J=2.4 Hz, 1H); 9.06 (d, J=2.2 Hz, 2H).

Mass spectrum (LC/MS method C): Retention time Tr (min)=1.02; [M+H]+: m/z 495; [M−H]−: m/z 493.

Stage 4: In a 100 ml round-bottomed flask, 202 μl of 1M sodium hydroxide and then 187 μl of a solution of hydrogen peroxide at 30% are successively added, at ambient temperature, to a mixture of 50 mg of 5-(3-chloro-4-quinolin-3-ylindazol-1-yl)-3-(trans-4-hydroxycyclohexylamino)pyridine-2-carbonitrile obtained according to the preceding stage, in 1.0 ml of dimethyl sulphoxide and 3.0 ml of ethanol. After stirring for 20 minutes, 20 ml of distilled water are added. The aqueous phase is extracted 3 times with 20 ml of ethyl acetate, salting out with sodium chloride. The combined organic phases are washed with 30 ml of a saturated solution of sodium chloride, dried over magnesium sulphate and then evaporated to dryness under vacuum. 45 mg of 5-(3-chloro-4-quinolin-3-ylindazol-1-yl)-3-(trans-4-hydroxycyclohexylamino)pyridine-2-carboxamide are obtained in the form of a pale yellow foam, the characteristics of which are the following:

¹H NMR spectrum (400 MHz, O in ppm, DMSO-d6): 1.13 (m, 4H); 1.83 (d, J=12.7 Hz, 2H); 2.03 (d, J=13.0 Hz, 2H); 3.50 (m, 2H); 4.55 (d, J=4.2 Hz, 1H); 7.47 (d, J=6.8 Hz, 1H); 7.50 to 7.53 (m, 2H); 7.73 (m, 2H); 7.86 (ddd, J=1.7 and 7.0 and 8.4 Hz, 1H); 7.99 (d, J=8.6 Hz, 1H); 8.07 (broad s, 1H); 8.12 (t, J=7.6 Hz, 2H); 8.16 (d, J=2.2 Hz, 1H); 8.55 (d, J=2.4 Hz, 1H); 8.75 (d, J=8.1 Hz, 1H); 9.07 (d, J=2.2 Hz, 1H).

Mass spectrum (LC/MS method C): Retention time Tr (min)=0.96; [+H]+: m/z 513.

### EXAMPLE 27

### Synthesis of 5-(3-bromo-4-quinolin-3-ylindazol-1-yl)-3-(2-hydroxy-2-methylpropylamino)pyridine-2-carboxamide

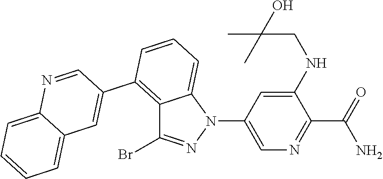

Stage 1: In a 100 ml round-bottomed flask, argon is bubbled, for 10 minutes, into a mixture of 323 mg of 3-bromo-4-iodo-1H-indazole, 260 mg of 3-quinolineboronic acid and 318 mg of sodium carbonate in a mixture of 10 ml of ethanol, 10 ml of toluene and 1.5 ml of distilled water. 173 mg of tetrakis(triphenylphosphine)palladium(0) are added under argon and the reaction medium is heated at 95° C. for 4 hours. After cooling to ambient temperature, the reaction medium is evaporated to dryness under vacuum and the residue is taken up with 50 ml of a saturated solution of sodium chloride and the aqueous phase is extracted with four times 50 ml of ethyl acetate. The combined organic phases are washed with 50 ml of a saturated solution of sodium chloride, dried over magnesium sulphate and evaporated to dryness under vacuum. The residue is chromatographed on silica gel (15-40 μm), elution being carried out with a mixture of ethyl acetate and n-heptane (20:80 v/v), 175 mg of 3-(3-bromo-1H-indazol-4-yl)quinoline are obtained in the form of a pale yellow solid, the characteristics of which are the following:

¹H NMR spectrum (400 MHz, δ in ppm, DMSO-d6): 7.24 (dd, J=0.7 and 7.1 Hz, 1H); 7.57 (dd, J=7.0 and 8.4 Hz, 1H); 7.69 (m, 2H); 7.82 (ddd, J=1.6 and 6.8 and 8.4 Hz, 1H); 8.10 (t, J=8.4 Hz, 2H); 8.47 (d, J=2.0 Hz, 1H); 9.01 (d, J=2.2 Hz, 1H); 13.69 (s, 1H).

Mass spectrum (LC/MS method C): Retention time Tr (min)=0.79; [M+H]+: m/z 324; [M−H]−: m/z 322.

Stage 2: In a 20 ml round-bottomed flask, 15 mg of sodium hydride as a dispersion at 60% in petroleum jelly are added, under argon at ambient temperature, to a mixture of 81 mg of 3-(3-bromo-1H-indazol-4-yl)quinoline obtained according to the preceding stage, in 3 ml of anhydrous dimethylformamide. The reaction medium is heated at 50° C. and a solution of 58 mg of 5-fluoro-3-(2-hydroxy-2-methylpropylamino)pyridine-2-carbonitrile obtained according to stage 1 of Example 9, in 2 ml of anhydrous dimethylformamide, is added at this temperature of 50° C., and then the reaction medium is brought to 80° C. under argon for 4 hours. After cooling to ambient temperature, the reaction medium is evaporated to dryness under vacuum and the residue is chromatographed on silica gel (15-40 μm), elution being carried out with a mixture of ethyl acetate and n-heptane (20:80 then 50:50 v/v). 27 mg of 5-(3-bromo-4-quinolin-3-ylindazol-1-yl)-3-(2-hydroxy-2-methylpropylamino)pyridine-2-carbonitrile are obtained in the form of a pale yellow solid, the characteristics of which are the following:

¹H NMR spectrum (400 MHz, δ in ppm, DMSO-d6): 1.20 (s, 6H); 3.25 (masked m, 2H); 4.80 (s, 1H); 6.42 (t, J=6.0 Hz, 1H); 7.45 (d, J=7.1 Hz, 1H); 7.73 (m, 2H); 7.85 (m, 2H); 8.11 (m, 3H); 8.32 (d, J=2.0 Hz, 1H); 8.52 (d, J=1.5 Hz, 1H); 9.04 (d, J=2.2 Hz, 1H).

Mass spectrum (LC/MS method C): Retention time Tr (min)=[M+H]+: m/z 513; [M−H]−: m/z 511.

Stage 3: In a 25 ml round-bottomed flask, 100 μl of 1M sodium hydroxide and then 100 μl of a solution of hydrogen peroxide at 30% are successively added, at ambient temperature, to a mixture of 27 mg of 5-(3-bromo-4-quinolin-3-ylindazol-1-yl)-3-(2-hydroxy-2-methylpropylamino)pyridine-2-carbonitrile obtained according to the preceding stage, in 1.0 ml of dimethyl sulphoxide and 3.0 ml of ethanol. After stirring for 15 minutes, 20 ml of distilled water are added. The aqueous phase is extracted four times with 20 ml of ethyl acetate, salting out with sodium chloride. The combined organic phases are washed with 25 ml of a saturated solution of sodium chloride, dried over magnesium sulphate and then evaporated to dryness under vacuum. 25 mg of 5-(3-bromo-4-quinolin-3-ylindazol-1-yl)-3-(2-hydroxy-2-methylpropylamino)pyridine-2-carboxamide are obtained in the form of a pale yellow solid, the characteristics of which are the following:

¹H NMR spectrum (400 MHz, δ in ppm, DMSO-d6): 1.21 (s, 6H); 3.19 (d, J=5.6 Hz, 2H); 4.64 (s, 1H); 7.43 (d, J=6.8 Hz, 1H); 7.47 (broad s, 1H); 7.55 (d, J=2.4 Hz, 1H); 7.72 (m, 2H); 7.85 (ddd, J=1.7 and 7.0 and 8.4 Hz, 1H); 8.05 (m, 2H); 8.12 (t, J=7.8 Hz, 2H); 8.15 (d, J=2.2 Hz, 1H); 8.53 (d, J=2.4 Hz, 1H); 8.96 (t, J=5.6 Hz, 1H); 9.05 (d, J=2.2 Hz, 1H).

Mass spectrum (LC/MS method C): Retention time Tr (min)=1.06; [M+H]+: m/z 531; [M−H]−: m/z 529.

### EXAMPLE 28

### Pharmaceutical Composition

Tablets corresponding to the following formula were prepared:

| | |
|---|---|
| Product of Example 9 | 0.2 g |
| Excipient for a tablet with a final weight of | 1 g |

(details of the excipient: lactose, talc, starch, magnesium stearate).

### EXAMPLE 29

### Pharmaceutical Composition

Tablets corresponding to the following formula were prepared:

| | |
|---|---|
| Product of Example 21 | 0.2 g |
| Excipient for a tablet with a final weight of | 1 g |

(details of the excipient: lactose, talc, starch, magnesium stearate).

The present invention also comprises all the pharmaceutical compositions prepared with any product of formula (I) as appropriate, according to the present invention.

Biological tests for biologically characterizing the products of the invention:

### 1) Biochemical Activity:

The biochemical activity of the compounds can in particular be evaluated by means of the “Hsp82/ATPase” test described below:

The inorganic phosphate released during the hydrolysis of ATP by the ATPase activity of Hsp82 is quantified by the malachite green method. In the presence of this reagent, formation of the inorganic phosphate-molybdate-malachite green complex, which absorbs at a wavelength of 620 nm, occurs.

The products to be evaluated are incubated in a reaction volume of 30 μl, in the presence of 1 μM Hsp82 and of 250 μM of substrate (ATP) in a buffer composed of 50 mM Hepes-NaOH (pH 7.5), 1 mM DTT, 5 mM MgCl₂ and 50 mM KCl at 37° C. for 60 min. In parallel, an inorganic phosphate range of between 1 and 40 μM is made up in the same buffer. The ATPase activity is then revealed by adding 60 μl of biomol green reagent (Tebu), After incubation at ambient temperature for 20 min, the absorbance of the various wells is measured using a microplate reader at 620 nm. The inorganic phosphate concentration of each sample is then calculated from the standard curve. The ATPase activity of Hsp82 is expressed as concentration of inorganic phosphate produced in 60 minutes. The effect of the various products tested is expressed as percentage inhibition of the ATPase activity.

The formation of ADP due to the ATPase activity of Hsp82 was used to develop another method for evaluating the enzyme activity of this enzyme by application of an enzymatic coupling system involving pyruvate kinase (PK) and lactate dehydrogenase (LDH). In this kinetic-type spectrophotometric method, PK catalyses the formation of ATP and of pyruvate from phosphoenol pyruvate (PEP) and the ADP produced by Hsp82. The pyruvate formed, which is a substrate for LDH, is subsequently converted to lactate in the presence of NADH. In this case, the decrease in the NADH concentration, measured through the decrease in absorbance at the wavelength of 340 nm, is proportional to the concentration of ADP produced by Hsp82.

The products tested are incubated in a reaction volume of 100 μl of buffer composed of 100 mM Hepes-NaOH (pH 7.5), 5 mM MgCl₂, 1 mM DTT, 150 mM KCl, 0.3 mM NADH, 2.5 mM PEP and 250 μM ATP. This mixture is preincubated at 37° C. for 30 minutes before the addition of 3.77 units of LDH and 3.77 units of PK. The reaction is initiated by addition of the product to be evaluated, in varying concentrations, and of Hsp82, at the concentration of 1 μM. The enzymatic activity of Hsp82 is then measured continuously, in a microplate reader, at 37° C., at the wavelength of 340 nm. The initial rate of the reaction is obtained by measuring the slope of the tangent to the origin of the curve recorded. The enzymatic activity is expressed in μM of ADP formed per minute. The effect of the various products tested is expressed as 50% inhibitory concentration (1050) with respect to the ATPase activity, according to the codification below:

### A: IC₅₀<1 μM

### 1 μM<IC₅₀<10 μM

### C: 10 μM<IC₅₀<100 μM

n.d.: not determined

### 2) Cellular Activity:

The cellular activity of the compounds can in particular be evaluated by means of the phenotypic “SKBr₃/HER2” cell test described below:

The SKBr₃ mammalian adenocarcinoma cells, overexpressing the Her2 tyrosine kinase receptor, originate from the ATCC (HTB-30) and are cultured in McCoy's 5A medium supplemented with 10% FBS and 1% L-glutamine.

The cells are seeded in 12-well plates at a proportion of 125 000 cells per well in 1 ml of complete medium. The following day, the products are added at varying concentrations. After incubation for 24 h, the cells are trypsinized, washed with PBS and incubated with 100 ng of anti-Her2 antibody coupled to PE (phycoerythrin) (BD 340552) for 30 minutes at 4° C. in the dark. The fluorescence due to the expression of the Her2 receptor at the surface of the cells is then read using a FACS Calibur flow cytometer (Becton-Dickinson). The percentage inhibition of Her2 expression as a function of the concentrations of product tested is fitted by the nonlinear regression technique (XLfit, equation 205) in order to measure the IC₅₀ for each product.

The activity of the products is codified as follows:

### A: IC₅₀<1 μM

### B: 1 μM<IC₅₀<10 μM

### C: 10 μM<IC₅₀<100 μM

n.d.: not determined

The summarizing table below gives the biochemical and cellular activities of representative compounds of the invention.

Table of results

| | | Hsp82 | SKBR3 |
|---|---|---|---|
| | | ATPase | HER2 |
| | | IC50 | IC50 |
| Ex | Structure | μM | μM |
| 1 | 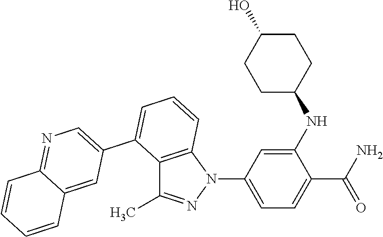 | A | A |
| 2 | 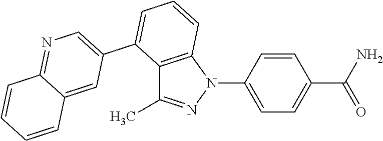 | B | C |
| 3 | 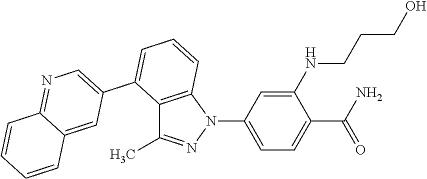 | A | A |
| 4 | 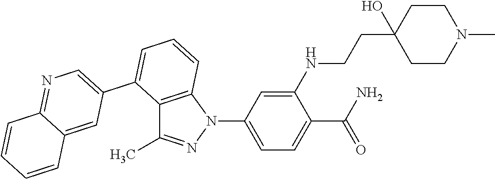 | n. d. | A |
| 5 | 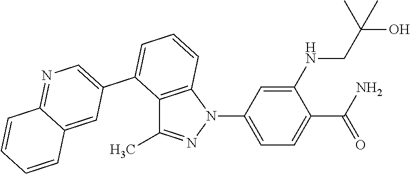 | A | A |
| 6 | 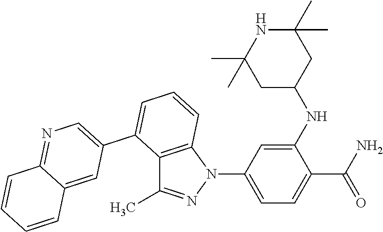 | A | A |
| 7 | 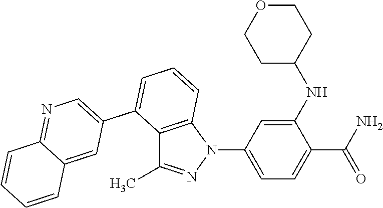 | A | A |
| 8 | 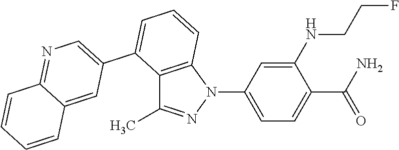 | n. d. | A |
| 9 | 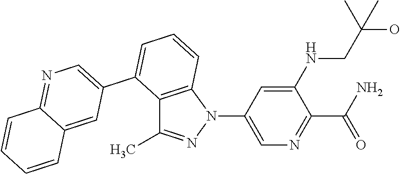 | n. d. | A |
| 10 | 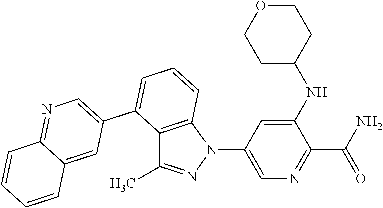 | A | A |
| 11 | 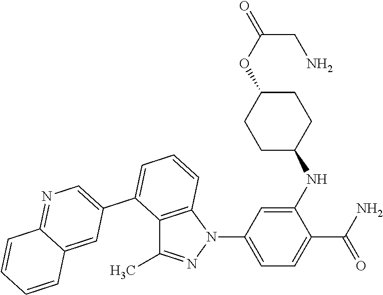 | A | A |
| 12 | 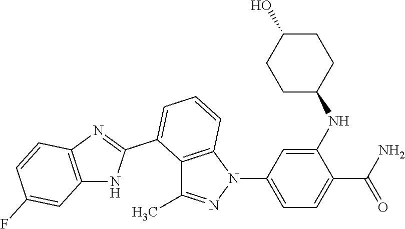 | A | A |
| 13 | 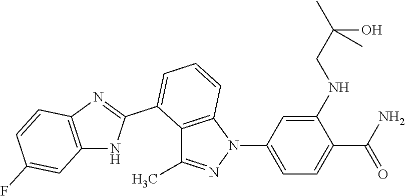 | n. d. | A |
| 14 | 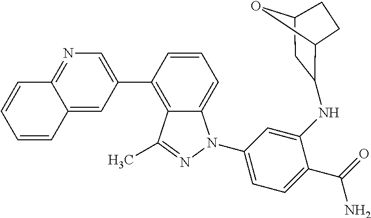 | n. d. | A |
| 15 | 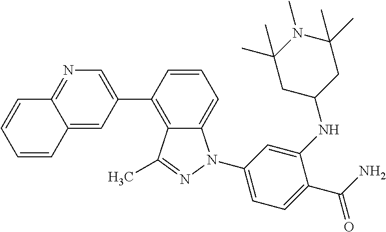 | B | A |
| 16 | 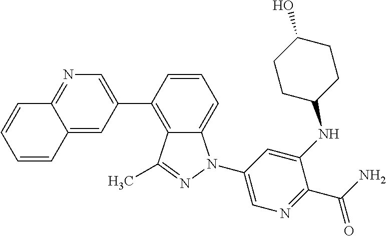 | n. d. | A |
| 17 | 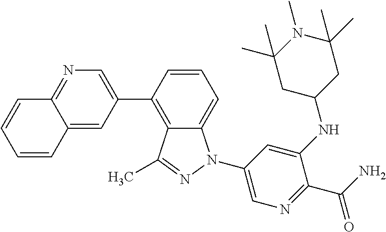 | n. d. | A |
| 18 | 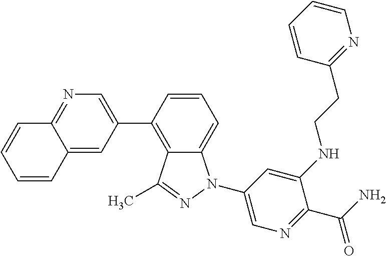 | A | B |
| 19 | 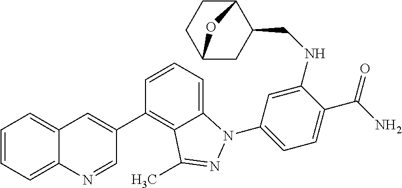 | n. d. | A |
| 20 | 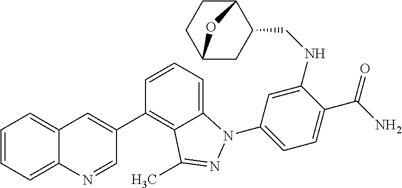 | n. d. | A |
| 21 | 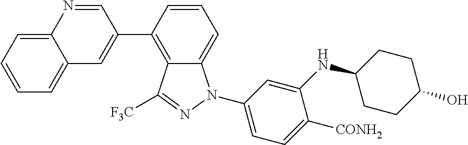 | n. d. | A |
| 22 | 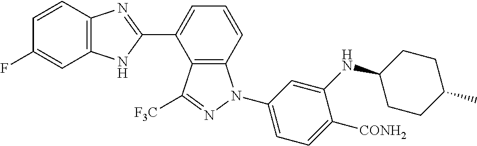 | n. d. | B |
| 23 | 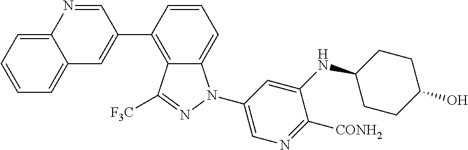 | n. d. | A |
| 24 | 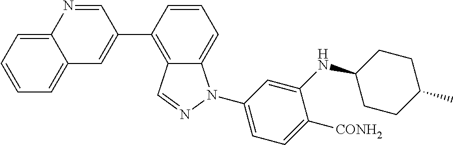 | n. d. | A |
| 25 | 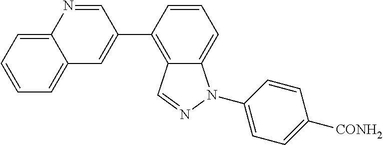 | n. d. | C |
| 26 | 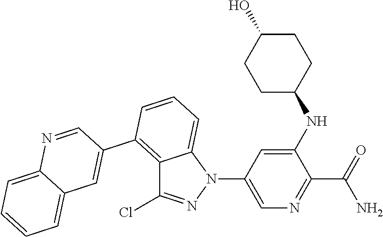 | n. d. | A |
| 27 | 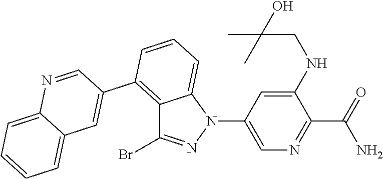 | n. d. | A |

1. A compound of formula (I): 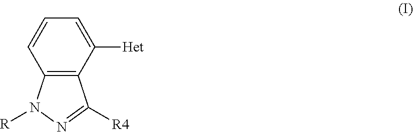
in which:
R4 represents H, CH₃, CH₂CH₃, CF₃, F, Cl, Br or I;
Het represents a monocyclic or bicyclic, aromatic or partially unsaturated heterocycle—of dihydro or tetrahydro type—, with from 5 to 11 ring members, containing from 1 to 4 heteroatoms chosen from N, O or S, optionally substituted with one or more radicals R1 or R′1, which may be identical or different, as described below,
R is chosen from the group constituted of 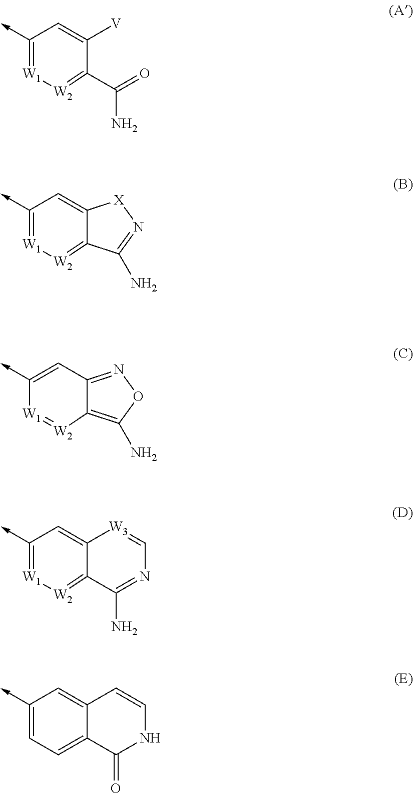
with R1 and/or R′1, which may be identical or different, chosen from the group constituted of H, halogen, CF₃, nitro, cyano, alkyl, hydroxyl, mercapto, amino, alkylamino, dialkylamino, alkoxy, phenylalkoxy, alkylthio, carboxyl in free form or esterified with an alkyl radical, carboxamide, CO—NH(alkyl), CON(alkyl)₂, NH—CO-alkyl, sulphonamide, NH—SO₂-alkyl, S(O)₂—NHalkyl and S(O₂)—N(alkyl)₂, all the alkyl, alkoxy and alkylthio radicals being themselves optionally substituted with one or more radicals, which may be identical or different, chosen from halogen, hydroxyl, alkoxy, amino, alkylamino and dialkylamino;
W1, W2 and W3 independently represent CH or N;
X represents an oxygen or sulphur atom, or an NR2, C(O), S(O) or S(O)₂ radical;
V represents a hydrogen atom or halogen atom or an —O—R2 radical or an —NH—R2 radical in which:
R2 represents a hydrogen atom or a C₁-C₆ alkyl radical, or a C₃-C₈ cycloalkyl radical or a C₃-C₁₀ heterocycloalkyl radical, which is monocyclic or bicyclic; these alkyl, cycloalkyl and heterocycloalkyl radicals being optionally substituted with one or more radicals, which may be identical or different, chosen from the radicals:
—O—PO₃H₂; —O—PO₃Na₂; —O—SO₃H₂; —O—SO₃Na₂; —O—CH₂—PO₃H₂; —O—CH₂—PO₃Na₂; —O—CO-alanine; —O—CO-glycine; —O—CO-serine; —O—CO-lysine; —O—CO-arginine; —O—CO-glycine-lysine; —O—CO-alanine-lysine;
halogen; hydroxyl; mercapto; amino; carboxamide (CONH₂); carboxyl;
heterocycloalkyl; cycloalkyl; heteroaryl; carboxyl esterified with an alkyl radical; —CO—NH(alkyl); —O—CO-alkyl; —NH—CO-alkyl; alkyl; alkoxy; alkylthio; alkylamino; dialkylamino; in all the latter radicals, the alkyl, alkoxy and alkylthio radicals being themselves optionally substituted with one or more radicals, which may be identical or different, chosen from hydroxyl, mercapto, amino, alkylamino, dialkylamino, CO₂alkyl, NHCO₂alkyl and heterocycloalkyl radicals; in all these radicals, the cycloalkyl, heterocycloalkyl and heteroaryl radicals being themselves optionally substituted with one or more radicals, which may be identical or different, chosen from hydroxyl, alkyl, alkoxy, CH₂OH, amino, alkylamino, dialkylamino, CO₂alkyl or NHCO₂alkyl radicals;
all the possible tautomeric and isomeric forms: racemic, enantiomeric and diastereoisomeric, and pharmaceutically acceptable salts thereof.

2. The compound of claim 1, in which:
R4 represents CH₃, CH₂CH₃, CF₃, F, Cl, Br or I;
Het is chosen from the group constituted of: 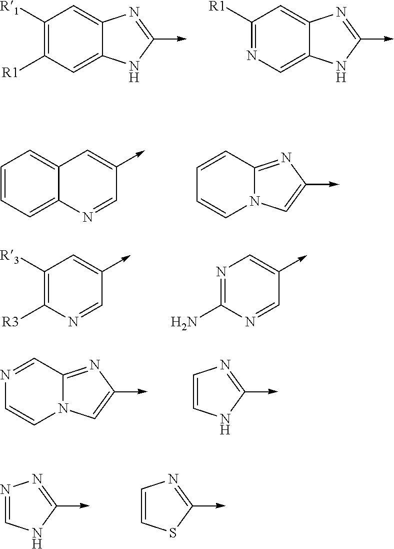
in which R′3 and R3 are such that one represents a hydrogen atom and the other is chosen from the values of R1 and R′1;
R1 and/or R′1, which may be identical or different, are chosen from the group constituted of H, halogen, CF₃, nitro, cyano, alkyl, hydroxyl, mercapto, amino, alkylamino, dialkylamino, alkoxy, phenylalkoxy, alkylthio, carboxyl in free form or esterified with an alkyl radical, carboxamide, CO—NH(alkyl), CON(alkyl)₂, NH—CO-alkyl, sulphonamide, NH—SO₂-alkyl, S(O)₂—NHalkyl and S(O₂)—N(alkyl)₂, all the alkyl, alkoxy and alkylthio radicals being themselves optionally substituted with one or more radicals, which may be identical or different, chosen from halogen, hydroxyl, alkoxy, amino, alkylamino and dialkylamino;
all the possible tautomeric and isomeric forms: racemic, enantiomeric or diastereoisomeric, and pharmaceutically acceptable salts thereof.

3. The compound of claim 1, in which:
R4 represents H, CH₃, CH₂CH₃, CF₃, F, Cl, Br or I;
Het is chosen from the group constituted of: 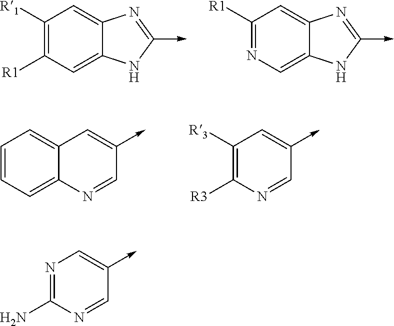
in which R′3 and R3 are such that one represents a hydrogen atom and the other is chosen from the radicals —NH₂, —CN, —CH₂—OH, —CF₃, —OH, —O—CH₂-phenyl, —O—CH₃ and —CO—NH₂;
R is chosen from the group constituted of: 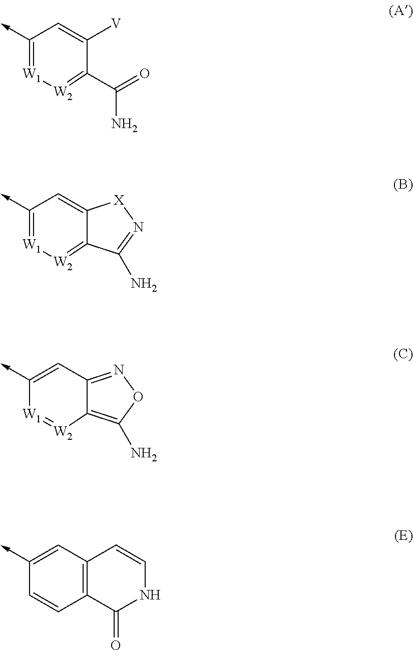
with R1 and/or R′1, which may be identical or different, chosen from the group constituted of H, halogen, CF₃, nitro, cyano, alkyl, hydroxyl, mercapto, amino, alkylamino, dialkylamino, alkoxy, alkylthio, carboxyl in free form or esterified with an alkyl radical; carboxamide, CO—NH(alkyl), CON(alkyl)₂, NH—CO-alkyl, sulphonamide, NH—SO₂-alkyl, S(O)₂—NHalkyl and S(O₂)—N(alkyl)₂, all the alkyl, alkoxy and alkylthio radicals being themselves optionally substituted with one or more radicals, which may be identical or different, chosen from halogen, hydroxyl, alkoxy, amino, alkylamino and dialkylamino;
W1 and W2 independently represent CH or N,
X represents an oxygen or sulphur atom, or an NR2, C(O), S(O) or S(O)₂ radical;
V represents a hydrogen atom or a halogen atom or an —O—R2 radical or an NH—R2 radical in which:
R2 represents a hydrogen atom or a C₁-C₆ alkyl radical, or a C₃-C₈ cycloalkyl radical or a C₃-C₁₀ heterocycloalkyl radical, which is monocyclic or bicyclic; these alkyl, cycloalkyl and heterocycloalkyl radicals being optionally substituted with one or more radicals, which may be identical or different, chosen from the radicals:
halogen; hydroxyl; mercapto; amino; carboxamide (CONH₂); carboxyl;
heterocycloalkyl; cycloalkyl; heteroaryl; carboxyl esterified with an alkyl radical; CO—NH(alkyl); —O—CO-alkyl; —NH—CO-alkyl; alkyl; alkoxy; alkylthio; alkylamino, dialkylamino; in all the latter radicals, the alkyl, alkoxy and alkylthio radicals being themselves optionally substituted with one or more radicals, which may be identical or different, chosen from hydroxyl, mercapto, amino, alkylamino, dialkylamino, CO₂alkyl, NHCO₂alkyl and heterocycloalkyl radicals; in all these radicals, the cycloalkyl, heterocycloalkyl and heteroaryl radicals being themselves optionally substituted with one or more radicals, which may be identical or different, chosen from hydroxyl, alkyl, alkoxy, CH₂OH, amino, alkylamino, dialkylamino, CO₂alkyl or NHCO₂alkyl radicals;
all the possible tautomeric and isomeric forms: racemic, enantiomeric and diastereoisomeric, and pharmaceutically acceptable salts thereof.

4. The compound of claim 1, in which:
R4 represents H, CH₃, CH₂CH₃, CF₃, F, Cl, Br or I;
Het is chosen from the group constituted of: 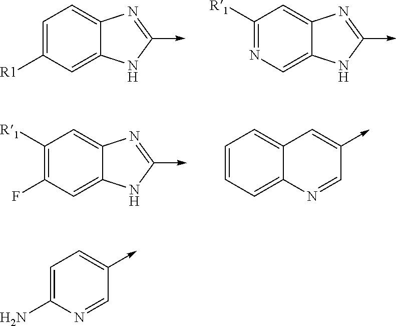
R is chosen from the group constituted of: 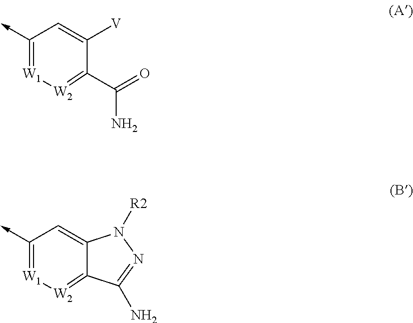
R1 is chosen from the group constituted of H, F, Cl, Br, CF₃, NO₂, CN, CH₃, OH, OCH₃, OCF₃, CO₂Me, CONH₂, CONHMe, CONH—(CH₂)₃—OMe, CONH—(CH₂)₃—N(Me)₂, NHC(O)Me, SO₂NH₂ and SO₂N(Me)₂;
R′1 is chosen from the group constituted of H, CONH₂, CONHMe and OMe;
R″1 is chosen from the group constituted of F, Cl, OH, OMe, CN, O—(CH₂)₃—OMe and O—(CH₂)₃—N(Me)₂;
W1 and W2, which may be identical or different, represent CH or N;
V represents a hydrogen atom or an —NH—R2 radical in which:
R2 represents a hydrogen atom or a C₁-C₆ alkyl, C₃-C₈ cycloalkyl or C₄-C₈ heterocycloalkyl radical, all these alkyl, cycloalkyl and heterocycloalkyl radicals being optionally substituted with one or more radicals, which may be identical or different, chosen from the radicals:
halogen; hydroxyl; amino; carboxamide; carboxyl;
7-oxabicyclo[2.2.1]hept-2-yl; azetidinyl; oxetanyl; tetrahydrofuranyl; tetrahydropyranyl; piperazinyl; alkylpiperazinyl; pyrrolidinyl; morpholinyl; homopiperidinyl; homopiperazinyl; quinuclidinyl; piperidinyl and pyridyl, all these cyclic radicals being themselves optionally substituted with one or more radicals chosen from hydroxyl and alkyl radicals;
carboxyl esterified with an alkyl radical, CO—NH(alkyl), O—CO-alkyl, NH—CO-alkyl, alkyl, alkoxy, methylthio, alkylamino, dialkylamino, all the latter alkyl and alkoxy radicals being themselves optionally substituted with a hydroxyl, mercapto, amino, alkylamino, dialkylamino, azetidino, oxetano, pyrrolidino, tetrahydrofuranyl, piperidino, tetrahydropyranyl, piperazino, morpholino, homopiperidino, homopiperazino or quinuclidino radical;
all the possible tautomeric and isomeric forms: racemic, enantiomeric and diastereoisomeric, and pharmaceutically acceptable salts thereof.

5. The compound of claim 1, in which:
R4 represents H, CH₃, CF₃, Cl or Br;
Het is chosen from the group constituted of: 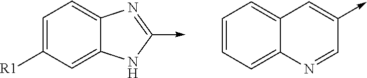
where R1 is chosen from H, F, Cl, Br, CF₃, NO₂, CN, CH₃, OH, OCH₃, OCF₃, CO₂Me, CONH₂, CONHMe, CONH—(CH₂)₃—OMe, CONH—(CH₂)₃—N(Me)₂, NHC(O)Me, SO₂NH, or SO₂N(Me)₂; 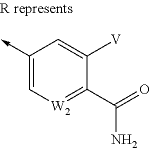
where W2 represents CH or N,
V represents a hydrogen atom or an —NH—R2 radical in which:
R2 represents a C₁-C₄ alkyl radical, a C₃-C₆ cycloalkyl radical or a C₅-C₇ heterocycloalkyl radical, all these alkyl, cycloalkyl and heterocycloalkyl radicals being optionally substituted with one or more radicals, which may be identical or different, chosen from the radicals:
halogen; hydroxyl; amino; carboxamide (CONH₂); carboxyl;
heterocycloalkyl such as tetrahydrofuranyl; piperidinyl; 7-oxabicyclo[2.2.1]hept-2-yl; tetrahydropyranyl; piperazinyl; alkylpiperazinyl; morpholinyl; homopiperidinyl; homopiperazinyl; quinuclidinyl; pyridyl; —O—CO-alkyl; alkyl; alkoxy; alkylamino; dialkylamino; in all these radicals, the alkyl radicals being themselves optionally substituted with one or more radicals, which may be identical or different, chosen from hydroxyl, amino, alkylamino and dialkylamino radicals; the piperidyl radical being itself optionally substituted with one or more radicals, which may be identical or different, chosen from hydroxyl, alkyl, alkoxy, CH₂OH, amino, alkylamino and dialkylamino radicals;
all the possible isomeric forms: tautomeric, racemic, enantiomeric and diastereoisomeric, and pharmaceutically acceptable salts thereof.

6. The compound of claim 1, corresponding to the following names:
2-(trans-4-hydroxycyclohexylamino)-4-(3-methyl-4-quinolin-3-yl)indazol-1-yl)benzamide.
4-(3-methyl-4-quinolin-3-ylindazol-1-yl)benzamide.
2-(3-hydroxypropylamino)-4-(3-methyl-4-quinolin-3-ylindazol-1-yl)benzamide.
2-[2-(4-hydroxy-1-methylpiperidin-4-yl)ethylamino]-4-(3-methyl-4-quinolin-3-ylindazol-1-yl)benzamide.
2-(2-hydroxy-2-methylpropylamino)-4-(3-methyl-4-quinolin-3-ylindazol-1-yl)benzamide.
4-(3-methyl-4-quinolin-3-ylindazol-1-yl)-2-(2,2,6,6-tetramethylpiperidin-4-ylamino)benzamide.
4-(3-methyl-4-quinolin-3-ylindazol-1-yl)-2-(tetrahydropyran-4-ylamino)benzamide.
2-(2-fluoroethylamino)-4-(3-methyl-4-quinolin-3-ylindazol-1-yl)benzamide.
3-(2-hydroxy-2-methylpropylamino)-5-(3-methyl-4-quinolin-3-ylindazol-1-yl)pyridine-2-carboxamide.
5-(3-methyl-4-quinolin-3-ylindazol-1-yl)-3-(tetrahydropyran-4-ylamino)pyridine-2-carboxamide.
trans-4-[2-carbamoyl-5-(3-methyl-4-quinolin-3-ylindazol-1-yDphenylamino]cyclohexyl ester of aminoacetic acid.
4-[4-(6-fluoro-1H-benzimidazol-2-yl)-3-methylindazol-1-yl]-2-(trans-4-hydroxy-cyclohexylamino)benzamide.
4-[4-(6-fluoro-1H-benzimidazol-2-yl)-3-methylindazol-1-yl]-2-(2-hydroxy-2-methyl-propylamino)benzamide.
4-(3-methyl-4-quinolin-3-ylindazol-1-yl)-2-[exo-(7-oxabicyclo[2.2.1]hept-2-yl)amino]benzamide.
4-(3-methyl-4-quinolin-3-ylindazol-1-yl)-2-(1,2,2,6,6-pentamethylpiperidin-4-ylamino)benzamide.
3-(trans-4-hydroxycyclohexylamino)-5-(3-methyl-4-quinolin-3-ylindazol-1-yl)pyridine-2-carboxamide.
5-[3-methyl-4-quinolin-3-ylindazol-1-yl]-3-(1,2,2,6,6-pentamethylpiperidin-1-ylamino)pyridine-2-carboxamide.
5-[3-methyl-4-quinolin-3-ylindazol-1-yl]-[2-pyridin-2-ylethylamino]pyridine-2-carboxamide.
4-(3-methyl-4-quinolin-3-ylindazol-1-yl)-2-{[exo-1-(7-oxabicyclo[2.2.1]hept-2-yl)methyl]amino}benzamide.
4-(3-methyl-4-quinolin-3-ylindazol-1-yl)-2-{[endo-1-(7-oxabicyclo[2.2.1]hept-2-yl)methyl]amino}benzamide.
2-(trans-4-hydroxycyclohexylamino)-4-(4-quinolin-3-yl-3-trifluoromethylindazol-1-yl)benzamide.
4-[4-(6-fluoro-1H-benzimidazol-2-yl)-3-trifluoromethylindazol-1-yl]-2-(trans-4-hydroxycyclohexylamino)benzamide.
3-(trans-4-hydroxycyclohexylamino)-5-(4-quinolin-3-yl-3-trifluoromethylindazol-1-yl)pyridine-2-carboxamide.
2-(trans-4-hydroxycyclohexylamino)-4-(4-quinolin-3-ylindazol-1-yl)benzamide.
4-(4-quinolin-3-ylindazol-1-yl)benzamide.
5-(3-chloro-4-quinolin-3-ylindazol-1-yl)-3-(trans-4-hydroxycyclohexylamino)pyridine-2-carboxamide.
5-(3-bromo-4-quinolin-3-ylindazol-1-yl)-3-(2-hydroxy-2-methylpropylamino)pyridine-2-carboxamide.
and also the addition salts with inorganic and organic acids or with inorganic and organic bases of said products of formula (I).

7. A process for preparing the compound of claim 1 according to scheme (I) hereinafter: 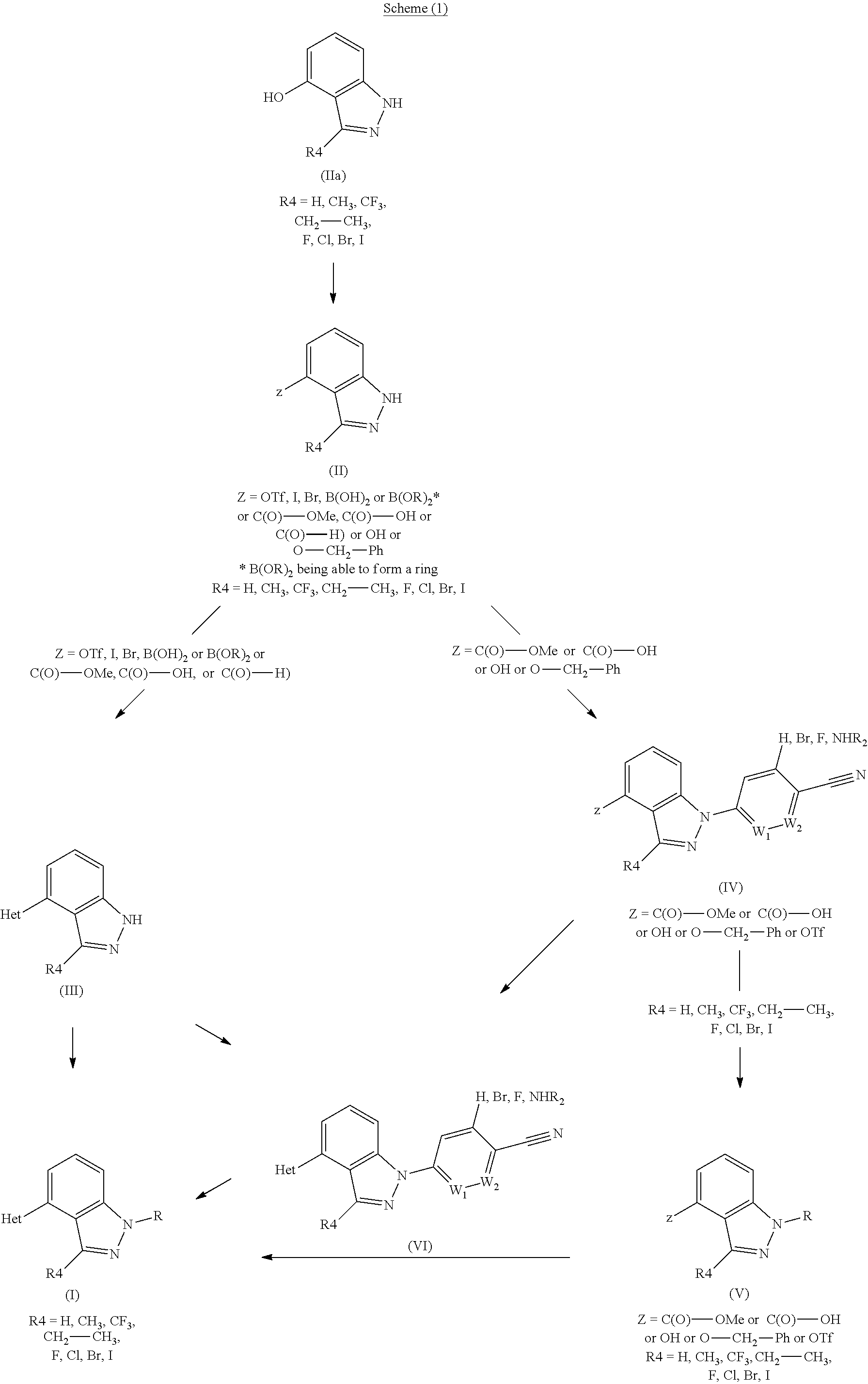
in which the substituents Het, R, R2, R4, W1 and W2 have the meanings indicated in claim 1, and z has the meaning indicated above in scheme (1).

8. A pharmaceutical composition comprising the compound of claim 1, and pharmaceutically acceptable salts thereof.

9. A pharmaceutical composition comprising the compound of claim 6, and pharmaceutically acceptable salts thereof.

10. A pharmaceutical composition containing, as active ingredient, at least one compound according to claim 1, or a pharmaceutically acceptable salt of said compound or a prodrug of said compound, and a pharmaceutically acceptable carrier.

11. (canceled)

12. A method of treating cancers in a patient in need thereof comprising administering to said patient a therapeutically effective amount of the pharmaceutical composition of claim 8.

13. The compound of claim 1, wherein said compound is an Hsp90 inhibitor.

14. A compound having one of the following formulas: 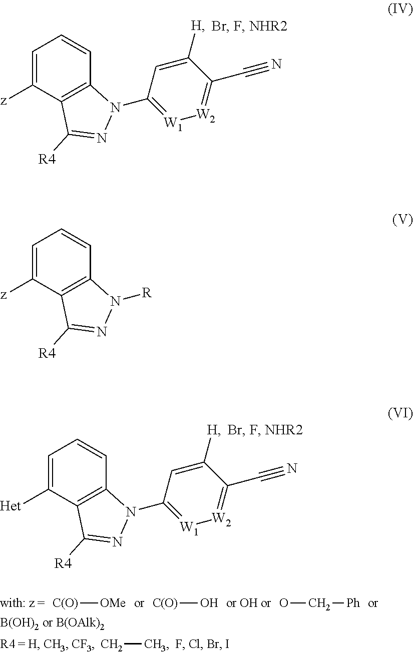
in which the substituents Het, R, R2, R4, W1 and W2 have the meanings indicated in claim 1.
